(19) United States
(12) Patent Application Publication
(10) Pub. No.: US 2024/0376121 A1
(43) Pub. Date: Nov. 14, 2024

(54) COMPOUNDS AND USES THEREOF

(71) Applicant: Foghorn Therapeutics Inc., Indianapolis, IN (US)

(72) Inventors: Matthew NETHERTON, Cambridge, MA (US); Francois BRUCELLE, Belmont, MA (US); Jing DENG, Cambridge, MA (US); Johannes H. VOIGT, Cambridge, MA (US); Kevin J. WILSON, Roslindale, MA (US)

(21) Appl. No.: 18/288,504

(22) PCT Filed: May 10, 2022
(86) PCT No.: PCT/US2022/028511

§ 371 Date: Oct. 26, 2023

Related U.S. Application Data
(60) Provisional application No. 63/325,716, filed on Mar. 31, 2022.
(60) Provisional application No. 63/186,550, filed on May 10, 2021.

(51) Int. Cl. C07D 495/04; A61K 31/5025; A61K 31/506; A61K 31/5377; C07D 495/14; C07D 519/00

(52) U.S. Cl. CPC C07D 495/04; A61K 31/5025; A61K 31/506; A61K 31/5377; C07D 495/14; C07D 519/00

(57) ABSTRACT

The present disclosure features compounds useful for the treatment of BAF complex-related disorders.

### BACKGROUND

The invention relates to compounds useful for modulating BRG1- or BRM-associated factors (BAF) complexes. In particular, the invention relates to compounds useful for treatment of disorders associated with BAF complex function.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 (Brahma-related gene-1) and BRM (Brahma). The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

### SUMMARY

The present invention features compounds useful for modulating a BAF complex. In some embodiments, the compounds are useful for the treatment of disorders associated with an alteration in a BAF complex, e.g., a disorder associated with an alteration in one or both of the BRG1 and BRM proteins. The compounds of the invention, alone or in combination with other pharmaceutically active agents, can be used for treating such disorders.

In an aspect, the invention features a compound, or a pharmaceutically acceptable salt thereof, having the structure of Formula I or II:

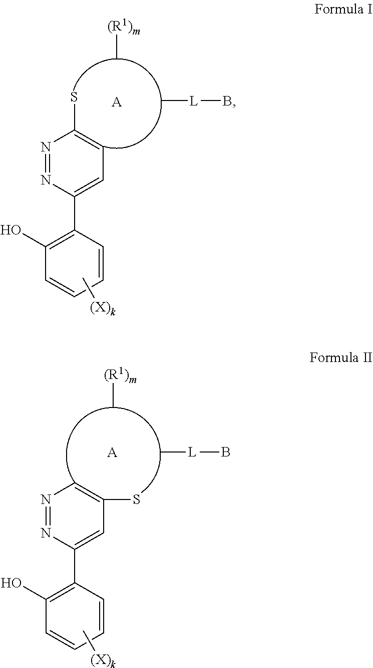

where
ring system A is a 5 to 9-membered heterocyclyl or heteroaryl;
m is 0, 1, 2, or 3;
k is 0, 1, or 2;
each R¹ is, independently, halo, optionally substituted C₁-C₆ alkyl, optionally substituted C₁-C₆ heteroalkyl, optionally substituted C₃-C₈ cycloalkyl, or optionally substituted C₂-C₉ heterocyclyl;
each X is, independently, halo;
L is a linker; and
B is a degradation moiety.

In some embodiments,
ring system A is a 5 to 9-membered heterocyclyl or heteroaryl;
m is 0, 1, 2, or 3;
k is 0, 1, or 2;
each X is, independently, halo;
L is a linker; and
B is a degradation moiety.

In some embodiments, the compound has the structure of Formula I-A or II-A:

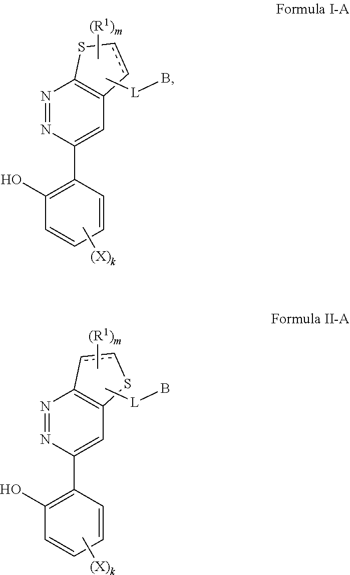

where the dashed bond represents a single or double bond.

In some embodiments, the compound has the structure of Formula I-B:

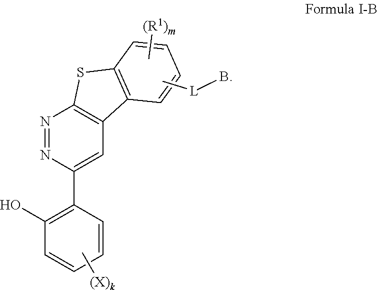

In some embodiments, the compound has the structure of Formula I-C:

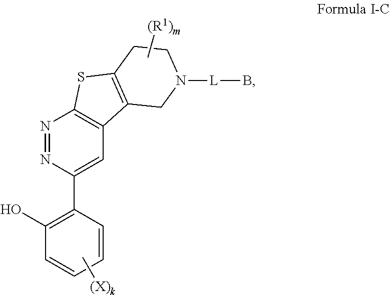

where each R¹ is, independently, optionally substituted C₁-C₆ alkyl.

In some embodiments, the compound has the structure of Formula I-D:

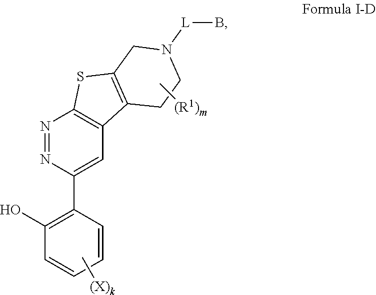

where each R¹ is, independently, optionally substituted C₁-C₆ alkyl.

In some embodiments, the compound has the structure of Formula I-E:

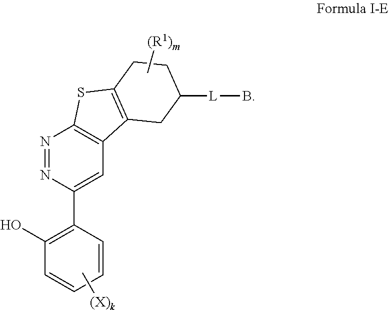

In some embodiments, the compound has the structure of Formula I-F:

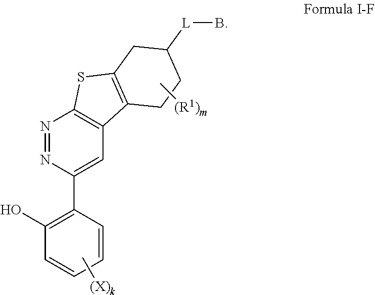

In some embodiments, m is 0.

In some embodiments, the compound has the structure of Formula I-G or II-G:

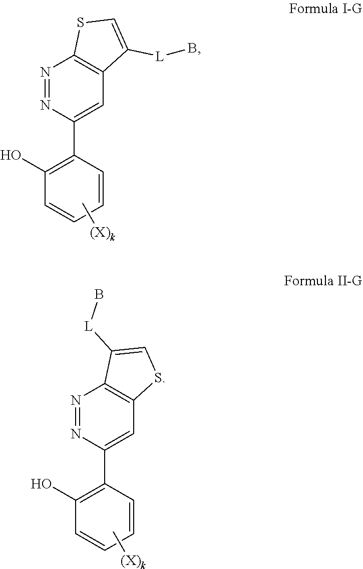

In some embodiments, the compound has the structure of Formula I-H or II-H:

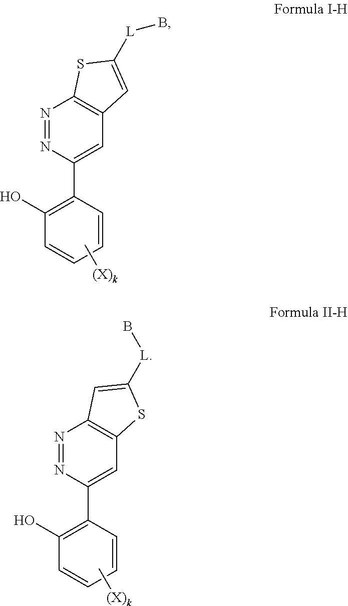

In some embodiments, the degradation moiety, B, has the structure of Formula A-1:

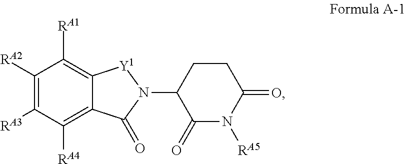

where
Y¹ is

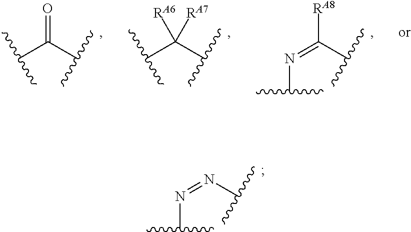

R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆ alkyl; or
R^{A6} and R^{A7}, together with the carbon atom to which each is bound, combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
each of R^{A1}, R^{A2}, R^{A3} and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

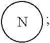

and

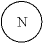

optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,
where one of R^{A1}, R^{A2}, R^{A3} and R^{A4} is A², or

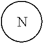

is substituted with A²; and
A² is a bond between the degradation moiety and the linker.

In some embodiments, R^{A5} is H or methyl. In some embodiments, R^{A5} is H.

In some embodiments, each of R^{A1}, R^{A2}, R^{A3}, and R^{A4} is, independently, H or A².

In some embodiments, R^{A1} is A² and each of R^{A2}, R^{A3}, and R^{A4} is H.

In some embodiments, R^{A2} is A² and each of R^{A1}, R^{A3}, and R^{A4} is H.

In some embodiments, R^{A3} is A² and each of R^{A1}, R^{A2} and R^{A4} is H.

In some embodiments, R^{A4} is A² and each of R^{A1}, R^{A2} and R^{A3} is H.

In some embodiments, Y¹ is

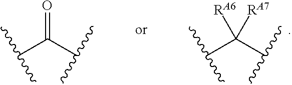

In some embodiments, R^{A6} is H. In some embodiments, R^{A7} is H.

In some embodiments, Y¹ is

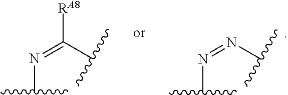

In some embodiments, R^{A8} is H or optionally substituted C₁-C₆ alkyl. In some embodiments, R^{A8} is H or methyl. In some embodiments, R^{A8} is methyl.

In some embodiments, the degradation moiety includes the structure of Formula A2:

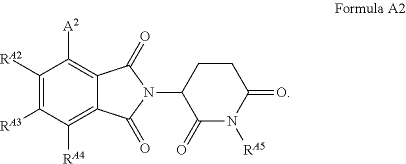

In some embodiments, where the degradation moiety is

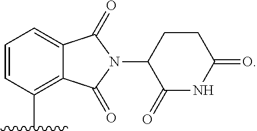 [SMILES: *c1cccc2c1C(=O)N(C1CCC(=O)NC1=O)C2=O]

In some embodiments, the degradation moiety includes the structure of Formula A4:

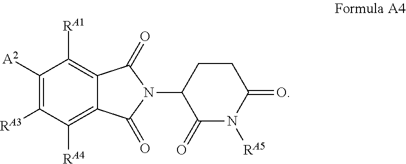

In some embodiments, the degradation moiety is

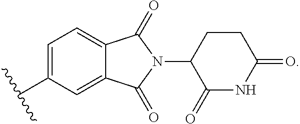 [SMILES: *c1ccc2c(c1)C(=O)N(C1CCC(=O)NC1=O)C2=O]

In some embodiments, the degradation moiety includes the structure of Formula A5:

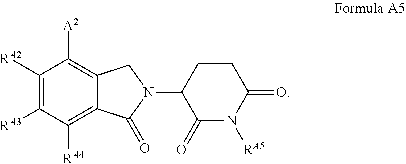

In some embodiments, the degradation moiety includes the structure of Formula A6:

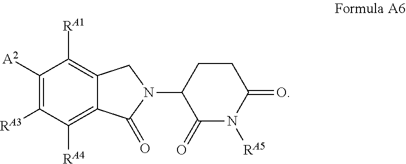

In some embodiments, the degradation moiety includes the structure of Formula A8:

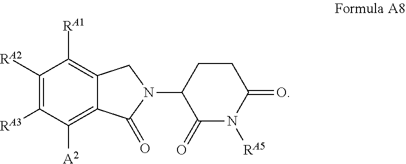

In some embodiments, the degradation moiety includes the structure of Formula A10:

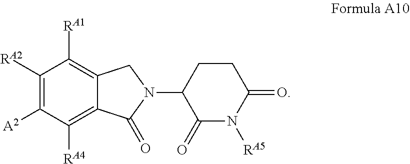

In some embodiments, the degradation moiety includes the structure of

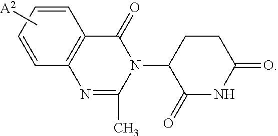

In some embodiments, the degradation moiety includes the structure of

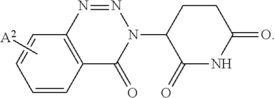

In some embodiments, the degradation moiety has the structure of Formula C:

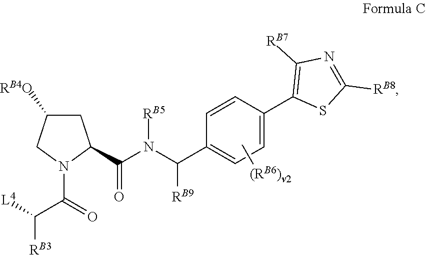

where
L⁴ is —N(R^{B1})(R^{B2}),

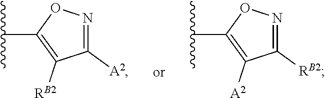

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
where one and only one of R^{B1}, R^{B3}, and R^{B6} is A²,
or a pharmaceutically acceptable salt thereof.

In some embodiments, the degradation moiety has the structure of Formula C′:

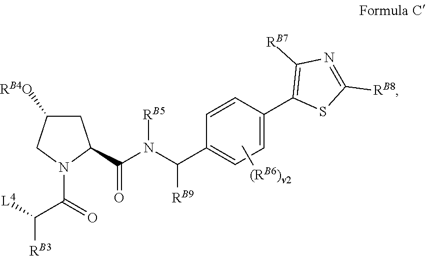

where
L⁴ is —N(R^{B1})(R^{B2}),

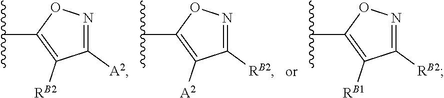

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₂-C₉ heterocyclyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
where one and only one of R^{B1}, R^{B3}, and R^{B6} is A²,
or a pharmaceutically acceptable salt thereof.

In some embodiments, the degradation moiety has the structure of Formula C1:

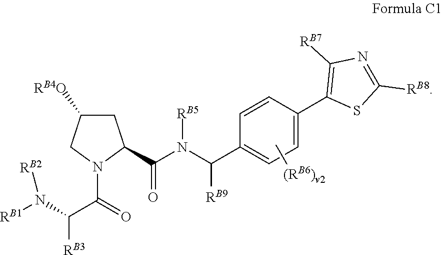

In some embodiments, the degradation moiety is

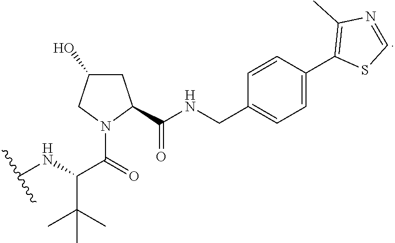 [SMILES: *N[C@H](C(=O)N1C[C@H](O)C[C@H]1C(=O)NCc1ccc(-c2scnc2C)cc1)C(C)(C)C]

In some embodiments, the degradation moiety is

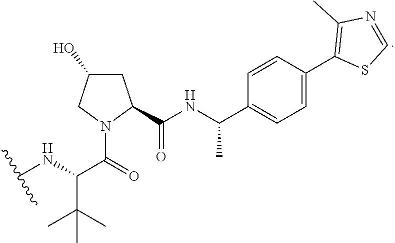 [SMILES: *N[C@H](C(=O)N1C[C@H](O)C[C@H]1C(=O)N[C@@H](C)c1ccc(-c2scnc2C)cc1)C(C)(C)C]

In some embodiments the degradation moiety is

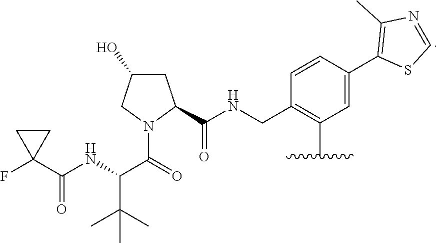 [SMILES: *c1cc(-c2scnc2C)ccc1CNC(=O)[C@@H]1C[C@@H](O)CN1C(=O)[C@@H](NC(=O)C1(F)CC1)C(C)(C)C]

In some embodiments, the degradation moiety has the structure of Formula C2:

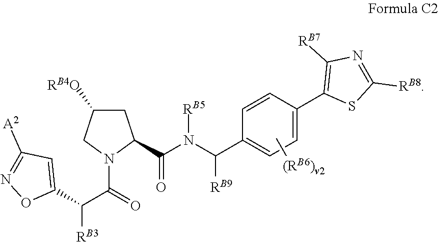

In some embodiments, R^{B9} is optionally substituted C₁-C₆ alkyl. In some embodiments, R^{B9} is methyl.

In some embodiments, R^{B9} is bonded to (S)-stereogenic center.

In some embodiments, the degradation moiety is

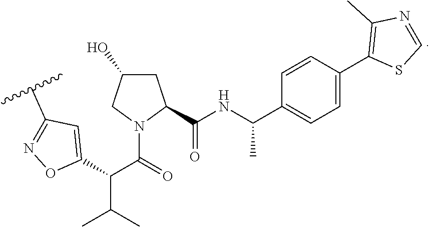 [SMILES: *c1cc([C@H](C(=O)N2C[C@H](O)C[C@H]2C(=O)N[C@@H](C)c2ccc(-c3scnc3C)cc2)C(C)C)on1]

In some embodiments, the linker has the structure of Formula III:

A¹-(B¹)_{f}—(C¹)_{g}—(B²)ₕ-(D)-(B³)ᵢ—(C²)ⱼ—(B⁴)ₖ-A², Formula III
or a pharmaceutically acceptable salt thereof,
where
A¹ is a bond between the linker and ring system A;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₂-C₆ heterocyclyl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
each of C₁ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In some embodiments, the linker has the structure of Formula III:

A¹-(B¹)_{f}—(C¹)_{g}—(B²)ₕ-(D)-(B³)ᵢ—(C²)ⱼ—(B⁴)ₖ-A², Formula III
or a pharmaceutically acceptable salt thereof,
where
A¹ is a bond between the linker and ring system A;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₂-C₆ heterocyclyl, optionally substituted C₂-C₉ heteroaryl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In some embodiments, each of B¹, B², B³, and B⁴ is, independently, optionally substituted C₁-C₂ alkyl, optionally substituted C₁-C₃ heteroalkyl, optionally substituted C₂-C₆ heterocyclyl, or NR^{N}.

In some embodiments, each R^{N} is, independently, H or optionally substituted C₁-C₄ alkyl.

In some embodiments, each R^{N} is, independently, H or CH₃.

In some embodiments, each of B¹ and B⁴ is, independently,

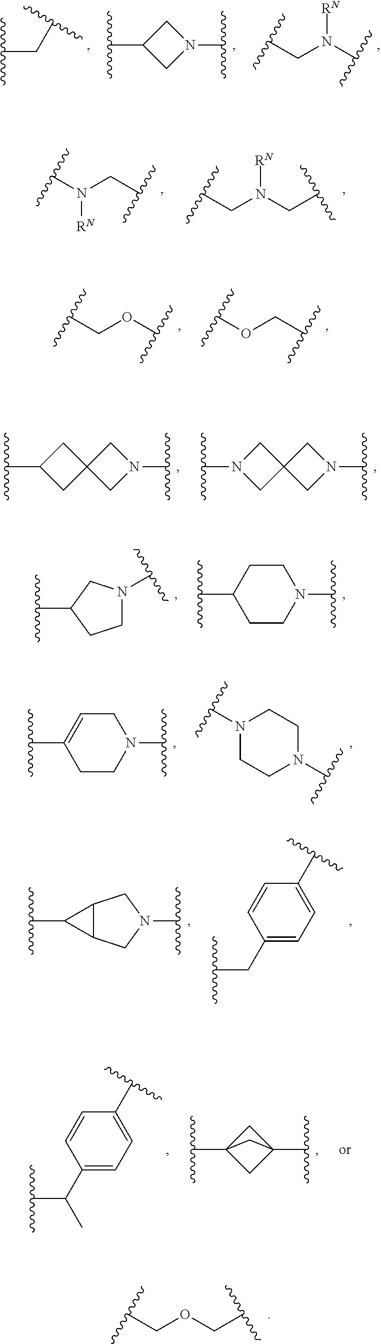

In some embodiments, each of B¹ and B⁴ is, independently,

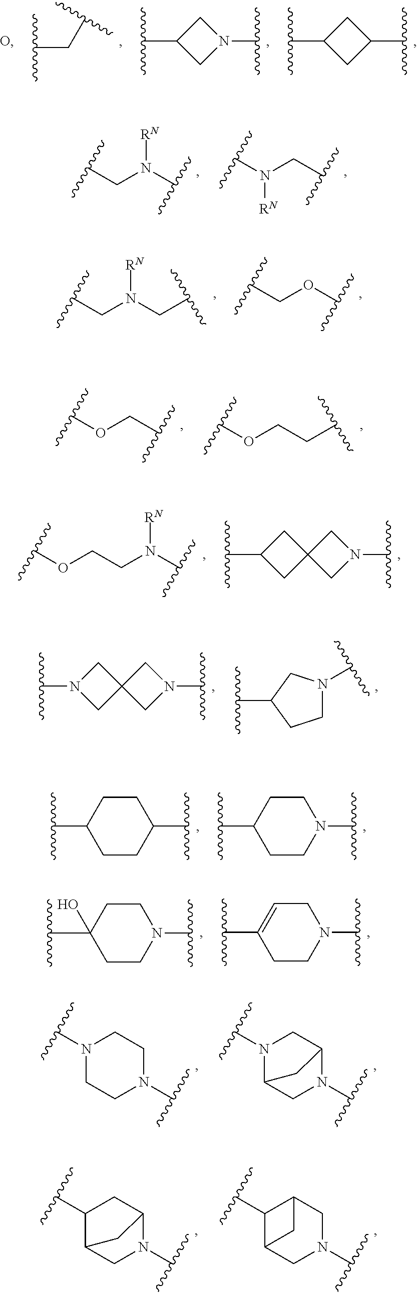

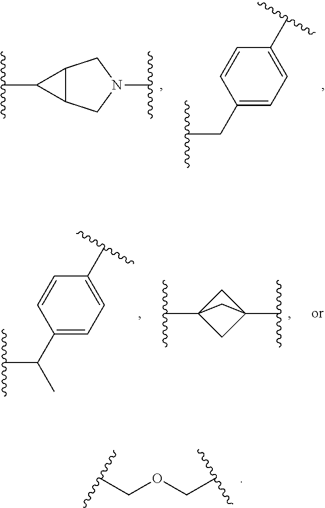

In some embodiments, B¹ is,

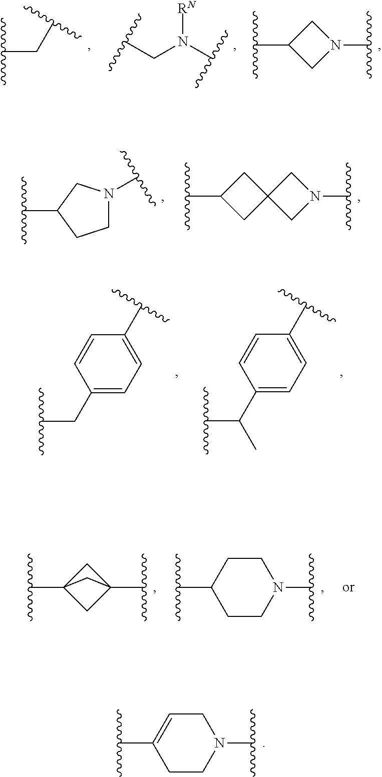

In some embodiments, B¹ is

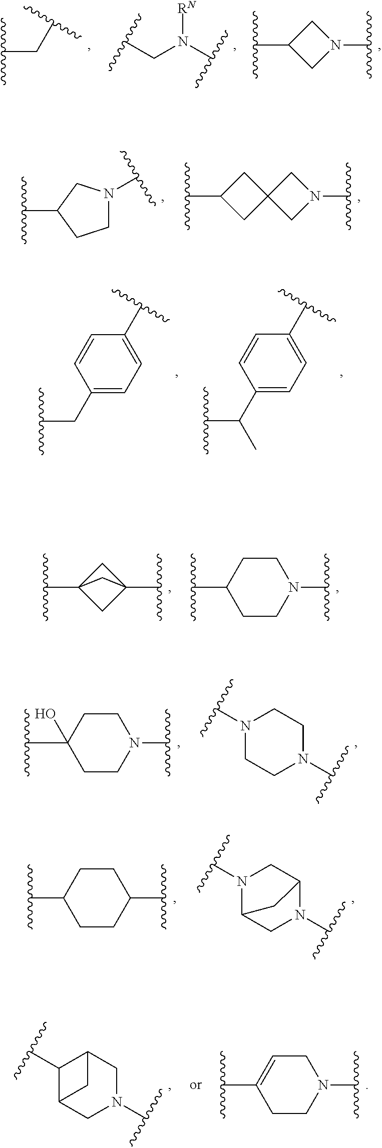

In some embodiments, B⁴ is

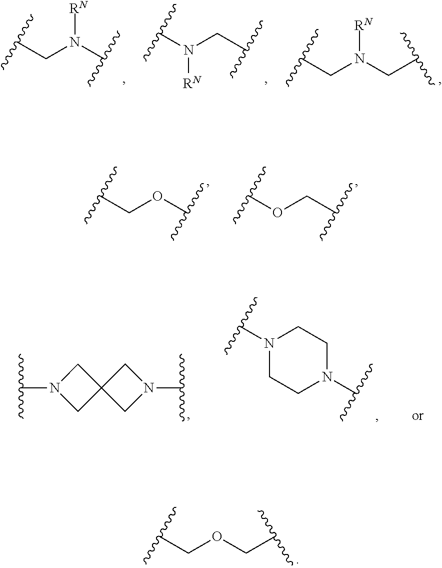

In some embodiments, B⁴ is O,

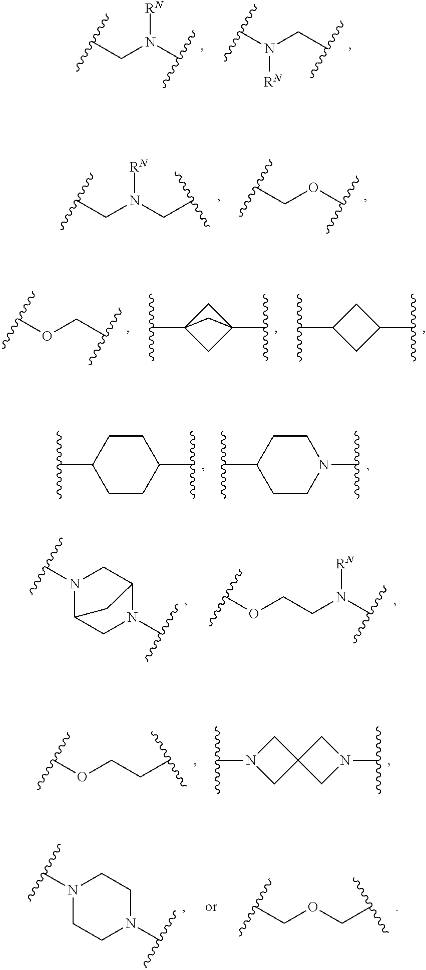

In some embodiments, each of C¹ and C² is

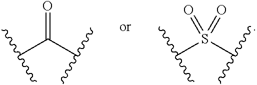

In some embodiments, C¹ is

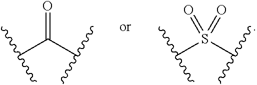

In some embodiments, C² is

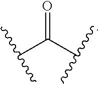 [SMILES: *C(*)=O]

In some embodiments, B² is optionally substituted C₁-C₄ alkyl. In some embodiments, B² is optionally substituted C₂-C₆ heterocyclyl.

In some embodiments, B² is

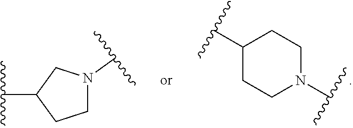 [SMILES: *C1CCN(*)C1 ; *C1CCN(*)CC1]

In some embodiments, B² is optionally substituted C₂-C₉ heteroaryl. In some embodiments, B² is

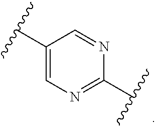 [SMILES: *c1cnc(*)nc1]

In some embodiments, B³ is optionally substituted C₃-C₁₀ cycloalkyl. In some embodiments, B³ is

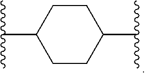 [SMILES: *C1CCC(*)CC1]

In some embodiments, D is optionally substituted C₁-C₁₀ alkyl.

In some embodiments, f is 1. In some embodiments, g is 0. In some embodiments, g is 1. In some embodiments, h is 0. In some embodiments, h is 1. In some embodiments, i is 0. In some embodiments, i is 1. In some embodiments, j is 0. In some embodiments, j is 1. In some embodiments, k is 0. In some embodiments, k is 1.

In some embodiments, the linker has the structure of

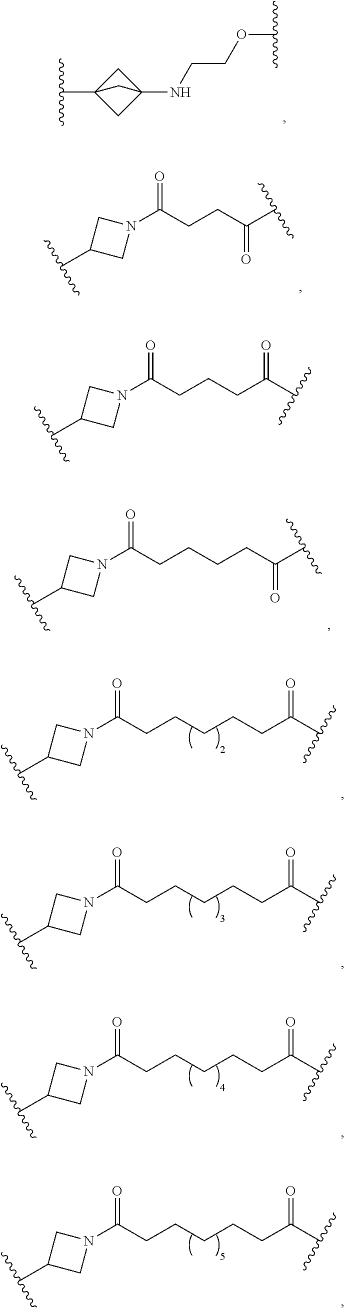

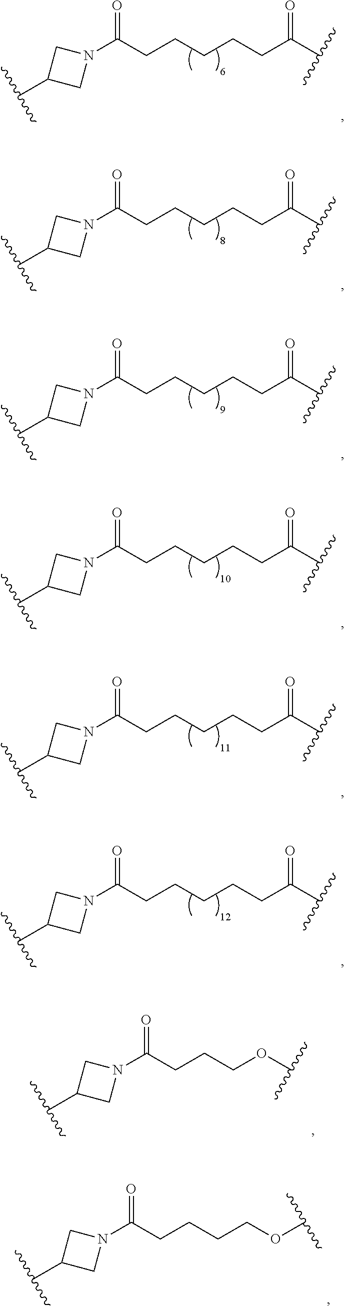

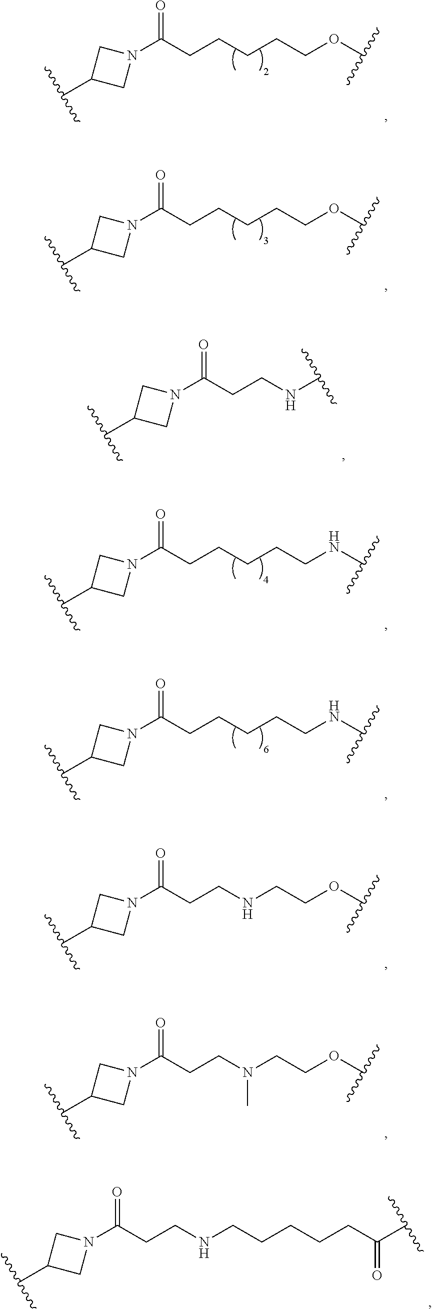

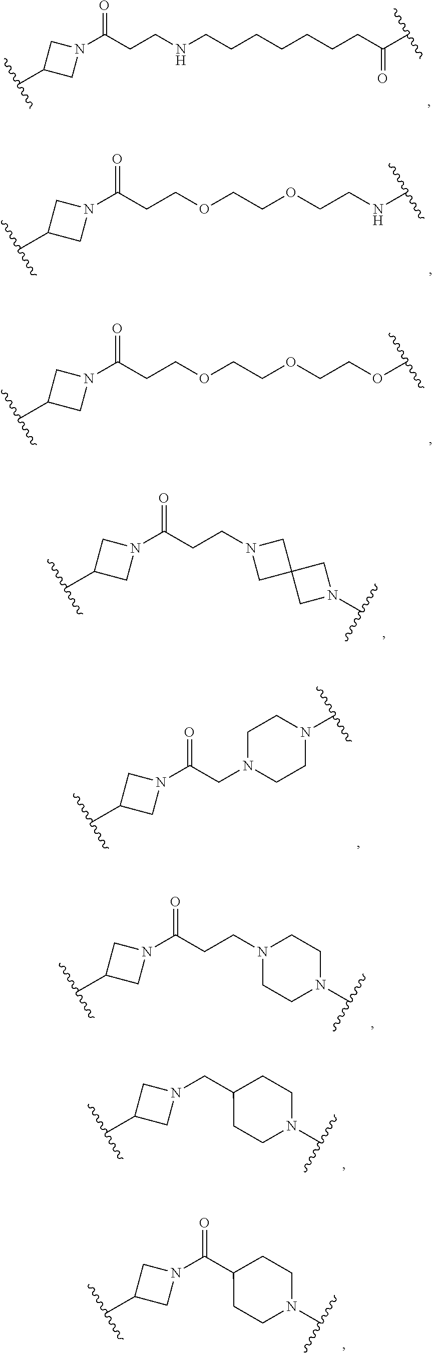

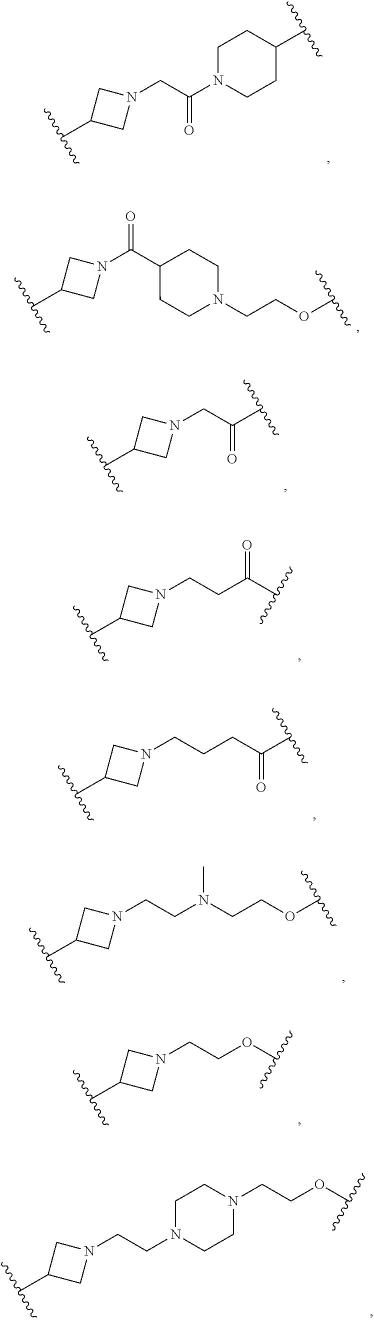

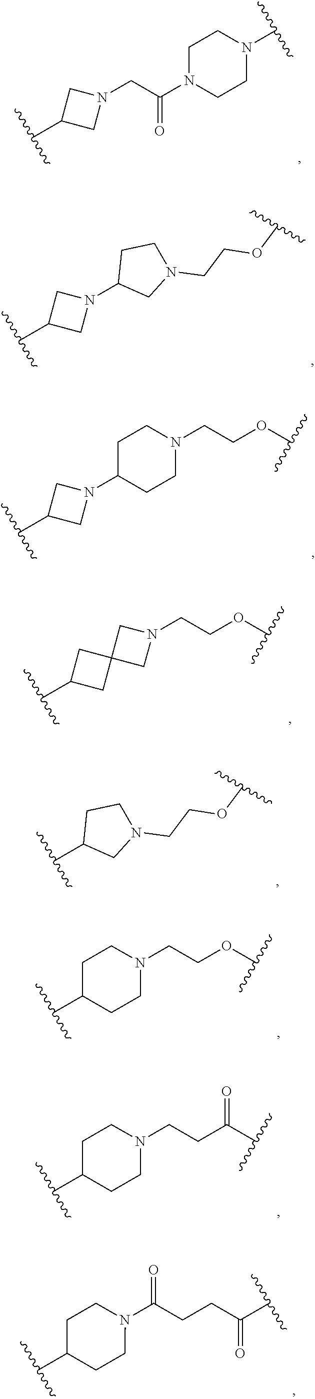

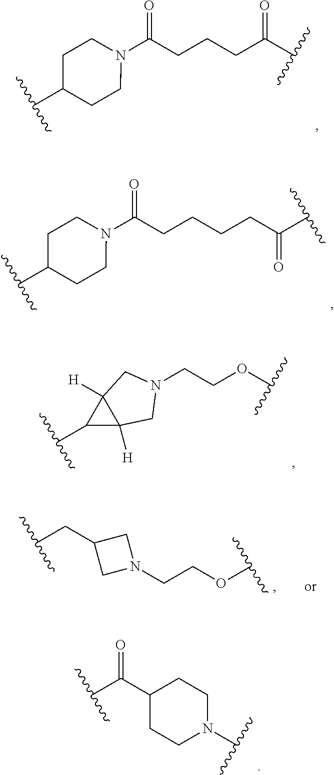 [SMILES: *C(=O)CCCC(=O)N1CCC(*)CC1 ; *C(=O)CCCCC(=O)N1CCC(*)CC1 ; *OCCN1CC2C(*)C2C1 ; *CC1CN(CCO*)C1 ; *C(=O)C1CCN(*)CC1]

In some embodiments, the linker has the structure of

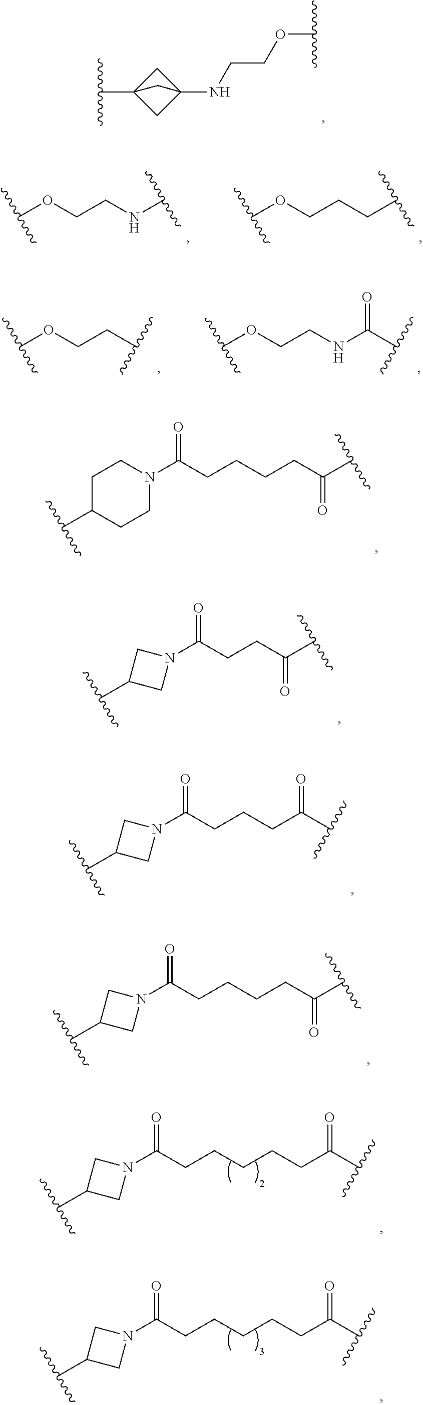

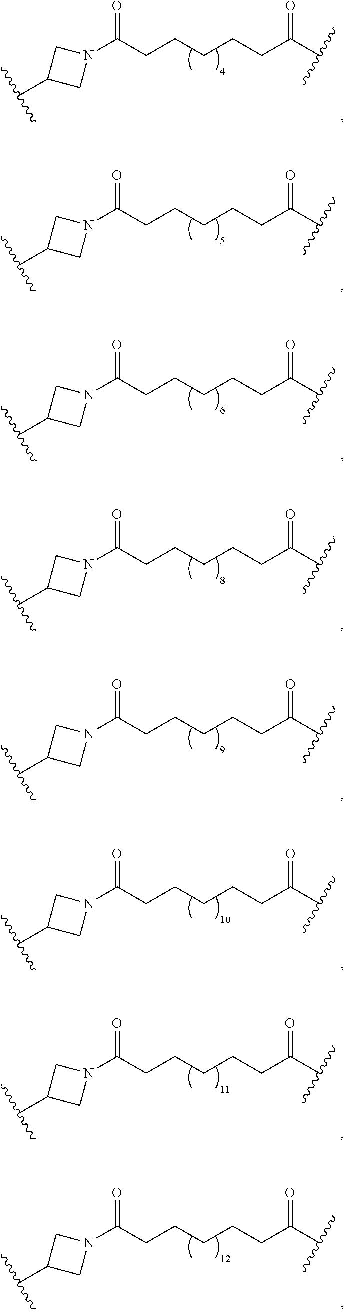

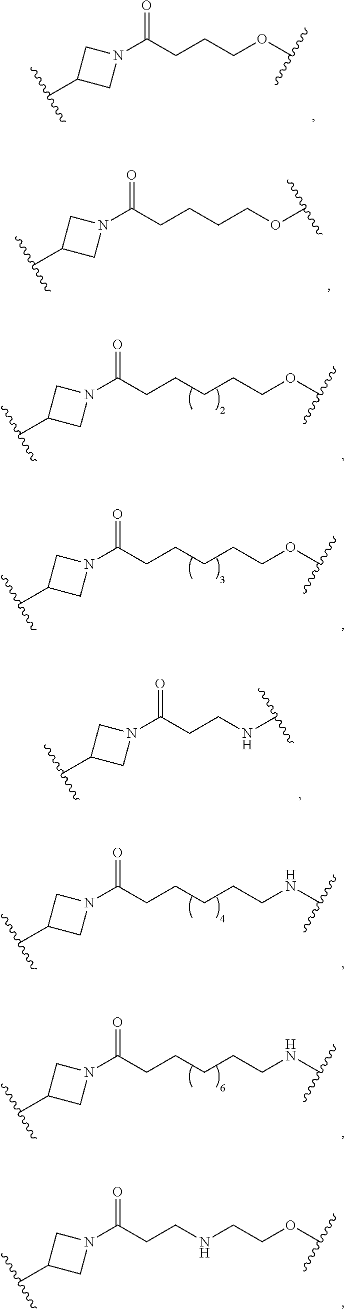

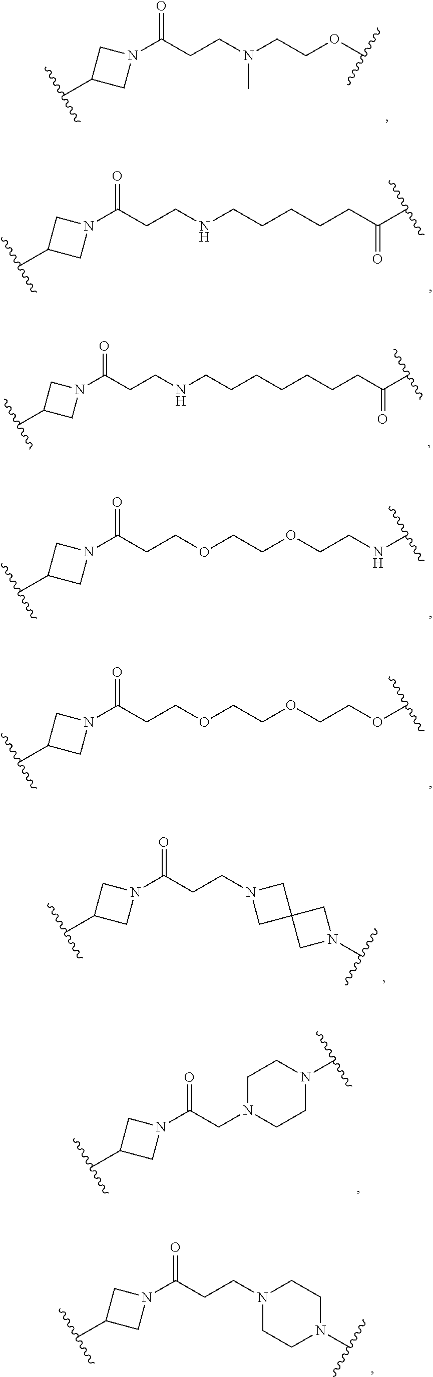

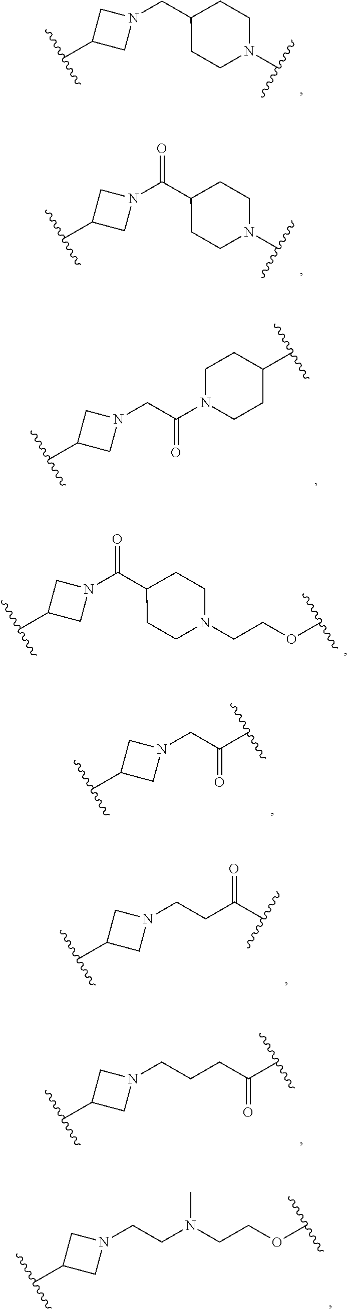

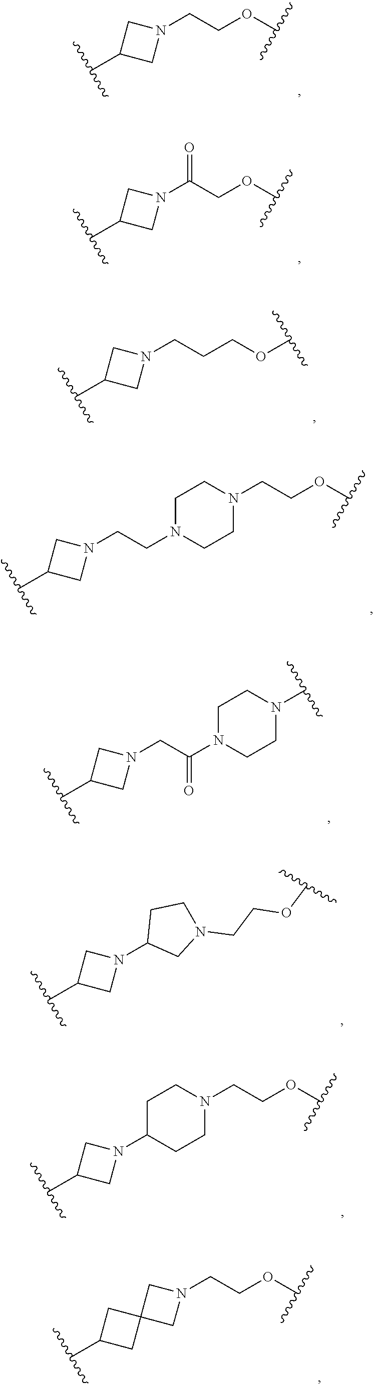

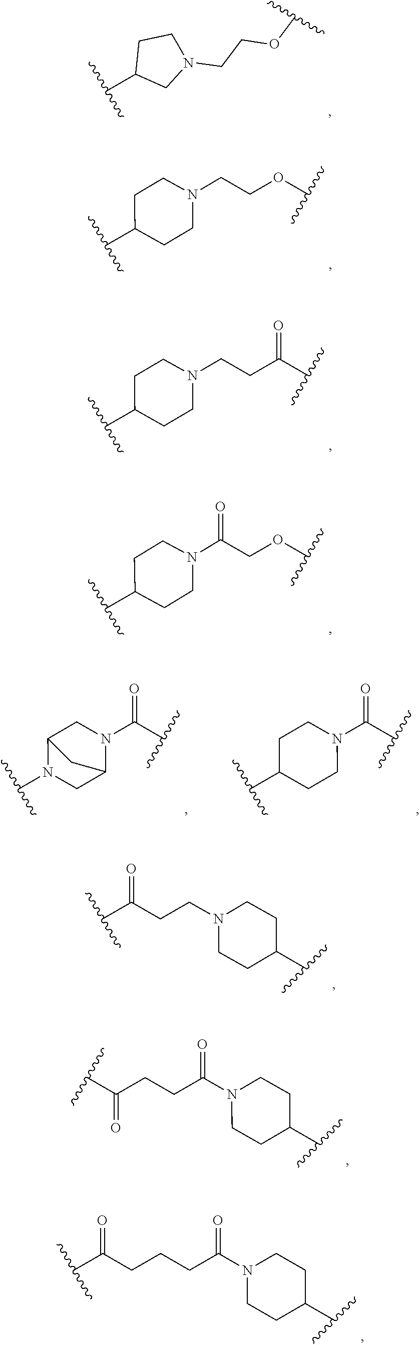

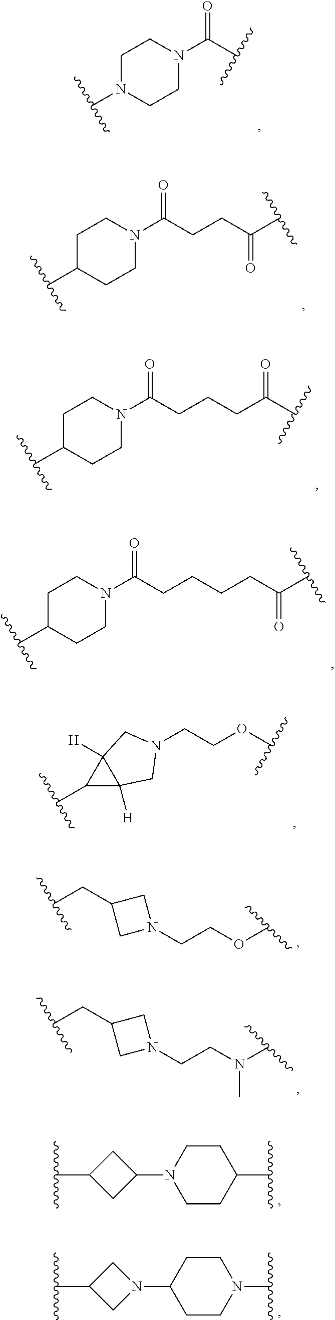

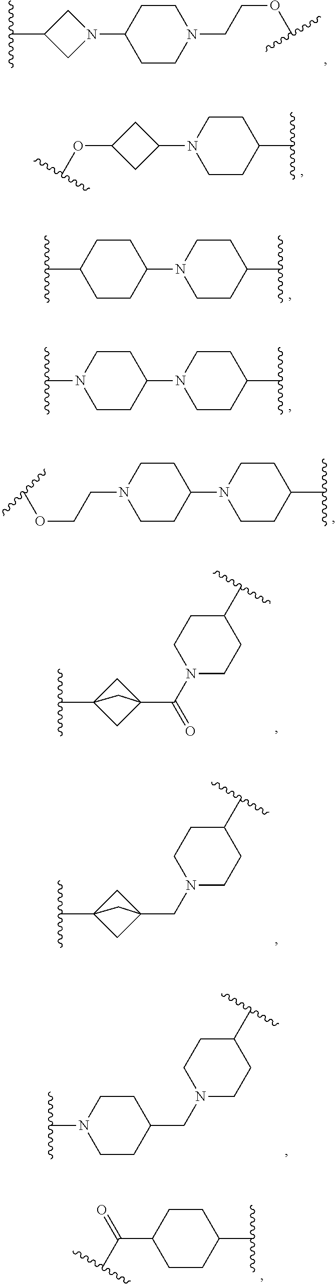

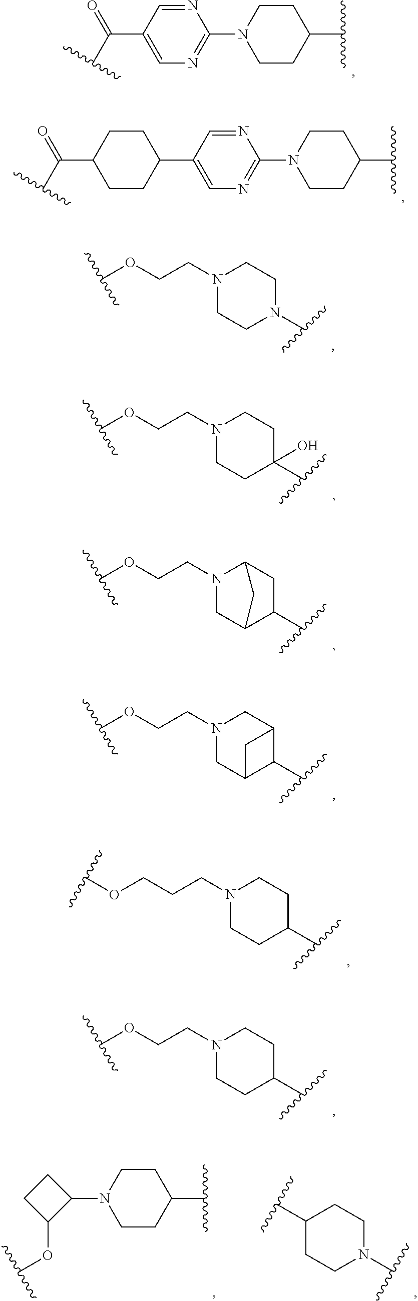

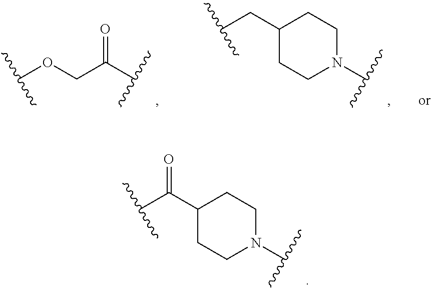 [SMILES: *OCC(*)=O ; *CC1CCN(*)CC1 ; *C(=O)C1CCN(*)CC1]

In some embodiments, the shortest chain of atoms connecting two valencies of the linker is 2 to 10 atoms long.

In some embodiments, the shortest chain of atoms connecting two valencies of the linker is 6 atoms long.

In some embodiments, the linker has the structure of

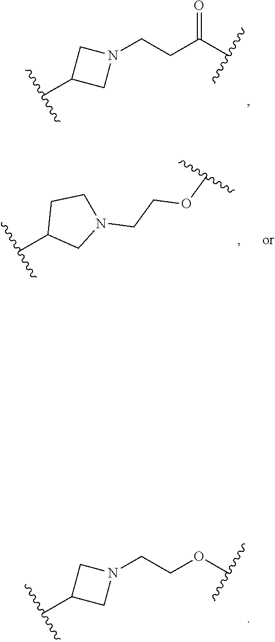 [SMILES: *C(=O)CCN1CC(*)C1 ; *OCCN1CCC(*)C1 ; *OCCN1CC(*)C1]

In an aspect, the invention features a compound selected from the group consisting of 1-33 in Table 1 and pharmaceutically acceptable salts thereof. In an aspect, the invention features a compound selected from the group consisting of 1-115 in Table 1 and pharmaceutically acceptable salts thereof.

TABLE 1
Compounds of the Invention

| # | Compound |
|---|---|
| 1 | 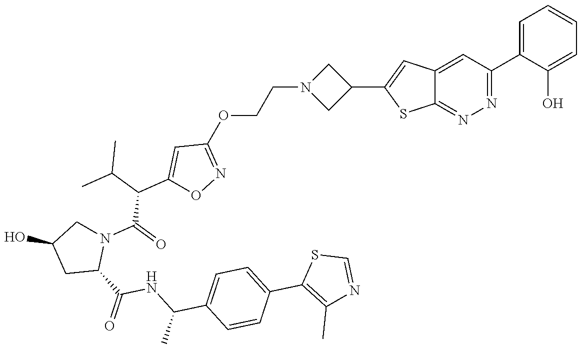 [SMILES: Cc1ncsc1-c1ccc([C@H](C)NC(=O)[C@@H]2C[C@@H](O)CN2C(=O)[C@@H](c2cc(OCCN3CC(c4cc5cc(-c6ccccc6O)nnc5s4)C3)no2)C(C)C)cc1] |
| 2 | 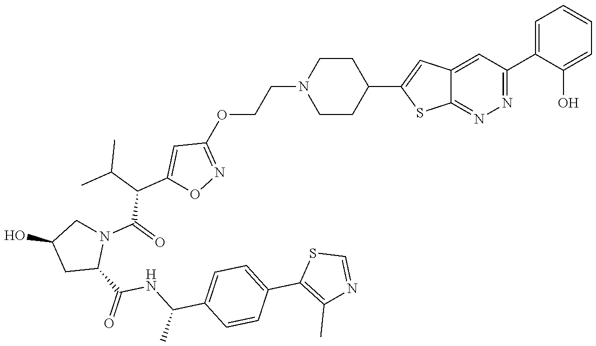 [SMILES: Cc1ncsc1-c1ccc([C@H](C)NC(=O)[C@@H]2C[C@@H](O)CN2C(=O)[C@@H](c2cc(OCCN3CCC(c4cc5cc(-c6ccccc6O)nnc5s4)CC3)no2)C(C)C)cc1] |
| 3 | 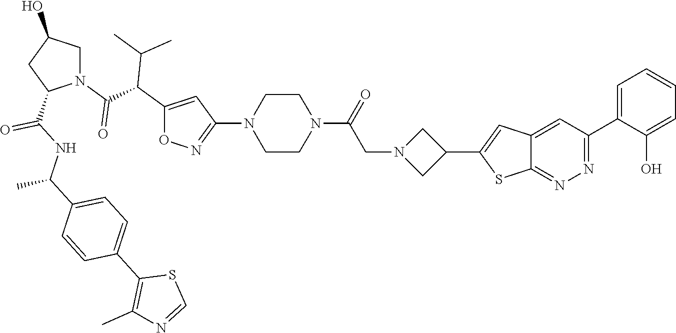 [SMILES: Cc1ncsc1-c1ccc([C@H](C)NC(=O)[C@@H]2C[C@@H](O)CN2C(=O)[C@@H](c2cc(N3CCN(C(=O)CN4CC(c5cc6cc(-c7ccccc7O)nnc6s5)C4)CC3)no2)C(C)C)cc1] |
| 4 | 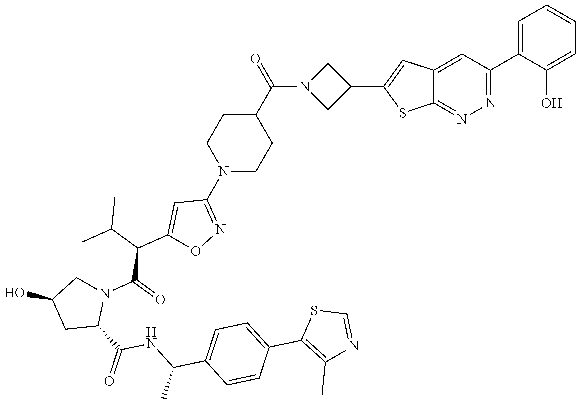 [SMILES: Cc1ncsc1-c1ccc([C@H](C)NC(=O)[C@@H]2C[C@@H](O)CN2C(=O)[C@H](c2cc(N3CCC(C(=O)N4CC(c5cc6cc(-c7ccccc7O)nnc6s5)C4)CC3)no2)C(C)C)cc1] |
| 5 | 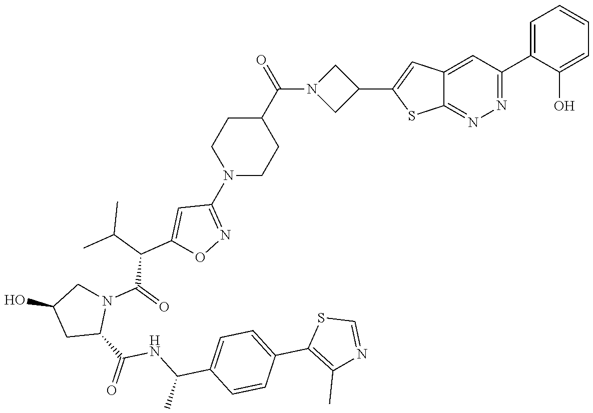 [SMILES: Cc1ncsc1-c1ccc([C@H](C)NC(=O)[C@@H]2C[C@@H](O)CN2C(=O)[C@@H](c2cc(N3CCC(C(=O)N4CC(c5cc6cc(-c7ccccc7O)nnc6s5)C4)CC3)no2)C(C)C)cc1] |
| 6 | 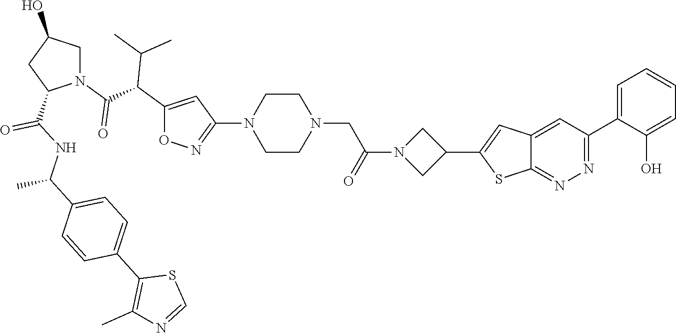 [SMILES: Cc1ncsc1-c1ccc([C@H](C)NC(=O)[C@@H]2C[C@@H](O)CN2C(=O)[C@@H](c2cc(N3CCN(CC(=O)N4CC(c5cc6cc(-c7ccccc7O)nnc6s5)C4)CC3)no2)C(C)C)cc1] |
| 7 | 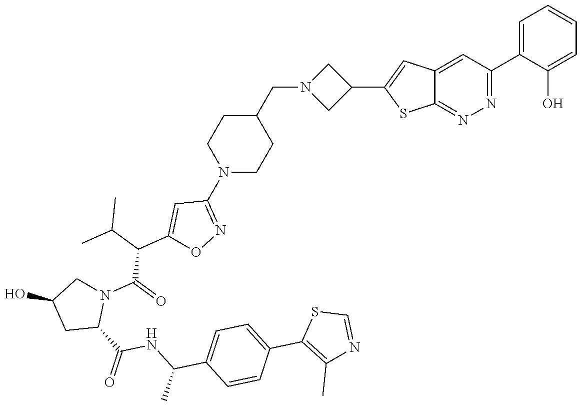 [SMILES: Cc1ncsc1-c1ccc([C@H](C)NC(=O)[C@@H]2C[C@@H](O)CN2C(=O)[C@@H](c2cc(N3CCC(CN4CC(c5cc6cc(-c7ccccc7O)nnc6s5)C4)CC3)no2)C(C)C)cc1] |
| 8 | 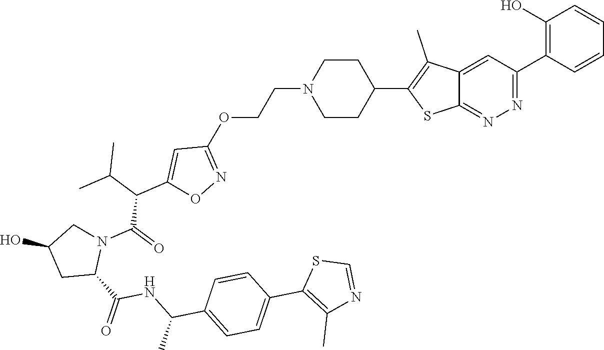 [SMILES: Cc1ncsc1-c1ccc([C@H](C)NC(=O)[C@@H]2C[C@@H](O)CN2C(=O)[C@@H](c2cc(OCCN3CCC(c4sc5nnc(-c6ccccc6O)cc5c4C)CC3)no2)C(C)C)cc1] |
| 9 | 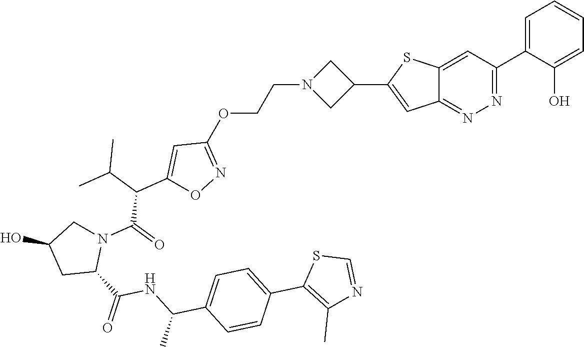 [SMILES: Cc1ncsc1-c1ccc([C@H](C)NC(=O)[C@@H]2C[C@@H](O)CN2C(=O)[C@@H](c2cc(OCCN3CC(c4cc5nnc(-c6ccccc6O)cc5s4)C3)no2)C(C)C)cc1] |
| 10 | 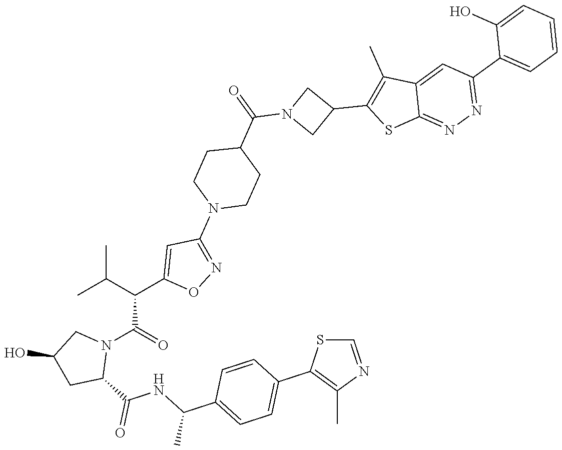 [SMILES: Cc1ncsc1-c1ccc([C@H](C)NC(=O)[C@@H]2C[C@@H](O)CN2C(=O)[C@@H](c2cc(N3CCC(C(=O)N4CC(c5sc6nnc(-c7ccccc7O)cc6c5C)C4)CC3)no2)C(C)C)cc1] |
| 11 | 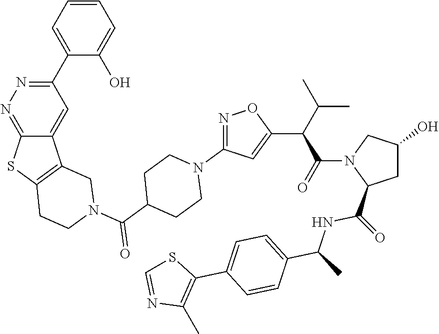 [SMILES: Cc1ncsc1-c1ccc([C@H](C)NC(=O)[C@@H]2C[C@@H](O)CN2C(=O)[C@@H](c2cc(N3CCC(C(=O)N4CCc5sc6nnc(-c7ccccc7O)cc6c5C4)CC3)no2)C(C)C)cc1] |
| 12 | 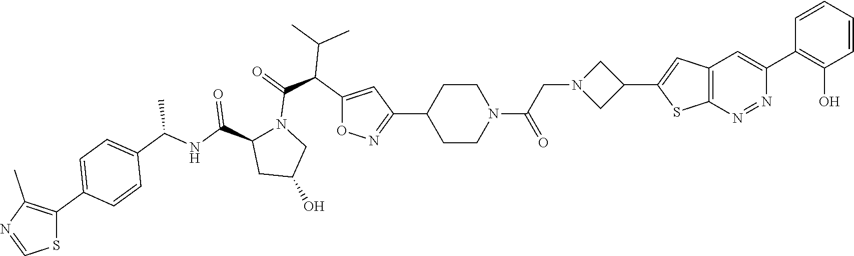 [SMILES: Cc1ncsc1-c1ccc([C@H](C)NC(=O)[C@@H]2C[C@@H](O)CN2C(=O)[C@H](c2cc(C3CCN(C(=O)CN4CC(c5cc6cc(-c7ccccc7O)nnc6s5)C4)CC3)no2)C(C)C)cc1] |
| 13 | 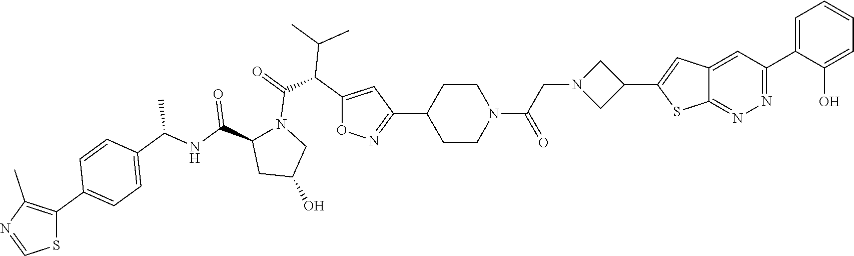 [SMILES: Cc1ncsc1-c1ccc([C@H](C)NC(=O)[C@@H]2C[C@@H](O)CN2C(=O)[C@@H](c2cc(C3CCN(C(=O)CN4CC(c5cc6cc(-c7ccccc7O)nnc6s5)C4)CC3)no2)C(C)C)cc1] |
| 14 | 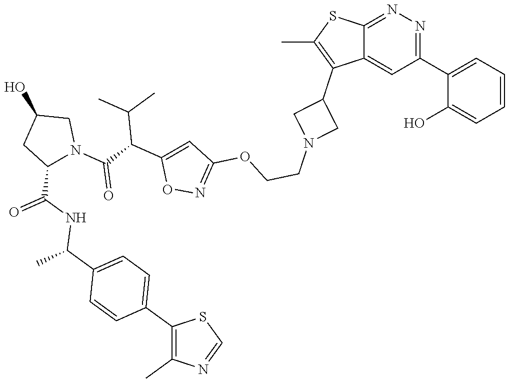 [SMILES: Cc1ncsc1-c1ccc([C@H](C)NC(=O)[C@@H]2C[C@@H](O)CN2C(=O)[C@@H](c2cc(OCCN3CC(c4c(C)sc5nnc(-c6ccccc6O)cc45)C3)no2)C(C)C)cc1] |
| 15 | 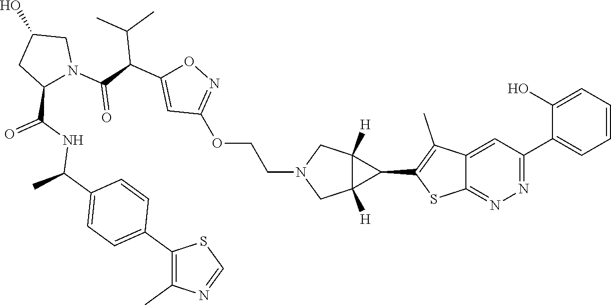 [SMILES: Cc1ncsc1-c1ccc([C@@H](C)NC(=O)[C@H]2C[C@H](O)CN2C(=O)[C@H](c2cc(OCCN3C[C@@H]4[C@H](C3)[C@H]4c3sc4nnc(-c5ccccc5O)cc4c3C)no2)C(C)C)cc1] |
| 16 | 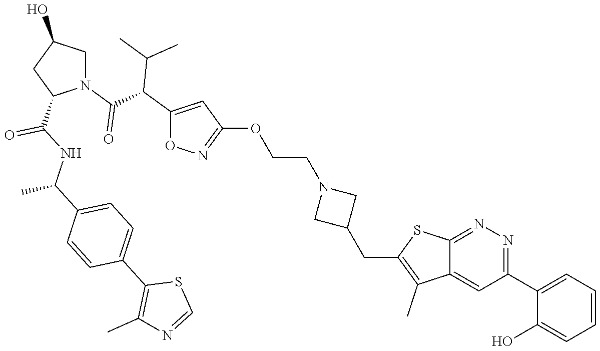 [SMILES: Cc1ncsc1-c1ccc([C@H](C)NC(=O)[C@@H]2C[C@@H](O)CN2C(=O)[C@@H](c2cc(OCCN3CC(Cc4sc5nnc(-c6ccccc6O)cc5c4C)C3)no2)C(C)C)cc1] |
| 17 | 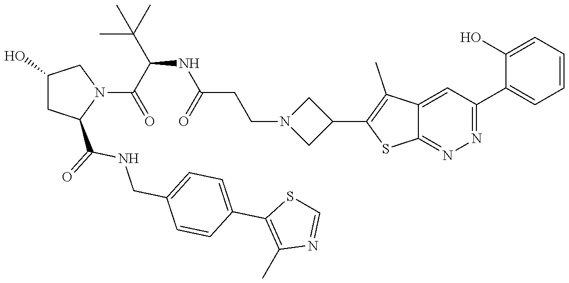 [SMILES: Cc1ncsc1-c1ccc(CNC(=O)[C@H]2C[C@H](O)CN2C(=O)[C@H](NC(=O)CCN2CC(c3sc4nnc(-c5ccccc5O)cc4c3C)C2)C(C)(C)C)cc1] |
| 18 | 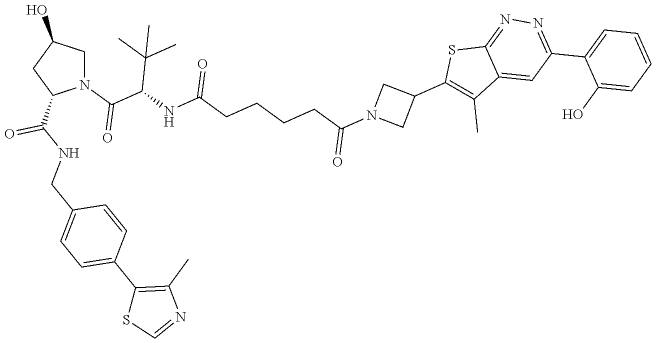 [SMILES: Cc1ncsc1-c1ccc(CNC(=O)[C@@H]2C[C@@H](O)CN2C(=O)[C@@H](NC(=O)CCCCC(=O)N2CC(c3sc4nnc(-c5ccccc5O)cc4c3C)C2)C(C)(C)C)cc1] |
| 19 | 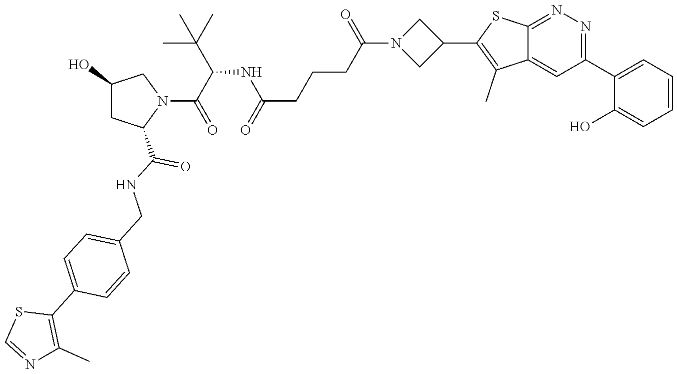 [SMILES: Cc1ncsc1-c1ccc(CNC(=O)[C@@H]2C[C@@H](O)CN2C(=O)[C@@H](NC(=O)CCCC(=O)N2CC(c3sc4nnc(-c5ccccc5O)cc4c3C)C2)C(C)(C)C)cc1] |
| 20 | 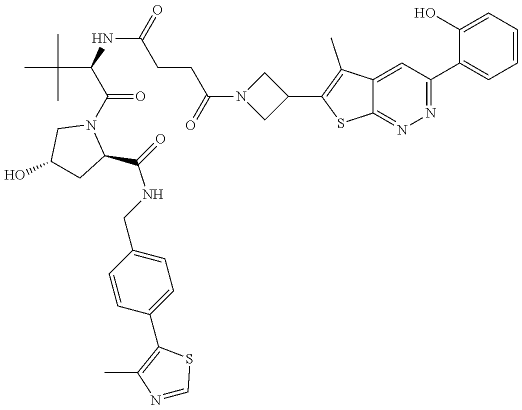 [SMILES: Cc1ncsc1-c1ccc(CNC(=O)[C@H]2C[C@H](O)CN2C(=O)[C@H](NC(=O)CCC(=O)N2CC(c3sc4nnc(-c5ccccc5O)cc4c3C)C2)C(C)(C)C)cc1] |
| 21 | 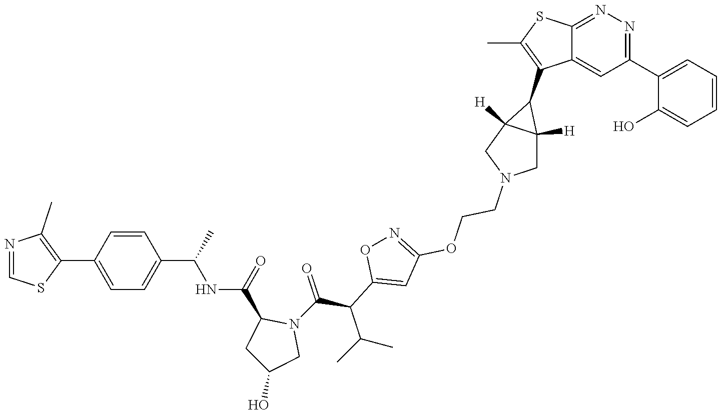 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3c(C)sc4nnc(-c5ccccc5O)cc34)no2)C(C)C)cc1] |
| 22 | 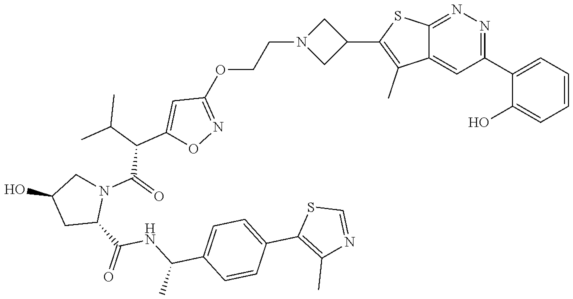 [SMILES: Cc1ncsc1-c1ccc([C@H](C)NC(=O)[C@@H]2C[C@@H](O)CN2C(=O)[C@@H](c2cc(OCCN3CC(c4sc5nnc(-c6ccccc6O)cc5c4C)C3)no2)C(C)C)cc1] |
| 23 | 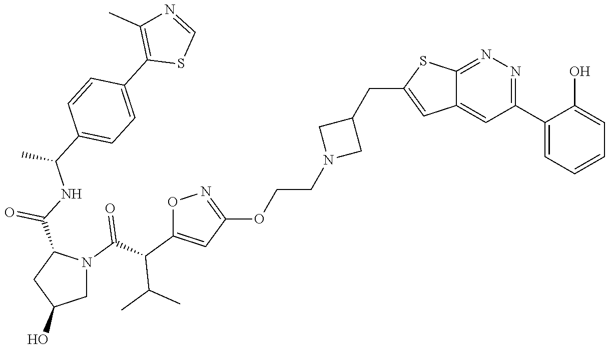 [SMILES: Cc1ncsc1-c1ccc([C@@H](C)NC(=O)[C@H]2C[C@H](O)CN2C(=O)[C@H](c2cc(OCCN3CC(Cc4cc5cc(-c6ccccc6O)nnc5s4)C3)no2)C(C)C)cc1] |
| 24 | 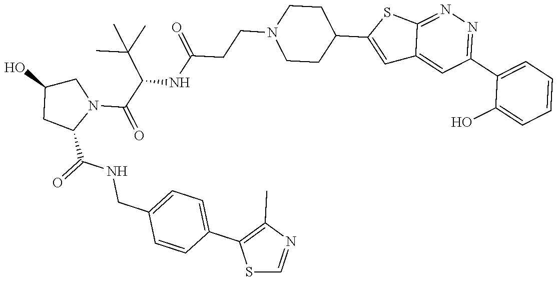 [SMILES: Cc1ncsc1-c1ccc(CNC(=O)[C@@H]2C[C@@H](O)CN2C(=O)[C@@H](NC(=O)CCN2CCC(c3cc4cc(-c5ccccc5O)nnc4s3)CC2)C(C)(C)C)cc1] |
| 25 | 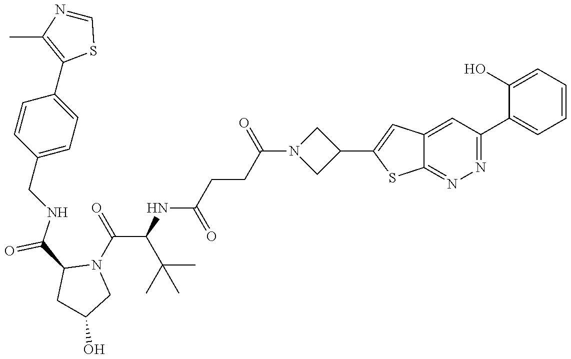 [SMILES: Cc1ncsc1-c1ccc(CNC(=O)[C@@H]2C[C@@H](O)CN2C(=O)[C@@H](NC(=O)CCC(=O)N2CC(c3cc4cc(-c5ccccc5O)nnc4s3)C2)C(C)(C)C)cc1] |
| 26 | 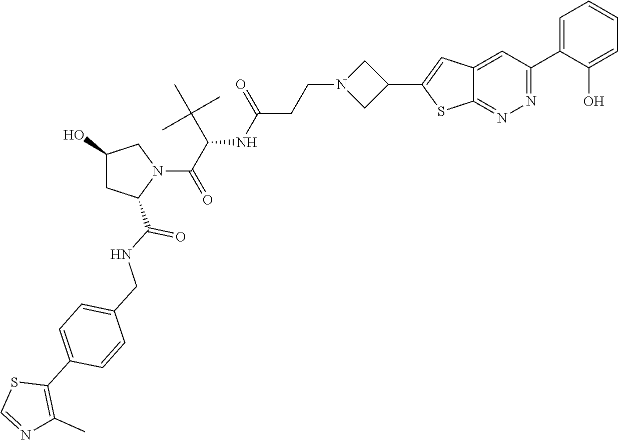 [SMILES: Cc1ncsc1-c1ccc(CNC(=O)[C@@H]2C[C@@H](O)CN2C(=O)[C@@H](NC(=O)CCN2CC(c3cc4cc(-c5ccccc5O)nnc4s3)C2)C(C)(C)C)cc1] |
| 27 | 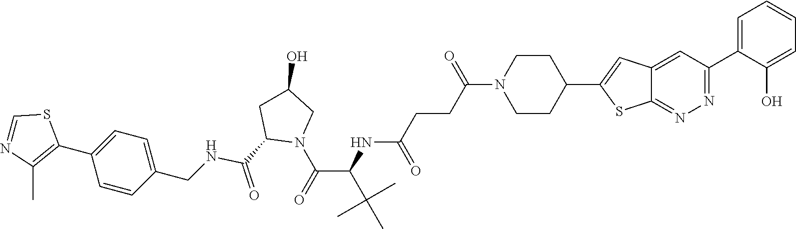 [SMILES: Cc1ncsc1-c1ccc(CNC(=O)[C@@H]2C[C@@H](O)CN2C(=O)[C@@H](NC(=O)CCC(=O)N2CCC(c3cc4cc(-c5ccccc5O)nnc4s3)CC2)C(C)(C)C)cc1] |
| 28 | 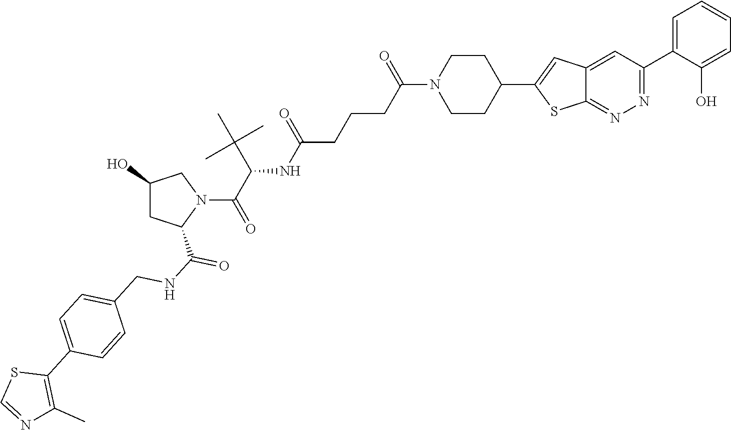 [SMILES: Cc1ncsc1-c1ccc(CNC(=O)[C@@H]2C[C@@H](O)CN2C(=O)[C@@H](NC(=O)CCCC(=O)N2CCC(c3cc4cc(-c5ccccc5O)nnc4s3)CC2)C(C)(C)C)cc1] |
| 29 | 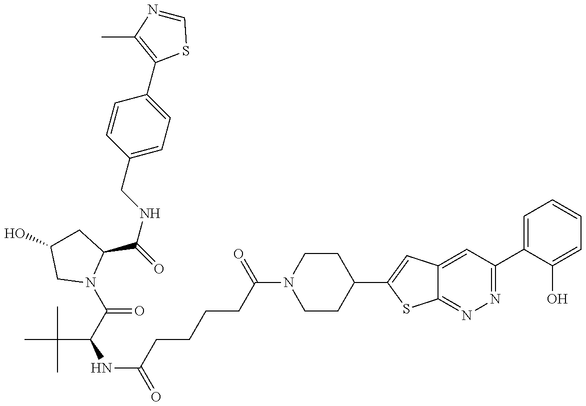 [SMILES: Cc1ncsc1-c1ccc(CNC(=O)[C@@H]2C[C@@H](O)CN2C(=O)[C@@H](NC(=O)CCCCC(=O)N2CCC(c3cc4cc(-c5ccccc5O)nnc4s3)CC2)C(C)(C)C)cc1] |
| 30 | 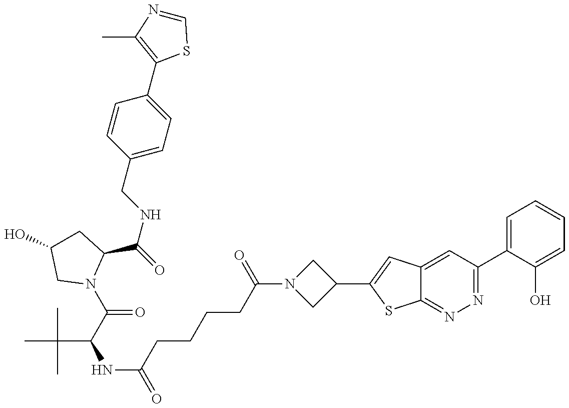 [SMILES: Cc1ncsc1-c1ccc(CNC(=O)[C@@H]2C[C@@H](O)CN2C(=O)[C@@H](NC(=O)CCCCC(=O)N2CC(c3cc4cc(-c5ccccc5O)nnc4s3)C2)C(C)(C)C)cc1] |
| 31 | 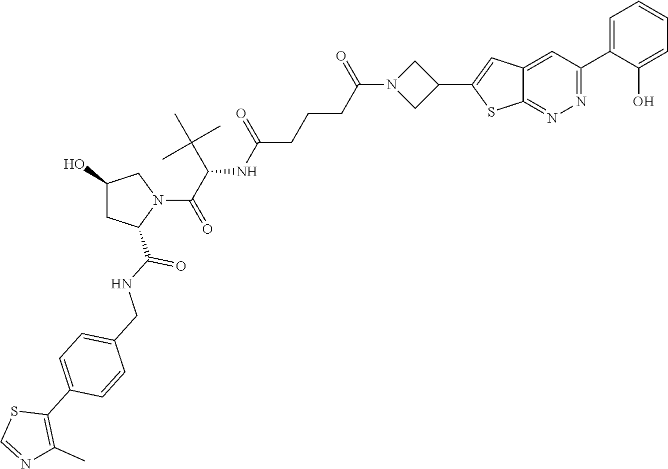 [SMILES: Cc1ncsc1-c1ccc(CNC(=O)[C@@H]2C[C@@H](O)CN2C(=O)[C@@H](NC(=O)CCCC(=O)N2CC(c3cc4cc(-c5ccccc5O)nnc4s3)C2)C(C)(C)C)cc1] |
| 32 | 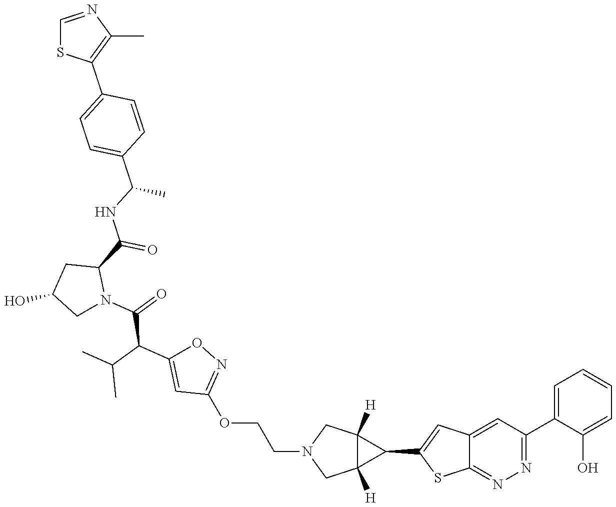 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3cc4cc(-c5ccccc5O)nnc4s3)no2)C(C)C)cc1] |
| 33 | 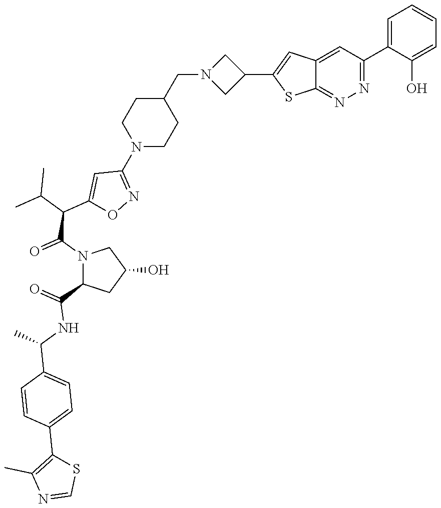 [SMILES: Cc1ncsc1-c1ccc([C@H](C)NC(=O)[C@@H]2C[C@@H](O)CN2C(=O)[C@H](c2cc(N3CCC(CN4CC(c5cc6cc(-c7ccccc7O)nnc6s5)C4)CC3)no2)C(C)C)cc1] |
| 34 | 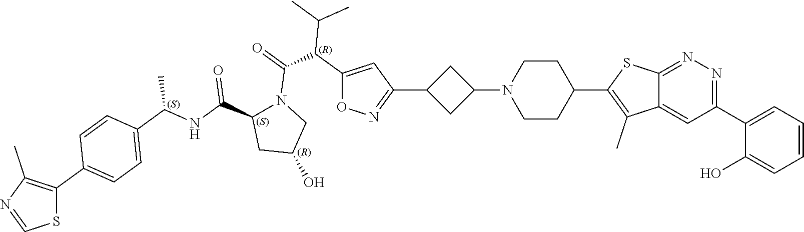 |
| 35 | 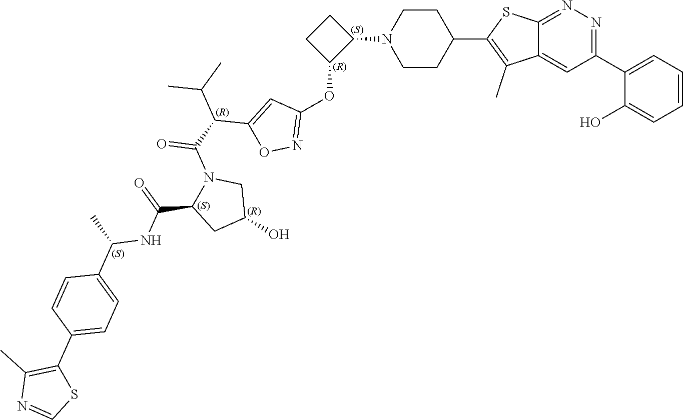 |
| 36 | 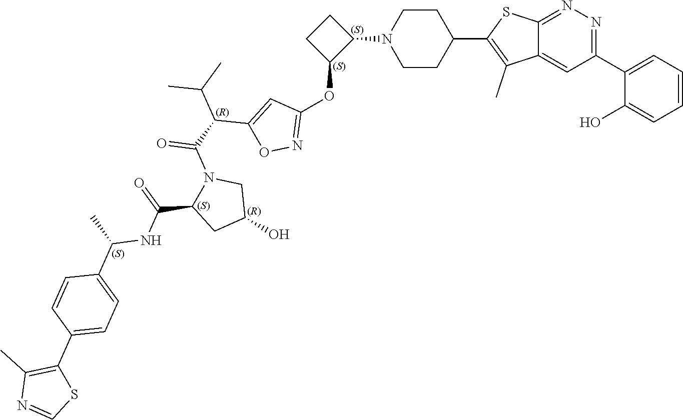 |
| 37 | 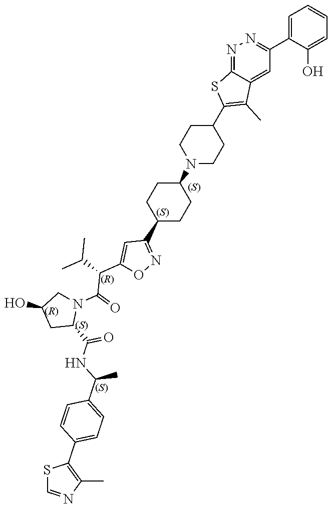 |
| 38 | 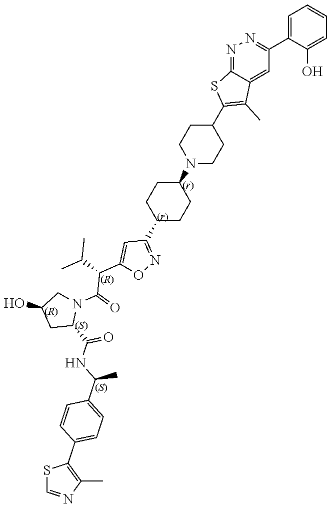 |
| 39 | 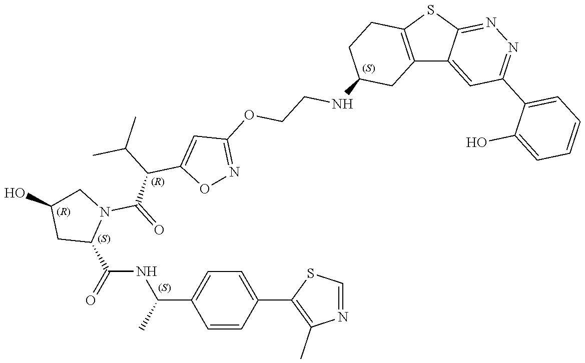 |
| 40 | 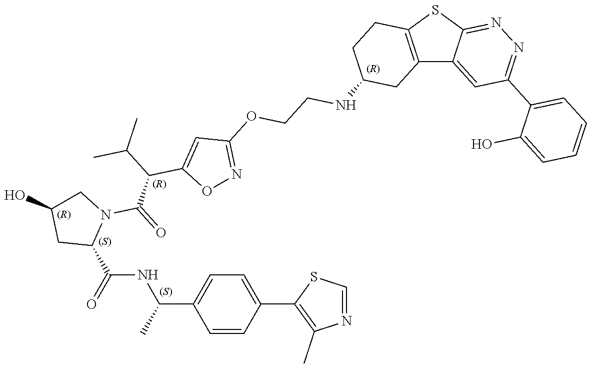 |
| 41 | 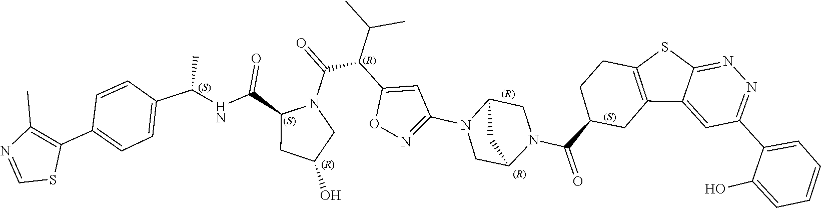 |
| 42 | 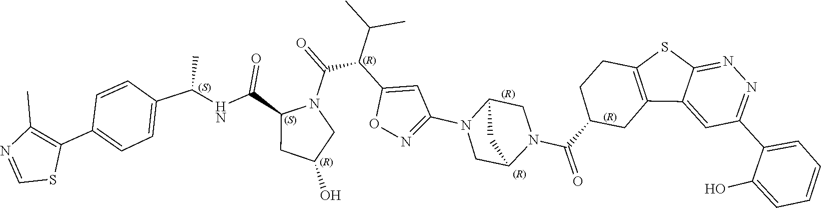 |
| 43 | 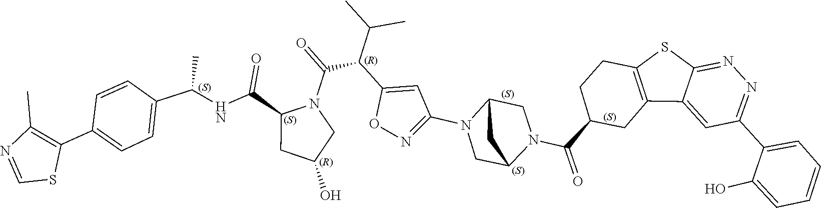 |
| 44 | 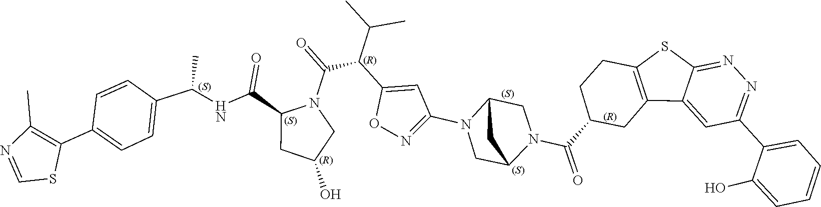 |
| 45 | 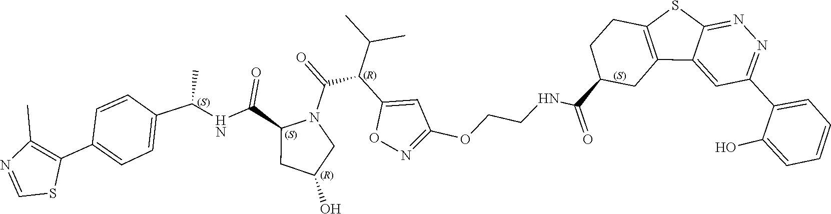 |
| 46 | 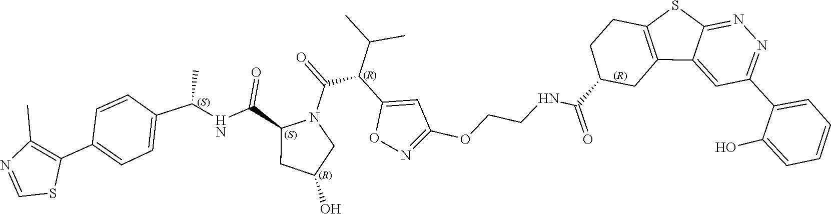 |
| 47 | 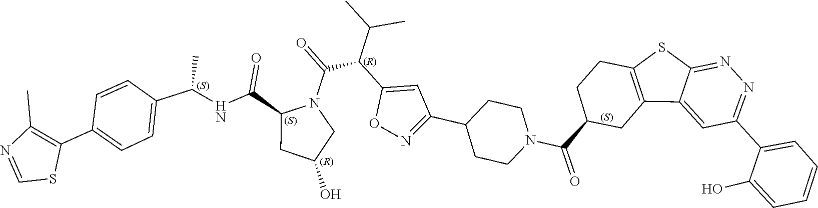 |
| 48 | 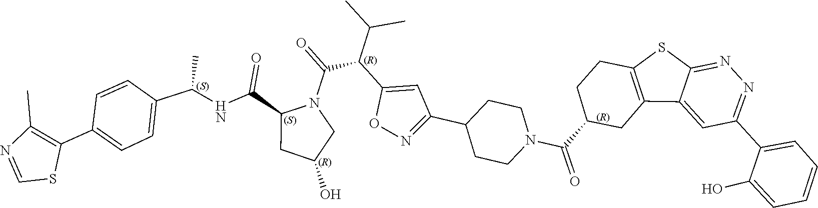 |
| 49 | 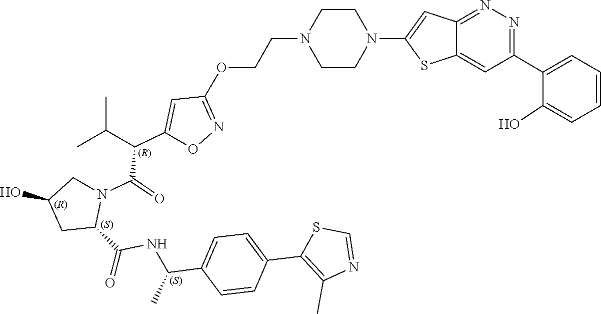 |
| 50 | 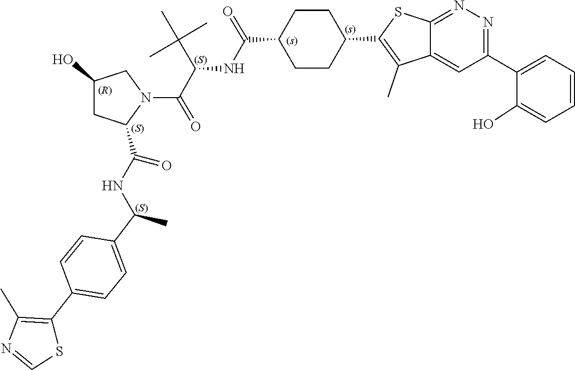 |
| 51 | 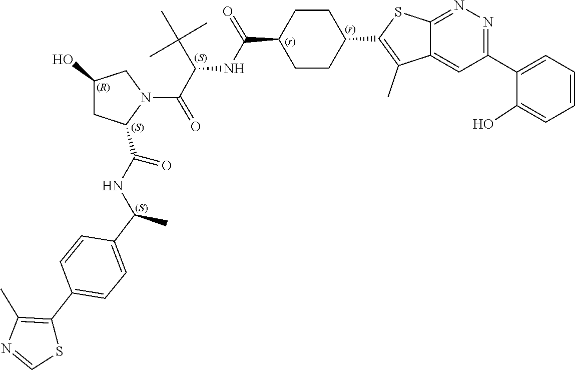 |
| 52 | 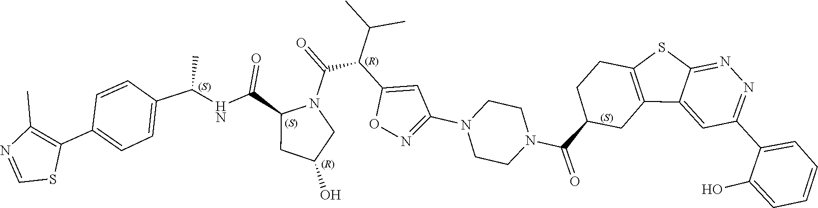 |
| 53 | 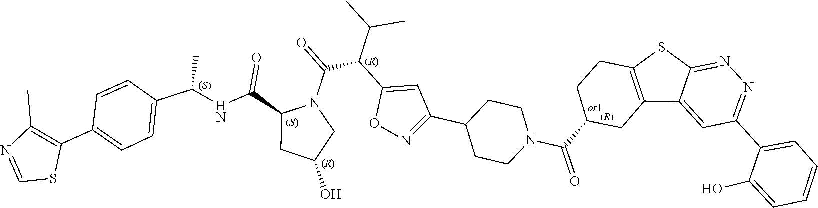 |
| 54 | 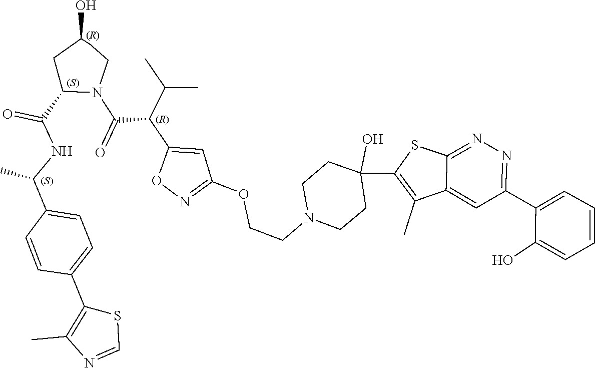 |
| 55 | 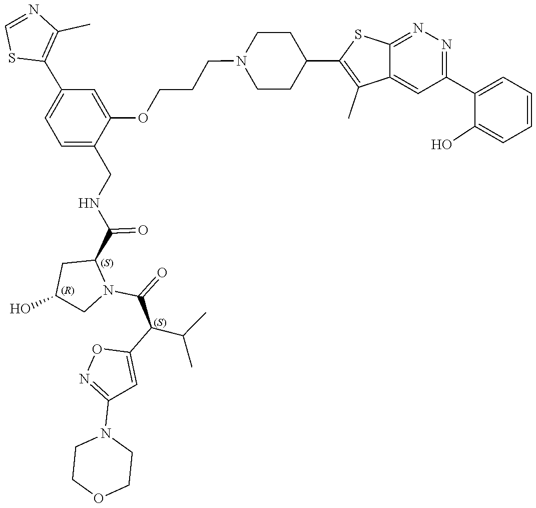 |
| 56 | 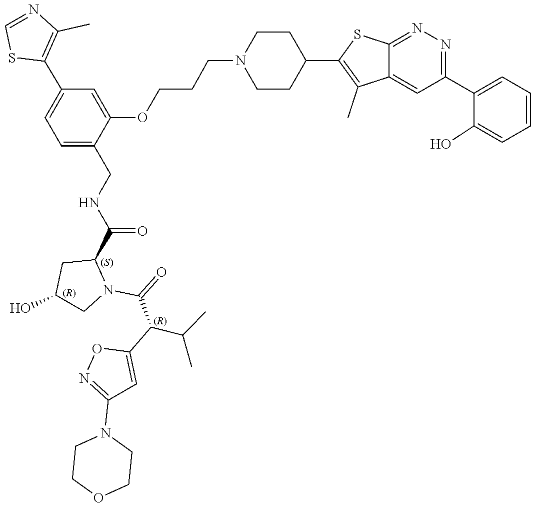 |
| 57 | 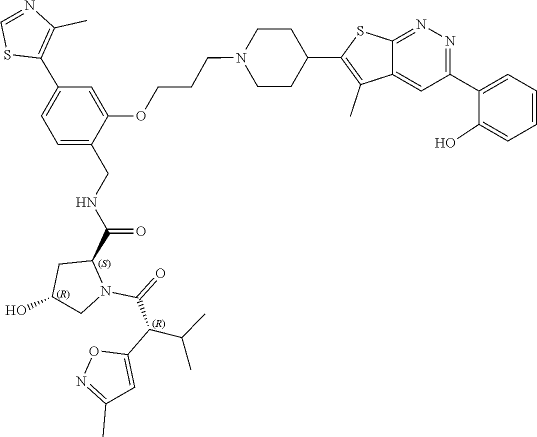 |
| 58 | 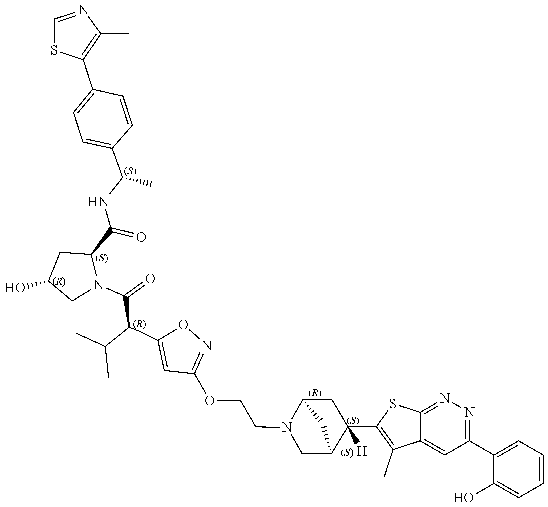 |
| 59 | 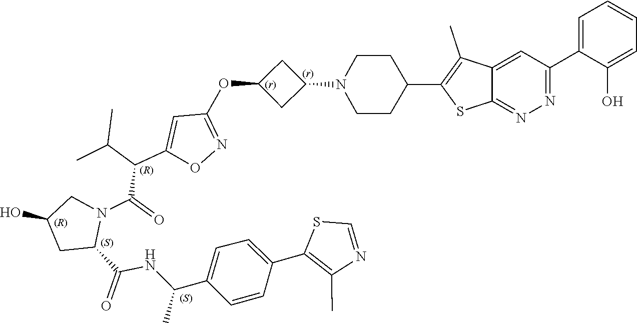 |
| 60 | 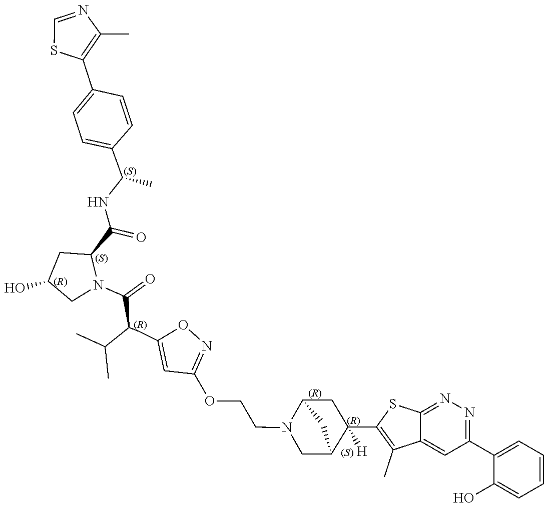 |
| 61 | 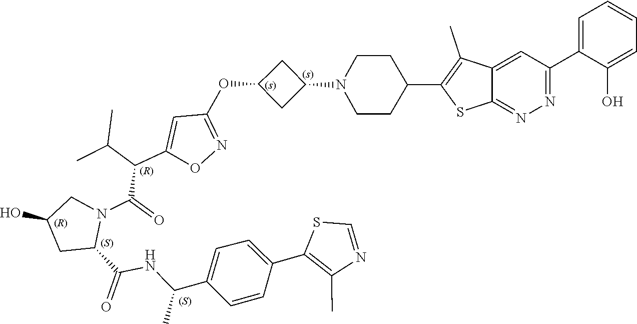 |
| 62 | 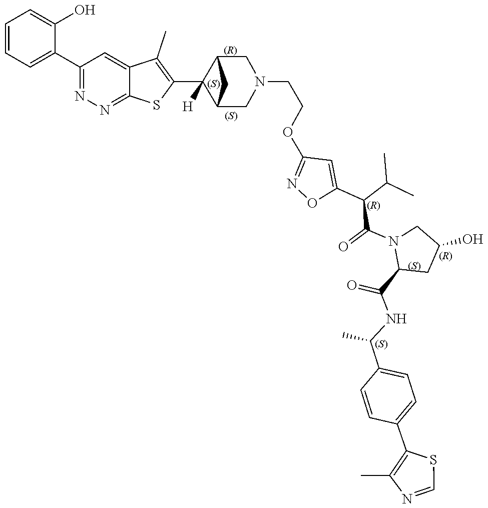 |
| 63 | 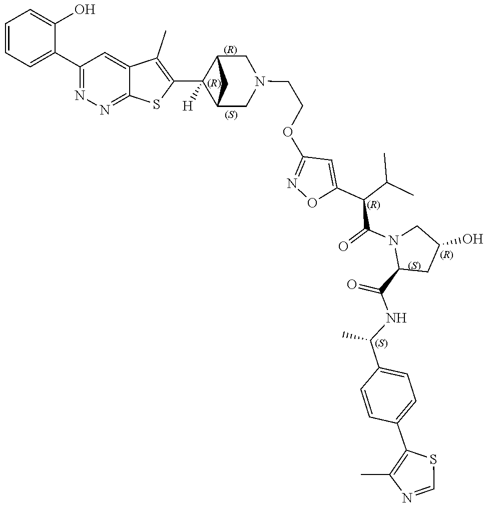 |
| 64 | 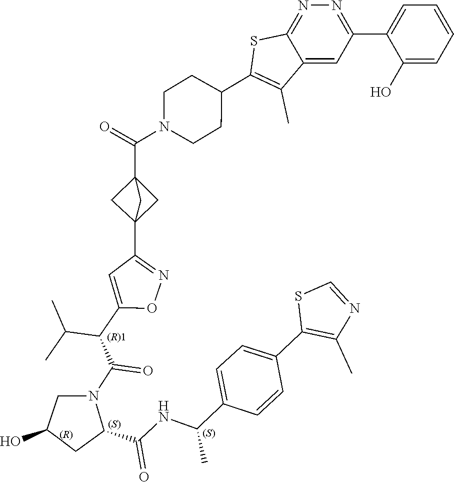 |
| 65 | 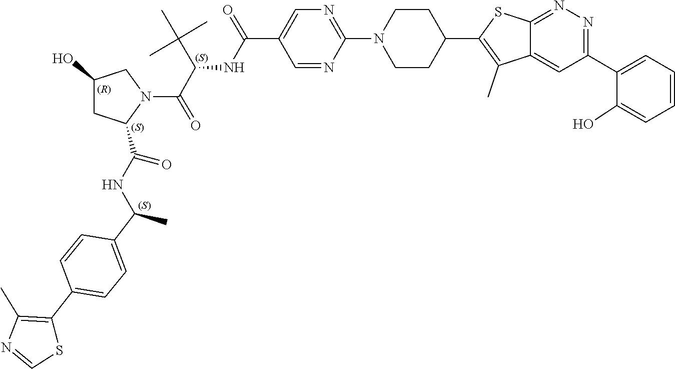 |
| 66 | 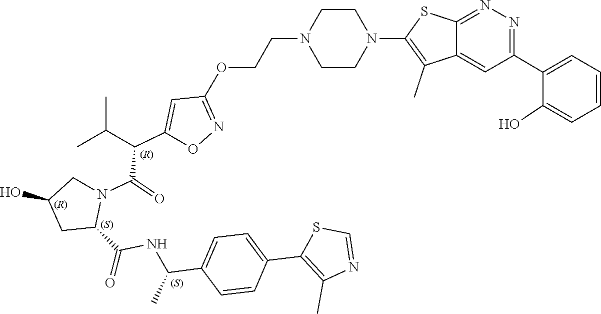 |
| 67 | 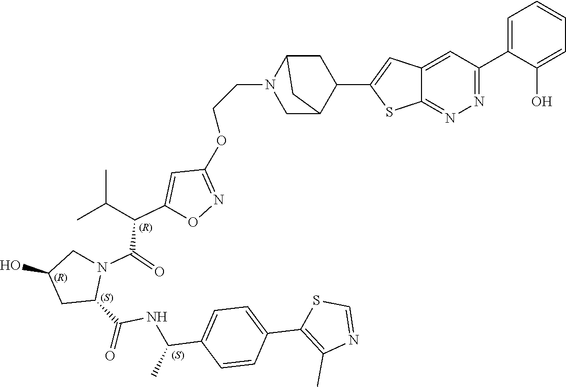 |
| 68 | 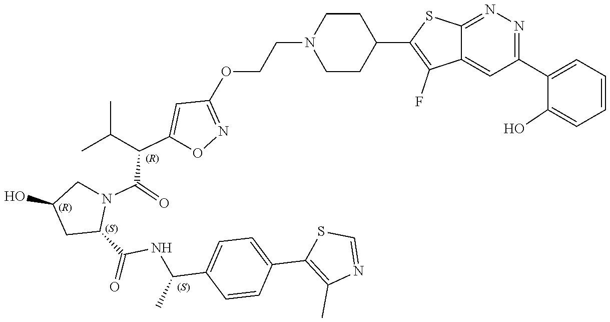 |
| 69 | 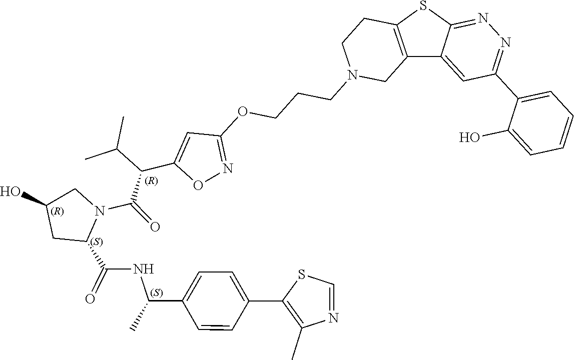 |
| 70 | 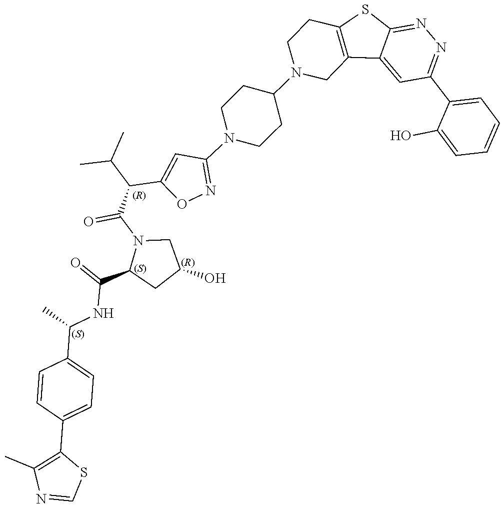 |
| 71 | 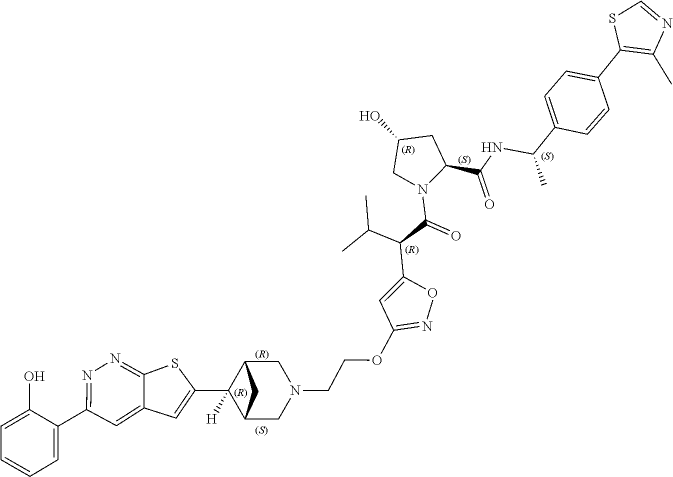 |
| 72 | 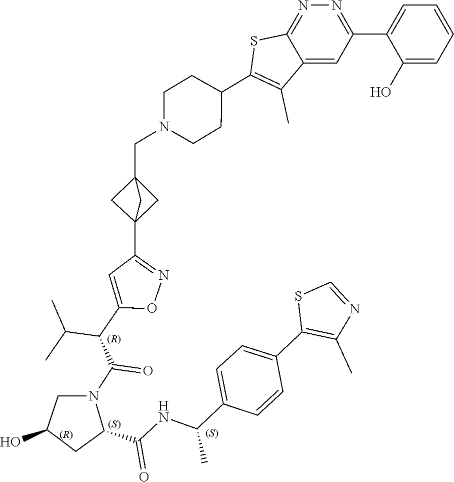 |
| 73 | 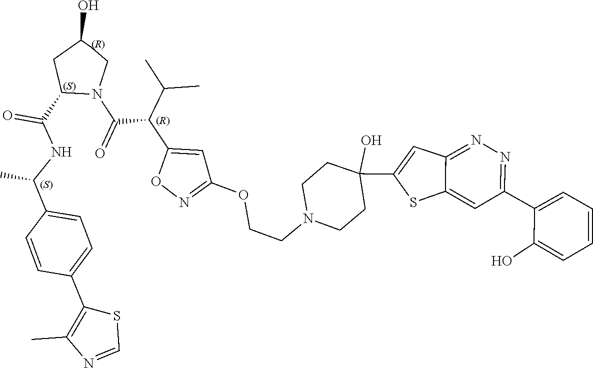 |
| 74 | 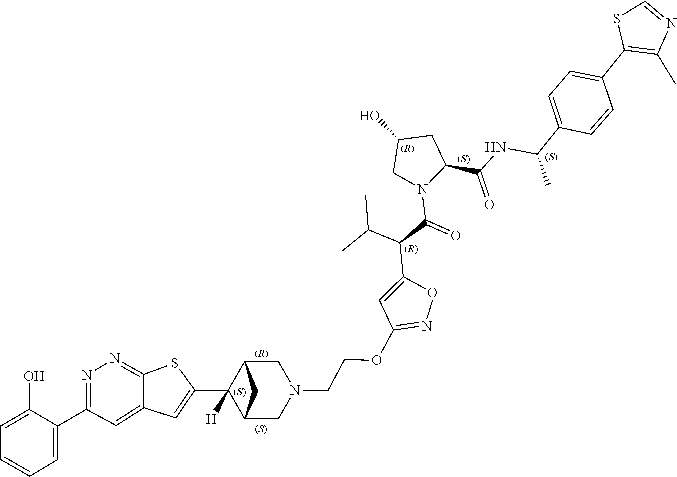 |
| 75 | 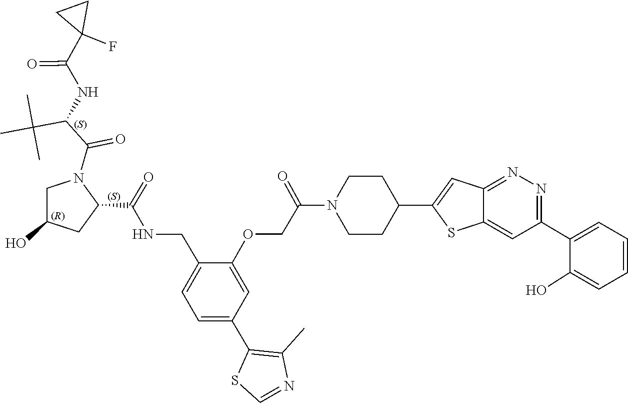 |
| 76 | 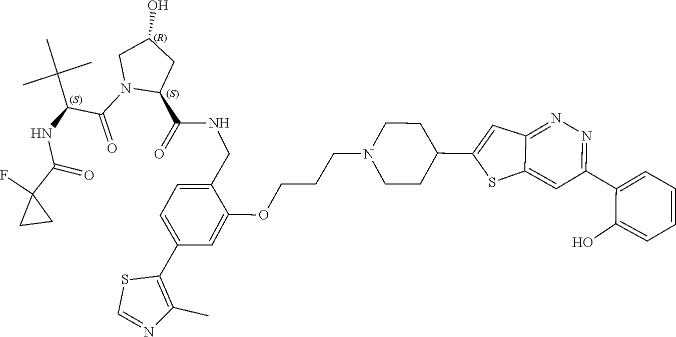 |
| 77 | 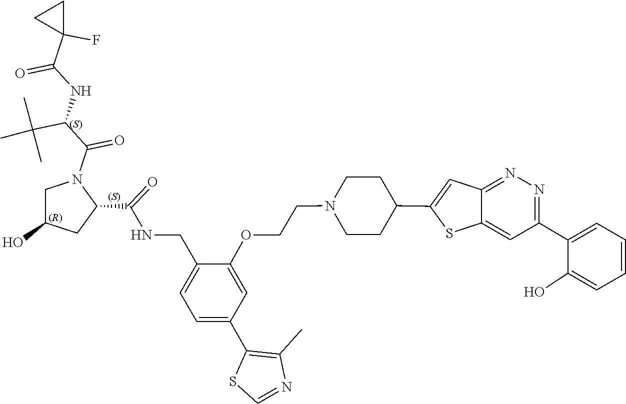 |
| 78 | 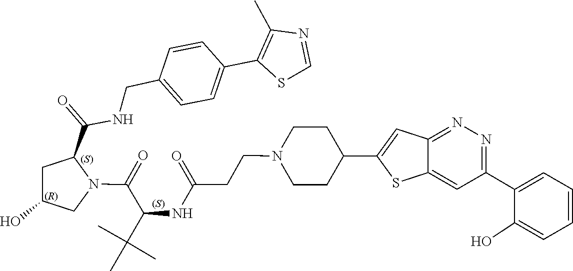 |
| 79 | 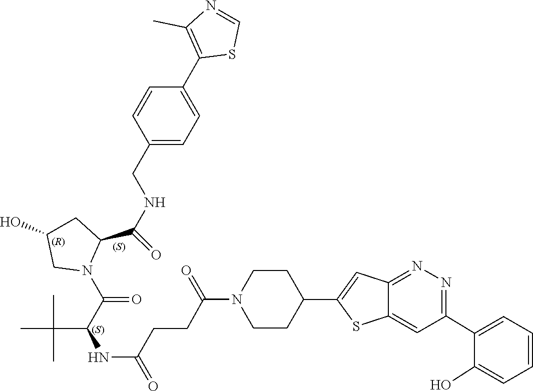 |
| 80 | 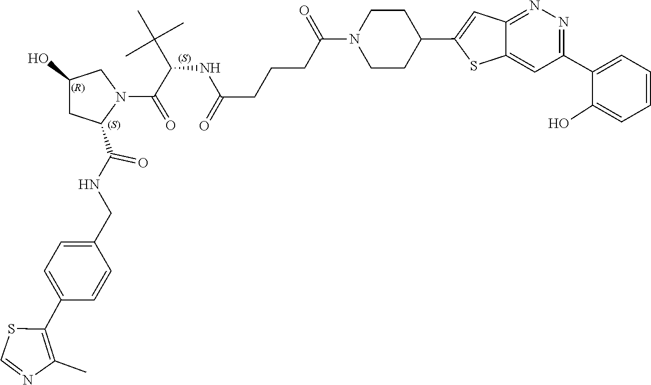 |
| 81 | 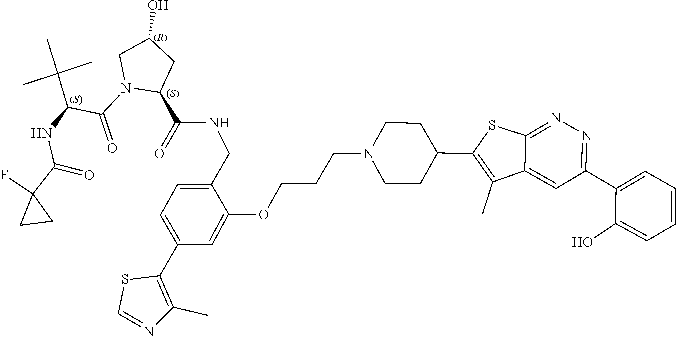 |
| 82 | 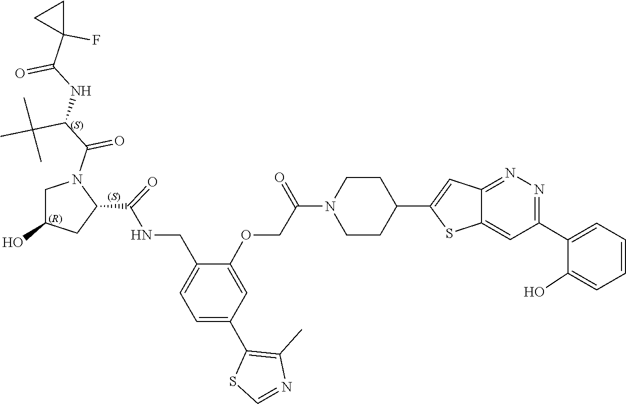 |
| 83 | 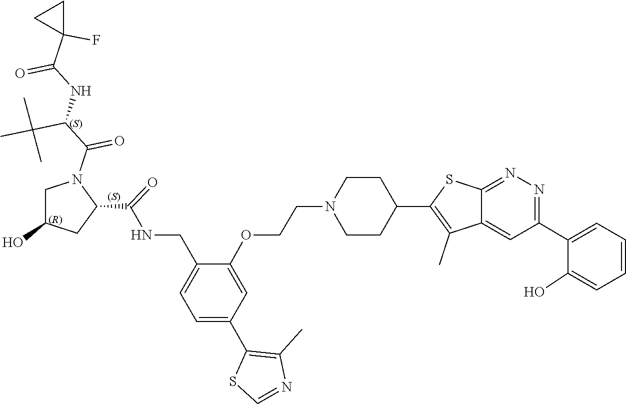 |
| 84 | 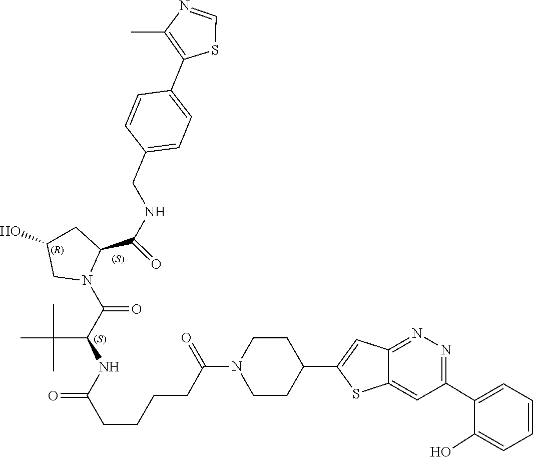 |
| 85 | 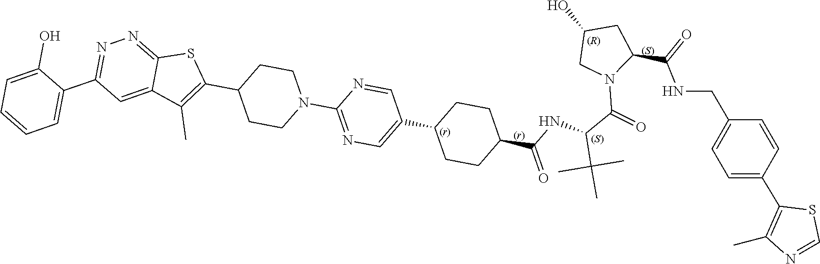 |
| 86 | 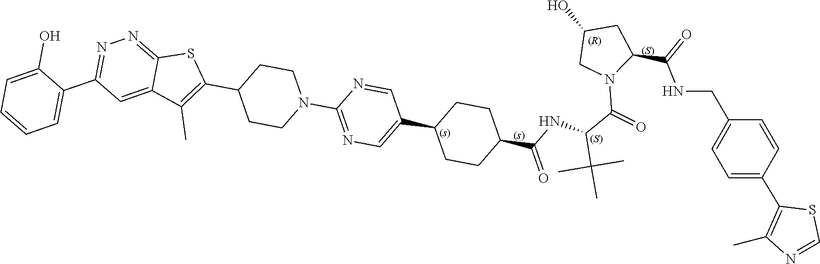 |
| 87 | 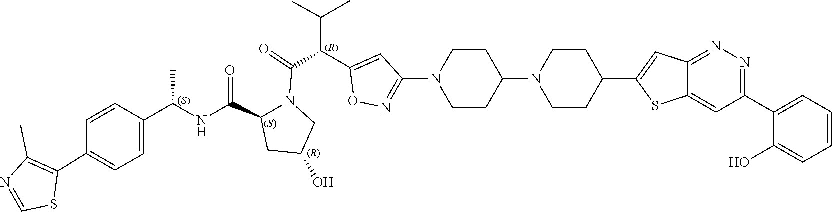 |
| 88 | 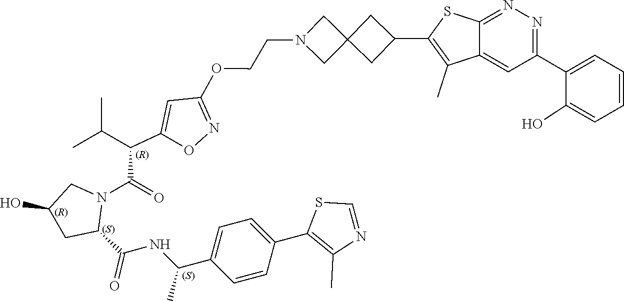 |
| 89 | 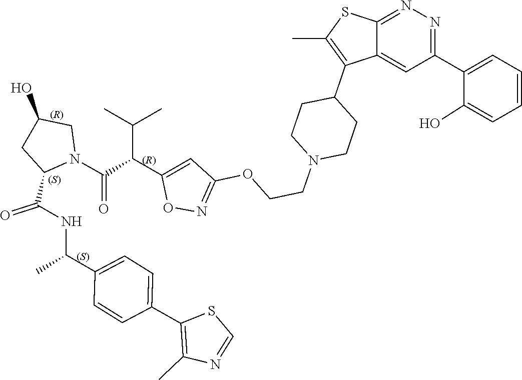 |
| 90 | 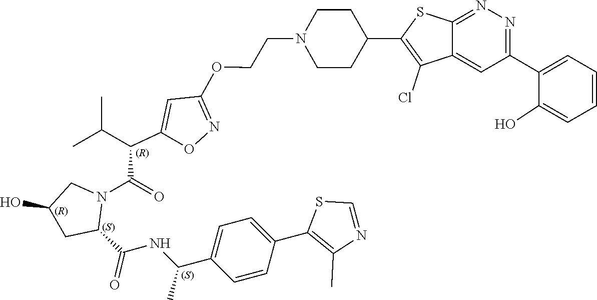 |
| 91 | 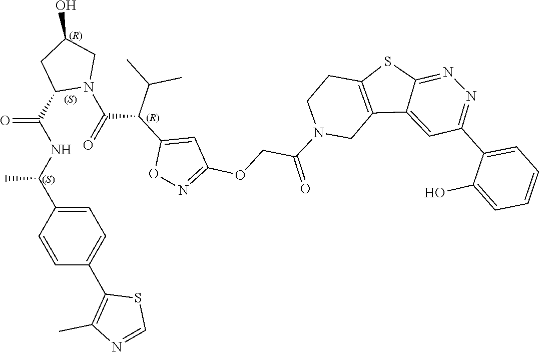 |
| 92 | 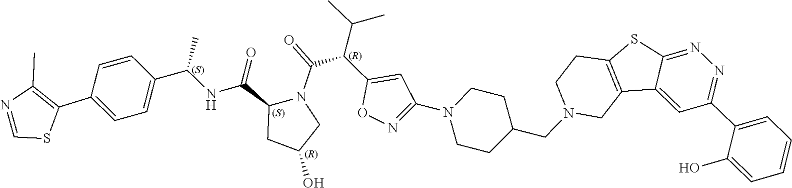 |
| 93 | 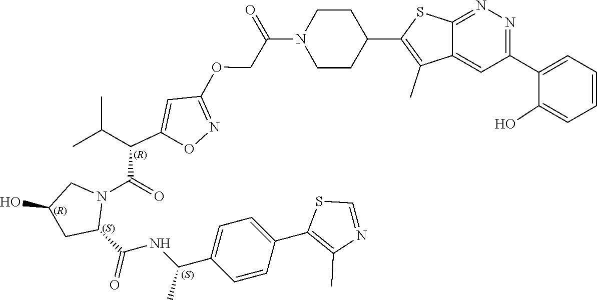 |
| 94 | 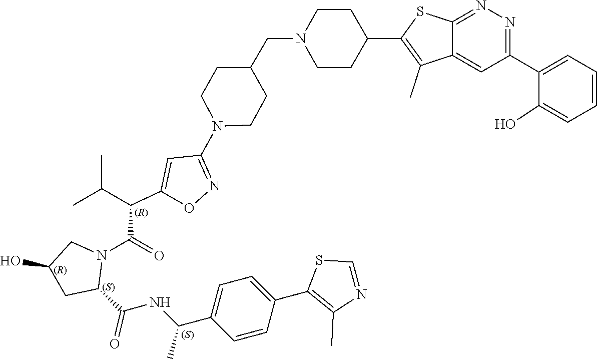 |
| 95 | 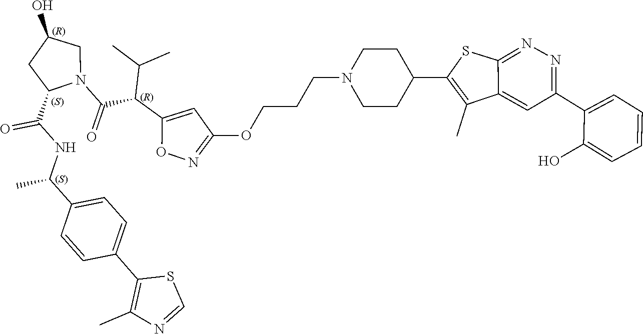 |
| 96 | 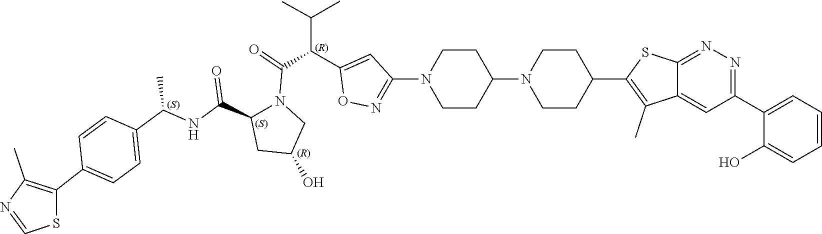 |
| 97 | 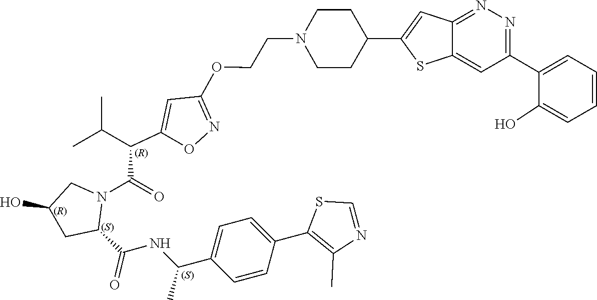 |
| 98 | 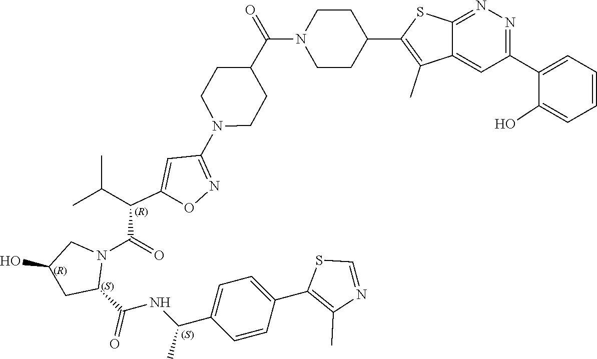 |
| 99 | 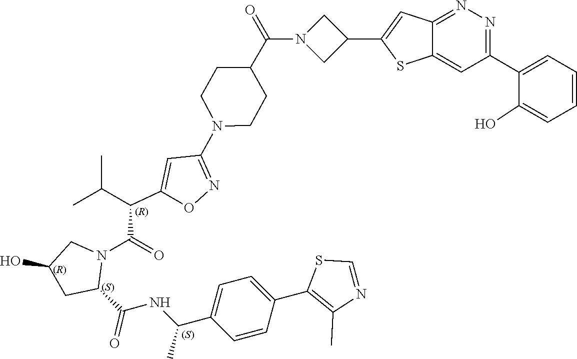 |
| 100 | 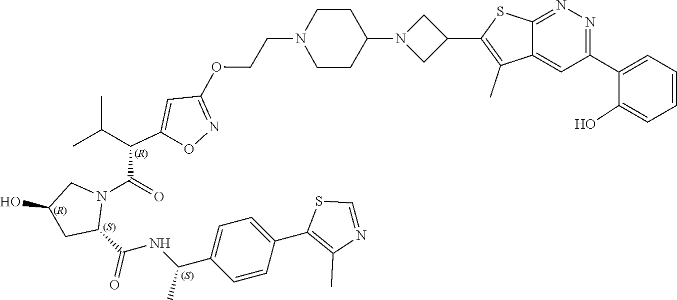 |
| 101 | 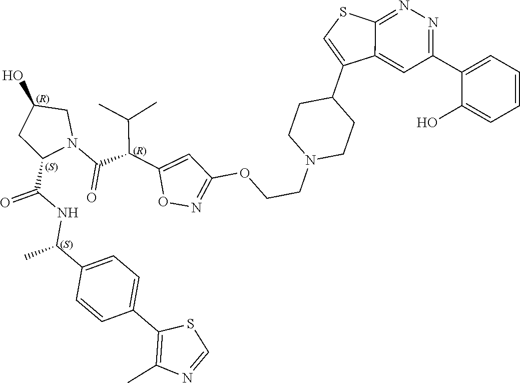 |
| 102 | 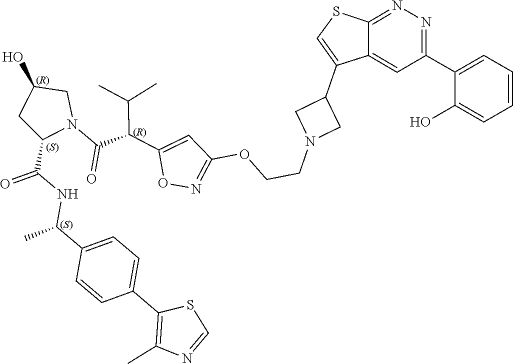 |
| 103 | 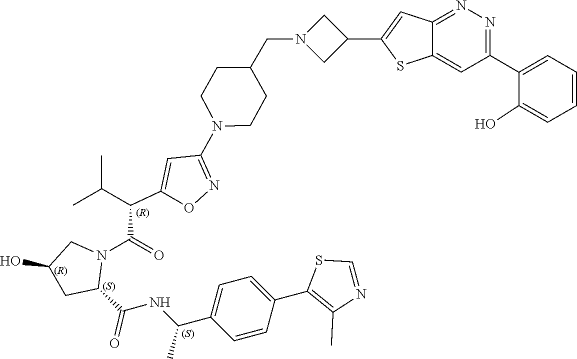 |
| 104 | 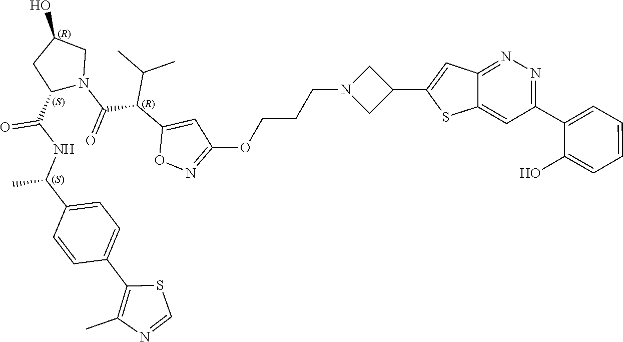 |
| 105 | 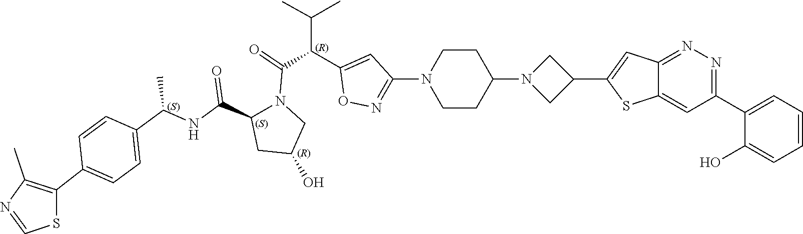 |
| 106 | 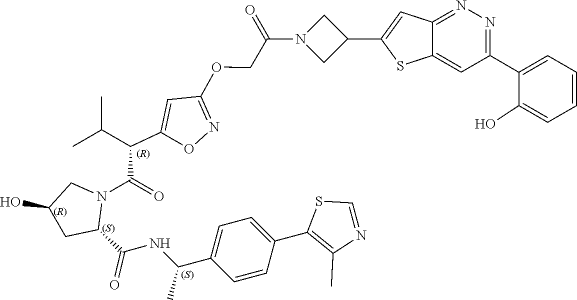 |
| 107 | 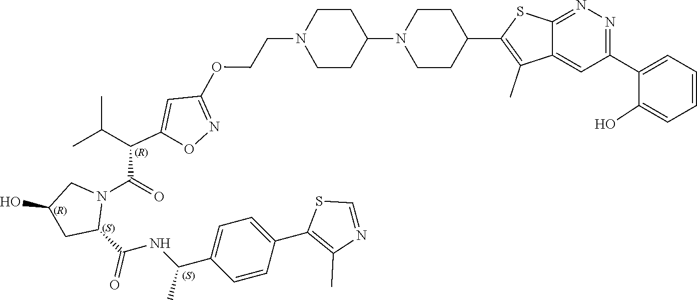 |
| 108 | 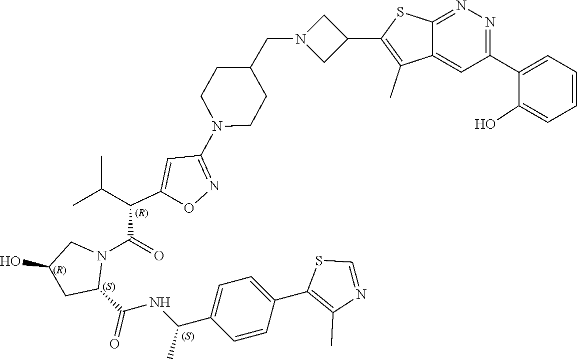 |
| 109 | 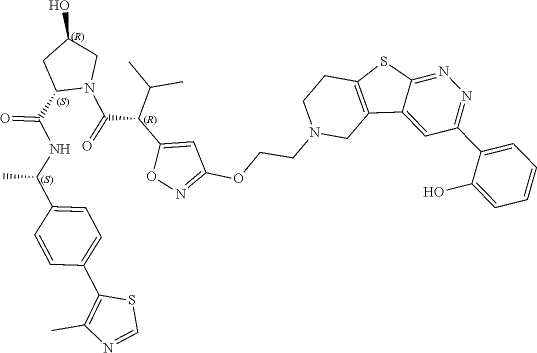 |
| 110 | 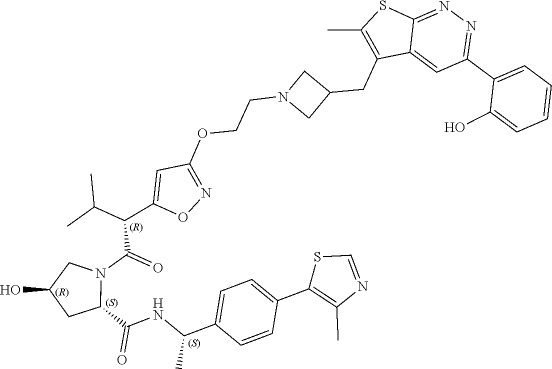 |
| 111 | 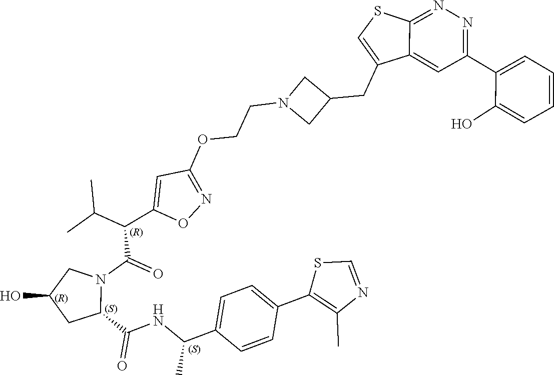 |
| 112 | 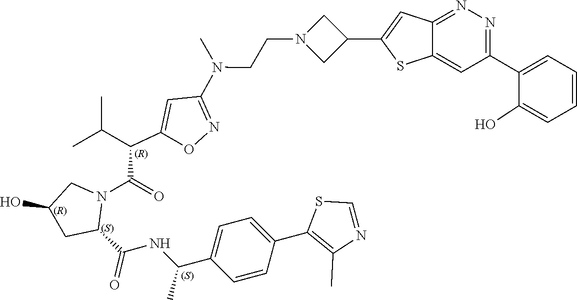 |
| 113 | 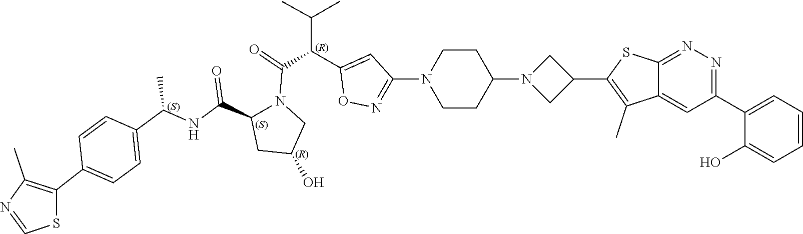 |
| 114 | 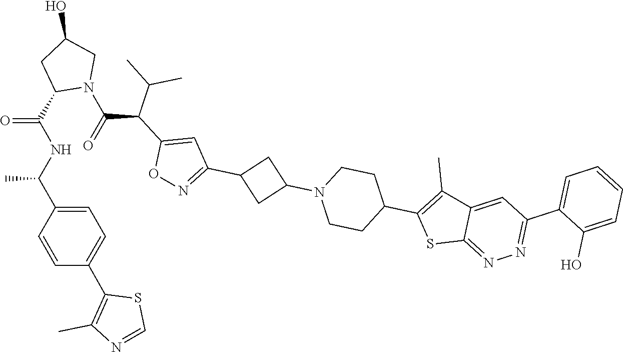 [SMILES: Cc1ncsc1-c1ccc([C@H](C)NC(=O)[C@@H]2C[C@@H](O)CN2C(=O)[C@H](c2cc(C3CC(N4CCC(c5sc6nnc(-c7ccccc7O)cc6c5C)CC4)C3)no2)C(C)C)cc1] |
| 115 | 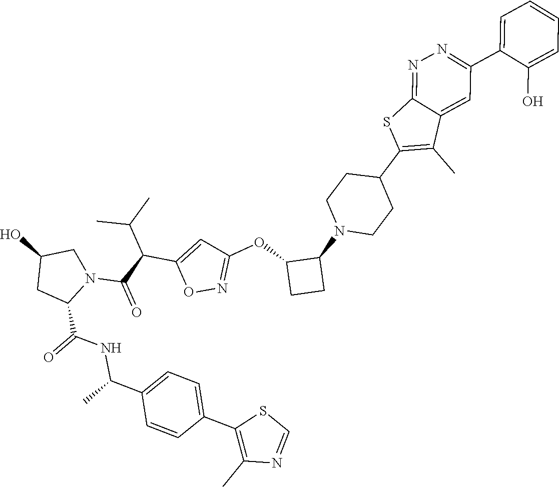 [SMILES: Cc1ncsc1-c1ccc([C@H](C)NC(=O)[C@@H]2C[C@@H](O)CN2C(=O)[C@H](c2cc(O[C@H]3CC[C@@H]3N3CCC(c4sc5nnc(-c6ccccc6O)cc5c4C)CC3)no2)C(C)C)cc1] |

In some embodiments, the compound as a ratio of BRG1 IC₅₀ to BRM IC₅₀ of at least 5. In some embodiments, the compound has a ratio of BRG1 IC₅₀ to BRM IC₅₀ of at least 7. In some embodiments, the compound has a ratio of BRG1 IC₅₀ to BRM IC₅₀ of at least 10. In some embodiments, the compound has a ratio of BRG1 IC₅₀ to BRM IC₅₀ of at least 15. In some embodiments, the compound has a ratio of BRG1 IC₅₀ to BRM IC₅₀ of at least 20. In some embodiments, the compound has a ratio of BRG1 IC₅₀ to BRM IC₅₀ of at least 25. In some embodiments, the compound has a ratio of BRG1 IC₅₀ to BRM IC₅₀ of at least 30.

In an aspect, the invention features a pharmaceutical composition comprising any of the foregoing compounds and a pharmaceutically acceptable excipient.

In another aspect, the invention features a method of decreasing the activity of a BAF complex in a cell, the method involving contacting the cell with an effective amount of any of the foregoing compounds or a pharmaceutical composition thereof.

In some embodiments, the cell is a cancer cell.

In another aspect, the invention features a method of treating a BAF complex-related disorder in a subject in need thereof, the method involving administering to the subject an effective amount of any of the foregoing compounds (e.g., a BRM/BRG1 dual inhibitor compound or a BRM-selective compound) or a pharmaceutical composition thereof.

In some embodiments, the BAF complex-related disorder is cancer.

In a further aspect, the invention features a method of inhibiting BRM, the method involving contacting a cell with an effective amount of any of the foregoing compounds (e.g., a BRM/BRG1 dual inhibitor compound or a BRM-selective compound) or a pharmaceutical composition thereof.

In some embodiments, the cell is a cancer cell.

In another aspect, the invention features a method of inhibiting BRG1, the method involving contacting the cell with an effective amount of any of the foregoing compounds or a pharmaceutical composition thereof.

In some embodiments, the cell is a cancer cell.

In a further aspect, the invention features a method of inhibiting BRM and BRG1, the method involving contacting the cell with an effective amount of any of the foregoing compounds or a pharmaceutical composition thereof.

In some embodiments, the cell is a cancer cell.

In another aspect, the invention features a method of treating a disorder related to a BRG1 loss of function mutation in a subject in need thereof, the method involving administering to the subject an effective amount of any of the foregoing compounds (e.g., a BRM/BRG1 dual inhibitor compound or a BRM-selective compound) or a pharmaceutical composition thereof.

In some embodiments, the disorder related to a BRG1 loss of function mutation is cancer. In other embodiments, the subject is determined to have a BRG1 loss of function disorder, for example, is determined to have a BRG1 loss of function cancer (for example, the cancer has been determined to include cancer cells with loss of BRG1 function).

In another aspect, the invention features a method of inducing apoptosis in a cell, the method involving contacting the cell with an effective amount of any of the foregoing compounds (e.g., a BRM/BRG1 dual inhibitor compound or a BRM-selective compound) or a pharmaceutical composition thereof.

In some embodiments, the cell is a cancer cell.

In a further aspect, the invention features a method of treating cancer in a subject in need thereof, the method including administering to the subject an effective amount of any of the foregoing compounds (e.g., a BRM/BRG1 dual inhibitor compound or a BRM-selective compound) or a pharmaceutical composition thereof.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, or penile cancer.

In some embodiments of any of the foregoing methods, the cancer is a drug resistant cancer or has failed to respond to a prior therapy (e.g., vemurafenib, dacarbazine, a CTLA4 inhibitor, a PD1 inhibitor, interferon therapy, a BRAF inhibitor, a MEK inhibitor, radiotherapy, temozolomide, irinotecan, a CAR-T therapy, Herceptin®, Perjeta®, tamoxifen, Xeloda®, docetaxol, platinum agents such as carboplatin, taxanes such as paclitaxel and docetaxel, ALK inhibitors, MET inhibitors, Alimta®, Abraxane®, Adriamycin®, gemcitabine, Avastin®, Halaven®, neratinib, a PARP inhibitor, ARN810, an mTOR inhibitor, topotecan, Gemzar®, a VEGFR2 inhibitor, a folate receptor antagonist, demcizumab, fosbretabulin, or a PDL1 inhibitor).

In some embodiments of any of the foregoing methods, the cancer has or has been determined to have BRG1 mutations. In some embodiments of any of the foregoing methods, the BRG1 mutations are homozygous. In some embodiments of any of the foregoing methods, the cancer does not have, or has been determined not to have, an epidermal growth factor receptor (EGFR) mutation. In some embodiments of any of the foregoing methods, the cancer does not have, or has been determined not to have, an anaplastic lymphoma kinase (ALK) driver mutation. In some embodiments of any of the foregoing methods, the cancer has, or has been determined to have, a KRAS mutation. In some embodiments of any of the foregoing methods, the BRG1 mutation is in the ATPase catalytic domain of the protein. In some embodiments of any of the foregoing methods, the BRG1 mutation is a deletion at the C-terminus of BRG1.

In another aspect, the disclosure provides a method treating a disorder related to BAF (e.g., cancer or viral infections) in a subject in need thereof. This method includes contacting a cell with an effective amount of any of the foregoing compounds (e.g., a BRM/BRG1 dual inhibitor compound or a BRM-selective compound), or pharmaceutically acceptable salts thereof, or any of the foregoing pharmaceutical compositions. In some embodiments, the disorder is a viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), Togaviridae family (e.g., Rubella virus). In some embodiments, the disorder is Coffin Siris, Neurofibromatosis (e.g., NF-1, NF-2, or Schwannomatosis), or Multiple Meningioma.

In another aspect, the disclosure provides a method for treating a viral infection in a subject in need thereof. This method includes administering to the subject an effective amount of any of the foregoing compounds (e.g., a BRM/BRG1 dual inhibitor compound or a BRM-selective compound), or pharmaceutically acceptable salts thereof, or any of the foregoing pharmaceutical compositions. In some embodiments, the viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), or Togaviridae family (e.g., Rubella virus).

In some embodiments of any of the foregoing aspects, the compound is a BRM-selective compound. In some embodiments, the BRM-selective compound inhibits the level and/or activity of BRM at least 10-fold greater than the compound inhibits the level and/or activity of BRG1 and/or the compound binds to BRM at least 10-fold greater than the compound binds to BRG1. For example, in some embodiments, a BRM-selective compound has an IC₅₀ or IP₅₀ that is at least 10-fold lower than the IC₅₀ or IP₅₀ against BRG1. In some embodiments of any of the foregoing aspects, the compound is a BRM/BRG1 dual inhibitor compound. In some embodiments, the BRM/BRG1 dual inhibitor compound has similar activity against both BRM and BRG1 (e.g., the activity of the compound against BRM and BRG1 with within 10-fold (e.g., less than 5-fold, less than 2-fold). In some embodiments, the activity of the BRM/BRG1 dual inhibitor compound is greater against BRM. In some embodiments, the activity of the BRM/BRG1 dual inhibitor compound is greater against BRG1. For example, in some embodiments, a BRM/BRG1 dual inhibitor compound has an IC₅₀ or IP₅₀ against BRM that is within 10-fold of the IC₅₀ or IP₅₀ against BRG1.

In another aspect, the invention features a method of treating melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of reducing tumor growth of melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of suppressing metastatic progression of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of suppressing metastatic colonization of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of reducing the level and/or activity of BRG1 and/or BRM in a melanoma, prostate cancer, breast cancer, bone cancer, renal cell carcinoma, or hematologic cancer cell, the method including contacting the cell with an effective amount of any of the foregoing compounds or pharmaceutical compositions thereof.

In some embodiments of any of the above aspects, the melanoma, prostate cancer, breast cancer, bone cancer, renal cell carcinoma, or hematologic cell is in a subject.

In some embodiments of any of the above aspects, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRG1 by at least 50% (e.g., 55%, 60%, 65%, 70%, 75%, 80%, 85%, 90%, or 95%) as compared to a reference. In some embodiments, the effective amount of the compound that reduces the level and/or activity of BRG1 by at least 90% (e.g., 91%, 92%, 93%, 94%, 95%, 96%, 97%, 98%, or 99%).

In some embodiments, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments of any of the above aspects, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRM by at least 50% (e.g., 55%, 60%, 65%, 70%, 75%, 80%, 85%, 90%, or 95%) as compared to a reference. In some embodiments, the effective amount of the compound that reduces the level and/or activity of BRM by at least 90% (e.g., 91%, 92%, 93%, 94%, 95%, 96%, 97%, 98%, or 99%).

In some embodiments, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments, the subject has cancer. In some embodiments, the cancer expresses BRG1 and/or BRM protein and/or the cell or subject has been identified as expressing BRG1 and/or BRM. In some embodiments, the cancer expresses BRG1 protein and/or the cell or subject has been identified as expressing BRG1. In some embodiments, the cancer expresses BRM protein and/or the cell or subject has been identified as expressing BRM. In some embodiments, the cancer is melanoma (e.g., uveal melanoma, mucosal melanoma, or cutaneous melanoma). In some embodiments, the cancer is prostate cancer. In some embodiments, the cancer is a hematologic cancer, e.g., multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia (e.g., T-cell acute lymphoblastic leukemia or B-cell acute lymphoblastic leukemia), diffuse large cell lymphoma, or non-Hodgkin's lymphoma. In some embodiments, the cancer is breast cancer (e.g., an ER positive breast cancer, an ER negative breast cancer, triple positive breast cancer, or triple negative breast cancer). In some embodiments, the cancer is a bone cancer (e.g., Ewing's sarcoma). In some embodiments, the cancer is a renal cell carcinoma (e.g., a Microphthalmia Transcription Factor (MITF) family translocation renal cell carcinoma (tRCC)). In some embodiments, the cancer is metastatic (e.g., the cancer has spread to the liver). The metastatic cancer can include cells exhibiting migration and/or invasion of migrating cells and/or include cells exhibiting endothelial recruitment and/or angiogenesis. In other embodiments, the migrating cancer is a cell migration cancer. In still other embodiments, the cell migration cancer is a non-metastatic cell migration cancer. The metastatic cancer can be a cancer spread via seeding the surface of the peritoneal, pleural, pericardial, or subarachnoid spaces. Alternatively, the metastatic cancer can be a cancer spread via the lymphatic system, or a cancer spread hematogenously. In some embodiments, the effective amount of an agent that reduces the level and/or activity of BRG1 and/or BRM is an amount effective to inhibit metastatic colonization of the cancer to the liver.

In some embodiments the cancer harbors a mutation in GNAQ. In some embodiments the cancer harbors a mutation in GNA11. In some embodiments the cancer harbors a mutation in PLCB4. In some embodiments the cancer harbors a mutation in CYSLTR2. In some embodiments the cancer harbors a mutation in BAP1. In some embodiments the cancer harbors a mutation in SF3B1. In some embodiments the cancer harbors a mutation in EIF1AX. In some embodiments the cancer harbors a TFE3 translocation. In some embodiments the cancer harbors a TFEB translocation. In some embodiments the cancer harbors a MITF translocation. In some embodiments the cancer harbors an EZH2 mutation. In some embodiments the cancer harbors a SUZ12 mutation. In some embodiments the cancer harbors an EED mutation.

In some embodiments, the method further includes administering to the subject or contacting the cell with an anticancer therapy, e.g., a chemotherapeutic or cytotoxic agent, immunotherapy, surgery, radiotherapy, thermotherapy, or photocoagulation. In some embodiments, the anticancer therapy is a chemotherapeutic or cytotoxic agent, e.g., an antimetabolite, antimitotic, antitumor antibiotic, asparagine-specific enzyme, bisphosphonates, antineoplastic, alkylating agent, DNA-Repair enzyme inhibitor, histone deacetylase inhibitor, corticosteroid, demethylating agent, immunomodulatory, janus-associated kinase inhibitor, phosphinositide 3-kinase inhibitor, proteasome inhibitor, or tyrosine kinase inhibitor.

In some embodiments, the compound of the invention is used in combination with another anti-cancer therapy used for the treatment of uveal melanoma such as surgery, a MEK inhibitor, and/or a PKC inhibitor. For example, in some embodiments, the method further comprises performing surgery prior to, subsequent to, or at the same time as administration of the compound of the invention. In some embodiments, the method further comprises administration of a MEK inhibitor and/or a PKC inhibitor prior to, subsequent to, or at the same time as administration of the compound of the invention.

In some embodiments, the anticancer therapy and the compound of the invention are administered within 28 days of each other and each in an amount that together are effective to treat the subject.

In some embodiments, the subject or cancer has and/or has been identified as having a BRG1 loss of function mutation.

In some embodiments, the cancer is resistant to one or more chemotherapeutic or cytotoxic agents (e.g., the cancer has been determined to be resistant to chemotherapeutic or cytotoxic agents such as by genetic markers, or is likely to be resistant, to chemotherapeutic or cytotoxic agents such as a cancer that has failed to respond to a chemotherapeutic or cytotoxic agent). In some embodiments, the cancer has failed to respond to one or more chemotherapeutic or cytotoxic agents. In some embodiments, the cancer is resistant or has failed to respond to dacarbazine, temozolomide, cisplatin, treosulfan, fotemustine, IMCgp100, a CTLA-4 inhibitor (e.g., ipilimumab), a PD-1 inhibitor (e.g., Nivolumab or pembrolizumab), a PD-L1 inhibitor (e.g., atezolizumab, avelumab, or durvalumab), a mitogen-activated protein kinase (MEK) inhibitor (e.g., selumetinib, binimetinib, or tametinib), and/or a protein kinase C (PKC) inhibitor (e.g., sotrastaurin or IDE196).

In some embodiments, the cancer is resistant to or failed to respond to a previously administered therapeutic used for the treatment of uveal melanoma such as a MEK inhibitor or PKC inhibitor. For example, in some embodiments, the cancer is resistant to or failed to respond to a mitogen-activated protein kinase (MEK) inhibitor (e.g., selumetinib, binimetinib, or tametinib), and/or a protein kinase C (PKC) inhibitor (e.g., sotrastaurin or IDE196).

### Chemical Terms

The terminology employed herein is for the purpose of describing particular embodiments and is not intended to be limiting.

For any of the following chemical definitions, a number following an atomic symbol indicates that total number of atoms of that element that are present in a particular chemical moiety. As will be understood, other atoms, such as H atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

The term “acyl,” as used herein, represents a H or an alkyl group that is attached to a parent molecular group through a carbonyl group, as defined herein, and is exemplified by formyl (i.e., a carboxaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6 carbon atoms, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6 carbon atoms, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged polycycloalkyl,” as used herein, refers to a bridged polycyclic group of 5 to 20 carbons, containing from 1 to 3 bridges.

The term “cyano,” as used herein, represents a —CN group.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, and monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to a mono- or polycyclic radical of 5 to 12 atoms having at least one aromatic ring and containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a mono- or polycyclic radical having 3 to 12 atoms having at least one ring containing 1, 2, 3, or 4 ring atoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “oxo,” as used herein, represents a divalent oxygen atom (e.g., the structure of oxo may be shown as =O). For example, a carbonyl group is a carbon (e.g., alkyl carbon, alkenyl carbon, alkynyl carbon, heteroalkyl carbon, heteroalkenyl carbon, heteroalkynyl carbon, carbocyclyl carbon, etc.) substituted with oxo. Alternatively, sulfur may be substituted with one or two oxo groups (e.g., —SO— or —SO₂— within a substituted heteroalkyl, heteroalkenyl, heteroalkynyl, or heterocyclyl group).

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Another exemplary substituent is oxo. For example, a carbonyl group is a carbon (e.g., alkyl carbon, alkenyl carbon, alkynyl carbon, heteroalkyl carbon, heteroalkenyl carbon, heteroalkynyl carbon, carbocyclyl carbon, etc.) substituted with oxo. Alternatively, sulfur may be substituted with one or two oxo groups (e.g., —SO— or —SO₂— within a substituted heteroalkyl, heteroalkenyl, heteroalkynyl, or heterocyclyl group). Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)). In some embodiments, alkyl, alkenyl, alkynyl, heteroalkyl, heteroalkenyl, and heteroalkynyl are optionally substituted with 1, 2, 3, 4, or 5 substituents independently selected from the group consisting of aryl (e.g., substituted and unsubstituted phenyl), carbocyclyl (e.g., substituted and unsubstituted cycloalkyl), halo (e.g., fluoro), hydroxyl, heteroaryl, heterocyclyl, amino (e.g., NH₂ or mono- or dialkyl amino), azido, cyano, nitro, thiol, and oxo. In some embodiments, the substituents are themselves unsubstituted.

In some embodiments, the alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will be 1, 2, 3, 4, or 5 substituents present, valency permitting, unless otherwise specified. The 1 to 5 substituents are each, independently, selected from the group consisting of acyl, alkyl (e.g., unsubstituted and substituted, where the substituents include any group described herein, e.g., aryl, halo, hydroxy), alkenyl, alkynyl, aryl (e.g., substituted and unsubstituted phenyl), cycloalkyl (e.g., substituted and unsubstituted cycloalkyl), halo (e.g., fluoro), hydroxyl, heteroalkyl (e.g., substituted and unsubstituted methoxy, ethoxy, or thioalkoxy), heteroalkenyl, heteroalkynyl, heteroaryl, heterocyclyl, amino (e.g., NH₂ or mono- or dialkyl amino), azido, cyano, nitro, thiol, and oxo. Each of the substituents is unsubstituted or substituted with unsubstituted substituent(s) as defined herein for each respective group. In some embodiments, alkyl, alkenyl, alkynyl, heteroalkyl, heteroalkenyl, and heteroalkynyl are optionally substituted with 1, 2, 3, 4, or 5 substituents independently selected from the group consisting of aryl (e.g., substituted and unsubstituted phenyl), cycloalkyl (e.g., substituted and unsubstituted cycloalkyl), halo (e.g., fluoro), hydroxyl, heteroaryl, heterocyclyl, amino (e.g., NH₂ or mono- or dialkyl amino), azido, cyano, nitro, thiol, and oxo. Each of the substituents is unsubstituted or substituted with unsubstituted substituent(s) as defined herein for each respective group. In some embodiments, the substituents are themselves unsubstituted.

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent. Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HRBM-associated factors complex in a human cell.

As used herein, the term “BAF complex-related disorder” refers to a disorder that is caused or affected by the level of activity of a BAF complex.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

By “determining the level” of a protein or RNA is meant the detection of a protein or an RNA, by methods known in the art, either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure RNA levels are known in the art and include, but are not limited to, quantitative polymerase chain reaction (qPCR) and Northern blot analyses.

By “decreasing the activity of a BAF complex” is meant decreasing the level of an activity related to a BAF complex, or a related downstream effect. A non-limiting example of decreasing an activity of a BAF complex is Sox2 activation. The activity level of a BAF complex may be measured using any method known in the art, e.g., the methods described in Kadoch et al. Cell, 2013, 153, 71-85, the methods of which are herein incorporated by reference.

As used herein, the term “degrader” refers to a small molecule compound including a degradation moiety, wherein the compound interacts with a protein (e.g., BRG1 and/or BRM) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRG1 and/or BRM. In one example, the moiety binds to a protease or a ubiquitin ligase that metabolizes the protein, e.g., BRG1 and/or BRM.

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

By “reducing the activity of BRG1 and/or BRM,” is meant decreasing the level of an activity related to an BRG1 and/or BRM, or a related downstream effect. A non-limiting example of inhibition of an activity of BRG1 and/or BRM is decreasing the level of a BAF complex in a cell. The activity level of BRG1 and/or BRM may be measured using any method known in the art. In some embodiments, an agent which reduces the activity of BRG1 and/or BRM is a small molecule BRG1 and/or BRM degrader.

By “reducing the level of BRG1 and/or BRM,” is meant decreasing the level of BRG1 and/or BRM in a cell or subject. The level of BRG1 and/or BRM may be measured using any method known in the art.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

As used herein, the term “inhibiting BRM” refers to blocking or reducing the level or activity of the ATPase catalytic binding domain or the bromodomain of the protein. BRM inhibition may be determined using methods known in the art, e.g., a BRM ATPase assay, a Nano DSF assay, or a BRM Luciferase cell assay.

The term “pharmaceutical composition,” as used herein, represents a composition containing a compound described herein formulated with a pharmaceutically acceptable excipient and appropriate for administration to a mammal, for example a human. Typically, a pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration.

As used herein, the term “pharmaceutically acceptable salt” means any pharmaceutically acceptable salt of a compound, for example, any compound of Formula I or II. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

By a “reference” is meant any useful reference used to compare protein or RNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound of the invention; a sample from a subject that has been treated by a compound of the invention; or a sample of a purified protein or RNA (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound of the invention. In preferred embodiments, the reference sample, standard, or level is matched to the sample subject sample by at least one of the following criteria: age, weight, sex, disease stage, and overall health. A standard curve of levels of a purified protein or RNA, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean therapeutic treatment or any measures whose object is to slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. Compounds of the invention may also be used to “prophylactically treat” or “prevent” a disorder, for example, in a subject at increased risk of developing the disorder.

The details of one or more embodiments of the invention are set forth in the description below. Other features, objects, and advantages of the invention will be apparent from the description and from the claims.

### DETAILED DESCRIPTION

The present disclosure features compounds useful for the inhibition of BRG1 and optionally BRM. These compounds may be used to modulate the activity of a BAF complex, for example, for the treatment of a BAF-related disorder, such as cancer (e.g., BRG1-loss of function disorders). Exemplary compounds described herein include compounds having a structure according to Formula I, or a pharmaceutically acceptable salt thereof.

Formula I:

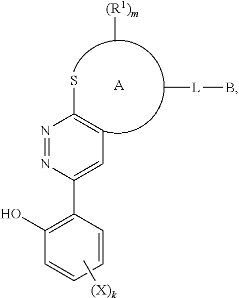

where
ring system A is a 5 to 9-membered heterocyclyl or heteroaryl;
m is 0, 1, 2, or 3;
k is 0, 1, or 2;
each R¹ is, independently, halo, optionally substituted C₁-C₆ alkyl, optionally substituted C₁-C₆ heteroalkyl, optionally substituted C₃-C₈ cycloalkyl, or optionally substituted C₂-C₉ heterocyclyl;
each X is, independently, halo;
L is a linker; and
B is a degradation moiety.

Exemplary compounds described herein include compounds having a structure according to Formula II, or a pharmaceutically acceptable salt thereof.

Formula II:

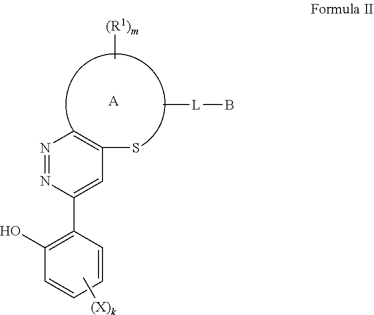

where
ring system A is a 5 to 9-membered heterocyclyl or heteroaryl;
m is 0, 1, 2, or 3;
k is 0, 1, or 2;
each R¹ is, independently, halo, optionally substituted C₁-C₆ alkyl, optionally substituted C₁-C₆ heteroalkyl, optionally substituted C₃-C₈ cycloalkyl, or optionally substituted C₂-C₉ heterocyclyl;
each X is, independently, halo;
L is a linker; and
B is a degradation moiety.

In some embodiments of Formula I or II,
ring system A is a 5 to 9-membered heterocyclyl or heteroaryl;
m is 0, 1, 2, or 3;
k is 0, 1, or 2;
each X is, independently, halo;
L is a linker; and
B is a degradation moiety.

In some embodiments, the compound has the structure of any one of compounds 1-66 in Table 1, or pharmaceutically acceptable salt thereof.

Other embodiments, as well as exemplary methods for the synthesis of production of these compounds, are described herein.

### Pharmaceutical Uses

The compounds described herein are useful in the methods of the invention and, while not bound by theory, are believed to exert their ability to modulate the level, status, and/or activity of a BAF complex, i.e., by inhibiting the activity of the BRG1 and/or BRM proteins within the BAF complex in a mammal. BAF complex-related disorders include, but are not limited to, BRG1 loss of function mutation-related disorders.

An aspect of the present invention relates to methods of treating disorders related to BRG1 loss of function mutations such as cancer (e.g., non-small cell lung cancer, colorectal cancer, bladder cancer, cancer of unknown primary, glioma, breast cancer, melanoma, non-melanoma skin cancer, endometrial cancer, or penile cancer) in a subject in need thereof. In some embodiments, the compound is administered in an amount and for a time effective to result in one or more (e.g., two or more, three or more, four or more) of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a pharmaceutically acceptable salt of the invention.

Exemplary cancers that may be treated by the invention include, but are not limited to, non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer and penile cancer.

### Combination Formulations and Uses Thereof

The compounds of the invention can be combined with one or more therapeutic agents. In particular, the therapeutic agent can be one that treats or prophylactically treats any cancer described herein.

### Combination Therapies

A compound of the invention can be used alone or in combination with an additional therapeutic agent, e.g., other agents that treat cancer or symptoms associated therewith, or in combination with other types of treatment to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, Adriamycin® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), ABraxane®, cremophor-free, albumin-engineered nanoparticle formulation of paclitaxel (American Pharmaceutical Partners, Schaumberg, Ill.), and Taxotere® doxetaxel (Rhone-Poulenc Rorer, Antony, France); chloranbucil; Gemzar® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al. (1999) Proc ASCO 18:233a and Douillard et al. (2000) Lancet 355:1041-7.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (Avastin®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response or antagonizes an antigen important for cancer. Such agents include Rituxan (Rituximab); Zenapax (Daclizumab); Simulect (Basiliximab); Synagis (Palivizumab); Remicade (Infliximab); Herceptin (Trastuzumab); Mylotarg (Gemtuzumab ozogamicin); Campath (Alemtuzumab); Zevalin (Ibritumomab tiuxetan); Humira (Adalimumab); Xolair (Omalizumab); Bexxar (Tositumomab-1-131); Raptiva (Efalizumab); Erbitux (Cetuximab); Avastin (Bevacizumab); Tysabri (Natalizumab); Actemra (Tocilizumab); Vectibix (Panitumumab); Lucentis (Ranibizumab); Soliris (Eculizumab); Cimzia (Certolizumab pegol); Simponi (Golimumab); Ilaris (Canakinumab); Stelara (Ustekinumab); Arzerra (Ofatumumab); Prolia (Denosumab); Numax (Motavizumab); ABThrax (Raxibacumab); Benlysta (Belimumab); Yervoy (Ipilimumab); Adcetris (Brentuximab Vedotin); Perjeta (Pertuzumab); Kadcyla (Ado-trastuzumab emtansine); and Gazyva (Obinutuzumab). Also included are antibody-drug conjugates.

The second agent may be a therapeutic agent which is a non-drug treatment. For example, the second therapeutic agent is radiation therapy, cryotherapy, hyperthermia and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab). In some embodiments, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The compounds of the invention are preferably formulated into pharmaceutical compositions for administration to a mammal, preferably, a human, in a biologically compatible form suitable for administration in vivo. Accordingly, in an aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, ortransdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard- or soft-shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers. A compound of the invention may also be administered parenterally. Solutions of a compound of the invention can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20th ed.) and in The United States Pharmacopeia: The National Formulary (USP 24 NF19), published in 1999. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

Alternatively, the dosage amount can be calculated using the body weight of the patient. For example, the dose of a compound, or pharmaceutical composition thereof, administered to a patient may range from 0.1-100 mg/kg (e.g., 0.25-25 mg/kg). In exemplary, non-limiting embodiments, the dose may range from 0.5-5.0 mg/kg (e.g., 0.5, 1.0, 1.5, 2.0, 2.5, 3.0, 3.5, 4.0, 4.5, or 5.0 mg/kg) or from 5.0-20 mg/kg (e.g., 5.5, 6.0, 6.5, 7.0, 7.5, 8.0, 8.5, 9.0, 9.5, 10, 11, 12, 13, 14, 15, 16, 17, 18, 19, or 20 mg/kg).

### EXAMPLES

The following abbreviations are used throughout the Examples below.
Ac acetyl
ACN or MeCN acetonitrile
AcOH acetic acid
Ac₂O acetic anhydride
aq. aqueous
Boc tert-butoxycarbonyl
Bu or n-Bu butyl
CDI 1,1′-carbonyldiimidazole
DCE or 1,2-DCE 1,2-dichloroethane
DCM dichloromethane
DIAD diisopropyl azodicarboxylate
DIPEA or DIEA N,N-diisopropylethylamine
DMAP 4-(dimethylamino)pyridine
DME 1,2-dimethoxyethane
DMF N,N-dimethylformamide
DMSO dimethyl sulfoxide
EA or EtOAc ethyl acetate
EDCI N-(3-dimethylaminopropyl)-N′-ethylcarbodiimide hydrochloride
equiv equivalents
Et₃N or TEA triethylamine
EtOH ethyl alcohol
FA formic acid
h or hr hour
HATU 1-[bis(dimethylamino)methylene]-1H-1,2,3-triazolo[4,5-b]pyridinium 3-oxid hexafluorophosphate
HOAt 1-hydroxy-7-azabenzotriazole
HOBt or HOBT 1-hydroxybenzotriazole hydrate
iPr Isopropyl
MeOH methyl alcohol
Me₄t-BuXphos ditert-butyl-[2,3,4,5-tetramethyl-6-(2,4,6-triisopropylphenyl)phenyl]phosphane
min minute
MTBE tert-butyl methyl ether
n-BuLi n-butylithium
NMP 1-methyl-2-pyrrolidinone
OAc acetate
Pd/C palladium on carbon
PDC pyridinium dichromate
PdCl₂(dtbpf) or Pd(dtbpf)Cl₂ dichloro[1,1′-bis(di-t-butylphosphino)ferrocene]palladium(II)
PdCl₂(dppf) or Pd(dppf)Cl₂ [1,1′-bis(diphenylphosphino)ferrocene]dichloropalladium(II)
Pd₂(dba)₃ tris(dibenzylideneacetone)dipalladium(0)
Pd(PPh₃)₄ tetrakis(triphenylphosphine)palladium(0
Pd(PPh₃)₂Cl₂ dichlorobis(triphenylphosphine)palladium(II)
PE petroleum ether
PPh₃ triphenylphosphine
Pr n-propyl
Py pyridine
rac racemic
Rf retention factor
r.t. or rt room temperature
sat. saturated
SFC supercritical fluid chromatography
t-Bu tert-butyl
tBuXphos-Pd-G3 or tBuXphos Pd G₃ or t-BuXphos-Pd (gen 3) [2-(2-aminophenyl)phenyl]-methylsulfonyloxypalladium;ditert-butyl-[2-(2,4,6-triisopropylphenyl)phenyl]phosphane
TFA trifluoroacetic acid
Tf₂O trifluoromethanesulfonic anhydride
THE tetrahydrofuran
TLC thin layer chromatography
Xantphos-Pd-G3 [2-(2-aminophenyl)phenyl]-methylsulfonyloxy-palladium;(5-diphenylphosphanyl-9,9-dimethyl-xanthen-4-yl)-diphenyl-phosphane

### Example 1. Preparation of Compounds

Preparation of methyl 2-(3-hydroxy-1,2-oxazol-5-yl)-3-methylbutanoate (I-1)

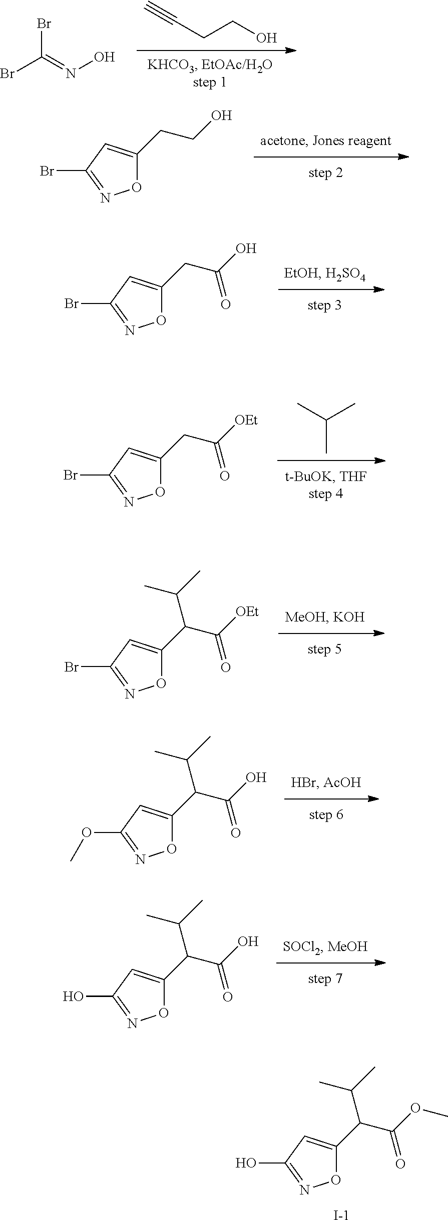

Step 1: Preparation of 2-(3-bromoisoxazol-5-yl)ethan-1-ol

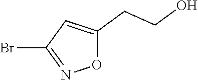

A solution of 3-butyn-1-ol (552.89 g, 7888.26 mmol, 4 equiv) and KHCO₃ (592.30 g, 5916.197 mmol, 3 equiv) in EtOAc (2600 mL) and H₂O (260 mL) was stirred at room temperature. To the above mixture was added 1-bromo-N-hydroxymethanecarbonimidoyl bromide (400.00 g in EA (840 mL), 1972.066 mmol, 1.00 equiv) dropwise over 60 min at room temperature. The resulting mixture was stirred for overnight at room temperature. The reaction mixture was washed with water (500 mL×2) and the combined organic layers dried over anhydrous Na₂SO₄. After filtration, the filtrate was concentrated under reduced pressure. The residue was purified by silica gel column chromatography, eluted with PE/EtOAc (30:1) to afford intermediate the title compound (338.2 g, 88.98%) as off-white solid. LCMS (ESI) m/z [M+H]⁺=192.

Step 2: Preparation of 2-(3-bromoisoxazol-5-yl)acetic acid

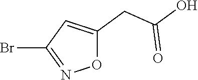

A solution of 2-(3-bromoisoxazol-5-yl)ethan-1-ol (360.00 g) in acetone (3600 mL) was stirred at 0 degrees C. under a nitrogen atmosphere. To the above mixture was added Jones' reagent (1760 mL) dropwise over 1 h at 0 degrees C. The resulting mixture was stirred overnight at room temperature. The reaction was quenched with water/Ice at 0 degrees C. The resulting mixture was extracted with EtOAc (1000 mL×3). The combined organic layers were washed with water (500 mL×2), and dried over anhydrous Na₂SO₄. After filtration, the filtrate was concentrated under reduced pressure to afford the title compound (348.6 g, crude) as a green solid that was used directly without further purification. (LCMS (ESI) m/z [M+H]⁺=206.

Step 3: Preparation of ethyl 2-(3-bromoisoxazol-5-yl)acetate

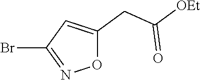 [SMILES: CCOC(=O)Cc1cc(Br)no1]

A solution of 2-(3-bromoisoxazol-5-yl)acetic acid (397.6 g, 1930.144 mmol, 1.00 equiv) and H₂SO₄ (18.92 g, 193.014 mmol, 0.1 equiv) in EtOH (2000 mL) was stirred for 2 h at 70 degrees C. The reaction mixture was concentrated under reduced pressure. The residue was diluted with EtOAc (3000 mL), washed with water (500 mL×2), and dried over anhydrous Na₂SO₄. After filtration, the filtrate was concentrated under reduced pressure. The residue was purified by silica gel column chromatography, eluted with PE/EtOAc (35:1) to afford the title compound (355 g, 78.61%) as a colorless oil. (LCMS (ESI) m/z [M+H]⁺=234.

Step 4: Preparation of ethyl 2-(3-bromoisoxazol-5-yl)-3-methylbutanoate

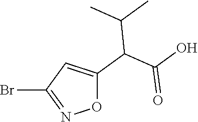

To a stirred solution of t-BuOK (244.51 g, 2179.031 mmol, 1.5 equiv) and ethyl 2-(3-bromoisoxazol-5-yl)acetate (340.00 g, 1452.687 mmol, 1.00 equiv) in THF (2000 mL) was added 2-iodopropane (321.03 g, 1888.493 mmol, 1.3 equiv) dropwise at 0 degrees C. under a nitrogen atmosphere. The resulting mixture was stirred for overnight at room temperature, then diluted with Water/Ice at 0 degrees C. The resulting mixture was extracted with EtOAc (1000 mL×2). The combined organic layers were washed with water (500 mL×1), dried over anhydrous Na₂SO₄. After filtration, the filtrate was concentrated under reduced pressure. The residue was purified by silica gel column chromatography, eluted with PE/THF (10:1) to afford the title compound (284.1 g, 70.82%) as a colorless oil. (LCMS (ESI) m/z [M+H]⁺=276.

Step 5: Preparation of 2-(3-methoxy-1,2-oxazol-5-yl)-3-methylbutanoic acid

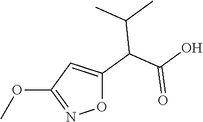

To a stirred solution of Preparation of ethyl 2-(3-bromoisoxazol-5-yl)-3-methylbutanoate (90.00 g, 325.933 mmol, 1.00 equiv) in MeOH (270 mL) was added a solution of KOH (274.30 g, 4888.995 mmol, 15.00 equiv) in MeOH (210 mL) at 0 degrees C. The reaction mixture was stirred overnight at 80 degrees C. The resulting solution was acidified to pH 4 with 1M solution of HCl (aq.) and concentrated under reduced pressure. The resulting mixture was diluted with EtOAc (1800 mL) and filtered. The filter cake was washed with EtOAc (100 mL×3). The filtrate was concentrated under reduced pressure to afford the title compound (62.9 g, 96.88%) as a yellow oil that was used directly without further purification. LCMS (ESI) m/z: [M+H]⁺=200.

Step 6: Preparation of 2-(3-hydroxy-1,2-oxazol-5-yl)-3-methylbutanoic acid

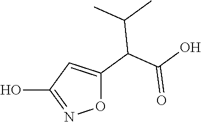

To a stirred solution of 2-(3-methoxy-1,2-oxazol-5-yl)-3-methylbutanoic acid (62.90 g, 315.754 mmol, 1.00 equiv) in HOAc (450.00 mL) was added 48% HBr (450.00 mL) at room temperature. The resulting mixture was stirred for 16 h at 60 degrees C. The resulting mixture was concentrated under reduced pressure, and the residue purified by flash C18-flash chromatography, elution gradient 0 to 100% MeCN in water (containing 0.05% FA). Pure fractions were evaporated to dryness to afford the title compound (43.3 g, 74.05%) as a white solid. LCMS (ESI) m/z: [M+H]⁺=186.

Step 7: Preparation of methyl 2-(3-hydroxy-1,2-oxazol-5-yl)-3-methylbutanoate (I-1)

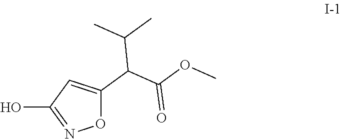

To a stirred solution of 2-(3-hydroxy-1,2-oxazol-5-yl)-3-methylbutanoic acid (20 g, 108.004 mmol, 1.00 equiv) in MeOH (72 mL) was added SOCl₂ (35.26 mL, 486.059 mmol, 4.50 equiv) at 0 degrees C. The resulting mixture was stirred for 16 h at room temperature. The resulting mixture was concentrated under reduced pressure and the residue was diluted with water (30 mL) and extracted with EtOAc (50 mL×3). The combined organic layers were washed with saturated aqueous NaCl (30 mL), dried over anhydrous Na₂SO₄ and filtered. The filtrate was concentrated under reduced pressure. The crude product was purified by silica gel column chromatography, elution gradient 0 to 100% THE in petroleum ether. Pure fractions were evaporated to dryness to afford compound I-1 (15.1 g, 70.18%) as an off-white solid. ¹H NMR (400 MHz, DMSO-d₆) δ 11.24 (s, 1H), 5.95 (s, 1H), 3.71-3.58 (m, 4H), 2.32-2.20 (m, 1H), 0.88 (dd, J=34.2, 6.7 Hz, 6H). LCMS (ESI) m/z: [M+H]⁺=200.

Preparation of (2S,4R)-1-{2-[3-(2,2-diethoxyethoxy)-1,2-oxazol-5-yl]-3-methylbutanoyl}-4-hydroxy-N-[(1S)-1-[4-(4-methyl-1,3-thiazol-5-yl)phenyl]ethyl]pyrrolidine-2-carboxamide (I-2)

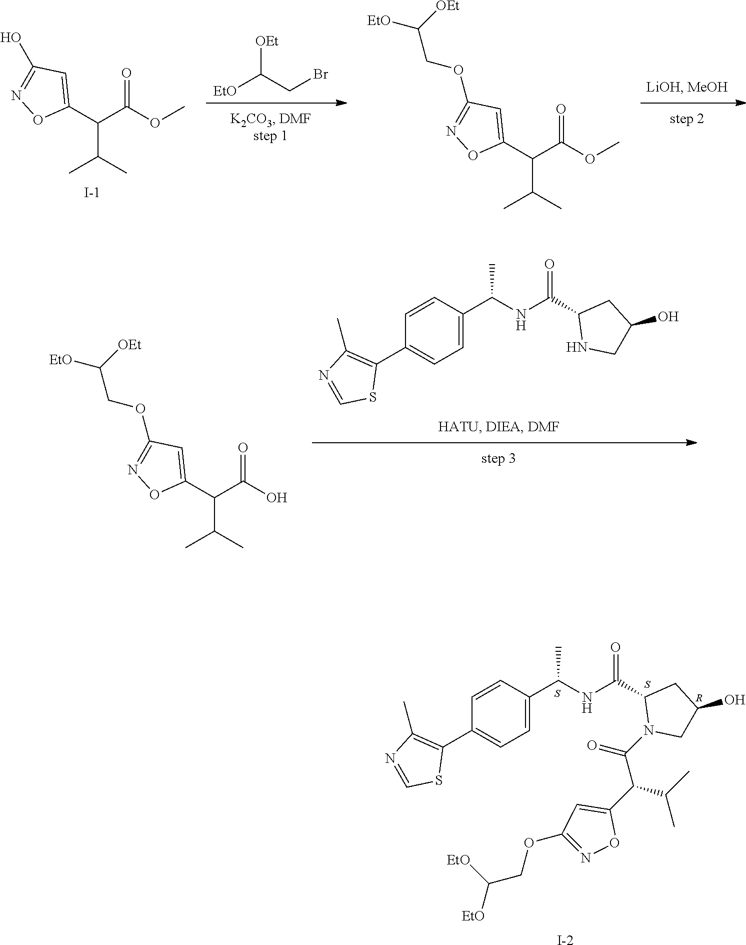

Step 1: methyl 2-[3-(2,2-diethoxyethoxy)-1,2-oxazol-5-yl]-3-methybutanoate

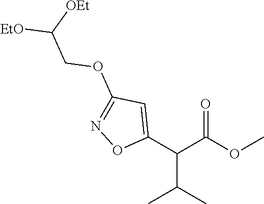 [SMILES: CCOC(COc1cc(C(C(=O)OC)C(C)C)on1)OCC]

To a stirred solution of methyl 2-(3-hydroxy-1,2-oxazol-5-yl)-3-methylbutanoate (I-1, 7 g, 35.140 mmol, 1.00 equiv) and 2-bromo-1,1-diethoxyethane (7.62 g, 38.654 mmol, 1.1 equiv) in DMF (70 mL) was added K₂CO₃ (9.71 g, 70.280 mmol, 2 equiv). The resulting mixture was stirred for overnight at 80 degrees C. then cooled to room temperature and diluted with EtOAc (300 mL). The organic layer was washed with water (300 mL), followed by brine (300 mL), then dried over anhydrous sodium sulfate, filtered and concentrated. The crude product was purified by Prep-HPLC to afford the title compound (5.2 g, 46.92%) as a brown solid. LCMS (ESI) m/z: [M+H]⁺=316.

Step 2: 2-[3-(2,2-diethoxyethoxy)-1,2-oxazol-5-yl]-3-methylbutanoic acid

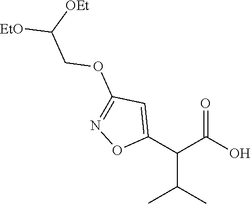 [SMILES: CCOC(COc1cc(C(C(=O)O)C(C)C)on1)OCC]

A mixture of methyl 2-[3-(2,2-diethoxyethoxy)-1,2-oxazol-5-yl]-3-methylbutanoate (5.2 g, 16.489 mmol, 1.00 equiv) and LiOH (1.97 g, 82.445 mmol, 5 equiv) in MeOH (15 mL) and H₂O (45 mL) was stirred for 2 h at room temperature. The mixture was acidified to pH 5 with conc. HCl, then extracted with EtOAc (300 mL×3). The combined organic layers were washed with brine (100 mL), dried over anhydrous Na₂SO₄ and filtered. The filtrate was concentrated under reduced pressure. The crude product mixture was used in the next step directly without further purification. LCMS (ESI) m/z: [M+H]⁺=302.

Step 3: (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)eth y)pyrrolidine-2-carboxamide (I-2)

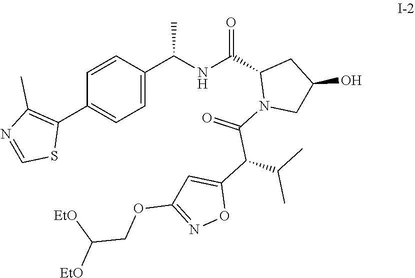 [SMILES: CCOC(COc1cc([C@H](C(=O)N2C[C@H](O)C[C@H]2C(=O)N[C@@H](C)c2ccc(-c3scnc3C)cc2)C(C)C)on1)OCC]

To a stirred solution of 2-[3-(2,2-diethoxyethoxy)-1,2-oxazol-5-yl]-3-methylbutanoic acid (4.95 g, 16.427 mmol, 1.00 equiv) and (2S,4R)-4-hydroxy-N-[(1S)-1-[4-(4-methyl-1,3-thiazol-5-yl)phenyl]ethyl]pyrrolidine-2-carboxamide (5.44 g, 16.427 mmol, 1 equiv) in DMF (50 mL) was added HATU (6.87 g, 18.070 mmol, 1.1 equiv) and DIEA (6.37 g, 49.281 mmol, 3 equiv). The resulting mixture was stirred for 2 h at room temperature then diluted with EtOAc (300 mL). The organic layer was washed with water (300 mL), followed by brine (300 mL) then dried over anhydrous sodium sulfate, filtered and concentrated. The residue was purified by silica gel column chromatography, eluted with PE/EA (1:1) to afford a crude product. The crude product (6.2 g) was purified by Chiral-SFC with Column: CHIRAL ART Amylose-SA, 3*25 cm, 5 μm; Mobile Phase A: CO2, Mobile Phase B: MeOH—HPLC; Flow rate: 50 mL/min; Gradient: isocratic 40% B; Column Temperature(° C.): 35 to provide 1-2 (2.8 g, 27.73%) as a white solid. 1H NMR (400 MHz, DMSO-d6) δ 8.99 (d, J=3.4 Hz, 1H), 8.43 (d, J=7.7 Hz, 1H), 7.49-7.41 (m, 2H), 7.41-7.31 (m, 2H), 6.14 (s, 1H), 5.10 (d, J=3.6 Hz, 1H), 4.97-4.87 (m, 1H), 4.81 (t, J=5.2 Hz, 1H), 4.37 (t, J=7.9 Hz, 1H), 4.32-4.23 (m, 1H), 4.09 (d, J=5.3 Hz, 2H), 3.73-3.49 (m, 6H), 3.45 (d, J=10.8 Hz, 1H), 2.46 (d, J=2.1 Hz, 3H), 2.31-2.15 (m, 1H), 2.03 (ddd, J=11.9, 8.1, 3.0 Hz, 1H), 1.78 (ddd, J=12.8, 8.0, 4.7 Hz, 1H), 1.41 (dd, J=29.6, 7.0 Hz, 3H), 1.13 (t, J=7.0 Hz, 6H), 0.96 (t, J=6.4 Hz, 3H), 0.81 (dd, J=14.4, 6.7 Hz, 3H). LCMS (ESI) m/z: [M+H]⁺=615.35.

Preparation of (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (I-3)

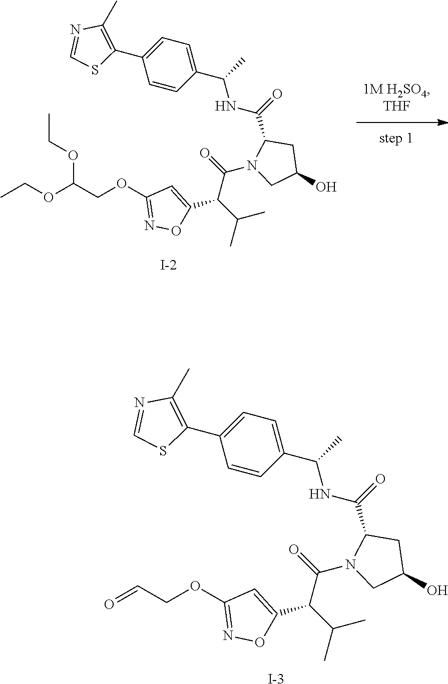

To a stirred solution of H₂SO₄ (1M) (6.00 mL) and THF (6.00 mL) was added (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (300.00 mg, 0.499 mmol, 1.00 equiv) at room temperature. The resulting mixture was stirred for 8 h at 50 degrees C. The reaction was quenched with water/Ice at 0 degrees C., then the mixture was basified to pH 7 with saturated NaHCO₃ (aq.). The resulting mixture was extracted with EtOAc (3×100 mL). The combined organic layers were washed with brine (2×100 mL), dried over anhydrous Na₂SO₄, and filtered. The filtrate was concentrated under reduced pressure to 1-3 (256 mg, 97.3%) as a white solid that was used directly without further purification. LCMS (ESI) m/z: [M+H]⁺=541.

The following intermediates in Table 2 were prepared in a similar manner as described in the preparation of intermediate I-3 starting with methyl 2-(3-hydroxy-1,2-oxazol-5-yl)-3-methylbutanoate and the appropriate alkyl bromides.

TABLE 2

| | Intermediate | | LCMS |
|---|---|---|---|
| Structure | No. | Name | (ESI) m/z |
| 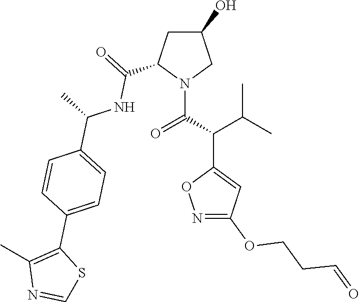 [SMILES: Cc1ncsc1-c1ccc([C@H](C)NC(=O)[C@@H]2C[C@@H](O)CN2C(=O)[C@@H](c2cc(OCCC=O)no2)C(C)C)cc1] | I-60 | (2S,4R)-4-hydroxy-1-((R)-3- methyl-2-(3-(2- oxopropoxy)isoxazol-5- yl)butanoyl)-N-((S)-1-(4-(4- methylthiazol-5- yl)phenyl)ethyl)pyrrolidine-2- carboxamide | 555 |
| 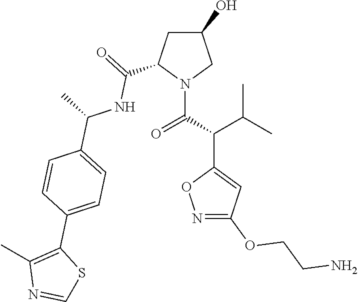 [SMILES: Cc1ncsc1-c1ccc([C@H](C)NC(=O)[C@@H]2C[C@@H](O)CN2C(=O)[C@@H](c2cc(OCCN)no2)C(C)C)cc1] | I-61 | (2S,4R)-1-((R)-2-(3-(2- aminoethoxy)isoxazol-5-yl)-3- methylbutanoyl)-4-hydroxy-N- ((S)-1-(4-(4-methylthiazol-5- yl)phenyl)ethyl)pyrrolidine-2- carboxamide | 542 |

Preparation of (2S,4R)-4-hydroxy-1-((R)-2-(3-(2-(3-(3-(2-hydroxyphenyl)thieno[2,3-c]pyridazin-6-yl)azetidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

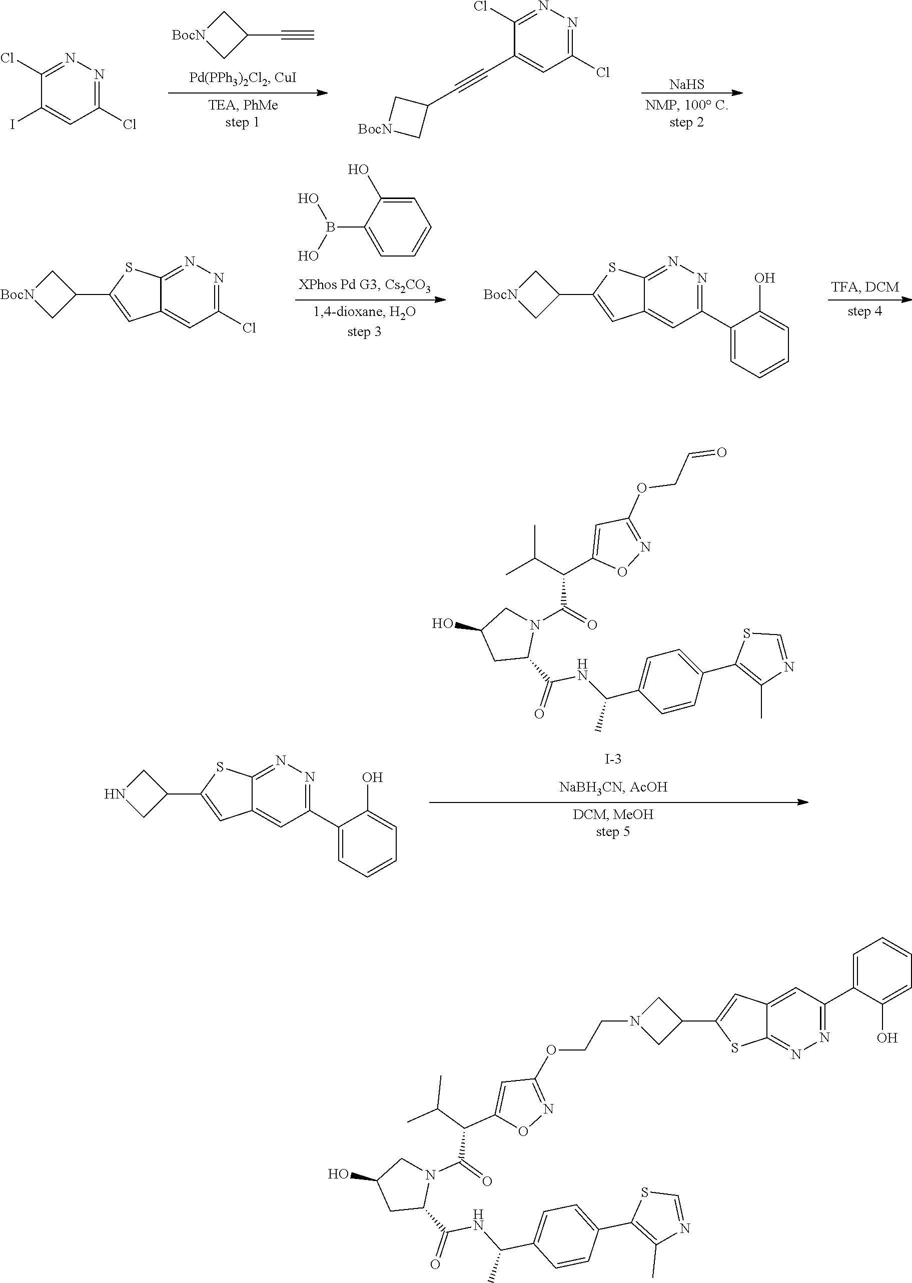

Step 1: Preparation of tert-butyl 3-[2-(3,6-dichloropyridazin-4-yl)ethynyl]azetidine-1-carboxylate

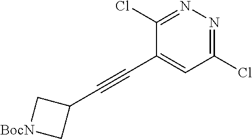 [SMILES: CC(C)(C)OC(=O)N1CC(C#Cc2cc(Cl)nnc2Cl)C1]

To a stirred mixture of 3,6-dichloro-4-iodopyridazine (200 mg, 0.728 mmol, 1.00 equiv) and tert-butyl 3-ethynylazetidine-1-carboxylate (145.06 mg, 0.801 mmol, 1.1 equiv) in toluene (5.00 mL) was added Pd(PPh₃)₂Cl₂ (76.61 mg, 0.109 mmol, 0.15 equiv), CuI (27.71 mg, 0.146 mmol, 0.2 equiv) and TEA (220.88 mg, 2.184 mmol, 3 equiv) at room temperature under a nitrogen atmosphere. The resulting mixture was stirred for 2 h at room temperature then filtered. The filtrate was concentrated under reduced pressure and the residue purified by reverse phase flash chromatography with the following conditions: column, C18; mobile phase, MeCN in water (0.05% FA), 40% to 60% gradient. This provided the title compound (170 mg, 64.07%) as a yellow solid. LCMS (ESI) m/z: [M+H]⁺=328.

Step 2: Preparation of tert-butyl 3-{3-chlorothieno[2,3-c]pyridazin-6-yl}azetidine-1-carboxylate

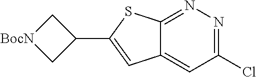 [SMILES: CC(C)(C)OC(=O)N1CC(c2cc3cc(Cl)nnc3s2)C1]

To a stirred mixture of tert-butyl 3-[2-(3,6-dichloropyridazin-4-yl)ethynyl]azetidine-1-carboxylate (160 mg, 0.488 mmol, 1.00 equiv) in NMP (5 mL) was added sodium hydrosulfide (32.80 mg, 0.586 mmol, 1.2 equiv) at room temperature under a nitrogen atmosphere. The resulting mixture was stirred for 2 h at 100 degrees C., cooled, and filtered. The filtrate was purified by reverse phase flash chromatography with the following conditions: column, C18; mobile phase, MeCN in water (0.05% FA), 30% to 50% gradient. This provided the title compound (122 mg, 71.43%) as a white solid. LCMS (ESI) m/z [M+H]⁺=326.

Step 3: Preparation of tert-butyl 3-[3-(2-hydroxyphenyl)thieno[2,3-c]pyridazin-6-yl]azetidine-1-carboxylate

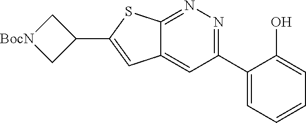 [SMILES: CC(C)(C)OC(=O)N1CC(c2cc3cc(-c4ccccc4O)nnc3s2)C1]

To a stirred mixture of tert-butyl 3-{3-chlorothieno[2,3-c]pyridazin-6-yl}azetidine-1-carboxylate (122 mg, 0.374 mmol, 1.00 equiv) and 2-hydroxyphenylboronic acid (154.94 mg, 1.122 mmol, 3 equiv) in dioxane (4 mL) and H₂O (1 mL) was added Cs₂CO₃ (244.01 mg, 0.748 mmol, 2 equiv) and XPhos Pd G3 (63.39 mg, 0.075 mmol, 0.2 equiv) at room temperature under a nitrogen atmosphere. The resulting mixture was stirred for 2 h at 80 degrees C., then filtered. The filtrated was concentrated under reduced pressure. The residue was purified by reverse flash chromatography with the following conditions: column, C18; mobile phase, MeCN in water (0.05% FA), 40% to 60% gradient. This provided the title compound (85 mg, 53.28%) as a white solid. LCMS (ESI) m/z: [M+H]⁺=384.

Step 4: Preparation of 2-[6-(azetidin-3-yl)thieno[2,3-c]pyridazin-3-yl]phenol

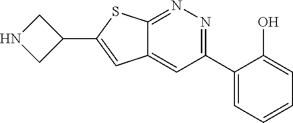 [SMILES: Oc1ccccc1-c1cc2cc(C3CNC3)sc2nn1]

To a stirred mixture of tert-butyl 3-[3-(2-hydroxyphenyl)thieno[2,3-c]pyridazin-6-yl]azetidine-1-carboxylate (85 mg, 0.222 mmol, 1.00 equiv) in DCM (4 mL) was added TFA (1 mL) dropwise at room temperature under a nitrogen atmosphere. The resulting mixture was stirred for 2 h then concentrated under reduced pressure. The crude product was used in the next step directly without further purification. LCMS (ESI) m/z: [M+H]⁺=284.

Step 2: Preparation of (2S,4R)-4-hydroxy-1-((R)-2-(3-(2-(3-(3-(2-hydroxyphenyl)thieno[2,3-c]pyridazin-6-yl)azetidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

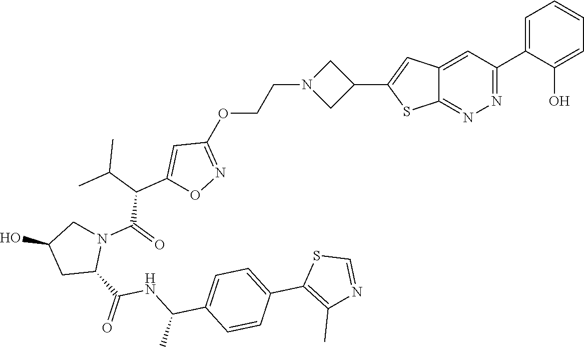 [SMILES: Cc1ncsc1-c1ccc([C@H](C)NC(=O)[C@@H]2C[C@@H](O)CN2C(=O)[C@@H](c2cc(OCCN3CC(c4cc5cc(-c6ccccc6O)nnc5s4)C3)no2)C(C)C)cc1]

To a stirred solution of 2-[6-(azetidin-3-yl)thieno[2,3-c]pyridazin-3-yl]phenol (15 mg, 0.053 mmol, 1.00 equiv) and 2-[6-(azetidin-3-yl)thieno[2,3-c]pyridazin-3-yl]phenol (28.62 mg, 0.053 mmol, 1 equiv) in DCM (1 mL) and MeOH (1 mL) were added NaBH₃CN (9.98 mg, 0.159 mmol, 3 equiv) and AcOH (0.15 mL). The mixture was stirred for 1 h at room temperature under a nitrogen atmosphere. Without any additional work-up, the mixture was purified by reverse phase flash column chromatography and the resulting crude product was purified by Chiral-Prep-HPLC with the following conditions (NB-Prep-HPLC-01): Column, XBridge Shield RP18 OBD Column, 19*150 mm, 5 μm; mobile phase, water (10 mmol/L NH₄HCO₃) and ACN (49% ACN up to 55% in 6 min to afford the title compound (15 mg, 35.07%) as a white solid. ¹H NMR (300 MHz, DMSO-d₆) δ 12.40 (s, 1H), 8.99 (s, 1H), 8.73 (d, J=4.9 Hz, 1H), 8.41 (d, J=7.7 Hz, 1H), 8.09-7.94 (m, 1H), 7.51-7.41 (m, 3H), 7.41-7.31 (m, 3H), 7.09-6.95 (m, 2H), 6.02 (d, J=50.5 Hz, 1H), 5.10 (s, 1H), 4.92 (t, J=7.2 Hz, 1H), 4.38 (t, J=7.8 Hz, 1H), 4.29 (s, 1H), 4.18 (t, J=5.3 Hz, 2H), 4.15-4.03 (m, 1H), 3.84-3.61 (m, 4H), 3.52-3.42 (m, 1H), 3.42-3.35 (m, 2H), 2.85 (t, J=5.4 Hz, 2H), 2.46 (d, J=3.1 Hz, 3H), 2.30-2.18 (m, 1H), 2.10-1.72 (m, 2H), 1.49-1.33 (m, 3H), 0.97 (d, J=6.7, 3.7 Hz, 3H), 0.82 (d, J=10.1, 6.6 Hz, 3H). LCMS (ESI) m/z: [M+H]⁺=808.6.

Preparation of (2S,4R)-4-hydroxy-1-((R)-2-(3-(2-(4-(3-(2-hydroxyphenyl)thieno[2,3-c]pyridazin-6-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

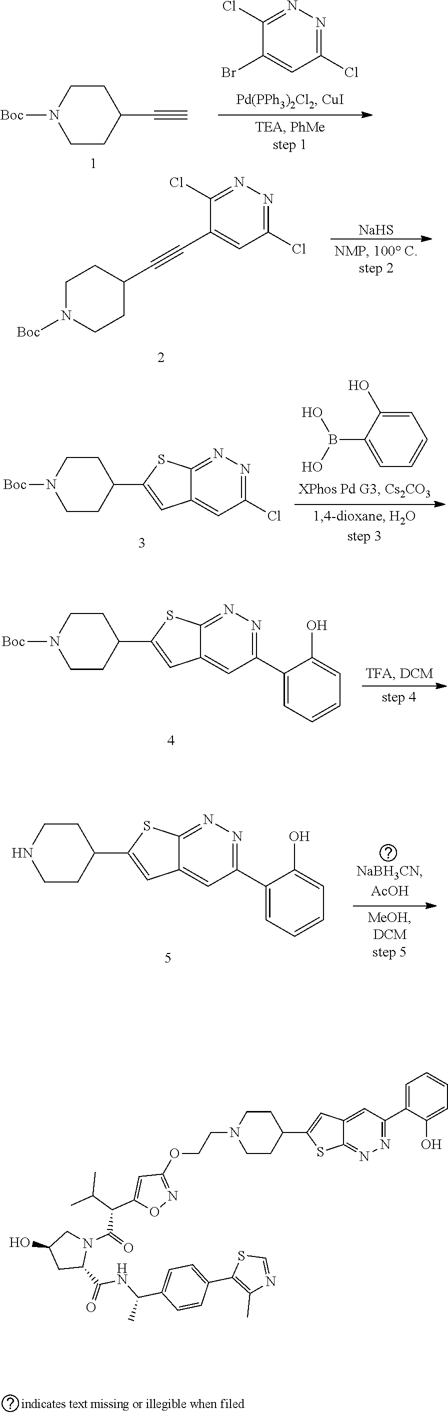

Step 1: Preparation of tert-butyl 4-((3,6-dichloropyridazin-4-yl)ethynyl)piperidine-1-carboxylate (intermediate 2)

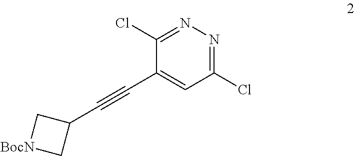 [SMILES: CC(C)(C)OC(=O)N1CC(C#Cc2cc(Cl)nnc2Cl)C1]

To a stirred mixture of tert-butyl 4-ethynylpiperidine-1-carboxylate (1.00 g, 4.778 mmol, 1.00 equiv) and 4-bromo-3,6-dichloropyridazine (1.20 g, 5.256 mmol, 1.1 equiv) in toluene (10.00 mL) were added Pd(PPh3)2Cl2 (0.50 g, 0.717 mmol, 0.15 equiv), CuI (0.18 g, 0.956 mmol, 0.2 equiv), and TEA (1.45 g, 14.334 mmol, 3 equiv) at room temperature under a nitrogen atmosphere. The resulting mixture was stirred overnight at room temperature then concentrated under reduced pressure. The residue was dissolved in DMF (15.00 mL) and purified by reverse flash chromatography with the following conditions: column, C18; mobile phase, MeCN in water (0.05% FA), 0% to 100% gradient in 25 min to afford intermediate 2 (596 mg, 33.61%) as a brown solid. LCMS (ESI) m/z: [M+H]+=356.25

Step 2: Preparation of tert-butyl 4-(3-chlorothieno[2,3-c]pyridazin-6-yl)piperidine-1-carboxylate (intermediate 3)

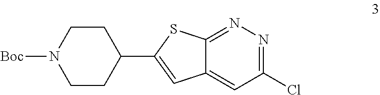 [SMILES: CC(C)(C)OC(=O)N1CCC(c2cc3cc(Cl)nnc3s2)CC1]

To a stirred solution of intermediate 2 (596.00 mg, 1.673 mmol, 1.00 equiv) in NMP (10.00 mL) was added NaSH (93.79 mg, 1.673 mmol, 1.0 equiv) at room temperature. The resulting mixture was stirred for 1 h at 100 degrees C., then allowed to cool down to room temperature. The resulting mixture was extracted with EtOAc (3×200 mL). The combined organic layers were washed with brine (100 mL), dried over anhydrous Na2SO4, and filtered. The filtrate was concentrated under reduced pressure. The residue was dissolved in DMF (10.00 mL) and was purified by reverse flash chromatography with the following conditions: column, C18; mobile phase, MeCN in water (0.05% FA), 0% to 100% gradient in 30 min; to afford intermediate 3 (356 mg, 49.91%) as a brown solid. LCMS (ESI) m/z: [M+H]+=353.87.

Step 3: Preparation of tert-butyl 4-(3-(2-hydroxyphenyl)thieno[2,3-c]pyridazin-6-yl)piperidine-1-carboxylate (intermediate 4)

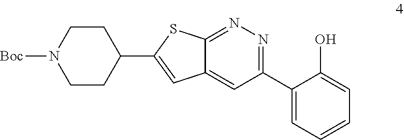 [SMILES: CC(C)(C)OC(=O)N1CCC(c2cc3cc(-c4ccccc4O)nnc3s2)CC1]

To a solution of intermediate 3 (350.00 mg, 0.989 mmol, 1.00 equiv) and 2-hydroxyphenylboronic acid (204.63 mg, 1.484 mmol, 1.5 equiv) in dioxane (5.00 mL) and H2O (1.00 mL) were added Cs2CO3 (644.51 mg, 1.978 mmol, 2.0 equiv) and XPhos Pd G3 (83.72 mg, 0.099 mmol, 0.1 equiv). After stirring overnight at 90 degrees C. under a nitrogen atmosphere the mixture was concentrated under reduced pressure. The residue was dissolved in DMF (10.00 mL) and purified by reverse flash chromatography with the following conditions: column, C18; mobile phase, MeCN in water (0.05% FA), 0% to 100% gradient in 20 min; to afford intermediate 4 (188 mg, 44.85%) as a brown solid. LCMS (ESI) m/z: [M+H]+=411.52.

Step 4: Preparation of 2-(6-(piperidin-4-yl)thieno[2,3-c]pyridazin-3-yl)phenol (intermediate 5)

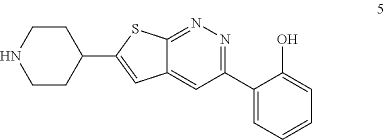 [SMILES: Oc1ccccc1-c1cc2cc(C3CCNCC3)sc2nn1]

To a stirred solution of intermediate 4 (188.00 mg, 0.457 mmol, 1.00 equiv) in DCM (9.00 mL) was added TFA (3.00 mL) at room temperature. The resulting mixture was stirred for 4 h, then concentrated under reduced pressure. The residue was purified by reverse flash chromatography with the following conditions: column, C18; mobile phase, MeCN in water (0.05 FA), 0% to 100% gradient in 20 min; to afford intermediate 5 (130 mg, 91.38%) as a light brown solid. LCMS (ESI) m/z: [M+H]+=311.40.

Step 5: Preparation of (2S,4R)-4-hydroxy-1-((R)-2-(3-(2-(4-(3-(2-hydroxyphenyl)thieno[2,3-c]pyridazin-6-yl)piperidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

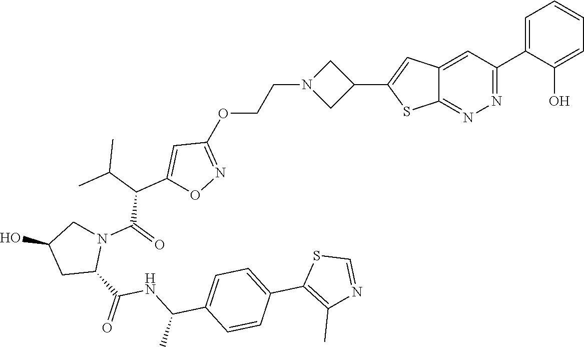 [SMILES: Cc1ncsc1-c1ccc([C@H](C)NC(=O)[C@@H]2C[C@@H](O)CN2C(=O)[C@@H](c2cc(OCCN3CC(c4cc5cc(-c6ccccc6O)nnc5s4)C3)no2)C(C)C)cc1]

A mixture of intermediate 5 (10.37 mg, 0.034 mmol, 1.2 equiv) and (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (I-3, 15.00 mg, 0.028 mmol, 1.00 equiv) in MeOH (1.00 mL) and DCM (1.00 mL) was stirred for 30 min at room temperature. NaBH3CN (5.23 mg, 0.084 mmol, 3.0 equiv) and AcOH (0.01 mL) were then added at room temperature and the mixture stirred overnight at room temperature. The resulting mixture was concentrated under reduced pressure, and the residue was purified by Prep-HPLC with the following conditions (Column: Xselect CSH F-Phenyl OBD column, 19*250 mm, 5 μm; Mobile Phase A: Water (0.05% FA), Mobile Phase B: ACN; Flow rate: 25 mL/min; Gradient: 20% B to 51% B in 7 min to afford the title compound (5.8 mg, 23.58%) as a white solid. 1H NMR (400 MHz, DMSO-d6) δ 12.37 (s, 1H), 8.98 (s, 1H), 8.72 (d, J=2.8 Hz, 1H), 8.41 (d, J=7.7 Hz, 1H), 8.01 (dd, J=8.0, 1.7 Hz, 1H), 7.44 (d, J=8.3 Hz, 2H), 7.37 (dd, J=8.4, 3.4 Hz, 4H), 7.02 (dd, J=12.4, 7.8 Hz, 2H), 6.13 (s, 1H), 5.10 (d, J=3.7 Hz, 1H), 4.91 (t, J=7.3 Hz, 1H), 4.38 (d, J=7.8 Hz, 1H), 4.29 (s, 3H), 3.74-3.64 (m, 2H), 3.45 (d, J=9.9 Hz, 1H), 3.16-3.01 (m, 3H), 2.82-2.70 (m, 2H), 2.46 (s, 3H), 2.29-2.15 (m, 3H), 2.13-1.98 (m, 3H), 1.86-1.72 (m, 3H), 1.38 (d, 3H), 0.97 (d, J=6.2 Hz, 3H), 0.83 (d, J=14.1, 6.7 Hz, 3H). LCMS (ESI) m/z: [M+H]+=836.50

The compounds in Table 3 were prepared using procedures similar to those above using the appropriate amine and aldehyde (or ketone).

TABLE 3

| | | LCMS | |
|---|---|---|---|
| Compound | | (ESI) | |
| No. | Name | m/z | ¹H NMR |
| 67 | (2S,4R)-4-hydroxy-1-((2R)-2-(3- | 848.45 | 1H NMR (400 MHz, Methanol-d4) δ 8.87 |
| | (2-(5-(3-(2- | | (s, 1H), 8.60 (s, 1H), 8.00-7.90 (m, 1H), |
| | hydroxyphenyl)thieno[2,3- | | 7.51-7.24 (m, 6H), 7.06-6.96 (m, 2H), |
| | c]pyridazin-6-yl)-2- | | 6.04 (s, 1H), 5.03 (q, J = 7.1 Hz, 1H), |
| | azabicyclo[2.2.1]heptan-2- | | 4.61 (s, 1H), 4.52 (t, J = 8.2 Hz, 1H), 4.42 |
| | yl)ethoxy)isoxazol-5-yl)-3- | | (d, J = 15.0 Hz, 1H), 4.33 (t, J = 5.6 Hz, |
| | methylbutanoyl)-N-((S)-1-(4-(4- | | 1H), 3.84 (dd, J = 10.9, 4.1 Hz, 1H), 3.69 |
| | methylthiazol-5- | | (d, J = 9.9 Hz, 1H), 3.62 (d, J = 11.6 Hz, |
| | yl)phenyl)ethyl)pyrrolidine-2- | | 1H), 3.55 (s, 1H), 3.14-2.93 (m, 3H), |
| | carboxamide | | 2.74 (s, 1H), 2.57 (dd, J = 35.5, 10.2 Hz, |
| | | | 2H), 2.48 (s, 2H), 2.45-2.32 (m, 2H), |
| | | | 2.18 (t, J = 10.8 Hz, 1H), 1.95 (ddd, J = |
| | | | 13.3, 8.8, 4.6 Hz, 1H), 1.85 (t, J = 12.8 |
| | | | Hz, 2H), 1.74 (d, J = 10.6 Hz, 1H), 1.56 |
| | | | (dd, J = 31.9, 7.0 Hz, 3H), 1.28 (s, 1H), |
| | | | 1.06 (d, J = 6.5 Hz, 3H), 0.91 (dd, J = |
| | | | 10.2, 6.7 Hz, 3H). |
| 71 | (2S,4R)-4-hydroxy-N-[(1S)-1-[4- | 848.3 | 1H NMR (300 MHz, Methanol-d4) δ 8.87 |
| | (4-methyl-1,3-thiazol-5- | | (d, J = 13.5 Hz, 1H), 8.64 (s, 1H), 7.99 (d, |
| | yl)phenyl]ethyl]-1-[(2R)-3-methyl- | | J = 8.0 Hz, 1H), 7.48-7.34 (m, 6H), 7.04 |
| | 2-(3-{2-[(1R,5S,6r)-6-[3-(2- | | (d, J = 8.2 Hz, 2H), 6.06 (s, 1H), 4.65- |
| | hydroxyphenyl)thieno[2,3- | | 4.56 (m, 2H), 4.51-4.39 (m, 2H), 3.75- |
| | c]pyridazin-6-yl]-3- | | 3.49 (m, 3H), 3.24 (s, 4H), 3.10 (s, 2H), |
| | azabicyclo[3.1.1]heptan-3- | | 2.78 (s, 2H), 2.48 (d, J = 8.6 Hz, 4H), |
| | yl]ethoxy}-1,2-oxazol-5- | | 2.41-2.10 (m, 1H), 2.09-1.73 (m, 1H), |
| | yl)butanoyl]pyrrolidine-2- | | 1.57 (dd, J = 21.7, 7.1 Hz, 3H), 1.30 (s, |
| | carboxamide | | 3H), 1.07 (d, J = 6.5 Hz, 3H), 0.98-0.93 |
| | | | (m, 1H), 0.91 (d, J = 6.8 Hz, 3H). |
| 74 | (2S,4R)-4-hydroxy-1-((R)-2-(3-(2- | 848.3 | 1H NMR (400 MHz, Methanol-d4) δ 8.86 |
| | ((1R,5S,6S)-6-(3-(2- | | (d, J = 7.9 Hz, 1H), 8.54 (s, 1H), 8.03- |
| | hydroxyphenyl)thieno[2,3- | | 7.94 (m, 1H), 7.48-7.28 (m, 5H), 7.26- |
| | c]pyridazin-6-yl)-3- | | 7.18 (m, 1H), 7.06-6.96 (m, 2H), 5.51 |
| | azabicyclo[3.1.1]heptan-3- | | (s, 1H), 5.04-4.99 (m, 1H), 4.48 (m, |
| | yl)ethoxy)isoxazol-5-yl)-3- | | 2H), 4.08 (t, J = 5.2 Hz, 2H), 3.73 (s, 2H), |
| | methylbutanoyl)-N-((S)-1-(4-(4- | | 3.54-3.47 (m, 1H), 3.43-3.36 (m, 2H), |
| | methylthiazol-5- | | 2.92-2.83 (m, 6H), 2.46 (d, J = 11.0 Hz, |
| | yl)phenyl)ethyl)pyrrolidine-2- | | 3H), 2.30 (s, 2H), 2.28 (t, J = 5.1 Hz, 1H), |
| | carboxamide | | 2.19-2.10 (m, 1H), 1.93 (ddd, J = 14.1, |
| | | | 9.1, 5.0 Hz, 1H), 1.51 (t, J = 7.2 Hz, 3H), |
| | | | 1.36 (d, J = 8.9 Hz, 1H), 0.98 (t, J = 6.5 |
| | | | Hz, 3H), 0.79 (dd, J = 13.9, 6.7 Hz, 3H). |
| 101 | (2S,4R)-4-hydroxy-1-((R)-2-(3-(2- | 836.15 | 1H NMR (400 MHz, Methanol-d4) δ 8.86 |
| | (4-(3-(2-hydroxyphenyl)thieno[2,3- | | (d, J = 5.4 Hz, 1H), 8.76 (d, J = 3.0 Hz, |
| | c]pyridazin-5-yl)piperidin-1- | | 1H), 8.10 (dt, J = 7.4, 2.7 Hz, 1H), 7.93 |
| | yl)ethoxy)isoxazol-5-yl)-3- | | (d, J = 3.8 Hz, 1H), 7.48-7.29 (m, 5H), |
| | methylbutanoyl)-N-((S)-1-(4-(4- | | 7.03 (t, J = 7.4 Hz, 2H), 6.01 (d, J = 23.9 |
| | methylthiazol-5- | | Hz, 1H), 5.09-4.94 (m, 2H), 4.61-4.34 |
| | yl)phenyl)ethyl)pyrrolidine-2- | | (m, 4H), 3.84 (dd, J = 10.8, 4.1 Hz, 1H), |
| | carboxamide | | 3.65 (dd, J = 27.5, 10.5 Hz, 2H), 3.24- |
| | | | 3.09 (m, 3H), 2.91 (q, J = 5.7 Hz, 2H), |
| | | | 2.46 (d, J = 11.3 Hz, 3H), 2.42-2.30 (m, |
| | | | 2H), 2.26-1.89 (m, 6H), 1.56 (dd, J = |
| | | | 30.2, 7.0 Hz, 3H), 1.06 (d, J = 6.6 Hz, |
| | | | 3H), 0.91 (dd, J = 9.8, 6.7 Hz, 3H). |
| 102 | (2S,4R)-4-hydroxy-1-((R)-2-(3-(2- | 807.95 | 1H NMR (300 MHz, Methanol-d4) δ 8.95 |
| | (3-(3-(2-hydroxyphenyl)thieno[2,3- | | (d, J = 7.2 Hz, 1H), 8.83 (d, J = 3.5 Hz, |
| | c]pyridazin-5-yl)azetidin-1- | | 1H), 8.21-8.05 (m, 2H), 7.64-7.33 (m, |
| | yl)ethoxy)isoxazol-5-yl)-3- | | 5H), 7.14-7.02 (m, 2H), 6.07 (d, J = 18.8 |
| | methylbutanoyl)-N-((S)-1-(4-(4- | | Hz, 1H), 5.16-5.02 (m, 1H), 4.59 (t, J = |
| | methylthiazol-5- | | 8.2 Hz, 1H), 4.52 (s, 1H), 4.39 (q, J = 5.2, |
| | yl)phenyl)ethyl)pyrrolidine-2- | | 4.8 Hz, 2H), 4.24 (t, J = 7.6 Hz, 1H), 4.07 |
| | carboxamide | | (t, J = 7.5 Hz, 2H), 3.92 (dd, J = 10.8, 4.2 |
| | | | Hz, 1H), 3.85-3.65 (m, 2H), 3.64-3.52 |
| | | | (m, 2H), 3.08 (t, J = 5.2 Hz, 2H), 2.55 (d, |
| | | | J = 9.8 Hz, 3H), 2.44 (dq, J = 9.7, 6.6 Hz, |
| | | | 1H), 2.26 (t, J = 10.7 Hz, 1H), 2.17-1.97 |
| | | | (m, 1H), 1.63 (dd, J = 15.6, 7.0 Hz, 3H), |
| | | | 1.13 (d, J = 6.5 Hz, 3H), 0.97 (t, J = 7.1 |
| | | | Hz, 3H). |
| 111 | (2S,4R)-4-hydroxy-1-((R)-2-(3-(2- | 822.2 | 1H NMR (300 MHz, DMSO-d6) δ 12.55 |
| | (3-((3-(2- | | (s, 1H), 8.99 (s, 1H), 8.83 (s, 1H), 8.42 |
| | hydroxyphenyl)thieno[2,3- | | (d, J = 7.7 Hz, 1H), 8.18 (d, 1H), 8.05 (s, |
| | c]pyridazin-5-yl)methyl)azetidin-1- | | 1H), 7.51-7.41 (m, 2H), 7.40-7.33 (m, |
| | yl)ethoxy)isoxazol-5-yl)-3- | | 3H), 7.10-6.99 (m, 2H), 6.07 (s, 1H), |
| | methylbutanoyl)-N-((S)-1-(4-(4- | | 5.10 (d, J = 3.6 Hz, 1H), 4.91 (t, J = 7.2 |
| | methylthiazol-5- | | Hz, 1H), 4.37 (t, J = 7.9 Hz, 1H), 4.32- |
| | yl)phenyl)ethyl)pyrrolidine-2- | | 4.23 (m, 1H), 4.16-4.03 (m, 2H), 3.76- |
| | carboxamide | | 3.53 (m, 2H), 3.50-3.35 (m, 3H), 3.18 |
| | | | (d, J = 7.5 Hz, 2H), 2.97 (t, J = 6.4 Hz, |
| | | | 2H), 2.92-2.80 (m, 1H), 2.79-2.69 (m, |
| | | | 2H), 2.46 (s, 3H), 2.32-2.15 (m, 1H), |
| | | | 2.09-1.97 (m, 1H), 1.85-1.71 (m, 1H), |
| | | | 1.38 (d, J = 7.0 Hz, 3H), 0.96 (d, J = 6.5 |
| | | | Hz, 3H), 0.79 (d, J = 6.6 Hz, 3H). |

Preparation of (2S,4R)-4-hydroxy-N-[(1S)-1-[4-(4-methyl-1,3-thiazol-5-yl)phenyl]ethyl]-1-[(2S)-3-methyl-2-[3-(piperazin-1-yl)-1,2-oxazol-5-yl]butanoyl]pyrrolidine-2-carboxamide (I-4) and (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (I-5)

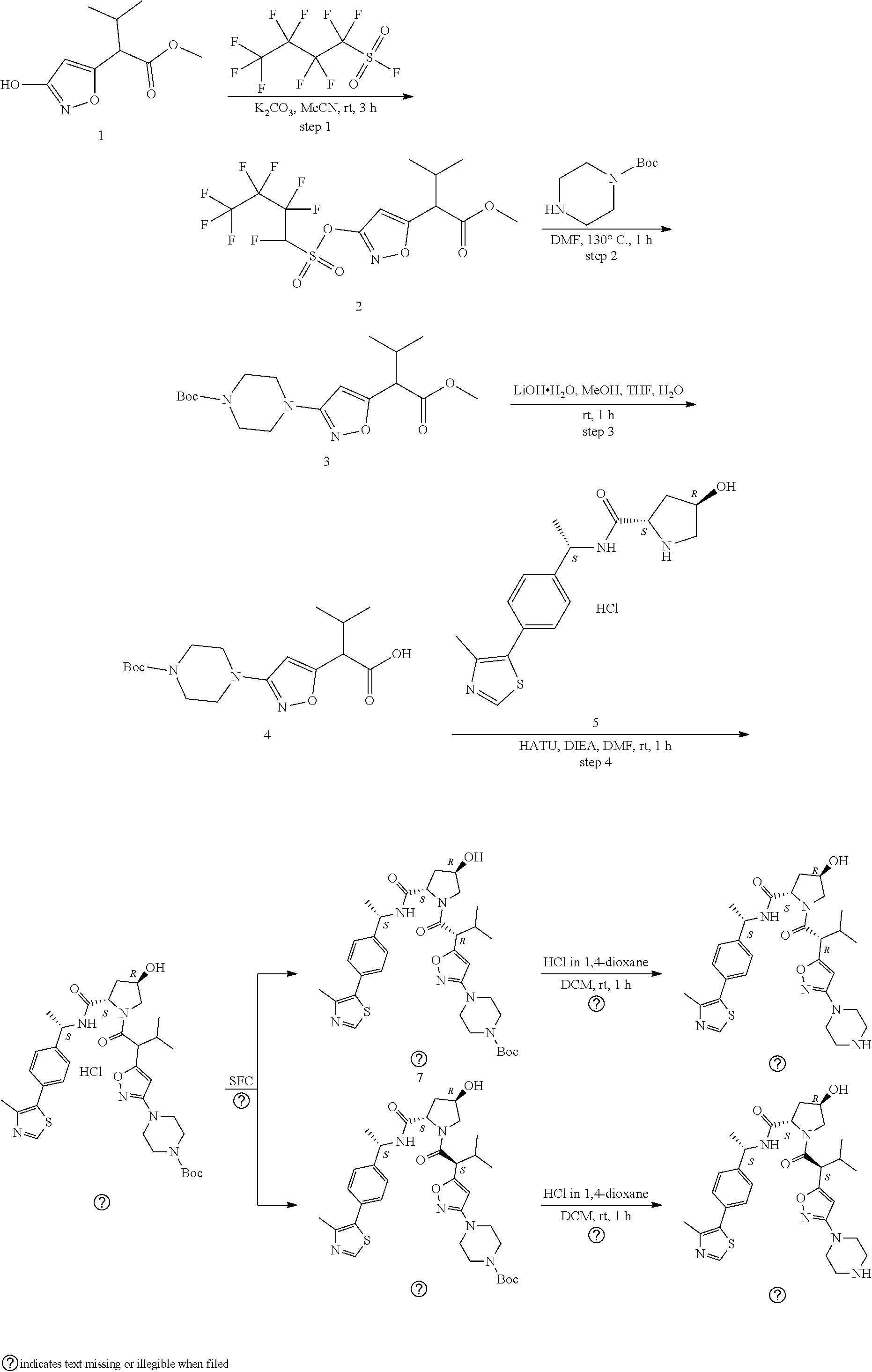

Step 1: Preparation of methyl 3-methyl-2-[3-[(1,1,2,2,3,3,4,4,4-nonafluorobutanesulfonyl)oxy]-1,2-oxazol-5-yl]butanoate (Intermediate 2)

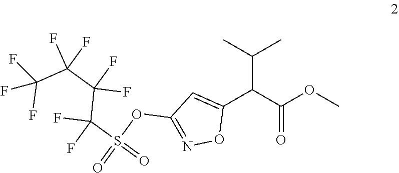 [SMILES: COC(=O)C(c1cc(OS(=O)(=O)C(F)(F)C(F)(F)C(F)(F)C(F)(F)F)no1)C(C)C]

To a stirred solution methyl 2-(3-hydroxy-1,2-oxazol-5-yl)-3-methylbutanoate (100.00 mg, 0.502 mmol, 1.00 equiv) in MeCN (0.50 mL) was added perfluorobutanesulfonyl fluoride (303.29 mg, 1.004 mmol, 2.00 equiv) and K₂CO₃ (208.13 mg, 1.506 mmol, 3.00 equiv) at room temperature. The resulting mixture was stirred for 3 h, then carefully quenched with water at 0 degrees C. The resulting mixture was extracted with EA (2×50 mL), and the combined organic layers were washed with brine (50 mL), and dried over anhydrous Na₂SO₄. After filtration, the filtrate was concentrated under reduced pressure, and the residue purified by silica gel column chromatography, eluted with PE/EA (2/1) to afford Intermediate 2 (217 mg, crude) as a white solid. LCMS (ESI) m/z: [M+H]⁺=482.

Step 2: Preparation of tert-butyl 4-[5-(1-methoxy-3-methyl-1-oxobutan-2-yl)-1,2-oxazol-3-yl]piperazine-1-carboxylate (Intermediate 3)

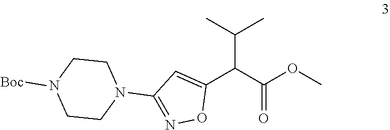 [SMILES: COC(=O)C(c1cc(N2CCN(C(=O)OC(C)(C)C)CC2)no1)C(C)C]

To a stirred solution of Intermediate 2 (217.00 mg, 0.451 mmol, 1.00 equiv) in DMF (3.00 mL) was added tert-butyl piperazine-1-carboxylate (83.98 mg, 0.451 mmol, 1.00 equiv) at room temperature. The resulting mixture was stirred for 1 h at 130 degrees C. The mixture was allowed to cool down to room temperature. The residue was purified by reverse flash chromatography with the following conditions: column, C18 silica gel; mobile phase, MeCN in water (0.1% FA), 0 to 100% gradient in 30 min. This provided intermediate 3 (54 mg, 32.59%) as a yellow oil. LCMS (ESI) m/z: [M+H]⁺=368.

Step 3: Preparation of 2-[3-[4-(tert-butoxycarbonyl)piperazin-1-yl]-1,2-oxazol-5-yl]-3-methylbutanoic acid (Intermediate 4)

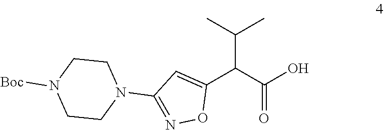 [SMILES: CC(C)C(C(=O)O)c1cc(N2CCN(C(=O)OC(C)(C)C)CC2)no1]

To a stirred solution of Intermediate 3 (54.00 mg, 0.147 mmol, 1.00 equiv) in MeOH (0.80 mL) was added THF (0.80 mL) and H₂O (0.80 mL) at room temperature, follow by addition of LiOH—H₂O (18.50 mg, 0.441 mmol, 3.00 equiv). The resulting mixture was stirred for an additional 1 h at room temperature. The mixture was acidified to pH 6 with HCl (1M, aq.), then extracted with EA (2×50 mL). The combined organic layers were washed with saturated brine (50 mL), dried over anhydrous Na₂SO₄, and filtered. The filtrate was concentrated under reduced pressure. This provided Intermediate 4 (52 mg, crude) as a yellow solid. LCMS (ESI) m/z: [M+H]⁺=354.

Step 4: Preparation of tert-butyl 4-(5-[1-[(2S,4R)-4-hydroxy-2-[[(1S)-1-[4-(4-methyl-1,3-thiazol-5-yl)phenyl]ethyl]carbamoyl]pyrrolidin-1-yl]-3-methyl-1-oxobutan-2-yl]-1,2-oxazol-3-yl)piperazine-1-carboxylate (Intermediate 6)

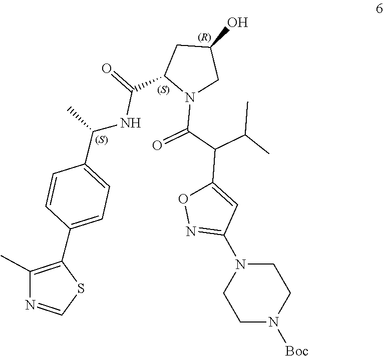

To a stirred solution of Intermediate 4 (52.00 mg, 0.119 mmol, 1.00 equiv) in DMF (2.00 mL) was added HATU (135.56 mg, 0.357 mmol, 3.00 equiv) and DIEA (76.80 mg, 0.595 mmol, 5.00 equiv) at room temperature. To the above mixture was added (2S,4R)-4-hydroxy-N-[(1S)-1-[4-(4-methyl-1,3-thiazol-5-yl)phenyl]ethyl]pyrrolidine-2-carboxamide (70.90 mg, 0.214 mmol, 1.80 equiv) at room temperature. The resulting mixture was stirred for an additional 1 h. The mixture was purified directly by reverse flash chromatography with the following conditions: column, C18 silica gel; mobile phase, MeCN in water (0.1% FA), 0 to 100% gradient in 30 min. This provided in Intermediate 6 (73 mg, 92.12%) as a white solid. LCMS (ESI) m/z: [M+H]⁺=667.

Step 5: Preparation of (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (Intermediate 7); (2S,4R)-4-hydroxy-N-[(1S)-1-[4-(4-methyl-1,3-thiazol-5-yl)phenyl]ethyl]-1-[(2S)-3-methyl-2-[3-(piperazin-1-yl)-1,2-oxazol-5-yl]butanoyl]pyrrolidine-2-carboxamide (Intermediate 8)

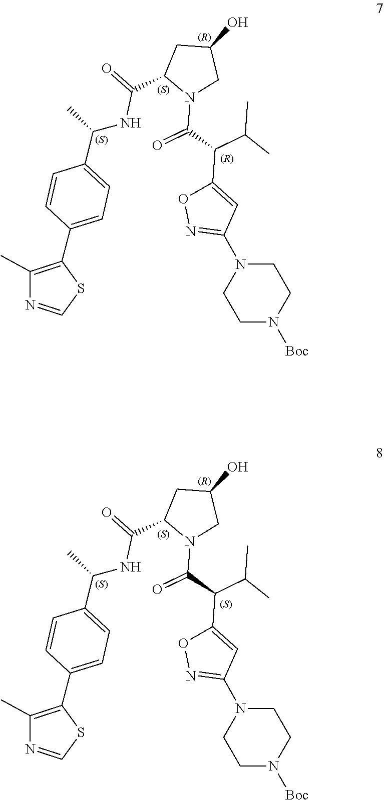

The Intermediate 6 (73 mg) was purified by SFC with the following conditions: Column, CHIRAL ART Amylose-C NEO, 3*25 cm, 5 um; mobile phase, MeOH. This provided Intermediate 7 (37 mg, second peak). LCMS (ESI) m/z: [M+H]⁺=667, and Intermediate 8 (34 mg, first peak). LCMS (ESI) m/z: [M+H]⁺=667.

Step 6: Preparation of (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (I-4)

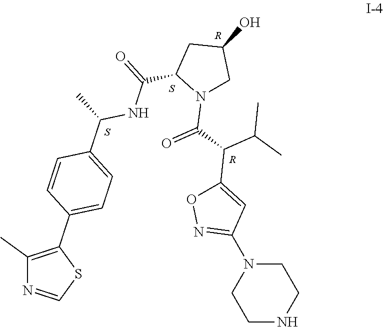

To a stirred solution of Intermediate 7 (37.00 mg, 0.055 mmol, 1.00 equiv) in DCM (1.50 mL) was added HCl in 1,4-dioxane (1.50 mL, 26.276 mmol, 473.57 equiv) at 0 degrees C. The resulting mixture was stirred for 1 h at room temperature, then concentrated under reduced pressure. This provided I-4 (45 mg, crude) as a yellow oil. LCMS (ESI) m/z: [M+H]⁺=567.

Step 7: Preparation of (2S,4R)-4-hydroxy-N-[(1S)-1-[4-(4-methyl-1,3-thiazol-5-yl)phenyl]ethyl]-1-[(2S)-3-methyl-2-[3-(piperazin-1-yl)-1,2-oxazol-5-yl]butanoyl]pyrrolidine-2-carboxamide (I-5)

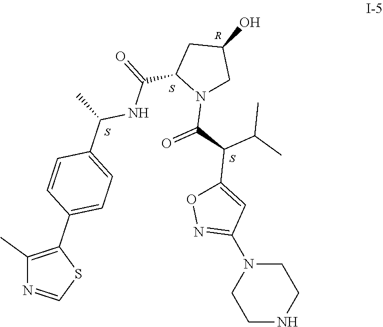

To a stirred solution of Intermediate 8 (34.00 mg, 0.051 mmol, 1.00 equiv) in DCM (1.50 mL) was added HC in 1,4-dioxane (1.50 mL, 26.276 mmol, 515.35 equiv) at 0 degrees C. The resulting mixture was stirred for 1 h at room temperature, then concentrated under reduced pressure. This provided I-5 (45 mg, crude) as a yellow oil. LCMS (ESI) m/z: [M+H]⁺=567.

Preparation of (2S,4R)-4-hydroxy-1-[(2R)-2-{3-[4-(2-{3-[3-(2-hydroxyphenyl)thieno[2,3-c]pyridazin-6-yl]azetidin-1-yl}acetyl)piperazin-1-yl]-1,2-oxazol-5-yl}-3-methylbutanoyl]-N-[(1S)-1-[4-(4-methyl-1,3-thiazol-5-yl)phenyl]ethyl]pyrrolidine-2-carboxamide

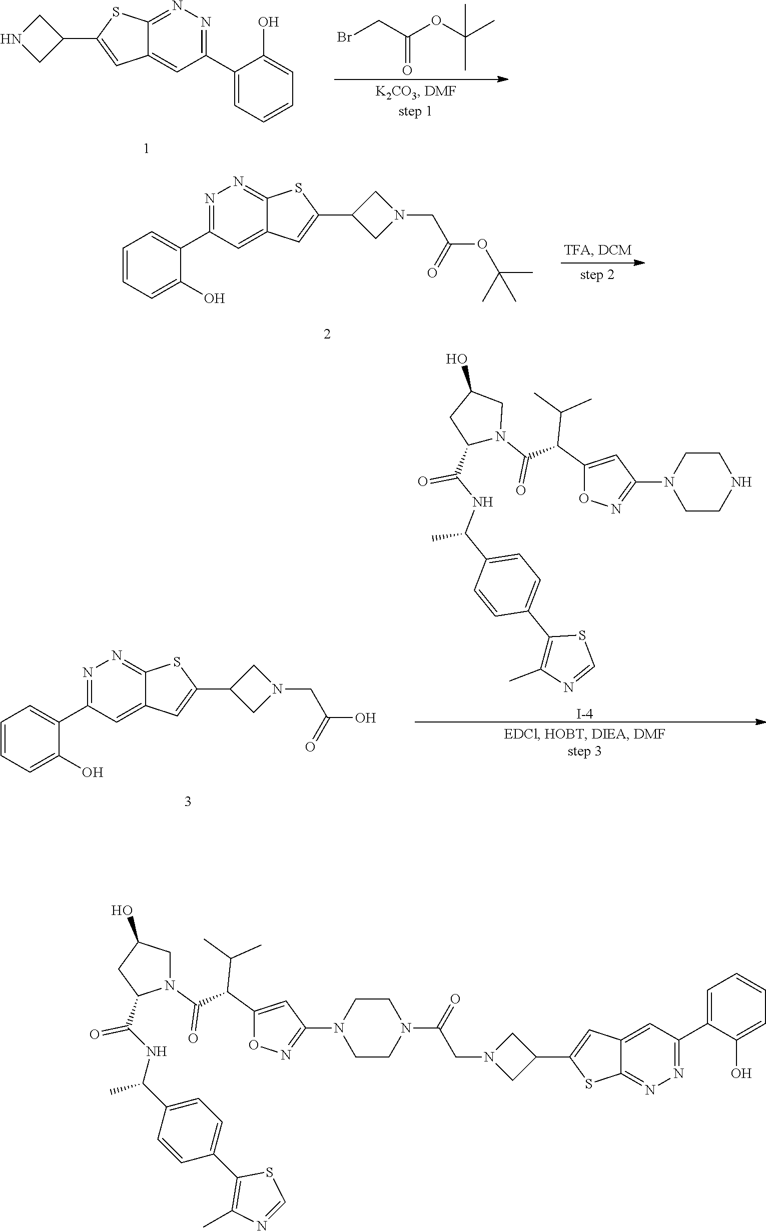

Step 1: Preparation of tert-butyl 2-(3-(3-(2-hydroxyphenyl)thieno[2,3-c]pyridazin-6-yl)azetidin-1-yl)acetate (Intermediate 2)

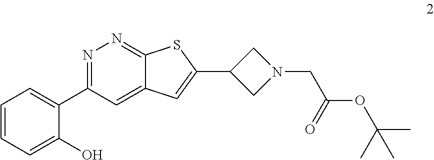 [SMILES: CC(C)(C)OC(=O)CN1CC(c2cc3cc(-c4ccccc4O)nnc3s2)C1]

To a stirred solution of 2-[6-(azetidin-3-yl)thieno[2,3-c]pyridazin-3-yl]phenol (30 mg, 0.106 mmol, 1.00 equiv) and K₂CO₃ (43.90 mg, 0.318 mmol, 3 equiv) in DMF (3 mL) was added tert-butyl 2-bromoacetate (24.78 mg, 0.127 mmol, 1.2 equiv) in portions at room temperature. The resulting mixture was stirred for 1 h at 80 degrees C., and the resulting mixture was diluted with water (3 mL) followed by extraction with EtOAc (3×10 mL). The combined organic layers were washed with brine (2×5 mL), dried over anhydrous Na₂SO₄, and filtered. The filtrate was concentrated under reduced pressure to afford intermediate 2 (46 mg, crude) as a yellow solid. (LCMS (ESI) m/z [M+H]⁺=398.

Step 2: Preparation of 2-(3-(3-(2-hydroxyphenyl)thieno[2,3-c]pyridazin-6-yl)azetidin-1-yl)acetic acid (Intermediate 3)

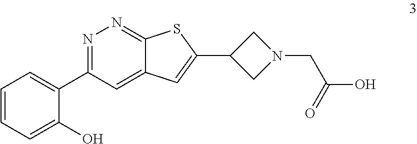 [SMILES: O=C(O)CN1CC(c2cc3cc(-c4ccccc4O)nnc3s2)C1]

A solution of tert-butyl 2-{3-[3-(2-hydroxyphenyl)thieno[2,3-c]pyridazin-6-yl]azetidin-1-yl}acetate (40 mg, 0.101 mmol, 1.00 equiv) and TFA (0.4 mL) in DCM (2 mL) was stirred for 1 h at room temperature under a nitrogen atmosphere. The resulting mixture was concentrated under reduced pressure to afford intermediate 3 (34.3 mg, crude) as a brown solid. (LCMS (ESI) m/z [M+H]⁺=342.

Step 3: Preparation of (2S,4R)-4-hydroxy-1-[(2R)-2-{3-[4-(2-{3-[3-(2-hydroxyphenyl)thieno[2,3-c]pyridazin-6-yl]azetidin-1-yl}acetyl)piperazin-1-yl]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 3)

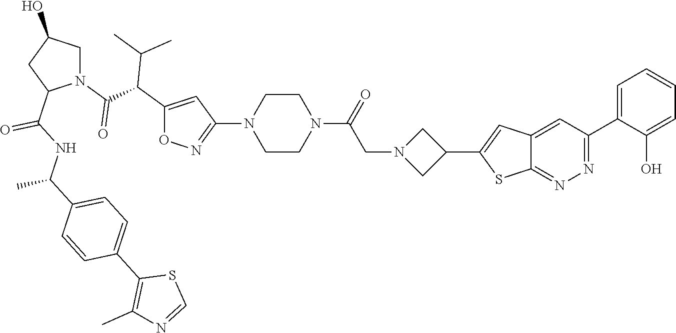 [SMILES: Cc1ncsc1-c1ccc([C@H](C)NC(=O)C2C[C@@H](O)CN2C(=O)[C@@H](c2cc(N3CCN(C(=O)CN4CC(c5cc6cc(-c7ccccc7O)nnc6s5)C4)CC3)no2)C(C)C)cc1]

To a stirred solution of {3-[3-(2-hydroxyphenyl)thieno[2,3-c]pyridazin-6-yl]azetidin-1-yl}acetic acid (34.3 mg, 0.100 mmol, 1.00 equiv) and (2S,4R)-4-methyl-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (I-4, 42.56 mg, 0.075 mmol, 0.75 equiv) in DMF (1.5 mL) were added EDCI (38.52 mg, 0.200 mmol, 2 equiv), HOBT (27.15 mg, 0.200 mmol, 2 equiv) and DIEA (51.94 mg, 0.400 mmol, 4 equiv). The resulting mixture was stirred for 3 h at room temperature. Without any additional work-up, the mixture was purified by reverse phase flash with the following conditions (Column: Xselect CSH F-Phenyl OBD column, 19*250 mm, 5 μm; Mobile Phase A: Water (0.05% FA), Mobile Phase B: MeOH—HPLC; Flow rate: 25 mL/min; Gradient: 40% B to 71% B in 8 min, 71% B; to afford the title compound (10.2 mg, 11.41%) as a light yellow solid. ¹H NMR (300 MHz, DMSO-d6) δ 10.70 (s, 1H), 9.00 (s, 1H), 8.80 (d, J=8.3 Hz, 1H), 8.40 (d, J=7.7 Hz, 1H), 8.04 (d, J=8.0, 1.6 Hz, 1H), 7.71 (d, J=18.1 Hz, 1H), 7.48-7.35 (m, 5H), 7.10-6.97 (m, 2H), 6.25 (s, 1H), 4.92 (t, J=7.2 Hz, 1H), 4.75-4.24 (m, 9H), 3.91-3.62 (m, 1H), 3.66-3.55 (m, 4H), 3.36-3.27 (m, 3H), 3.26-3.18 (m, 3H), 2.46 (s, 3H), 2.32-2.23 (m, 1H), 2.11-1.99 (m, 1H), 1.87-1.70 (m, 1H), 1.39 (d, J=6.9 Hz, 3H), 0.98 (d, 3H), 0.82 (d, 3H). LCMS (ESI) m/z: [M+H]⁺=890.55.

The compounds in Table 4 were prepared using procedures similar to those used above for the preparation of compound 3 using the appropriate amine and carboxylic acid.

TABLE 4

| | | LCMS | |
|---|---|---|---|
| Compound | | (ESI) | |
| No. | Name | m/z | ¹H NMR |
| 50 | (2S,4R)-4-hydroxy-1-((S)-2- | 795.25 | 1H NMR (300 MHz, DMSO-d6) δ 12.93 |
| | ((1s,4R)-4-(3-(2-hydroxyphenyl)-5- | | (s, 1H), 9.06 (d, J = 11.0 5z, 1H), 8.75 |
| | methylthieno[2,3-c]pyridazin-6- | | (s, 1H), 8.47 (d, J = 7.8 Hz, 1H), 8.24 |
| | yl)cyclohexane-1-carboxamido)-3,3- | | (d, J = 7.8 Hz, 1H), 7.78 (d, J = 9.4 Hz, |
| | dimethylbutanoyl)-N-((S)-1-(4-(4- | | 1H), 7.56-7.33 (m, 5H), 7.15-7.03 |
| | methylthiazol-5- | | (m, 2H), 5.22 (d, J = 3.5 Hz, 1H), 5.09- |
| | yl)phenyl)ethyl)pyrrolidine-2- | | 4.95 (m, 1H), 4.69 (d, J = 9.3 Hz, 1H), |
| | carboxamide | | 4.52 (t, J = 8.1 Hz, 1H), 4.38 (s, 1H), |
| | | | 3.73 (s, 2H), 3.35 (s, 1H), 2.81 (s, 1H), |
| | | | 2.52 (d, J = 7.2 Hz, 6H), 2.21-2.03 (m, |
| | | | 4H), 1.97-1.79 (m, 5H), 1.73-1.64 |
| | | | (m, 1H), 1.50-1.39 (m, 4H), 1.31 (s, |
| | | | 4H), 1.04 (s, 9H). |
| 51 | (2S,4R)-4-hydroxy-1-((S)-2-((1r,4S)- | 795.3 | 1H NMR (300 MHz, DMSO-d6) δ 12.91 |
| | 4-(3-(2-hydroxyphenyl)-5- | | (s, 1H), 9.07 (s, 1H), 8.76 (s, 1H), 8.48 |
| | methylthieno[2,3-c]pyridazin-6- | | (d, J = 7.8 Hz, 1H), 8.24 (dd, J = 8.4, |
| | yl)cyclohexane-1-carboxamido)-3,3- | | 1.6 Hz, 1H), 7.83 (d, J = 9.2 Hz, 1H), |
| | dimethylbutanoyl)-N-((S)-1-(4-(4- | | 7.55-7.39 (m, 5H), 7.13-7.04 (m, |
| | methylthiazol-5- | | 2H), 5.20 (d, J = 3.5 Hz, 1H), 5.18- |
| | yl)phenyl)ethyl)pyrrolidine-2- | | 4.95 (m, 1H), 4.62 (d, J = 9.2 Hz, 1H), |
| | carboxamide | | 4.51 (t, J = 8.1 Hz, 1H), 4.37 (s, 1H), |
| | | | 3.69 (s, 2H), 3.32 (s, 1H), 2.53 (s, 1H), |
| | | | 2.51 (d, J = 5.5 Hz, 6H), 2.12 (d, J = |
| | | | 11.9 Hz, 3H), 2.01 (d, J = 11.3 Hz, 1H), |
| | | | 1.96-1.80 (m, 2H), 1.79-1.59 (m, |
| | | | 4H), 1.46 (d, J = 7.0 Hz, 3H), 1.04 (s, |
| | | | 9H). |
| 65 | N-((S)-1-((2S,4R)-4-hydroxy-2-(((S)- | 874.2 | 1H NMR (300 MHz, DMSO-d6) δ 12.84 |
| | 1-(4-(4-methylthiazol-5- | | (s, 1H), 9.06 (d, J = 2.8 Hz, 1H), 8.92 |
| | yl)phenyl)ethyl)carbamoyl)pyrrolidin- | | (s, 2H), 8.78 (s, 1H), 8.50 (d, J = 7.6 |
| | 1-yl)-3,3-dimethyl-1-oxobutan-2-yl)- | | Hz, 1H), 8.29-8.09 (m, 2H), 7.48 (p, J = |
| | 2-(4-(3-(2-hydroxyphenyl)-5- | | 8.1 Hz, 5H), 7.10 (d, J = 7.8 Hz, 2H), |
| | methylthieno[2,3-c]pyridazin-6- | | 5.22 (s, 1H), 5.03 (d, J = 10.0 Hz, 3H), |
| | yl)piperidin-1-yl)pyrimidine-5- | | 4.82 (d, J = 9.2 Hz, 1H), 4.61-4.23 (m, |
| | carboxamide | | 2H), 3.76 (s, 3H), 3.25 (t, J = 12.8 Hz, |
| | | | 3H), 2.53 (d, J = 4.2 Hz, 4H), 2.16 (d, J = |
| | | | 13.1 Hz, 3H), 1.95-1.58 (m, 3H), |
| | | | 1.46 (d, J = 6.9 Hz, 3H), 1.10 (s, 9H). |
| 78 | (2S,4R)-4-hydroxy-1-((S)-2-(3-(4-(3- | 796.2 | 1H NMR (400 MHz, DMSO-d6) δ 13.02 |
| | (2-hydroxyphenyl)thieno[3,2- | | (s, 1H), 9.02 (s, 1H), 8.85 (s, 1H), 8.79 |
| | c]pyridazin-6-yl)piperidin-1- | | (d, J = 9.4 Hz, 1H), 8.63-8.56 (m, 1H), |
| | yl)propanamido)-3,3- | | 7.98 (dd, J = 8.1, 1.6 Hz, 1H), 7.66 (s, |
| | dimethylbutanoyl)-N-(4-(4- | | 1H), 7.41-7.30 (m, 3H), 7.29-7.16 |
| | methylthiazol-5- | | (m, 2H), 7.06-6.97 (m, 2H), 5.16 (d, J = |
| | yl)benzyl)pyrrolidine-2-carboxamide | | 3.5 Hz, 1H), 4.58 (d, J = 9.5 Hz, 1H), |
| | | | 4.51-4.40 (m, 2H), 4.40-4.33 (m, |
| | | | 1H), 4.23-4.13 (m, 1H), 3.73-3.58 |
| | | | (m, 2H), 3.17-2.97 (m, 3H), 2.70- |
| | | | 2.59 (m, 1H), 2.46-2.40 (m, 1H), 2.33 |
| | | | (s, 3H), 2.32-2.25 (m, 2H), 2.23-2.13 |
| | | | (m, 1H), 2.13-2.00 (m, 4H), 2.00- |
| | | | 1.79 (m, 3H), 0.98 (s, 9H). |
| 79 | (2S,4R)-4-hydroxy-1-((S)-2-(4-(4-(3- | 824.1 | 1H NMR (300 MHz, DMSO-d6) δ 9.16 |
| | (2-hydroxyphenyl)thieno[3,2- | | (s, 1H), 8.99 (s, 1H), 8.58 (s, 1H), 8.02 |
| | c]pyridazin-6-yl)piperidin-1-yl)-4- | | (d, J = 7.7 Hz, 1H), 7.94 (d, J = 9.2 Hz, |
| | oxobutanamido)-3,3- | | 1H), 7.73 (s, 1H), 7.47-7.32 (m, 6H), |
| | dimethylbutanoyl)-N-(4-(4- | | 7.11-7.01 (m, 2H), 4.60-4.30 (m, |
| | methylthiazol-5- | | 6H), 4.30-4.21 (m, 1H), 4.08-4.04 |
| | yl)benzyl)pyrrolidine-2-carboxamide | | (m, 1H), 3.71-3.56 (m, 4H), 2.65- |
| | | | 2.55 (m, 3H), 2.44 (s, 4H), 2.15-2.09 |
| | | | (m, 3H), 2.01-1.87 (m, 1H), 1.84- |
| | | | 1.39 (m, 2H), 0.95 (s, 9H). |
| 80 | (2S,4R)-4-hydroxy-1-((S)-2-(5-(4-(3- | 838.25 | 1H NMR (300 MHz, DMSO-d6) δ 13.01 |
| | (2-hydroxyphenyl)thieno[3,2- | | (s, 1H), 9.21 (s, 1H), 9.05 (s, 1H), 8.64 |
| | c]pyridazin-6-yl)piperidin-1-yl)-5- | | (t, J = 6.0 Hz, 1H), 8.09 (d, J = 7.8 Hz, |
| | oxopentanamido)-3,3- | | 1H), 7.99 (d, J = 9.2 Hz, 1H), 7.79 (s, |
| | dimethylbutanoyl)-N-(4-(4- | | 1H), 7.54-7.38 (m, 5H), 7.14-7.04 |
| | methylthiazol-5- | | (m, 2H), 5.21 (d, J = 3.5 Hz, 1H), 4.63 |
| | yl)benzyl)pyrrolidine-2-carboxamide | | (d, J = 9.3 Hz, 2H), 4.56-4.40 (m, 3H), |
| | | | 4.29 (dd, J = 15.8, 5.4 Hz, 1H), 4.05 (d, |
| | | | J = 13.6 Hz, 1H), 3.74 (brs, 2H), 3.33- |
| | | | 3.21 (m,1H) 2.79 (m, 1H), 2.51 (s, |
| | | | 3H), 2.47-2.26 (m, 5H), 2.25-2.04 |
| | | | (m, 3H), 2.05-1.90 (m, 1H), 1.86- |
| | | | 1.65 (m, 4H), 1.02 (brs, 9H). |
| 84 | (2S,4R)-4-hydroxy-1-((S)-2-(6-(4-(3- | 852.1 | 1H NMR (300 MHz, DMSO-d6) δ 13.01 |
| | (2-hydroxyphenyl)thieno[3,2- | | (brs, 1H), 9.21 (s, 1H), 9.05 (d, J = 2.9 |
| | c]pyridazin-6-yl)piperidin-1-yl)-6- | | Hz, 1H), 8.64 (t, J = 6.0 Hz, 1H), 8.13- |
| | oxohexanamido)-3,3- | | 8.04 (m, 1H), 7.96 (d, J = 9.3 Hz, 1H), |
| | dimethylbutanoyl)-N-(4-(4- | | 7.79 (s, 1H), 7.54-7.38 (m, 5H), 7.15- |
| | methylthiazol-5- | | 7.03 (m, 2H), 5.21 (d, J = 3.6 Hz, 1H), |
| | yl)benzyl)pyrrolidine-2-carboxamide | | 4.63 (d, J = 9.3 Hz, 2H), 4.58-4.45 (m, |
| | | | 2H), 4.42 (brs, 1H), 4.28 (dd, J = 15.8, |
| | | | 5.5 Hz, 1H), 4.09 (d, J = 13.6 Hz, 1H), |
| | | | 3.73 (brs, 2H), 3.47-3.43 (m, 1H), |
| | | | 3.32-3.18 (m, 1H), 2.75 (d, J = 12.5 |
| | | | Hz, 1H), 2.51 (d, J = 2.3 Hz, 3H), 2.43 |
| | | | (s, 3H), 2.19-2.05 (m, 4H), 2.05-1.91 |
| | | | (m, 1H), 1.78 (d, J = 12.0 Hz, 1H), 1.69- |
| | | | 1.51 (m, 5H), 1.01 (s, 9H). |
| 85 | (2S,4R)-4-hydroxy-1-((S)-2-((1r,4S)- | 942.4 | 1H NMR (300 MHz, DMSO-d6) δ 9.07 |
| | 4-(2-(4-(3-(2-hydroxyphenyl)-5- | | (s, 1H), 8.78 (s, 1H), 8.66 (s, 1H), 8.37 |
| | methylthieno[2,3-c]pyridazin-6- | | (s, 2H), 8.24 (d, J = 8.0 Hz, 1H), 7.88 |
| | yl)piperidin-1-yl)pyrimidin-5- | | (d, J = 9.3 Hz, 1H), 7.52-7.40 (m, 5H), |
| | yl)cyclohexane-1-carboxamido)-3,3- | | 7.10 (d, J = 7.7 Hz, 2H), 4.90 (d, J = |
| | dimethylbutanoyl)-N-(4-(4- | | 12.9 Hz, 2H), 4.64-4.25 (m, 5H), 3.76- |
| | methylthiazol-5- | | 3.66 (m, 3H), 3.12 (t, J = 12.4 Hz, |
| | yl)benzyl)pyrrolidine-2-carboxamide | | 2H), 2.52 (s, 3H), 2.50 (s, 3H), 2.50- |
| | | | 2.46 (m, 2H), 2.11 (d, J = 12.0 Hz, 3H), |
| | | | 1.96-1.93 (m, 5H), 1.75-1.39 (m, |
| | | | 6H), 1.03 (s, 9H). |
| 86 | (2S,4R)-4-hydroxy-1-((S)-2- | 942.4 | 1H NMR (400 MHz, DMSO-d6) δ 12.79 |
| | ((1s,4R)-4-(2-(4-(3-(2- | | (s, 1H), 8.98 (s, 1H), 8.70 (s, 1H), 8.56 |
| | hydroxyphenyl)-5-methylthieno[2,3- | | (t, J = 6.1 Hz, 1H), 8.26 (s, 2H), 8.20- |
| | c]pyridazin-6-yl)piperidin-1- | | 8.13 (m, 1H), 7.69 (d, J = 9.3 Hz, 1H), |
| | yl)pyrimidin-5-yl)cyclohexane-1- | | 7.46-7.33 (m, 5H), 7.06-6.98 (m, |
| | carboxamido)-3,3- | | 2H), 5.13 (d, J = 3.5 Hz, 1H), 4.83 (d, J = |
| | dimethylbutanoyl)-N-(4-(4- | | 13.0 Hz, 2H), 4.58 (d, J = 9.4 Hz, 1H), |
| | methylthiazol-5- | | 4.48-4.38 (m, 2H), 4.36 (s, 1H), 4.23 |
| | yl)benzyl)pyrrolidine-2-carboxamide | | (dd, J = 15.9, 5.4 Hz, 1H), 3.67 (s, 2H), |
| | | | 3.65-3.54 (m, 1H), 3.11-2.99 (m, |
| | | | 2H), 2.66 (d, J = 4.9 Hz, 1H), 2.49- |
| | | | 2.47 (m, 1H), 2.45 (s, 3H), 2.42 (s, 3H), |
| | | | 2.04 (d, J = 12.3 Hz, 3H), 2.00-1.71 |
| | | | (m, 5H), 1.65-1.44 (m, 6H), 0.95 (s, |
| | | | 9H). |

Preparation of 1-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperidine-4-carboxylic acid (I-6)

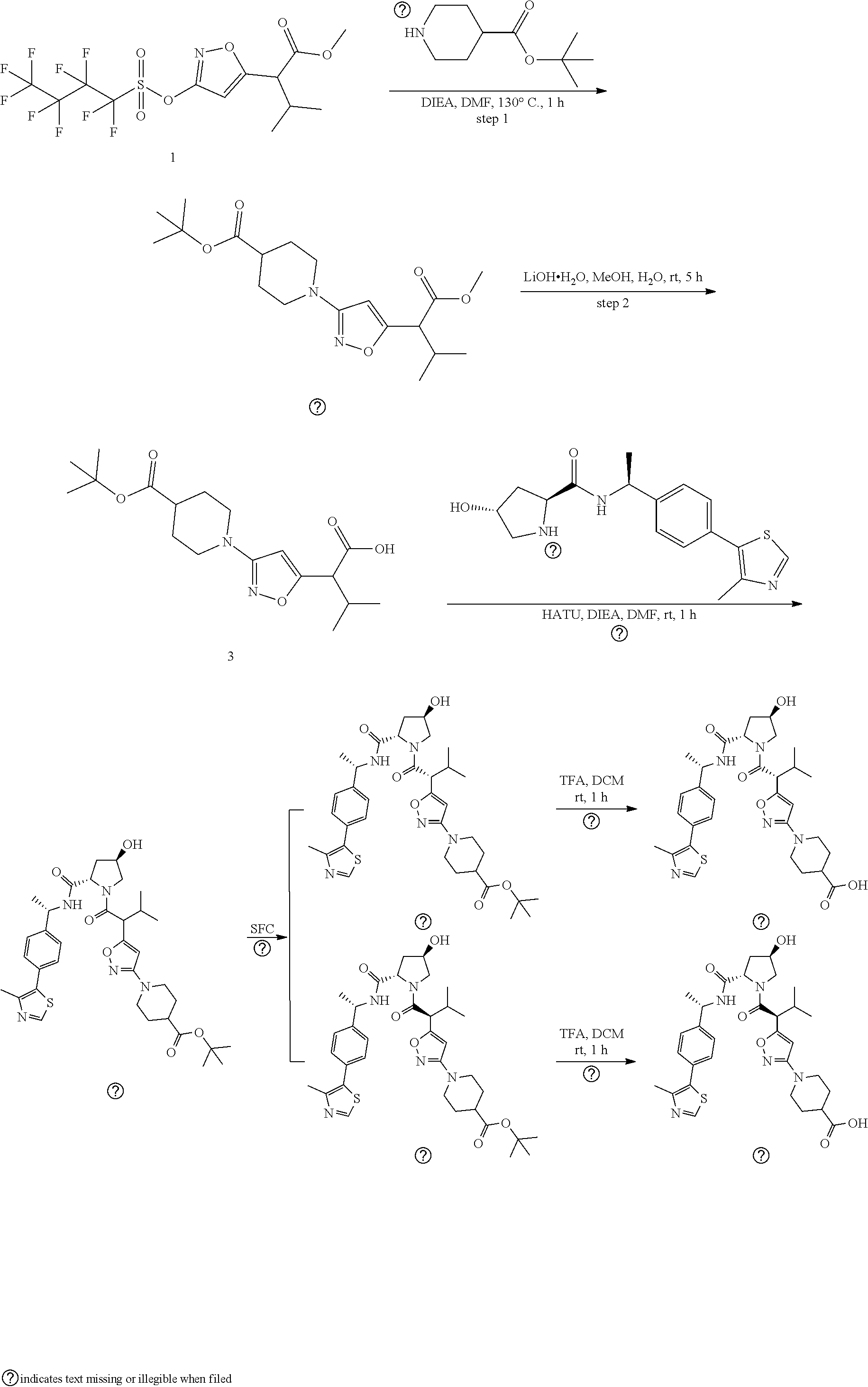

Step 1: Preparation of tert-butyl 1-[5-(1-methoxy-3-methyl-1-oxobutan-2-yl)-1,2-oxazol-3-yl]piperidine-4-carboxylate (Intermediate 2)

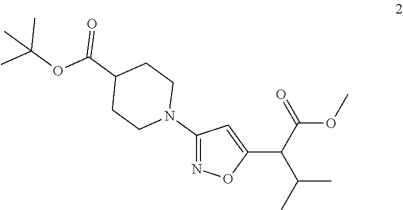 [SMILES: COC(=O)C(c1cc(N2CCC(C(=O)OC(C)(C)C)CC2)no1)C(C)C]

To a stirred solution of methyl 3-methyl-2-[3-[(1,1,2,2,3,3,4,4,4-nonafluorobutanesulfonyl)oxy]-1,2-oxazol-5-yl]butanoate (300.00 mg, 0.623 mmol, 1.00 equiv) and tert-butyl piperidine-4-carboxylate hydrochloride (414.63 mg, 1.869 mmol, 3.00 equiv) in DMF (3.00 mL) was added DIEA (402.80 mg, 3.115 mmol, 5.00 equiv) at room temperature. The resulting mixture was stirred for 1 h at 130 degrees C. The mixture was allowed to cool down to room temperature, and the resulting mixture purified by reverse flash chromatography with the following conditions: column, C18 silica gel; mobile phase, MeCN in water (0.1% FA), 0% to 100% gradient in 30 min. This resulted in Intermediate 2 (78 mg, 34.15%) as a yellow solid.

LCMS (ESI) m/z: [M+H]⁺=367.

Step 2: Preparation of 2-[3-[4-(tert-butoxycarbonyl)piperidin-1-yl]-1,2-oxazol-5-yl]-3-methylbutanoic acid (Intermediate 3)

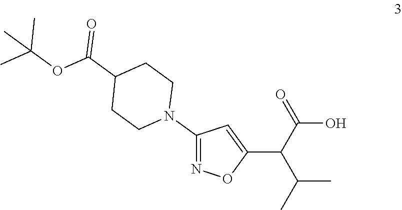 [SMILES: CC(C)C(C(=O)O)c1cc(N2CCC(C(=O)OC(C)(C)C)CC2)no1]

To a stirred solution of Intermediate 2 (78.00 mg, 0.199 mmol, 1.00 equiv) in MeOH (1.00 mL) and H₂O (1.00 mL) was added LiOH—H₂O (25.08 mg, 0.597 mmol, 3.00 equiv) at room temperature. The resulting mixture was stirred for 5 h at room temperature. The mixture was then acidified to pH 6 with 1 M HCl (aq.). The mixture was purified directly by reverse flash chromatography with the following conditions: column, C18 silica gel; mobile phase, MeCN in water (0.1% FA), 0 to 100% gradient in 30 min to give Intermediate 3 (63 mg, 89.74%) as a yellow oil. LCMS (ESI) m/z: [M+H]⁺=353.

Step 3: Preparation of tert-butyl 1-(5-[1-[(2S,4R)-4-hydroxy-2-[[(1S)-1-[4-(4-methyl-1,3-thiazol-5-yl)phenyl]ethyl]carbamoyl]pyrrolidin-1-yl]-3-methyl-1-oxobutan-2-yl]-1,2-oxazol-3-yl)piperidine-4-carboxylate (Intermediate 4)

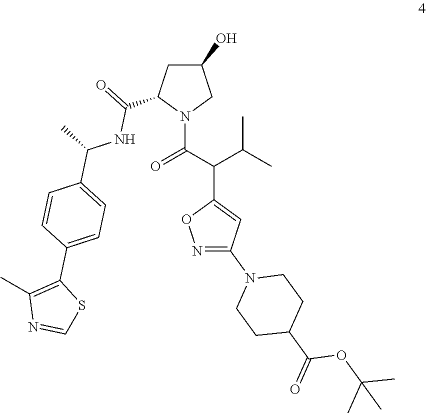 [SMILES: Cc1ncsc1-c1ccc([C@H](C)NC(=O)[C@@H]2C[C@@H](O)CN2C(=O)C(c2cc(N3CCC(C(=O)OC(C)(C)C)CC3)no2)C(C)C)cc1]

To a stirred solution of Intermediate 3 (63.00 mg, 0.179 mmol, 1.00 equiv) in DMF (2.00 mL) was added HATU (203.91 mg, 0.537 mmol, 3.00 equiv) and DIEA (115.52 mg, 0.895 mmol, 5.00 equiv) at room temperature. To the above mixture was added (2S,4R)-4-hydroxy-N-[(1S)-1-[4-(4-methyl-1,3-thiazol-5-yl)phenyl]ethyl]pyrrolidine-2-carboxamide hydrochloride (118.37 mg, 0.322 mmol, 1.80 equiv), and the resulting mixture stirred for an additional 1 h. The mixture was purified directly by reverse flash chromatography with the following conditions: column, C18 silica gel; mobile phase, MeCN in water (0.1% FA), 0 to 100% gradient in 30 min; to provide Intermediate 4 (93 mg, 78.13%) as a white solid. LCMS (ESI) m/z: [M+H]⁺=666.

Step 4: Preparation of tert-butyl 1-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperidine-4-carboxylate (Intermediate 5) and tert-butyl 1-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperidine-4-carboxylate (Intermediate 5′)

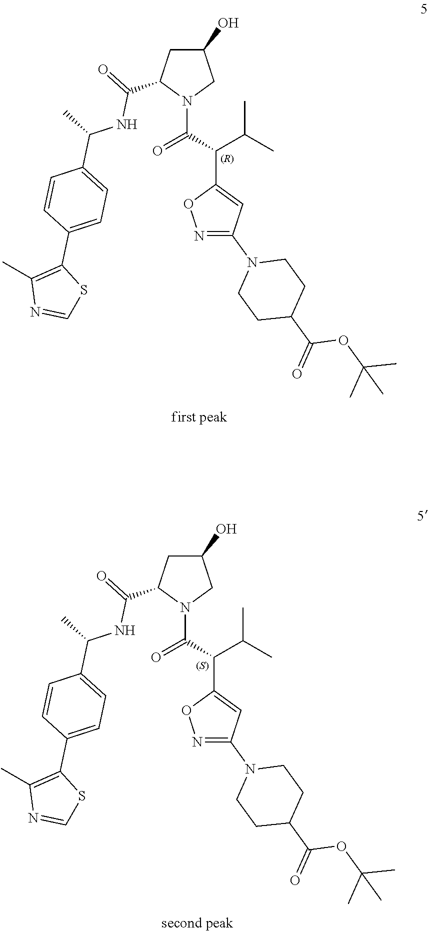

Intermediate 4 (93 mg) was purified by SFC with the following conditions: Column, NB_CHIRALPAK AD-H, 3*25 cm, 5 um; mobile phase, MeOH; Detector, UV 254/220 nm. This resulted in Intermediate 5 (peak1, 41 mg, 44.09%) as a white solid, LCMS (ESI) m/z: [M+H]⁺=666; and 5′ (peak 2, 35 mg, 37.63%) as a white solid, LCMS (ESI) m/z: [M+H]⁺=666.

Step 5: Preparation of 1-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperidine-4-carboxylic acid (I-6) and 1-{5-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperidine-4-carboxylic acid (I-7)

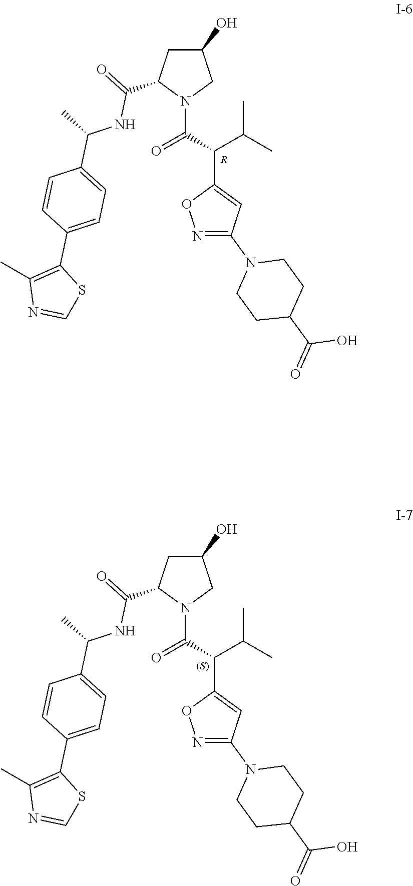

To a stirred solution of Intermediate 5 (41.00 mg, 0.048 mmol, 1.00 equiv) in DCM (2.00 mL) was added TFA (1.00 mL) at room temperature. The resulting mixture was stirred for 1 h at room temperature. The resulting mixture was concentrated under reduced pressure. This resulted in 1-6 (35 mg, crude) as a brown oil. LCMS (ESI) m/z: [M+H]⁺=610.

Intermediate I-7 (29 mg, crude) was obtained as a brown oil using the same method. LCMS (ESI) m/z: [M+H]⁺=610.

Preparation of (2S,4R)-4-hydroxy-1-((S)-2-(3-(4-(3-(3-(2-hydroxyphenyl)thieno[2,3-c]pyridazin-6-yl)azetidine-1-carbonyl)piperidin-1-yl)isoxazol-5-yl)-3-methylbutanoyl)-N—((S)-1-(4-(4-methylthiazol-5-yl)phenyl)ethyl)pyrrolidine-2-carboxamide

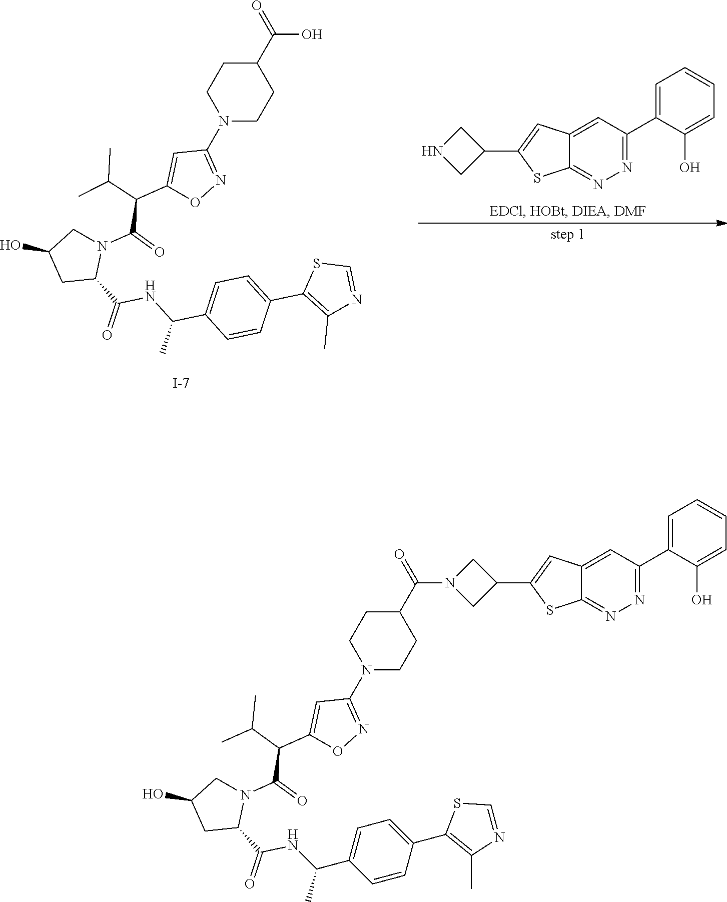

To a stirred solution of 1-(5-((S)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)piperidine-4-carboxylic acid (I-7, 10.0 mg, 0.016 mmol, 1.00 equiv) and 2-[6-(azetidin-3-yl)thieno[2,3-c]pyridazin-3-yl]phenol (4.7 mg, 0.016 mmol, 1.00 equiv) in DMF (1 mL) were added EDCI (6.3 mg, 0.032 mmol, 2.00 equiv), HOBT (4.4 mg, 0.032 mmol, 2.00 equiv) and DIEA (20.6 mg, 0.160 mmol, 10.00 equiv). The resulting mixture was stirred for 2 h. The crude mixture was purified directly by Prep-HPLC with the following conditions (Column: XBridge Prep OBD C18 Column, 30*150 mm, 5 μm; Mobile Phase A: Water with 10 mM NH₄HCO₃), Mobile Phase B: ACN; Flow rate: 25 mL/min; Gradient: 32% B to 60% B in 8 min, 60% B; to afford the title compound (1.3 mg, 8.89%) as an off-white solid. ¹H NMR (300 MHz, Methanol-d₄) δ 8.88 (s, 1H), 8.68 (s, 1H), 7.98 (dd, J=8.4, 1.7 Hz, 1H), 7.55-7.30 (m, 6H), 7.10-6.95 (m, 2H), 6.16 (s, 1H), 5.01 (q, J=6.9 Hz, 1H), 4.83-4.78 (m, 1H), 4.64-4.33 (m, 5H), 4.28-4.15 (m, 1H), 3.82-3.59 (m, 5H), 3.01-2.81 (m, 2H), 2.57 (s, 1H), 2.48 (s, 3H), 2.46-2.34 (m, 1H), 2.28-2.17 (m, 1H), 2.07-1.93 (m, 1H), 1.88-1.69 (m, 4H), 1.56 (dd, J=28.2, 7.0 Hz, 3H), 1.07 (d, J=6.6 Hz, 3H), 1.00-0.83 (m, 3H). LCMS (ESI) m/z: [M+H]⁺=875.55

Preparation of (2S,4R)-4-hydroxy-1-((R)-2-(3-(4-(3-(3-(2-hydroxyphenyl)thieno[2,3-c]pyridazin-6-yl)azetidine-1-carbonyl)piperidin-1-yl)isoxazol-5-yl)-3-methylbutanoyl)-N—((S)-1-(4-(4-methylthiazol-5-yl)phenyl)ethyl)pyrrolidine-2-carboxamide

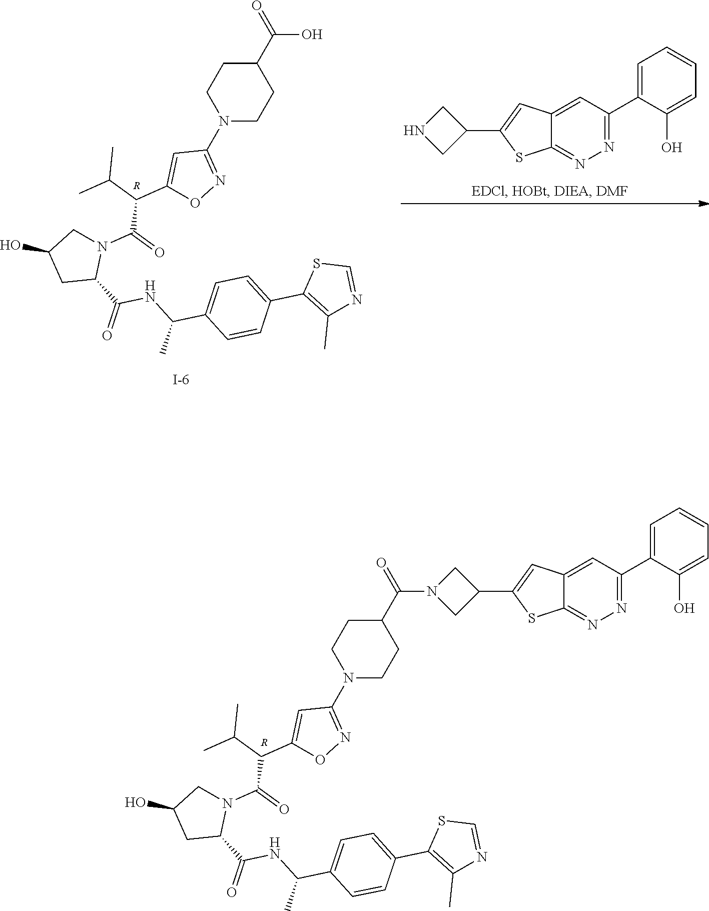

To a stirred solution of 1-(5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)piperidine-4-carboxylic acid (I-6, 15.0 mg, 0.025 mmol, 1.00 equiv) and 2-[6-(azetidin-3-yl)thieno[2,3-c]pyridazin-3-yl]phenol (10.5 mg, 0.037 mmol, 1.50 equiv) in DMF (1 mL) were added EDCI (9.4 mg, 0.050 mmol, 2.00 equiv), HOBT (6.7 mg, 0.050 mmol, 2.00 equiv) and DIEA (31.8 mg, 0.250 mmol, 10.00 equiv). The resulting mixture was stirred for 2 h. The crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 25 mL/min; Gradient: 32% B to 60% B in 8 min to afford the title compound (2.1 mg, 9.65%) as a white solid. ¹H NMR (300 MHz, Methanol-d4) δ 8.89 (s, 1H), 8.68 (s, 1H), 8.04-7.94 (m, 1H), 7.56-7.31 (m, 6H), 7.16-6.95 (m, 2H), 6.13 (s, 1H), 5.05 (q, J=6.8 Hz, 1H), 4.83-4.74 (m, 1H), 4.60-4.32 (m, 5H), 4.28-4.16 (m, 1H), 3.91-3.70 (m, 3H), 3.67-3.46 (m, 2H), 3.01-2.85 (m, 2H), 2.69-2.52 (m, 1H), 2.49 (s, 3H), 2.46-2.28 (m, 1H), 2.25-2.13 (m, 1H), 2.06-1.91 (m, 1H), 1.88-1.72 (m, 4H), 1.63-1.48 (m, 3H), 1.07 (d, J=6.5 Hz, 3H), 0.97-0.85 (m, 3H). LCMS (ESI) m/z: [M+H]⁺=875.50.

Preparation of (2S,4R)-4-hydroxy-1-[(2R)-2-{3-[4-(2-{3-[3-(2-hydroxyphenyl)thieno[2,3-c]pyridazin-6-yl]azetidin-1-yl}-2-oxoethyl)piperazin-1-yl]-1,2-oxazol-5-yl}-3-methylbutanoyl]-N-[(1S)-1-[4-(4-methyl-1,3-thiazol-5-yl)phenyl]ethyl]pyrrolidine-2-carboxamide

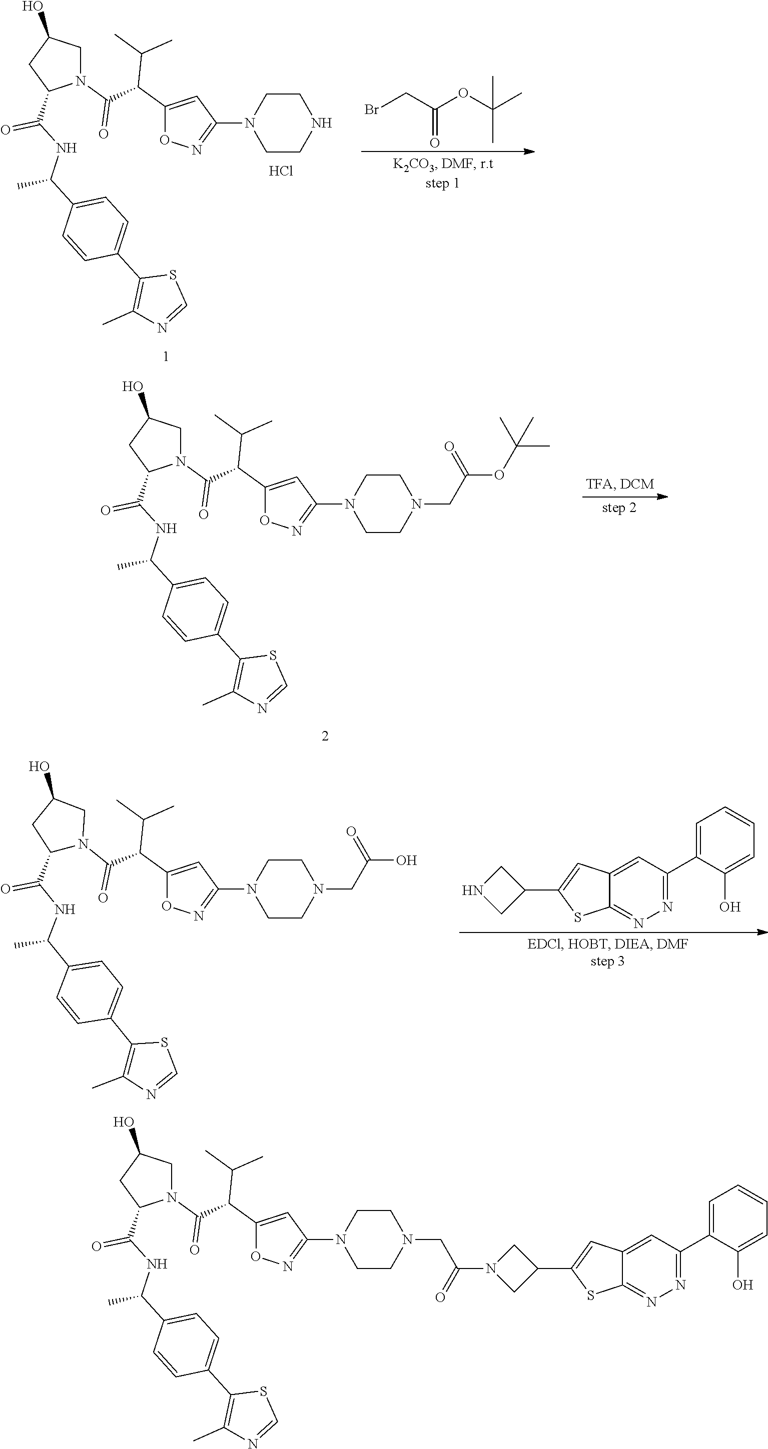

Step 1: Preparation of tert-butyl 2-(4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazin-1-yl)acetate (Intermediate 2)

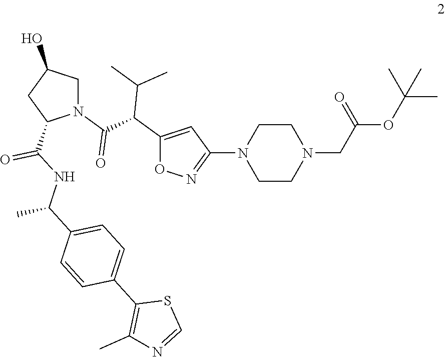 [SMILES: Cc1ncsc1-c1ccc([C@H](C)NC(=O)[C@@H]2C[C@@H](O)CN2C(=O)[C@@H](c2cc(N3CCN(CC(=O)OC(C)(C)C)CC3)no2)C(C)C)cc1]

A mixture of (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (80 mg, 0.141 mmol, 1.00 equiv), tert-buty 2-bromoacetate (1.0 equiv.), and K₂CO₃ (39.02 mg, 0.282 mmol, 2 equiv) in DMF (2 mL) was stirred for 3 h at room temperature. The mixture was purified by directly by reverse flash chromatography with the following conditions: column, C18 silica gel; mobile phase, ACN in water, 0% to 100% gradient in 20 min; to afford intermediate 2 (42 mg, 43.70%) as an off-white solid. LCMS (ESI) m/z [M+H]⁺=670.

Step 2: Preparation of (4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperazin-1-yl)acetic acid (I-8)

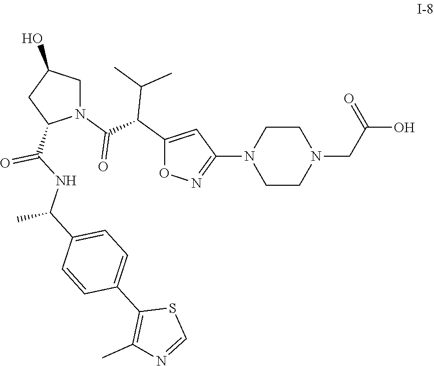 [SMILES: Cc1ncsc1-c1ccc([C@H](C)NC(=O)[C@@H]2C[C@@H](O)CN2C(=O)[C@@H](c2cc(N3CCN(CC(=O)O)CC3)no2)C(C)C)cc1]

A mixture of intermediate 2 (37 mg, 0.054 mmol, 1.00 equiv) and TFA (0.5 mL) in DCM (1.5 mL) was stirred for 2 h at room temperature. Without any additional work-up, the resulting mixture was concentrated under reduced pressure to afford 1-8 (33 mg, crude) as a brown oil. LCMS (ESI) m/z [M+H]⁺=625.

Step 3: Preparation of (2S,4R)-4-hydroxy-1-[(2R)-2-{3-[4-(2-{3-[3-(2-hydroxyphenyl)thieno[2,3-c]pyridazin-6-yl]azetidin-1-yl}-2-oxoethyl)piperazin-1-yl]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 6)

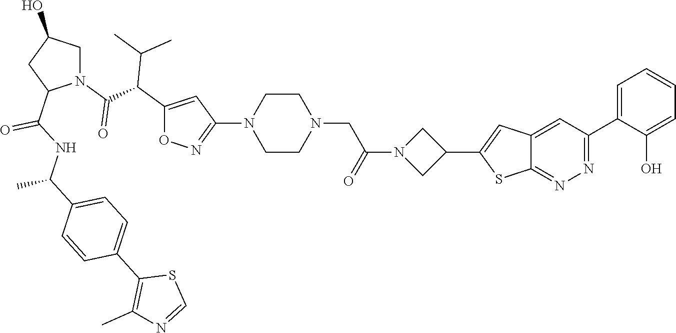 [SMILES: Cc1ncsc1-c1ccc([C@H](C)NC(=O)C2C[C@@H](O)CN2C(=O)[C@@H](c2cc(N3CCN(CC(=O)N4CC(c5cc6cc(-c7ccccc7O)nnc6s5)C4)CC3)no2)C(C)C)cc1]

A mixture of I-8 (27 mg, 0.043 mmol, 1.00 equiv), EDCI (16.57 mg, 0.086 mmol, 2 equiv), HOBT (11.68 mg, 0.086 mmol, 2 equiv) and DIEA (55.85 mg, 0.430 mmol, 10 equiv) in DMF (1 mL) was stirred for 5 min at room temperature. 2-[6-(azetidin-3-yl)thieno[2,3-c]pyridazin-3-yl]phenol (14.69 mg, 0.052 mmol, 1.2 equiv) was then added and the resulting mixture stirred for 2 h at room temperature. Without any additional work-up, the crude product was purified by Prep-HPLC with the following conditions (Column: XBridge Shield RP18 OBD Column, 30*150 mm, 5 μm; Mobile Phase A: Water (10 mmol/L NH₄HCO₃), Mobile Phase B: ACN; Flow rate: 25 mL/min; Gradient: 40% B to 48% B in 8 min; to afford the title compound (8.1 mg, 20.78%) as an off-white solid. ¹H NMR (300 MHz, DMSO-d6) δ 12.21 (s, 1H), 8.99 (s, 1H), 8.75 (s, 1H), 8.40 (d, J=7.7 Hz, 1H), 8.02 (dd, J=7.9, 1.7 Hz, 1H), 7.58 (s, 1H), 7.44 (d, J=8.3 Hz, 2H), 7.41-7.33 (m, 3H), 7.04-6.97 (m, 2H), 6.16 (s, 1H), 5.11 (s, 1H), 4.99-4.88 (m, 1H), 4.85-4.62 (m, 1H), 4.53-4.32 (m, 4H), 4.29 (s, 1H), 4.14-4.01 (m, 1H), 3.72 (dd, J=10.5, 4.4 Hz, 1H), 3.58 (d, J=9.9 Hz, 1H), 3.49-3.39 (m, 1H), 3.26-3.16 (m, 4H), 3.16-3.04 (m, 2H), 2.61-2.54 (m, 4H), 2.46 (d, J=1.6 Hz, 3H), 2.27-2.11 (m, 1H), 2.10-1.92 (m, 1H), 1.79-1.72 (m, 1H), 1.42 (dd, J=(22.2, 7.0 Hz, 3H), 0.96 (d, J=6.3 Hz, 3H), 0.81 (d, J=6.4 Hz, 3H). LCMS (ESI) m/z [M+H]⁺=890.10.

The compounds in Table 5 were prepared using procedures similar to those used above for the preparation of compound 6 using the appropriate amine and carboxylic acid.

TABLE 5

| | | LCMS | |
|---|---|---|---|
| | | (ESI) | |
| No. | Name | m/z | ¹H NMR |
| 64 | (2S,4R)-4-hydroxy-1-((R)-2-(3-(3- | 900.2 | 1H NMR (400 MHz, DMSO-d6) δ 12.75 (s, |
| | (4-(3-(2-hydroxyphenyl)-5- | | 1H), 9.01-8.96 (m, 1H), 8.71 (s, 1H), 8.41 |
| | methylthieno[2,3-c]pyridazin-6- | | (d, J = 7.7 Hz, 1H), 8.20-8.13 (m, 1H), |
| | yl)piperidine-1- | | 7.48-7.35 (m, 5H), 7.06-6.98 (m, 2H), |
| | carbonyl)bicyclo[1.1.1]pentan-1- | | 6.34 (s, 1H), 5.10 (d, J = 3.7 Hz, 1H), 4.92 |
| | yl)isoxazol-5-yl)-3-methylbutanoyl)- | | (p, J = 7.1 Hz, 1H), 4.52 (d, J = 13.0 Hz, |
| | N-((S)-1-(4-(4-methylthiazol-5- | | 1H), 4.40-4.27 (m, 3H), 3.78 (d, J = 9.7 |
| | yl)phenyl)ethyl)pyrrolidine-2- | | Hz, 1H), 3.75-3.67 (m, 1H), 3.65-3.58 |
| | carboxamide | | (m, 1H), 3.53-3.42 (m, 1H), 3.29-3.23 |
| | | | (m, 1H), 2.78 (t, J = 12.3 Hz, 1H), 2.48 (s, |
| | | | 3H), 2.47-2.39 (m, 9H), 2.29-2.17 (m, |
| | | | 1H), 2.05-2.00 (m, 3H), 1.84-1.73 (m, |
| | | | 1H), 1.65-1.49 (m, 2H), 1.42 (dd, J = 33.5, |
| | | | 7.0 Hz, 3H), 1.01-0.94 (m, 3H), 0.79 (dd, |
| | | | J = 12.1, 6.7 Hz, 3H). |
| 75 | (2S,4R)-1-((S)-2-(1- | 884.2 | 1H NMR (400 MHz, DMSO-d6) δ 12.92 (s, |
| | fluorocyclopropane-1- | | 1H), 9.16 (s, 1H), 8.98 (s, 1H), 8.61 (t, J = |
| | carboxamido)-3,3- | | 5.9 Hz, 1H), 8.03 (m, J = 8.0, 1.7 Hz, 1H), |
| | dimethylbutanoyl)-4-hydroxy-N-(2- | | 7.71 (s, 1H), 7.45-7.33 (m, 2H), 7.29 (d, J = |
| | (2-(4-(3-(2- | | 9.2 Hz, 1H), 7.07-6.94 (m, 4H), 5.19 (d, |
| | hydroxyphenyl)thieno[3,2- | | J = 3.5 Hz, 1H), 5.05 (m, J = 15.2, 5.9 Hz, |
| | c]pyridazin-6-yl)piperidin-1-yl)-2- | | 2H), 4.59 (d, J = 9.2 Hz, 1H), 4.51 (t, J = 8.3 |
| | oxoethoxy)-4-(4-methylthiazol-5- | | Hz, 2H), 4.33 (m, J = 16.3, 5.8 Hz, 3H), 4.05 |
| | yl)benzyl)pyrrolidine-2- | | (d, J = 13.4 Hz, 1H), 3.71-3.54 (m, 2H), |
| | carboxamide | | 3.49 (d, J = 10.4 Hz, 1H), 3.29-3.17 (m, |
| | | | 1H), 2.82 (t, J = 12.6 Hz, 1H), 2.45 (s, 3H), |
| | | | 2.12 (s, 3H), 1.92 (m, J = 13.1, 8.9, 4.5 Hz, |
| | | | 1H), 1.80 (d, J = 12.3 Hz, 1H), 1.61 (d, J = |
| | | | 12.4 Hz, 1H), 1.42-1.32 (m, 2H), 1.26- |
| | | | 1.17 (m, 3H), 0.95 (s, 9H). |
| 82 | (2S,4R)-1-((S)-2-(1- | 898.2 | 1H NMR (300 MHz, DMSO-d6) δ 12.77 (s, |
| | fluorocyclopropane-1- | | 1H), 9.00 (s, 1H), 8.71 (s, 1H), 8.61 (s, 1H), |
| | carboxamido)-3,3- | | 8.17 (d, J = 7.7 Hz, 1H), 7.48-7.26 (m, |
| | dimethylbutanoyl)-4-hydroxy-N-(2- | | 3H), 7.02 (dd, J = 14.0, 7.8 Hz, 4H), 5.22- |
| | (2-(4-(3-(2-hydroxyphenyl)-5- | | 4.92 (m, 3H), 4.55 (d, J = 19.6 Hz, 3H), 4.35 |
| | methylthieno[2,3-c]pyridazin-6- | | (s, 3H), 4.05 (s, 1H), 3.63 (d, J = 8.9 Hz, |
| | yl)piperidin-1-yl)-2-oxoethoxy)-4- | | 3H), 2.82 (s, 1H), 2.48 (s, 6H), 2.02 (d, J = |
| | (4-methylthiazol-5- | | 13.2 Hz, 3H), 1.80-1.61 (m, 1H), 1.60- |
| | yl)benzyl)pyrrolidine-2- | | 1.38 (m, 1H), 1.36 (d, J = 17.7 Hz, 2H), 1.23 |
| | carboxamide | | (s, 3H), 0.96 (s, 9H). |
| 93 | (2S,4R)-4-hydroxy-1-((R)-2-(3-(2- | 864.1 | 1H NMR (400 MHz, Methanol-d4) δ 8.87 (s, |
| | (4-(3-(2-hydroxyphenyl)-5- | | 1H), 8.54 (d, J = 6.9 Hz, 1H), 8.06 (dd, J = |
| | methylthieno[2,3-c]pyridazin-6- | | 8.3, 1.7 Hz, 1H), 7.50-7.29 (m, 5H), 7.09- |
| | yl)piperidin-1-yl)-2- | | 6.95 (m, 2H), 6.23-6.03 (m, 1H), 5.12 (d, |
| | oxoethoxy)isoxazol-5-yl)-3- | | J = 14.7 Hz, 1H), 5.07-4.96 (m, 2H), 4.77- |
| | methylbutanoyl)-N-((S)-1-(4-(4- | | 4.65 (m, 1H), 4.60-4.49 (m, 1H), 4.44 (s, |
| | methylthiazol-5- | | 1H), 4.01 (d, J = 13.7 Hz, 1H), 3.85 (dd, J = |
| | yl)phenyl)ethyl)pyrrolidine-2- | | 10.8, 4.1 Hz, 1H), 3.71 (d, J = 9.8 Hz, 1H), |
| | carboxamide | | 3.63 (d, J = 10.9 Hz, 2H), 3.38 (s, 1H), 2.90 |
| | | | (t, J = 12.8 Hz, 1H), 2.49 (d, J = 12.7 Hz, |
| | | | 3H), 2.46-2.33 (m, 3H), 2.18 (t, J = 11.5 |
| | | | Hz, 1H), 2.09 (s, 2H), 2.01-1.87 (m, 2H), |
| | | | 1.75 (s, 1H), 1.62-1.49 (m, 3H), 1.36 (dd, |
| | | | J = 7.0, 5.6 Hz, 1H), 1.07 (d, J = 6.6 Hz, 3H), |
| | | | 0.91 (d, J = 6.7 Hz, 3H). |
| 98 | (2S,4R)-4-hydroxy-1-((R)-2-(3-(4- | 917 | 1H NMR (400 MHz, DMSO-d6) δ 12.75 (s, |
| | (4-(3-(2-hydroxyphenyl)-5- | | 1H), 8.99 (d, J = 2.1 Hz, 1H), 8.71 (s, 1H), |
| | methylthieno[2,3-c]pyridazin-6- | | 8.41 (d, J = 7.7 Hz, 1H), 8.15 (t, J = 8.2 Hz, |
| | yl)piperidine-1-carbonyl)piperidin- | | 1H), 7.50-7.37 (m, 2H), 7.37 (d, J = 8.2 |
| | 1-yl)isoxazol-5-yl)-3- | | Hz, 3H), 7.06-6.98 (m, 2H), 6.15 (s, 1H), |
| | methylbutanoyl)-N-((S)-1-(4-(4- | | 5.11 (d, J = 3.8 Hz, 1H), 4.92 (t, J = 7.3 Hz, |
| | methylthiazol-5- | | 1H), 4.60 (d, J = 13.4 Hz, 1H), 4.38 (t, J = |
| | yl)phenyl)ethyl)pyrrolidine-2- | | 7.9 Hz, 1H), 4.29 (brs, 1H), 4.18 (d, J = 13.2 |
| | carboxamide | | Hz, 1H), 3.76-3.64 (m, 3H), 3.58 (d, J = |
| | | | 9.7 Hz, 2H), 3.48-3.41 (m, 1H), 3.26- |
| | | | 3.20 (m, 1H), 2.88 (d, J = 10.6 Hz, 3H), 2.74 |
| | | | (d, J = 12.9 Hz, 1H), 2.48 (s, 3H), 2.46 (s, |
| | | | 3H), 2.27-2.19 (m, 2H), 2.10-1.96 (m, |
| | | | 3H), 1.84-1.74 (m, 1H), 1.73-1.56 (m, |
| | | | 5H), 1.38 (d, J = 7.0 Hz, 3H), 0.97 (t, J = 6.7 |
| | | | Hz, 3H), 0.82 (dd, J = 15.7, 6.7 Hz, 3H). |
| 99 | (2S,4R)-4-hydroxy-1-((R)-2-(3-(4- | 875 | 1H NMR (300 MHz, DMSO-d6) δ 12.87 |
| | (3-(3-(2-hydroxyphenyl)thieno[3,2- | | (brs, 1H), 9.25 (s, 1H), 9.06 (s, 1H), 8.48 (d, |
| | c]pyridazin-6-yl)azetidine-1- | | J = 7.7 Hz, 1H), 8.14-8.06 (m, 1H), 8.04 |
| | carbonyl)piperidin-1-yl)isoxazol-5- | | (s, 1H), 7.58-7.40 (m, 5H), 7.10 (t, J = 8.4 |
| | yl)-3-methylbutanoyl)-N-((S)-1-(4- | | Hz, 2H), 6.23 (s, 1H), 5.18 (d, J = 3.7 Hz, |
| | (4-methylthiazol-5- | | 1H), 4.99 (t, J = 7.1 Hz, 1H), 4.86-4.76 (m, |
| | yl)phenyl)ethyl)pyrrolidine-2- | | 1H), 4.56-4.30 (m, 5H), 4.18-4.06 (m, |
| | carboxamide | | 1H), 3.77 (t, J = 11.2 Hz, 4H), 3.65 (d, J = |
| | | | 9.9 Hz, 1H), 2.90 (t, J = 12.1 Hz, 2H), 2.53 |
| | | | (s, 3H), 2.38-2.03 (m, 3H), 1.94-1.72 (m, |
| | | | 3H), 1.72-1.52(m, 2H), 1.49 (dd, J = 22.6, |
| | | | 7.0 Hz, 3H), 1.03 (d, J = 6.4 Hz, 3H), 0.88 |
| | | | (d, J = 6.7 Hz, 3H). |
| 106 | (2S,4R)-4-hydroxy-1-((R)-2-(3-(2- | 822.2 | 1H NMR (300 MHz, DMSO-d6) δ 9.19 (s, |
| | (3-(3-(2-hydroxyphenyl)thieno[3,2- | | 1H), 8.99 (s, 1H), 8.44 (d, J = 7.6 Hz, 1H), |
| | c]pyridazin-6-yl)azetidin-1-yl)-2- | | 8.03 (dd, J = 8.0, 1.7 Hz, 1H), 7.97 (s, 1H), |
| | oxoethoxy)isoxazol-5-yl)-3- | | 7.47-7.36 (m, 5H), 7.11-6.97 (m, 2H), |
| | methylbutanoyl)-N-((S)-1-(4-(4- | | 6.20 (s, 1H), 4.90 (q, J = 7.0 Hz, 1H), 4.83- |
| | methylthiazol-5- | | 4.65 (m, 3H), 4.51-4.37 (m, 4H), 4.29 (s, |
| | yl)phenyl)ethyl)pyrrolidine-2- | | 1H), 4.14 (s, 1H), 3.77-3.64 (m, 2H), 3.45 |
| | carboxamide | | (s, 2H), 2.46 (s, 3H), 2.24 (s, 1H), 2.07- |
| | | | 2.01 (m, 1H), 1.86-1.72 (m, 1H), 1.41 (dd, |
| | | | J = 21.3, 7.0 Hz, 3H), 0.97 (d, J = 6.4 Hz, |
| | | | 3H), 0.85-0.76 (m, 3H). |

Preparation of (2S,4R)-1-[(2R)-2-[3-(4-formylpiperidin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (I-9)

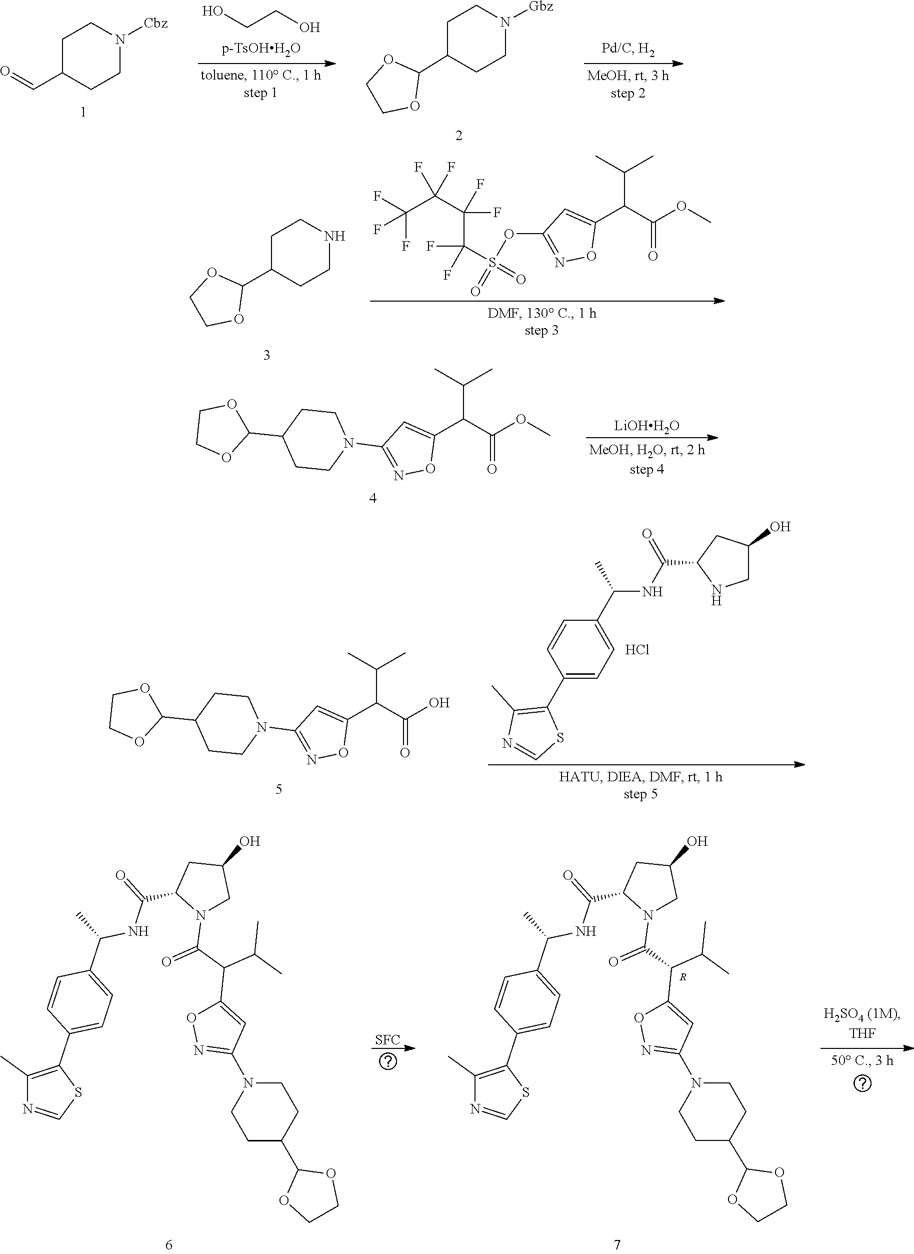

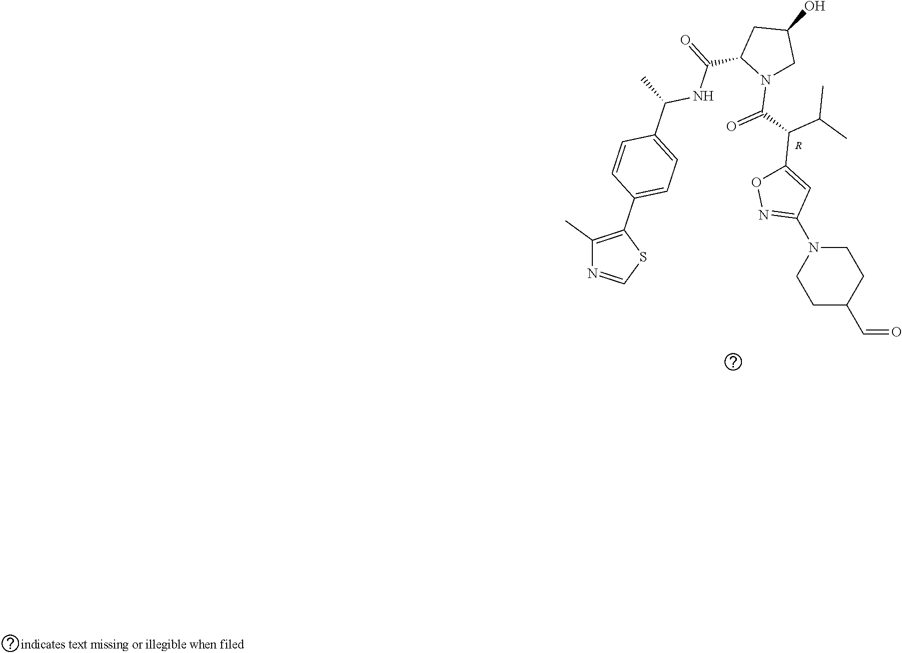

Step 1: Preparation of benzyl 4-(1,3-dioxolan-2-yl)piperidine-1-carboxylate (Intermediate 2)

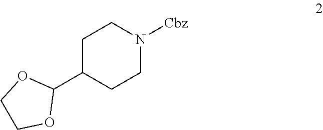 [SMILES: O=C(OCc1ccccc1)N1CCC(C2OCCO2)CC1]

To a stirred solution of benzyl 4-formylpiperidine-1-carboxylate (2.00 g, 8.088 mmol, 1.00 equiv) and ethylene glycol (2.00 mL) in toluene (40.00 mL) was added p-TsOH·H₂O (153.71 mg, 0.809 mmol, 0.10 equiv) at room temperature. The resulting mixture was stirred for 1 h at 110 degrees C. The mixture was allowed to cool down to room temperature and was concentrated under reduced pressure. The residue was purified by reverse flash chromatography with the following conditions: column, C18 silica gel; mobile phase, MeCN in water (0.1% FA), 0 to 100% gradient in 30 min. This provided Intermediate 2 (3.4 g, crude) as a brown-yellow oil. LCMS (ESI) m/z: [M+H]⁺=292.

Step 2: Preparation of 4-(1,3-dioxolan-2-yl)piperidine (Intermediate 3)

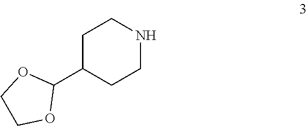 [SMILES: C1CC(C2OCCO2)CCN1]

To a stirred solution of Intermediate 2 (3.4 g, 11.670 mmol, 1.00 equiv) in MeOH (40 mL, 987.956 mmol, 84.66 equiv) was added 10% Pd/C (2.00 g, 18.793 mmol, 1.61 equiv) at room temperature. The resulting mixture was stirred for 3 h under 1 atm of hydrogen. The resulting mixture was filtered, and the filter cake was washed with MeOH. The filtrate was concentrated under reduced pressure to provide Intermediate 3 (1.7 g, 92.66%) as a grey oil. LCMS (ESI) m/z: [M+H]⁺=158.

Step 3: Preparation of methyl 2-{3-[4-(1,3-dioxolan-2-yl)piperidin-1-yl]-1,2-oxazol-5-yl}-3-methylbutanoate (Intermediate 4)

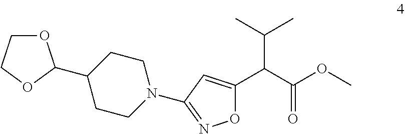 [SMILES: COC(=O)C(c1cc(N2CCC(C3OCCO3)CC2)no1)C(C)C]

To a stirred solution of methyl 3-methyl-2-{3-[(1,1,2,2,3,3,4,4,4-nonafluorobutanesulfonyl)oxy]-1,2-oxazol-5-yl}butanoate (1.50 g, 3.117 mmol, 1.00 equiv) in DMF (10.00 mL) was added Intermediate 3 (979.95 mg, 6.234 mmol, 2.00 equiv) at room temperature. The resulting mixture was stirred for 1 h at 130 degrees C. then allowed to cool to room temperature. The mixture was purified by reverse flash chromatography with the following conditions: column, C18 silica gel; mobile phase, MeCN in water (0.1% FA), 0 to 100% gradient in 30 min; to provide Intermediate 4 (298 mg, 28.26%) as a brown oil. LCMS (ESI) m/z: [M+H]⁺=339.

Step 4: Preparation of 2-{3-[4-(1,3-dioxolan-2-yl)piperidin-1-yl]-1,2-oxazol-5-yl}-3-methylbutanoic acid (Intermediate 5)

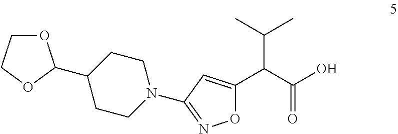 [SMILES: CC(C)C(C(=O)O)c1cc(N2CCC(C3OCCO3)CC2)no1]

To a stirred solution of Intermediate 4 (298 mg, 0.851 mmol, 1.00 equiv) in MeOH (5.00 mL) and H₂O (5.00 mL) was added LiOH—H₂O (107.14 mg, 2.553 mmol, 3.00 equiv) at room temperature. The resulting mixture was stirred for 2 h at room temperature. The mixture was then acidified to pH 6 with HCl aqueous (1 M) and extracted with EA (2×50 mL). The combined organic layers were washed with brine (50 mL), dried over anhydrous Na₂SO, and filtered. The filtrate was concentrated under reduced pressure to provide Intermediate 5 (376 mg, crude) as a yellow oil. LCMS (ESI) m/z: [M+H]⁺=325.

Step 5: Preparation of (2S,4R)-1-[2-{3-[4-(1,3-dioxolan-2-yl)piperidin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Intermediate 6)

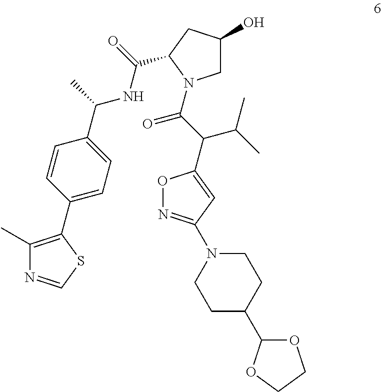 [SMILES: Cc1ncsc1-c1ccc([C@H](C)NC(=O)[C@@H]2C[C@@H](O)CN2C(=O)C(c2cc(N3CCC(C4OCCO4)CC3)no2)C(C)C)cc1]

To a stirred solution of Intermediate 5 (376 mg, 1.116 mmol, 1.00 equiv) in DMF (5.00 mL) were added HATU (1.30 g, 3.348 mmol, 3.00 equiv) and DIEA (721.17 mg, 5.580 mmol, 5.00 equiv) at room temperature. To the above mixture was added (2S,4R)-4-hydroxy-N-[(1S)-1-[4-(4-methyl-1,3-thiazol-5-yl)phenyl]ethyl]pyrrolidine-2-carboxamide hydrochloride (492.67 mg, 1.339 mmol, 1.20 equiv), and the resulting mixture was stirred for an additional 1 h. The mixture was purified directly by reverse flash chromatography with the following conditions: column, C18 silica gel; mobile phase, MeCN in water (0.1% FA), 0 to 100% gradient in 30 min; providing Intermediate 6 (330 mg, 46.36%) as a white solid. LCMS (ESI) m/z: [M+H]⁺=638.

Step 6: Preparation of (2S,4R)-1-[(2R)-2-{3-[4-(1,3-dioxolan-2-yl)piperidin-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (Intermediate 7)

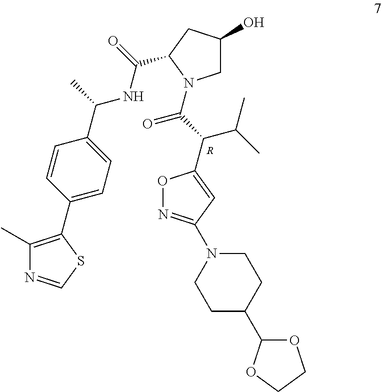

Intermediate 6 (330 mg) was purified by SFC with the following conditions: Column, CHIRAL ART Amylose-C NED, 3*25 cm, 5 um; mobile phase, MeOH; Detector, UV 254/220 nm. Thi provided Intermediate 7 (second peak eluted, 142 mg, 43.03%) as a white solid. LCMS (ESI) m/z: [M+H]⁺=638.

Step 7: Preparation of (2S,4R)-1-[(2R)-2-[3-(4-formylpiperidin-1-yl)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (I-9)

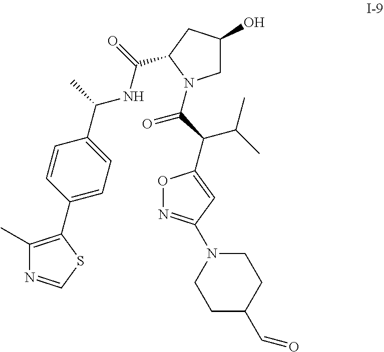 [SMILES: Cc1ncsc1-c1ccc([C@H](C)NC(=O)[C@@H]2C[C@@H](O)CN2C(=O)[C@H](c2cc(N3CCC(C=O)CC3)no2)C(C)C)cc1]

To a stirred solution of Intermediate 7 (137 mg, 0.215 mmol, 1.00 equiv) in THF (3 mL) was added H₂SO₄ (3.00 mL, 1.0 mol/L) at room temperature. The resulting mixture was stirred for 3 h at 50 degrees C., then cooled to room temperature. The mixture was basified to pH 8 with saturated NaHCO₃ (aq.) and extracted with EA (2×100 mL). The combined organic layers were washed with brine (100 mL), and dried over anhydrous Na₂SO₄. After filtration, the filtrate was concentrated under reduced pressure. This provided Intermediate I-9 (176 mg, crude) as a white solid. LCMS (ESI) m/z: [M+H]⁺=594.

The following intermediates in Table 6 were prepared in a similar manner as described in the preparation of intermediate I-9 starting with methyl 3-methyl-2-[3-[(1,1,2,2,3,3,4,4,4-nonafluorobutanesulfonyl)oxy]-1,2-oxazol-5-yl]butanoate and the appropriate amines.

TABLE 6

| | Intermediate | | LCMS |
|---|---|---|---|
| Structure | No. | Name | (ESI) m/z |
| 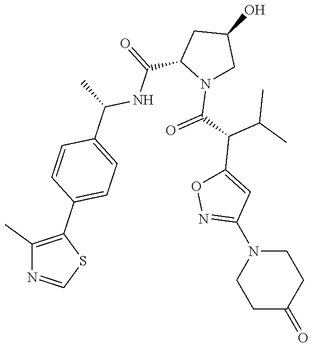 [SMILES: Cc1ncsc1-c1ccc([C@H](C)NC(=O)[C@@H]2C[C@@H](O)CN2C(=O)[C@@H](c2cc(N3CCC(=O)CC3)no2)C(C)C)cc1] | I-75 | (2S,4R)-4-hydroxy-1-((R)-3- methyl-2-(3-(4-oxopiperidin-1- yl)isoxazol-5-yl)butanoyl)-N- ((S)-1-(4-(4-methylthiazol-5- yl)pehnyl)ethyl)pyrrolidine-2- carboxamide | 580 |
| 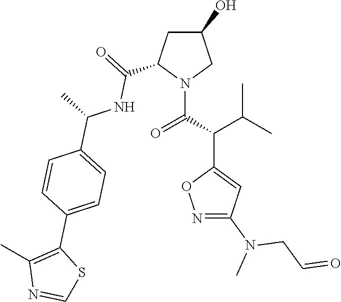 [SMILES: Cc1ncsc1-c1ccc([C@H](C)NC(=O)[C@@H]2C[C@@H](O)CN2C(=O)[C@@H](c2cc(N(C)CC=O)no2)C(C)C)cc1] | I-79 | (2S,4R)-4-hydroxy-1-((R)-3- methyl-2-(3-(methyl(2- oxoethyl)amino)isoxazol-5- yl)butanoyl)-N-((S)-1-(4-(4- methythiazol-5- yl)phenyl)ethyl)pyrrolidine-2- carboxamide | 554 |
| 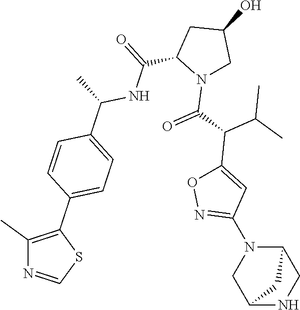 [SMILES: Cc1ncsc1-c1ccc([C@H](C)NC(=O)[C@@H]2C[C@@H](O)CN2C(=O)[C@@H](c2cc(N3C[C@H]4C[C@@H]3CN4)no2)C(C)C)cc1] | I-77 | (2S,4R)-1-[(2R)-2-{3-[(1R,4R)- 2,5-diazabicyclo[2.2.1]heptan- 2-yl]-1,2-oxazol-5-yl}-3- methylbutanoyl]-4-hydroxy-N- [(1S)-1-[4-(4-methyl-1,3- thiazol-5- yl)phenyl]ethyl]pyrrolidine-2- carboxamide | 579 |
| 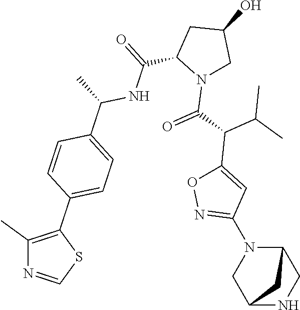 [SMILES: Cc1ncsc1-c1ccc([C@H](C)NC(=O)[C@@H]2C[C@@H](O)CN2C(=O)[C@@H](c2cc(N3C[C@@H]4C[C@H]3CN4)no2)C(C)C)cc1] | I-78 | (2S,4R)-1-((R)-2-(3-((1S,4S)- 2,5-diazabicyclo[2.2.1]heptan- 2 -yl)isoxazol-5-yl)-3- methylbutanoyl)-4-hydroxy-N- ((S)-1-(4-(4-methylthiazol-5- yl)phenyl)ethyl)pyrrolidine-2- carboxamide | 579 |

Preparation of (2S,4R)-4-hydroxy-1-((R)-2-(3-(4-((3-(3-(2-hydroxyphenyl)thieno[2,3-c]pyridazin-6-5-yl)phenyl)ethyl)pyrrolidine-2-carboxamide

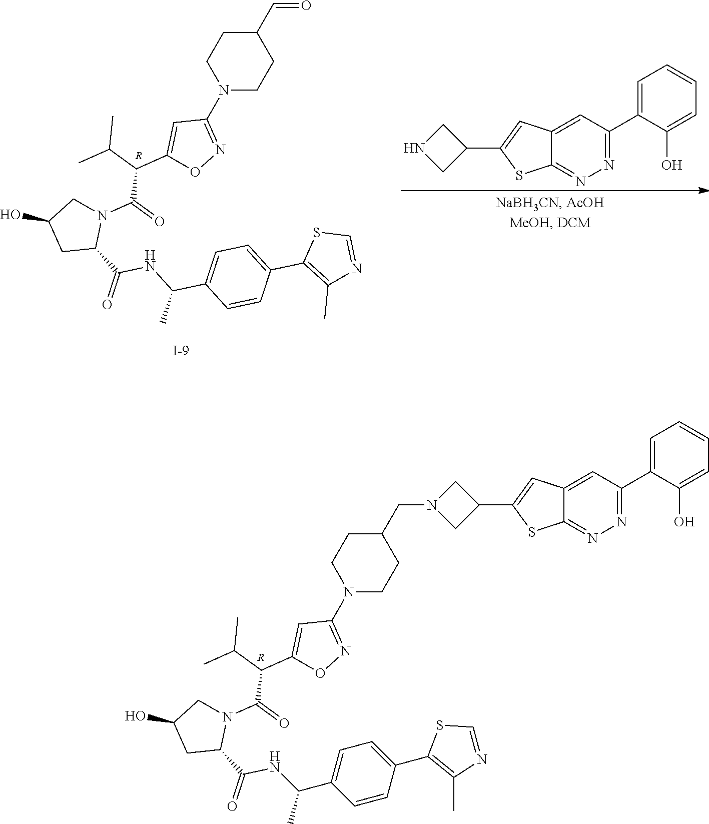

To a stirred solution of (2S,4R)-1-((R)-2-(3-(4-formylpiperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (I-9, 15.00 mg, 0.025 mmol, 1.00 equiv) in MeOH (0.50 mL) and DCM (0.50 mL) was added 2-[6-(azetidin-3-yl)thieno[2,3-c]pyridazin-3-yl]phenol (7.16 mg, 0.025 mmol, 1.00 equiv) at room temperature. The resulting mixture was stirred for 30 min followed by addition of NaBH3CN (7.94 mg, 0.125 mmol, 5.00 equiv) and AcOH (100.00 mg, 1.665 mmol, 65.91 equiv). The resulting mixture was stirred for additional 1 h at room temperature, then quenched with water at 0 degrees C. The resulting mixture was concentrated under reduced pressure, and the crude product was purified by Chiral-Prep-HPLC with the following conditions: Column, Kinetex EVO C18 Column, 21.2*150.5 um; mobile phase, Water (10 mmol/L NH₄HCO₃) and ACN (44% ACN up to 62% in 7 min); to provide the title compound (11.8 mg, 53.75%) as a white solid. ¹H NMR (400 MHz, DMSO-d⁶) δ 12.43 (s, 1H), 8.98 (s, 1H), 8.72 (s, 1H), 8.39 (d, J=7.7 Hz, 1H), 8.02 (dd, J=8.0, 1.6 Hz, 1H), 7.48-7.41 (m, 3H), 7.40-7.33 (m, 3H), 7.06-6.98 (m, 2H), 6.12 (s, 1H), 5.10 (d, J=3.7 Hz, 1H), 4.97-4.86 (m, 1H), 4.37 (t, J=7.8 Hz, 1H), 4.32-4.25 (m, 1H), 4.08-4.00 (m, 1H)), 3.76-3.52 (m, 6H), 3.48-3.37 (m, 1H), 3.30-3.24 (m, 3H), 2.80-2.69 (m, 2H), 2.46 (s, 3H), 2.41-2.35 (m, 2H), 2.28-2.12 (m, 1H), 2.08-1.96 (m, 1H), 1.84-1.72 (m, 3H), 1.56-1.43 (m, 1H), 1.38 (d, J=7.0 Hz, 3H), 1.27-1.12 (m, 2H), 0.96 (t, J=6.7 Hz, 3H), 0.81 (d, J=6.7 Hz, 3H). LCMS (ESI) m/z: [M+H]⁺=861.50.

Preparation of tert-butyl (2S,4R)-4-hydroxy-N-[(1S)-1-[4-(4-methyl-1,3-thiazol-5-yl)phenyl]ethyl]-1-(2R)-3-methyl-2-[3-(piperidin-4-yl)-1,2-oxazol-5-yl]butanoyl]pyrrolidine-2-carboxamide (I-80)

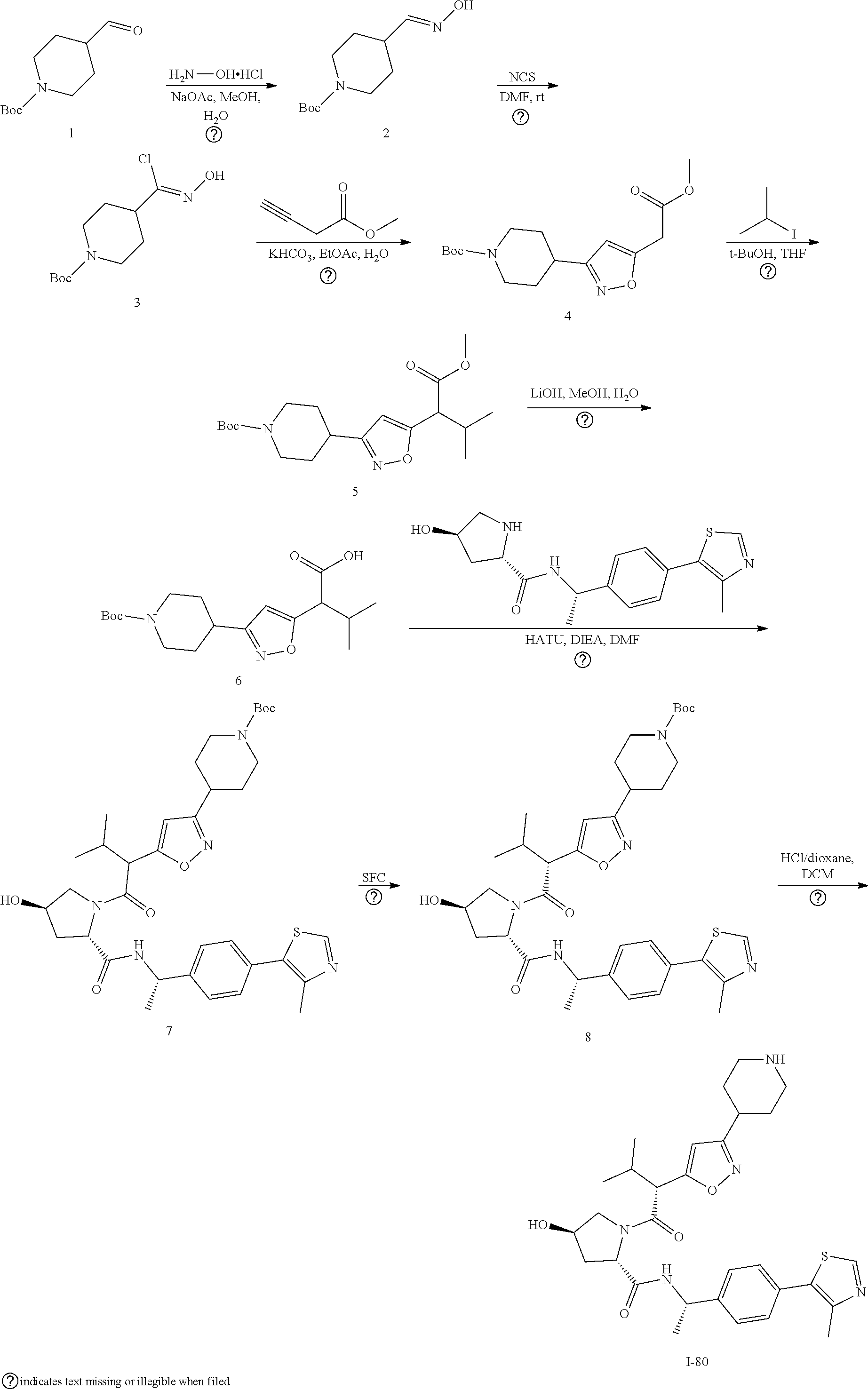

Step 1: Preparation of tert-butyl 4-[(1E)-(hydroxyimino)methyl]piperidine-1-carboxylate (Intermediate 2)

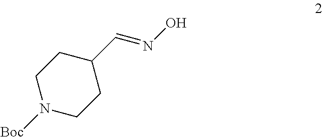 [SMILES: CC(C)(C)OC(=O)N1CCC(/C=N/O)CC1]

To a stirred solution of tert-butyl 4-formylpiperidine-1-carboxylate (5 g, 23.4 mmol, 1.00 equiv) in MeOH (10 mL) and H₂O (10 mL) was added hydroxylamine hydrochloride (1.95 g, 28.133 mmol, 1.2 equiv) and Na₂CO₃ (1.24 g, 11.722 mmol, 0.5 equiv) at 0 degrees C. The resulting mixture was stirred overnight at room temperature. The resulting mixture was concentrated under reduced pressure. The resulting mixture was extracted with EtOAc (3×20 mL). The combined organic layers were washed with brine (50 mL), and dried over anhydrous Na₂SO₄. After filtration, the filtrate was concentrated under reduced pressure to afford intermediate 2 (6 g, crude) as a colorless oil. LCMS (ESI) m/z: [M+H]⁺=229.

Step 2: Preparation of tert-butyl 4-[(1Z)-chloro(hydroxyimino)methyl]piperidine-1-carboxylate (Intermediate 3)

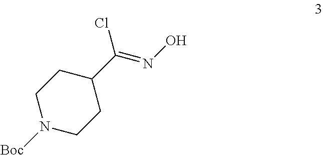 [SMILES: CC(C)(C)OC(=O)N1CCC(/C(Cl)=N/O)CC1]

A mixture of intermediate 2 and NCS (3.5 g, 26.282 mmol, 1.0 equiv) in DMF (20 mL) was stirred for 2 h at room temperature. The desired product could be detected by LCMS. The resulting mixture was diluted with water (50.00 mL) and extracted with EtOAc (3×50 mL). The combined organic layers were washed with brine (50 mL), and dried over anhydrous Na₂SO₄. After filtration, the filtrate was concentrated under reduced pressure to afford intermediate 3 (7.8 g, crude) as a colorless oil. LCMS (ESI) m/z [M+H]⁺=263.

Step 3: Preparation of tert-butyl 4-[5-(2-methoxy-2-oxoethyl)-1,2-oxazol-3-yl]piperidine-1-carboxylate (Intermediate 4)

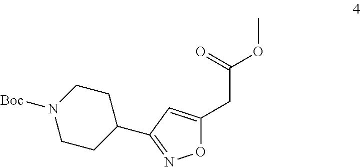 [SMILES: COC(=O)Cc1cc(C2CCN(C(=O)OC(C)(C)C)CC2)no1]

A mixture of intermediate 3 (7.8 g, crude) and NaHCO₃ (3.8 g, 45.675 mmol, 1.5 equiv) in EtOAc (100 mL) was stirred for 30 min at room temperature. To the above mixture was added methyl but-3-ynoate (2.99 g, 30.450 mmol, 1 equiv) at 0 degrees C. The resulting mixture was stirred overnight at room temperature. The desired product could be detected by LCMS. The resulting mixture was concentrated under reduced pressure. The residue was purified by reverse phase flash chromatography with the following conditions: column, C18 silica gel; mobile phase, MeCN in water (0.05% FA), 0% to 100% gradient in 30 min; detector, UV 254 nm. The resulting mixture was concentrated under reduced pressure to afford intermediate 4 (4.1 g, 41.51%) as a light yellow oil. LCMS (ESI) m/z: [M+H]⁺=325.

Step 4: Preparation of tert-butyl 4-[5-(1-methoxy-3-methyl-1-oxobutan-2-yl)-1,2-oxazol-3-yl]piperidine-1-carboxylate (Intermediate 5)

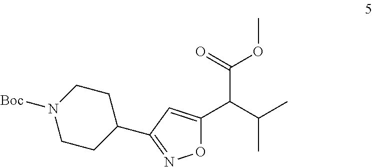 [SMILES: COC(=O)C(c1cc(C2CCN(C(=O)OC(C)(C)C)CC2)no1)C(C)C]

A mixture of intermediate 4 (1.0 g, 3.083 mmol, 1.5 equiv) and Na₂SO₄ (1.0 g) in THF (10 mL) was added t-BuOK (518.90 mg, 4.625 mmol, 1.5 equiv) and 2-iodopropane (628.87 mg, 3.700 mmol, 1.2 equiv) at 0 degrees C. under an atmosphere of dry nitrogen. The resulting mixture was stirred for 3 h at 0 degrees C. under an atmosphere of dry nitrogen. The desired product could be detected by LCMS. After filtration, the filtrate was concentrated under reduced pressure. The residue was purified by reverse phase flash chromatography with the following conditions: column, C18 silica gel; mobile phase, MeCN in water (0.05% FA), 0% to 100% gradient in 30 min; detector, UV 254 nm. The resulting mixture was concentrated under reduced pressure to afford intermediate 5 (330 mg, 29.21%) as a light yellow oil. LCMS (ESI) m/z: [M+H]⁺=367.

Step 5: Preparation of 2-{3-[1-(tert-butoxycarbonyl)piperidin-4-yl]-1,2-oxazol-5-yl}-3-methylbutanoic acid

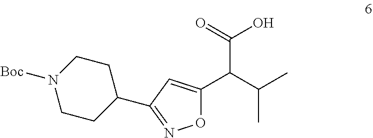 [SMILES: CC(C)C(C(=O)O)c1cc(C2CCN(C(=O)OC(C)(C)C)CC2)no1]

To a stirred solution of intermediate 5 (320 mg, 0.873 mmol, 1.00 equiv) in MeOH (5 mL) was added LiOH (62.74 mg, 2.619 mmol, 3 equiv) in H₂O (5 mL) dropwise at room temperature. The resulting mixture was stirred for 3 h at room temperature. The desired product could be detected by LCMS. The resulting mixture was concentrated under reduced pressure. To the above mixture was added aq. HCl (6M) adjusting pH to ˜5. The resulting mixture was extracted with EtOAc (3×50 mL). The combined organic layers were washed with brine (50 mL), and dried over anhydrous Na₂SO₄. After filtration, the filtrate was concentrated under reduced pressure to afford intermediate 6 (316 mg crude) as an off-white solid. LCMS (ESI) m/z: [M+H]⁺=353.

Step 6: Preparation of tert-butyl 4-(5-{1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl}-1,2-oxazol-3-yl)piperidine-1-carboxylate (Intermediate 7)

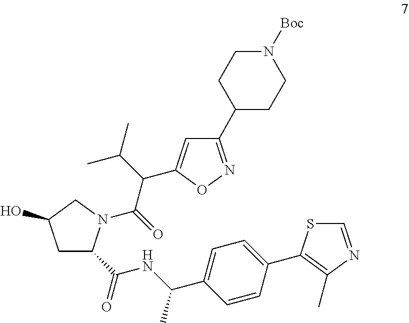 [SMILES: Cc1ncsc1-c1ccc([C@H](C)NC(=O)[C@@H]2C[C@@H](O)CN2C(=O)C(c2cc(C3CCN(C(=O)OC(C)(C)C)CC3)no2)C(C)C)cc1]

A mixture of intermediate 6 (310 mg, 0.880 mmol, 1.00 equiv) and HATU (668.90 mg, 1.760 mmol, 2 equiv) in DMF (5 mL) was stirred for 30 min at room temperature. To the above mixture was added (2S,4R)-4-hydroxy-N-[(1S)-1-[4-(4-methyl-1,3-thiazol-5-yl) phenyl]ethyl]pyrrolidine-2-carboxamide (291.53 mg, 0.880 mmol, 1 equiv) at room temperature. The resulting mixture was stirred for additional 2 h at room temperature. The desired product could be detected by LCMS. The residue was purified by reverse phase flash chromatography with the following conditions: column, C18 silica gel; mobile phase, MeCN in water (0.05% FA), 0% to 100% gradient in 30 min; detector, UV 254 nm. The resulting mixture was concentrated under reduced pressure to afford intermediate 7 (242 mg, 37.31%) as a light brown solid. LCMS (ESI) m/z: [M+H]⁺=666.

Step 7: Preparation of tert-butyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperidine-1-carboxylate (Intermediate 8)

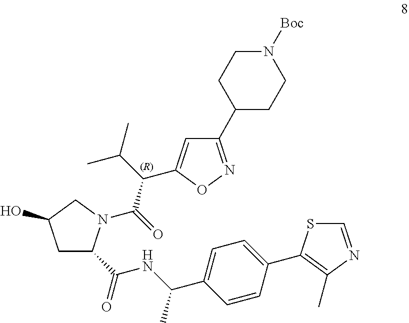

Intermediate 7 was purified by Prep-SFC with the following conditions (Column: CHIRAL ART Amylose-SA, 3*25 cm, 5 μm; Mobile Phase A: CO2, Mobile Phase B: MeOH—HPLC; Flow rate: 50 mL/min; Gradient: isocratic 45% B; Column Temperature(° C.): 35; Back Pressure(bar): 100; Wave Length: 205 nm; RT1(min): 3.65; RT2(min): 4.88; Sample Solvent: MeOH—HPLC; Injection Volume: 1 mL) to afford intermediate 8 (the second peak) (208.1 mg, 43.52%) as a light brown solid. LCMS (ESI) m/z: [M+H]⁺=666.

Step 8: Preparation of tert-butyl (2S,4R)-4-hydroxy-N-[(1S)-1-[4-(4-methyl-1,3-thiazol-5-yl)phenyl]ethyl]-1-[(2R)-3-methyl-2-[3-(piperidin-4-yl)-1,2-oxazol-5-yl]butanoyl]pyrrolidine-2-carboxamide (I-80)

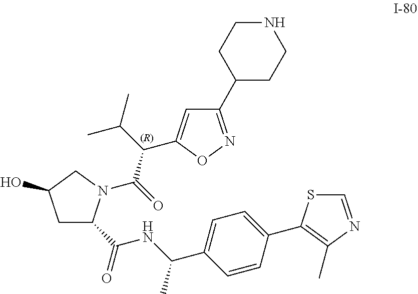

To a stirred solution of intermediate 8 (200 mg, 0.300 mmol, 1.00 equiv) in DCM (2 mL) was added 1 M HCl in 1,4-dioxane (2 mL) dropwise at room temperature. The resulting mixture was stirred for 1 h at room temperature. The desired product could be detected by LCMS. The resulting mixture was concentrated under reduced pressure to afford 1-80 (247.5 mg) as a light yellow solid. LCMS (ESI) m/z: [M+H]⁺=566.

Preparation of 2-((5-((R)-1-((2S,4R)-4-hydroxy-2-(((S)-1-(4-(2-methylthiazol-5-yl)phenyl)ethyl)carbamoyl)pyrrolidin-1-yl)-3-methyl-1-oxobutan-2-yl)isoxazol-3-yl)oxy)acetic acid (I-67)

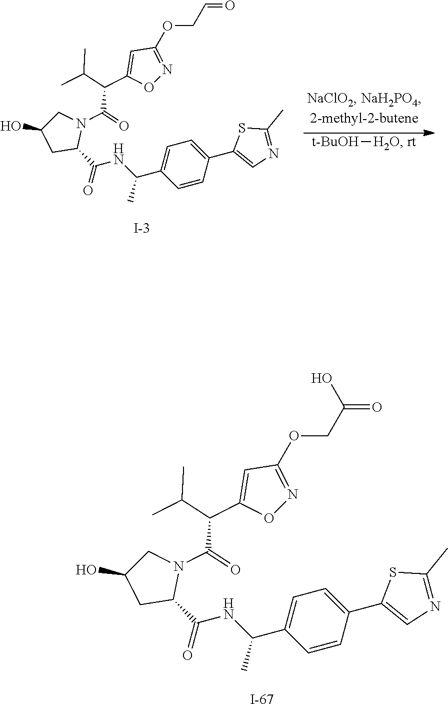

To a stirred solution of (2S,4R)-4-hydroxy-N-[(1S)-1-[4-(2-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (30.00 mg, 0.055 mmol, 1.00 equiv) and 2-methyl-2-butene (0.78 mg, 0.011 mmol, 0.20 equiv) in tert-butanol (2 mL) was added dropwise a solution of NaClO₂ (50.19 mg, 0.550 mmol, 10.00 equiv) and NaH₂PO₄ (78.77 mg, 0.550 mmol, 10.00 equiv) in water (2.00 mL) at 0° C. The mixture was stirred at 0° C. for 0.5 h, then warmed up to room temperature and stirred for 1.5 h. The reaction was quenched by addition of a mixture of saturated Na₂S₂O₃ solution and brine, extracted with CHCl₃ (20 mL×3). The combined organic extracts were dried over Na₂SO₄, filtered, concentrated in vacuo and purified by silica gel chromatography (PE/EtOAc=1:1 to 1:3). This provided intermediate I-67 (15.80 mg, 49.93%) as a colorless oil. LCMS (ESI) m/z: [M+H]⁺=557.

Preparation of (2S,4R)-4-hydroxy-1-[(2R)-2-[3-(2-{4-[3-(2-hydroxyphenyl)-5-methylthieno[2,3-c]pyridazin-6-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide

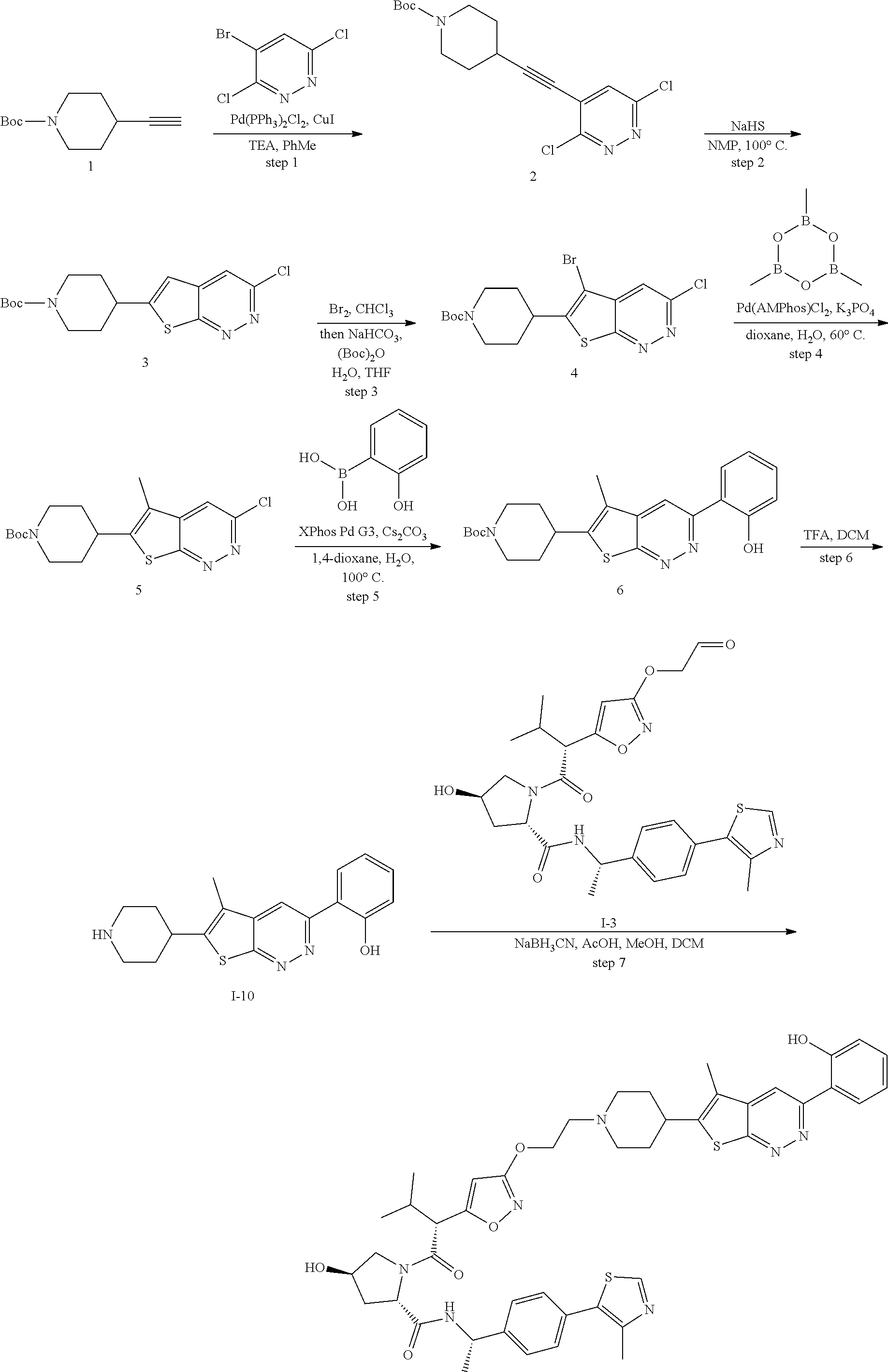

Step 1: Preparation of tert-butyl 4-((3,6-dichloropyridazin-4-yl)ethynyl)piperidine-1-carboxylate (intermediate 2)

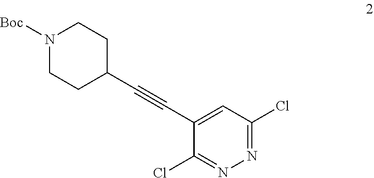 [SMILES: CC(C)(C)OC(=O)N1CCC(C#Cc2cc(Cl)nnc2Cl)CC1]

To a mixture of tert-butyl 4-ethynylpiperidine-1-carboxylate (8.00 g, 38.225 mmol, 1.00 equiv) and 4-bromo-3,6-dichloropyridazine (10.45 g, 45.870 mmol, 1.20 equiv) in toluene (80 mL) was added Pd(PPh₃)₂Cl₂ (4.02 g, 5.734 mmol, 0.15 equiv), CuI (14.56 g, 76.450 mmol, 2.00 equiv) and TEA (11.60 g, 114.675 mmol, 3.00 equiv) under a nitrogen atmosphere. The resulting mixture was stirred overnight at room temperature and concentrated under reduced pressure. The residue was purified by silica gel column chromatography, eluted with EtOAc in petroleum ether from 0% to 50% to afford intermediate 2 (5.00 g, 36.7%) as a yellow solid. LCMS (ESI) m/z: [M+H]+=356.

Step 2: Preparation of tert-butyl 4-(3-chlorothieno[2,3-c]pyridazin-6-yl)piperidine-1-carboxylate (intermediate 3)

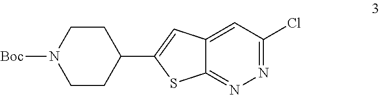 [SMILES: CC(C)(C)OC(=O)N1CCC(c2cc3cc(Cl)nnc3s2)CC1]

To a mixture of intermediate 2 (5.00 g, 14.035 mmol, 1.00 equiv) in NMP (50 mL) was added NaSH (0.79 g, 14.035 mmol, 1.0 equiv). The resulting mixture was stirred for an hour at 100 degrees C., colled, and filtered. The filtrate was purified by reverse flash chromatography with the following conditions: column, C18; mobile phase, MeOH in water, 10% to 50% gradient in 10 min; to afford intermediate 3 (1.80 g, 36.2%) as a yellow solid. LCMS (ESI) m/z: [M+H]+=354.

Step 3: Preparation of tert-butyl 4-(5-bromo-3-chlorothieno[2,3-c]pyridazin-6-yl)piperidine-1-carboxylate (intermediate 4)

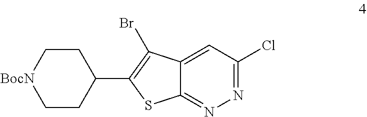 [SMILES: CC(C)(C)OC(=O)N1CCC(c2sc3nnc(Cl)cc3c2Br)CC1]

To a mixture of intermediate 3 (1.80 g, 5.087 mmol, 1.00 equiv) in CHCl3 (20 mL) was added Br2 (8.13 g, 50.870 mmol, 10.00 equiv). The resulting mixture was stirred overnight at room temperature, then basified with aqueous NaHCO₃. Boc2O (2.21 g, 10.174 mmol, 2.00 equiv) was then added and the mixture was stirred for 2 h. The mixture was extracted with EtOAc (3×100 mL) and the combined organic layers were washed with brine (50 mL), dried over anhydrous sodium sulfate, filtered, and concentrated under vacuum. The residue was purified by reverse phase flash chromatography with the following conditions: column, C18; mobile phase, MeOH in water, 10% to 50% gradient in 10 min; to afford intermediate 4 (710.0 mg, 32.4%) as a yellow solid. LCMS (ESI) m/z: [M+H]+=432.

Step 4: Preparation of tert-butyl 4-(3-chloro-5-methylthieno[2,3-c]pyridazin-6-yl)piperidine-1-carboxylate (intermediate 5)

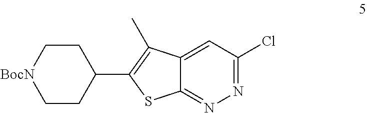 [SMILES: Cc1c(C2CCN(C(=O)OC(C)(C)C)CC2)sc2nnc(Cl)cc12]

To a mixture of intermediate 4 (710.0 mg, 1.641 mmol, 1.00 equiv), K3PO4 (696.50 mg, 3.282 mmol, 2.00 equiv) and Pd(AMPhos)Cl2 (174.25 mg, 0.246 mmol, 0.15 equiv) in dioxane (10 mL) and H₂O (2 mL) was added trimethyl-1,3,5,2,4,6-trioxatriborinane (411.90 mg, 3.282 mmol, 2.00 equiv), and the resulting mixture was stirred for an hour at 60 degrees C. under a nitrogen atmosphere. The reaction mixture was filtered through a short pad of Celite and eluted with EtOAc. The filtrate was concentrated under vacuum, and the residue purified by reverse phase flash chromatography with the following conditions: column, C18; mobile phase, MeOH in water, 10% to 50% gradient in 10 min; to afford intermediate 5 (450.0 mg, 74.5%) as a yellow solid. LCMS (ESI) m/z: [M+H]+=368.

Step 5: Preparation of tert-butyl 4-(3-(2-hydroxyphenyl)-5-methylthieno[2,3-c]pyridazin-6-yl)piperidine-1-carboxylate (intermediate 6)

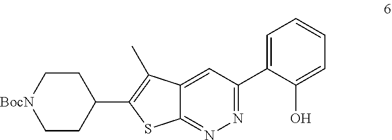 [SMILES: Cc1c(C2CCN(C(=O)OC(C)(C)C)CC2)sc2nnc(-c3ccccc3O)cc12]

To a mixture of intermediate 5 (450.0 mg, 1.223 mmol, 1.00 equiv) and 2-hydroxyphenylboronic acid (337.43 mg, 2.446 mmol, 2.00 equiv) in dioxane (10 mL) and H2O (2 mL) were added XPhos Pd G3 (155.31 mg, 0.183 mmol, 0.15 equiv) and Cs2CO3 (1.2 g, 3.669 mmol, 3.00 equiv), and the resulting mixture was stirred for an hour at 100 degrees C. under a nitrogen atmosphere. The reaction mixture was filtered through a short pad of Celite and eluted with EtOAc. The filtrate was concentrated under vacuum, and the residue was purified by reverse flash chromatography with the following conditions: column, C18; mobile phase, MeOH in water, 10% to 50% gradient in 10 min; to afford intermediate 6 (380.0 mg, 73.0%) as a yellow solid. LCMS (ESI) m/z: [M+H]+=426.

Step 6: Preparation of 2-(5-methyl-6-(piperidin-4-yl)thieno[2,3-c]pyridazin-3-yl)phenol (I-10)

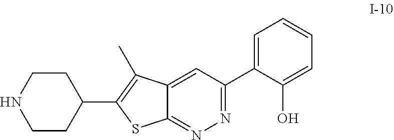 [SMILES: Cc1c(C2CCNCC2)sc2nnc(-c3ccccc3O)cc12]

To a mixture of intermediate 6 (380.0 mg, 0.893 mmol, 1.00 equiv) in DCM (6 mL) was added TFA (3 mL, 40.389 mmol, 45.23 equiv), and the resulting mixture was stirred for 2 h at room temperature. The reaction mixture was concentrated under reduced pressure, and the residue purified by reverse flash chromatography with the following conditions: column, C18; mobile phase, MeOH in water, 10% to 50% gradient in 10 min; detector to afford 1-10 (246.4 mg, 84.7%) as a yellow solid. 1H NMR (400 MHz, DMSO-d6) δ 8.69 (s, 1H), 8.22-8.11 (m, 1H), 7.43-7.34 (m, 1H), 7.07-6.99 (m, 2H), 3.67-3.53 (m, 1H), 3.43-3.35 (m, 2H), 3.10-3.00 (m, 2H), 2.46 (s, 3H), 2.13-2.04 (m, 2H), 1.91-1.78 (m, 2H). LCMS (ESI) m/z: [M+H]+=326.10.

Step 7: Preparation of (2S,4R)-4-hydroxy-1-[(2R)-2-[3-(2-{4-[3-(2-hydroxyphenyl)-5-methylthieno[2,3-c]pyridazin-6-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide

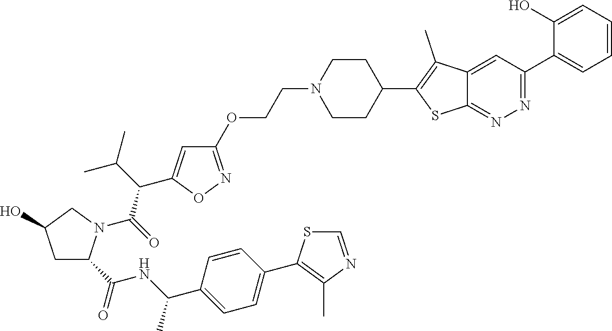 [SMILES: Cc1ncsc1-c1ccc([C@H](C)NC(=O)[C@@H]2C[C@@H](O)CN2C(=O)[C@@H](c2cc(OCCN3CCC(c4sc5nnc(-c6ccccc6O)cc5c4C)CC3)no2)C(C)C)cc1]

To a stirred mixture of (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (I-3, 15.00 mg, 0.028 mmol, 1.00 equiv) and compound I-10 (10.83 mg, 0.034 mmol, 1.2 equiv) in DCM (1.00 mL) and MeOH (1.00 mL) were added NaBH3CN (5.23 mg, 0.084 mmol, 3.0 equiv) and AcOH (0.02 mL, 0.349 mmol, 12.58 equiv) at room temperature. The resulting mixture was stirred for 12 h at room temperature, then concentrated under reduced pressure. The residue was purified by Prep-HPLC with the following conditions (Column: XBridge Prep Phenyl OBD Column, 19*150 mm, 5 μm 13 nm; Mobile Phase A: Water (0.05% NH3H₂O), Mobile Phase B: ACN; Flow rate: 25 mL/min; Gradient: 50% B to 63% B in 7 ml; To afford compound the title compound (4.4 mg, 18.53%) as a white solid. 1H NMR (400 MHz, DMSO-d6) δ 12.71 (d, J=65.8 Hz, 1H), 8.98 (s, 1H), 8.70 (s, 1H), 8.40 (d, J=7.8 Hz, 1H), 8.16 (d, J=7.9 Hz, 1H), 7.51-7.31 (m, 5H), 7.03 (d, J=7.9 Hz, 2H), 6.15 (s, 1H), 5.11 (d, J=3.5 Hz, 1H), 4.92 (t, J=7.3 Hz, 1H), 4.60-4.50 (m, 1H), 4.42-4.23 (m, 3H), 3.80-3.55 (m, 4H), 3.48 (t, J=12.2 Hz, 2H), 3.12-2.99 (m, 1H), 2.83-2.73 (m, 1H), 2.46 (s, 6H), 2.31-2.16 (m, 3H), 2.10-1.85 (m, 4H), 1.83-1.64 (m, 2H), 1.43 (d, J=7.0 Hz, 3H), 0.97 (d, J=6.5 Hz, 3H), 0.84 (d, J=6.6 Hz, 3H). LCMS (ESI) m/z: [M+H]+=850.40.

The compounds in Table 7 were prepared using procedures similar to those used above for the preparation of compound 8 using the appropriate amine and aldehyde (or ketone).

TABLE 7

| | | LCMS | |
|---|---|---|---|
| Compound | | (ESI) | |
| No. | Name | m/z | ¹H NMR |
| 54 | (2S,4R)-4-hydroxy-1-((R)-2-(3-(2- | 866.5 | 1H NMR (400 MHz, Methanol-d4) δ 8.86 |
| | (4-hydroxy-4-(3-(2- | | (d, J = 8.8 Hz, 1H), 8.53 (d, J = 5.2 Hz, |
| | hydroxyphenyl)-5- | | 1H), 8.06 (dd, J = 8.3, 1.6 Hz, 1H), 7.48- |
| | methylthieno[2,3-c]pyridazin-6- | | 7.31 (m, 5H), 7.02 (dd, J = 8.1, 5.7 Hz, |
| | yl)piperidin-1-yl)ethoxy)isoxazol-5- | | 2H), 6.05 (d, J = 1.0 Hz, 1H), 5.05-5.00 |
| | yl)-3-methylbutanoyl)-N-((S)-1-(4- | | (m, 1H), 4.59 (s, 1H), 4.52 (t, J = 8.2 Hz, |
| | (4-methylthiazol-5- | | 1H), 4.46-4.42 (m, 1H), 4.41-4.37 (m, |
| | yl)phenyl)ethyl)pyrrolidine-2- | | 3H), 3.84 (dd, J = 10.9, 4.2 Hz, 1H), 3.69 |
| | carboxamide | | (d, J = 9.8 Hz, 1H), 3.62 (d, J = 10.3 Hz, |
| | | | 1H), 3.50-3.42 (m, 1H), 3.37-3.32 (m, |
| | | | 4H), 2.97 (d, J = 7.8 Hz, 2H), 2.90 (t, J = |
| | | | 5.4 Hz, 2H), 2.70 (d, J = 12.1 Hz, 2H), |
| | | | 2.64 (d, J = 3.7 Hz, 3H), 2.47 (d, J = 5.9 |
| | | | Hz, 3H), 2.39 (td, J = 14.6, 13.4, 9.6 Hz, |
| | | | 3H), 2.23-2.13 (m, 1H), 1.96 (t, J = 11.7 |
| | | | Hz, 3H), 1.60 (d, J = 7.1 Hz, 1H), 1.52 (d, |
| | | | J = 7.0 Hz, 3H), 1.29 (s, 1H), 1.06 (d, J = |
| | | | 6.5 Hz, 3H), 0.91 (dd, J = 9.5, 6.6 Hz, |
| | | | 3H). |
| 58 | (2S,4R)-4-hydroxy-1-[(2R)-2-(3-{2- | 862.4 | 1H NMR (400 MHz, Methanol-d4) δ 8.83 |
| | [(1R,4S,5S)-5-[3-(2- | | (d, J = 29.6 Hz, 1H), 8.48 (d, J = 17.9 Hz, |
| | hydroxyphenyl)-5- | | 1H), 8.03 (td, J = 8.3, 1.7 Hz, 1H), 7.53- |
| | methylthieno[2,3-c]pyridazin-6-yl]- | | 7.28 (m, 5H), 7.06-6.94 (m, 2H), 6.01 |
| | 2-azabicyclo[2.2.1]heptan-2- | | (d, J = 27.1 Hz, 1H), 5.10-4.94 (m, 1H), |
| | yl]ethoxy}-1,2-oxazol-5-yl)-3- | | 4.59 (s, 1H), 4.52 (t, J = 8.2 Hz, 1H), 4.47- |
| | methylbutanoyl]-N-[(1S)-1-[4-(4- | | 4.28 (m, 3H), 3.89-3.72 (m, 1H), 3.66 |
| | methyl-1,3-thiazol-5- | | (dd, J = 27.0, 10.4 Hz, 2H), 3.56 (d, J = |
| | yl)phenyl]ethyl]pyrrolidine-2- | | 7.5 Hz, 1H), 3.51-3.40 (m, 1H), 3.15- |
| | carboxamide | | 2.93 (m, 3H), 2.74-2.55 (m, 3H), 2.48 |
| | | | (s, 2H), 2.46 (s, 2H), 2.44-2.34 (m, 2H), |
| | | | 2.17 (q, J = 10.2, 8.5 Hz, 1H), 1.96 (ddd, |
| | | | J = 13.0, 8.6, 4.5 Hz, 1H), 1.89 (d, J = |
| | | | 8.6 Hz, 1H), 1.72 (ddd, J = 13.4, 5.7, 2.8 |
| | | | Hz, 1H), 1.56 (dd, J = 30.7, 7.0 Hz, 3H), |
| | | | 1.39-1.23 (m, 1H), 1.06 (d, J = 6.6 Hz, |
| | | | 3H), 0.91 (dd, J = 9.5, 6.8 Hz, 3H). |
| 60 | (2S,4R)-4-hydroxy-1-((R)-2-(3-(2- | 862.5 | 1H NMR (400 MHz, Methanol-d4) δ 8.93- |
| | ((1R,4S,5R)-5-(3-(2- | | 8.78 (m, 1H), 8.51 (s, 1H), 8.05 (dd, J = |
| | hydroxyphenyl)-5- | | 8.3, 1.7 Hz, 1H), 7.52-7.29 (m, 5H), |
| | methylthieno[2,3-c]pyridazin-6-yl)- | | 7.08-6.95 (m, 2H), 6.01 (d, J = 23.4 Hz, |
| | 2-azabicyclo[2.2.1]heptan-2- | | 1H), 5.03 (q, J = 6.9 Hz, 1H), 4.52 (t, J = |
| | yl)ethoxy)isoxazol-5-yl)-3- | | 8.2 Hz, 1H), 4.47-4.29 (m, 3H), 3.84 |
| | methylbutanoyl)-N-((S)-1-(4-(4- | | (dd, J = 10.9, 4.1 Hz, 1H), 3.65 (dd, J = |
| | methylthiazol-5- | | 27.3, 10.2 Hz, 2H), 3.55 (s, 1H), 3.47 (q, |
| | yl)phenyl)ethyl)pyrrolidine-2- | | J = 7.9, 6.6 Hz, 1H), 3.11-2.94 (m, 3H), |
| | carboxamide | | 2.73-2.56 (m, 3H), 2.53-2.33 (m, 7H), |
| | | | 2.23-2.11 (m, 1H), 2.04-1.83 (m, 3H), |
| | | | 1.77-1.68 (m, 1H), 1.56 (dd, J = 30.5, |
| | | | 7.0 Hz, 3H), 1.06 (d, J = 6.5 Hz, 3H), |
| | | | 0.97-0.79 (m, 3H). |
| 62 | (2S,4R)-4-hydroxy-1-((R)-2-(3-(2- | 862.3 | 1H NMR (400 MHz, Methanol-d4) δ 8.87 |
| | ((1R,5S,6S)-6-(3-(2- | | (d, J = 6.1 Hz, 1H), 8.45 (s, 1H), 8.12- |
| | hydroxyphenyl)-5- | | 8.03 (m, 1H), 7.51-7.28 (m, 5H), 7.08- |
| | methylthieno[2,3-c]pyridazin-6-yl)- | | 6.95 (m, 2H), 5.42 (s, 1H), 5.05-4.99 |
| | 3-azabicyclo[3.1.1]heptan-3- | | (m, 1H), 4.55-4.46 (m, 1H), 4.43-4.39 |
| | yl)ethoxy)isoxazol-5-yl)-3- | | (m, 1H), 4.05 (t, J = 5.2 Hz, 2H), 3.78- |
| | methylbutanoyl)-N-((S)-1-(4-(4- | | 3.66 (m, 2H), 3.56-3.48 (m, 2H), 3.48- |
| | methylthiazol-5- | | 3.37 (m, 2H), 3.02-2.84 (m, 4H), 2.82- |
| | yl)phenyl)ethyl)pyrrolidine-2- | | 2.76 (m, 1H), 2.48 (s, 3H), 2.45-2.41 |
| | carboxamide | | (m, 3H), 2.37-2.19 (m, 4H), 1.93-1.89 |
| | | | (m, 1H), 1.51 (d, J = 6.9 Hz, 3H), 1.38 (d, |
| | | | J = 8.8 Hz, 1H), 1.00-0.86 (m, 3H), 0.78 |
| | | | (dd, J = 11.7, 6.7 Hz, 3H). |
| 63 | (2S,4R)-4-hydroxy-1-((R)-2-(3-(2- | 862.15 | 1H NMR (300 MHz, Methanol-d4) δ 9.02- |
| | ((1R,5S,6R)-6-(3-(2- | | 8.88 (m, 1H), 8.68-8.54 (m, 1H), 8.15 |
| | hydroxyphenyl)-5- | | (d, J = 7.4 Hz, 1H), 7.60-7.39 (m, 5H), |
| | methylthieno[2,3-c]pyridazin-6-yl)- | | 7.18-7.01 (m, 2H), 6.14 (s, 1H), 5.14- |
| | 3-azabicyclo[3.1.1]heptan-3- | | 5.07 (m, 1H), 4.66-4.45 (m, 3H), 3.96- |
| | yl)ethoxy)isoxazol-5-yl)-3- | | 3.88 (m, 1H), 3.82-3.65 (m, 2H), 3.65- |
| | methylbutanoyl)-N-((S)-1-(4-(4- | | 3.62 (m, 1H), 3.35-3.25 (m, 6H), 3.16 |
| | methylthiazol-5- | | (t, J = 8.5 Hz, 2H), 2.81-2.78 (m, 2H), |
| | yl)phenyl)ethyl)pyrrolidine-2- | | 2.73-2.62 (m, 1H), 2.55 (d, J = 7.8 Hz, |
| | carboxamide | | 3H), 2.47 (d, J = 5.6 Hz, 3H), 2.31-2.23 |
| | | | (m, 1H), 2.11-2.02 (m, 1H), 1.88-1.80 |
| | | | (m, 1H), 1.61 (dd, J = 21.7, 7.0 Hz, 3H), |
| | | | 1.15 (d, J = 6.5 Hz, 3H), 1.00 (t, J = 7.0 |
| | | | Hz, 3H). |
| 66 | (2S,4R)-4-hydroxy-1-((R)-2-(3-(2- | 851.5 | 1H NMR (400 MHz, DMSO-d6) δ 13.46 |
| | (4-(3-(2-hydroxyphenyl)-5- | | (s, 1H), 9.05-8.78 (m, 1H), 8.42 (q, J = |
| | methylthieno[2,3-c]pyridazin-6- | | 2.5 Hz, 2H), 8.17 (dd, J = 8.4, 1.7 Hz, |
| | yl)piperazin-1-yl)ethoxy)isoxazol- | | 1H), 7.49-7.40 (m, 2H), 7.40-7.31 (m, |
| | 5-yl)-3-methylbutanoyl)-N-((S)-1- | | 3H), 7.03-6.94 (m, 2H), 6.18-5.90 (m, |
| | (4-(4-methylthiazol-5- | | 1H), 5.11 (d, J = 3.6 Hz, 1H), 4.97-4.63 |
| | yl)phenyl)ethyl)pyrrolidine-2- | | (m, 1H), 4.42-4.18 (m, 4H), 3.78-3.52 |
| | carboxamide | | (m, 2H), 3.51-3.42 (m, 1H), 3.37-3.33 |
| | | | (m, 4H), 2.80 (t, J = 5.3 Hz, 2H), 2.70 (t, |
| | | | J = 4.9 Hz, 4H), 2.46 (s, 3H), 2.32 (d, J = |
| | | | 2.5 Hz, 3H), 2.28-2.15 (m, 1H), 2.12- |
| | | | 1.85 (m, 1H), 1.83-1.72 (m, 1H), 1.55- |
| | | | 1.32 (m, 3H), 0.97 (t, J = 6.2 Hz, 3H), |
| | | | 0.83 (dd, J = 14.2, 6.7 Hz, 3H). |
| 68 | (2S,4R)-1-((R)-2-(3-(2-(4-(5-fluoro- | 854 | 1H NMR (400 MHz, Methanol-d4) δ 8.86 |
| | 3-(2-hydroxyphenyl)thieno[2,3- | | (d, J = 10.5 Hz, 1H), 8.57 (d, J = 6.4 Hz, |
| | c]pyridazin-6-yl)piperidin-1- | | 1H), 8.00 (m, J = 8.2, 1.7 Hz, 1H), 7.48- |
| | yl)ethoxy)isoxazol-5-yl)-3- | | 7.32 (m, 5H), 7.06-6.98 (m, 2H), 6.01 |
| | methylbutanoyl)-4-hydroxy-N-((S)- | | (d, J = 24.7 Hz, 1H), 5.03 (q, J = 8.7, 7.8 |
| | 1-(4-(4-methylthiazol-5- | | Hz, 1H), 4.51 (d, J = 8.1 Hz, 1H), 4.44 (s, |
| | yl)phenyl)ethyl)pyrrolidine-2- | | 1H), 4.39 (q, J = 5.4 Hz, 2H), 3.84 (m, J = |
| | carboxamide | | 10.8, 4.2 Hz, 1H), 3.75-3.52 (m, 2H), |
| | | | 3.17 (d, J = 11.9 Hz, 2H), 2.87 (t, J = 5.3 |
| | | | Hz, 2H), 2.47 (d, J = 5.5 Hz, 3H), 2.42- |
| | | | 2.30 (m, 3H), 2.26-2.02 (m, 3H), 2.01- |
| | | | 1.81 (m, 3H), 1.56 (m, J = 30.2, 7.0 Hz, |
| | | | 3H), 1.06 (d, J = 6.6 Hz, 3H), 0.90 (m, J = |
| | | | 7.1, 2.4 Hz, 3H). |
| 88 | (2S,4R)-4-hydroxy-1-((R)-2-(3-(2- | 862.3 | 1H NMR (400 MHz, Methanol-d4) δ 8.85 |
| | (6-(3-(2-hydroxyphenyl)-5- | | (d, J = 15.7 Hz, 1H), 8.47 (d, J = 9.8 Hz, |
| | methylthieno[2,3-c]pyridazin-6-yl)- | | 1H), 8.03 (dt, J = 9.3, 2.8 Hz, 1H), 7.43- |
| | 2-azaspiro[3.3]heptan-2- | | 7.30 (m, 5H), 7.01 (ddd, J = 7.6, 4.0, 2.8 |
| | yl)ethoxy)isoxazol-5-yl)-3- | | Hz, 2H), 6.02 (s, 1H), 5.03 (d, J = 7.0 Hz, |
| | methylbutanoyl)-N-((S)-1-(4-(4- | | 1H), 4.51 (t, J = 8.2 Hz, 1H), 4.42 (d, J = |
| | methylthiazol-5- | | 15.8 Hz, 1H), 4.33-4.20 (m, 2H), 3.97 (p, |
| | yl)phenyl)ethyl)pyrrolidine-2- | | J = 8.5 Hz, 1H), 3.84 (dd, J = 10.9, 4.1 |
| | carboxamide | | Hz, 1H), 3.71 (dd, J = 24.4, 11.2 Hz, 1H), |
| | | | 3.65-3.49 (m, 3H), 3.42 (d, J = 9.7 Hz, |
| | | | 2H), 2.92 (d, J = 5.3 Hz, 2H), 2.80 (td, J = |
| | | | 8.6, 2.3 Hz, 2H), 2.47-2.34 (m, 9H), |
| | | | 2.21-2.15 (m, 1H), 1.95 (ddd, J = 13.2, |
| | | | 8.7, 4.5 Hz, 1H), 1.60-1.52 (m, 3H), 1.05 |
| | | | (d, J = 6.6 Hz, 3H), 0.90 (dd, J = 10.3, |
| | | | 6.6 Hz, 3H). |
| 89 | (2S,4R)-4-hydroxy-1-((R)-2-(3-(2- | 850.07 | 1H NMR (300 MHz, Methanol-d4) δ 8.90 |
| | (4-(3-(2-hydroxyphenyl)-6- | | (s, 2H), 8.22 (dd, J = 8.1, 1.6 Hz, 1H), |
| | methylthieno[2,3-c]pyridazin-5- | | 7.52-7.29 (m, 5H), 7.12-6.96 (m, 2H), |
| | yl)piperidin-1-yl)ethoxy)isoxazol-5- | | 6.10 (s, 1H), 5.06 (d, J = 7.0 Hz, 1H), |
| | yl)-3-methylbutanoyl)-N-((S)-1-(4- | | 4.62-4.39 (m, 4H), 3.87 (dd, J = 11.0, |
| | (4-methylthiazol-5- | | 4.3 Hz, 1H), 3.75-3.49 (m, 3H), 3.24 (d, |
| | yl)phenyl)ethyl)pyrrolidine-2- | | J = 12.7 Hz, 2H), 2.98 (t, J = 5.3 Hz, 2H), |
| | carboxamide | | 2.71 (d, J = 2.4 Hz, 3H), 2.50 (s, 3H), |
| | | | 2.47-2.30 (m, 5H), 2.22 (d, J = 13.2 Hz, |
| | | | 1H), 2.06-1.93 (m, 1H), 1.80 (d, J = 11.3 |
| | | | Hz, 2H), 1.58 (dd, J = 22.1, 7.0 Hz, 3H), |
| | | | 1.31 (s, 3H), 1.08 (d, J = 6.6 Hz, 3H), |
| | | | 0.94 (t, J = 6.9 Hz, 3H). |
| 90 | (2S,4R)-1-((R)-2-(3-(2-(4-(5- | 870.1 | 1H NMR (300 MHz, DMSO-d6) δ 11.51 |
| | chloro-3-(2- | | (s, 1H), 10.00 (s, 1H), 8.99 (s, 1H), 8.60 |
| | hydroxyphenyl)thieno[2,3- | | (s, 1H), 8.42 (d, J = 7.6 Hz, 1H), 8.15- |
| | c]pyridazin-6-yl)piperidin-1- | | 8.06 (m, 1H), 7.52-7.33 (m, 5H), 7.11- |
| | yl)ethoxy)isoxazol-5-yl)-3- | | 6.98 (m, 2H), 6.19 (s, 1H), 5.14 (d, J = |
| | methylbutanoyl)-4-hydroxy-N-((S)- | | 3.4 Hz, 1H), 4.92 (p, J = 7.0 Hz, 1H), |
| | 1-(4-(4-methylthiazol-5- | | 4.66-4.52 (m, 1H), 4.43-4.23 (m, 2H), |
| | yl)phenyl)ethyl)pyrrolidine-2- | | 3.77-3.67 (m, 3H), 3.61 (s, 2H), 3.54- |
| | carboxamide | | 3.42 (m, 2H), 3.20-2.99 (m, 1H), 2.88- |
| | | | 2.76 (m, 1H), 2.46 (s, 3H), 2.31-2.15 |
| | | | (m, 4H), 2.11-1.98 (m, 2H), 1.80 (dq, J = |
| | | | 12.8, 7.7, 6.1 Hz, 1H), 1.43 (dd, J = |
| | | | 28.3, 6.9 Hz, 3H), 1.24 (s, 1H), 0.98 (d, |
| | | | J = 6.5 Hz, 3H), 0.85 (dd, J = 11.4, 6.5 |
| | | | Hz, 3H). |
| | | | 1H NMR (400 MHz, DMSO-d6) δ 12.82 |
| 94 | (2S,4R)-4-hydroxy-1-((R)-2-(3-(4- | 903.55 | (s, 1H), 8.99 (d, J = 2.6 Hz, 1H), 8.69 (s, |
| | ((4-(3-(2-hydroxyphenyl)-5- | | 1H), 8.41 (d, J = 7.7 Hz, 1H), 8.17 (d, J = |
| | methylthieno[2,3-c]pyridazin-6- | | 7.9 Hz, 1H), 7.53-7.31 (m, 5H), 7.03 |
| | yl)piperidin-1-yl)methyl)piperidin-1- | | (d, J = 7.8 Hz, 2H), 6.13 (s, 1H), 5.11 (d, |
| | yl)isoxazol-5-yl)-3- | | J = 3.6 Hz, 1H), 5.03-4.85 (m, 1H), 4.44- |
| | methylbutanoyl)-N-((S)-1-(4-(4- | | 4.20 (m, 2H), 3.72 (d, J = 6.3 Hz, 1H), |
| | methylthiazol-5- | | 3.64 (d, J = 12.0 Hz, 2H), 3.57 (d, J = 9.9 |
| | yl)phenyl)ethyl)pyrrolidine-2- | | Hz, 1H), 3.43 (d, J = 10.2 Hz, 1H), 3.27- |
| | carboxamide | | 3.18 (m, 1H), 2.98 (d, J = 11.0 Hz, 2H), |
| | | | 2.76 (t, J = 12.0 Hz, 2H), 2.45 (d, J = 5.3 |
| | | | Hz, 6H), 2.21 (d, J = 6.8 Hz, 2H), 2.07 (q, |
| | | | J = 11.8 Hz, 3H), 1.95 (d, J = 12.3 Hz, |
| | | | 2H), 1.74 (dd, J = 26.1, 12.6 Hz, 6H), |
| | | | 1.42 (dd, J = 31.3, 7.0 Hz, 3H), 1.15 (s, |
| | | | 2H), 0.95 (d, J = 6.6 Hz, 3H), 0.82 (dd, J = |
| | | | 15.8, 6.6 Hz, 3H). |
| 95 | (2S,4R)-4-hydroxy-1-((R)-2-(3-(3- | 864.3 | 1H NMR (400 MHz, Methanol-d4) δ 8.85 |
| | (4-(3-(2-hydroxyphenyl)-5- | | (d, J = 15.6 Hz, 1H), 8.52 (d, J = 7.0 Hz, |
| | methylthieno[2,3-c]pyridazin-6- | | 1H), 8.05 (d, J = 8.1 Hz, 1H), 7.39 (dd, J = |
| | yl)piperidin-1-yl)propoxy)isoxazol- | | 35.2, 6.0 Hz, 5H), 7.01 (d, J = 8.0 Hz, |
| | 5-yl)-3-methylbutanoyl)-N-((S)-1- | | 2H), 5.98 (d, J = 25.3 Hz, 1H), 5.04 (t, J = |
| | (4-(4-methylthiazol-5- | | 7.2 Hz, 1H), 4.60 (s, 1H), 4.56-4.37 |
| | yl)phenyl)ethyl)pyrrolidine-2- | | (m, 2H), 4.28 (t, J = 6.0 Hz, 2H), 3.89- |
| | carboxamide | | 3.80 (m, 1H), 3.65 (dd, J = 24.0, 10.4 Hz, |
| | | | 2H), 3.16 (d, J = 12.4 Hz, 2H), 2.62 (t, J = |
| | | | 7.4 Hz, 2H), 2.52-2.43 (m, 6H), 2.42- |
| | | | 2.15 (m, 4H), 2.05 (d, J = 11.1 Hz, 4H), |
| | | | 1.98-1.85 (m, 2H), 1.56 (dd, J = 31.6, |
| | | | 7.0 Hz, 3H), 1.28 (s, 1H), 1.06 (d, J = 6.5 |
| | | | Hz, 3H), 0.90 (d, J = 6.8 Hz, 3H). |
| 96 | (2S,4R)-4-hydroxy-1-((R)-2-(3-(4- | 889.4 | 1H NMR (400 MHz, DMSO-d6) δ 12.79 |
| | (3-(2-hydroxyphenyl)-5- | | (s, 1H), 8.99 (d, J = 1.7 Hz, 1H), 8.69 (s, |
| | methylthieno[2,3-c]pyridazin-6-yl)- | | 1H), 8.41 (d, J = 7.7 Hz, 1H), 8.15 (dd, J = |
| | [1,4'-bipiperidin]-1'-yl)isoxazol-5- | | 8.0, 6.4 Hz, 1H), 7.44 (d, J = 8.3 Hz, |
| | yl)-3-methylbutanoyl)-N-((S)-1-(4- | | 2H), 7.37 (d, J = 8.0 Hz, 3H), 7.03 (d, J = |
| | (4-methylthiazol-5- | | 7.8 Hz, 2H), 6.16 (s, 1H), 5.12 (d, J = |
| | yl)phenyl)ethyl)pyrrolidine-2- | | 3.6 Hz, 1H), 5.03-4.89 (m, 1H), 4.40- |
| | carboxamide | | 4.24 (m, 2H), 3.78-3.66 (m, 3H), 3.58 |
| | | | (d, J = 9.8 Hz, 1H), 3.44 (s, 1H), 3.05 (s, |
| | | | 1H), 2.79 (t, J = 12.2 Hz, 2H), 2.45 (d, J = |
| | | | 5.0 Hz, 6H), 2.24 (q, J = 7.6, 7.2 Hz, |
| | | | 2H), 2.01 (d, J = 11.0 Hz, 3H), 1.87- |
| | | | 1.75 (m, 3H), 1.72 (s, 2H), 1.61-1.44 |
| | | | (m, 3H), 1.38 (d, J = 7.0 Hz, 3H), 1.23 (s, |
| | | | 1H), 0.96 (t, J = 6.7 Hz, 3H), 0.82 (dd, J = |
| | | | 15.9, 6.7 Hz, 4H). |
| 100 | (2S,4R)-4-hydroxy-1-((R)-2-(3-(2- | 905.05 | 1H NMR (400 MHz, Methanol-d4) δ 8.86 |
| | (4-(3-(3-(2-hydroxyphenyl)-5- | | (d, J = 6.7 Hz, 1H), 8.51 (d, J = 4.1 Hz, |
| | methylthieno[2,3-c]pyridazin-6- | | 1H), 8.08-8.01 (m, 1H), 7.48-7.37 (m, |
| | yl)azetidin-1-yl)piperidin-1- | | 4H), 7.39-7.31 (m, 1H), 7.01 (dt, J = |
| | yl)ethoxy)isoxazol-5-yl)-3- | | 7.5, 3.3 Hz, 2H), 6.02 (s, 1H), 5.03 (d, J = |
| | methylbutanoyl)-N-((S)-1-(4-(4- | | 7.1 Hz, 1H), 4.51 (t, J = 8.2 Hz, 1H), |
| | methylthiazol-5- | | 4.44 (s, 1H), 4.35 (t, J = 5.5 Hz, 2H), 4.23 |
| | yl)phenyl)ethyl)pyrrolidine-2- | | (q, J = 7.4 Hz, 1H), 3.84 (dt, J = 10.4, 5.8 |
| | carboxamide | | Hz, 3H), 3.77-3.65 (m, 1H), 3.65-3.58 |
| | | | (m, 1H), 3.34 (d, J = 7.5 Hz, 2H), 3.02- |
| | | | 2.98 (m, 2H), 2.82 (t, J = 5.7 Hz, 2H), |
| | | | 2.47 (d, J = 5.3 Hz, 3H), 2.43-2.33 (m, |
| | | | 4H), 2.31-2.13 (m, 4H), 1.95 (ddd, J = |
| | | | 13.3, 8.8, 4.6 Hz, 1H), 1.82 (d, J = 12.7 |
| | | | Hz, 2H), 1.63-1.49 (m, 3H), 1.43 (q, J = |
| | | | 11.1 Hz, 2H), 1.05 (d, J = 6.5 Hz, 3H), |
| | | | 0.90 (dd, J = 10.4, 6.7 Hz, 3H). |
| 107 | (2S,4R)-4-hydroxy-1-((R)-2-(3-(2- | 933.3 | 1H NMR (400 MHz, DMSO-d6) δ 12.74 |
| | (4-(3-(2-hydroxyphenyl)-5- | | (s, 1H), 9.03-8.78 (m, 1H), 8.69 (s, 1H), |
| | methylthieno[2,3-c]pyridazin-6-yl)- | | 8.51-8.10 (m, 2H), 7.48-7.41 (m, 2H), |
| | [1,4′-bipiperidin]-1′- | | 7.38 (m, J = 7.4, 6.6, 1.9 Hz, 3H), 7.06- |
| | yl)ethoxy)isoxazol-5-yl)-3- | | 6.98 (m, 2H), 6.11 (s, 1H), 5.11 (s, 1H), |
| | methylbutanoyl)-N-((S)-1-(4-(4- | | 4.97-4.60 (m, 1H), 4.37 (t, J = 7.9 Hz, |
| | methylthiazol-5- | | 1H), 4.26 (t, J = 5.4 Hz, 3H), 3.73-3.63 |
| | yl)phenyl)ethyl)pyrrolidine-2- | | (m, 1H), 3.61-3.50 (m, 1H), 3.46 (m, J = |
| | carboxamide | | 10.0, 5.6 Hz, 1H), 3.18 (s, 2H), 3.03 (d, |
| | | | J = 10.6 Hz, 2H), 2.73 (s, 2H), 2.70- |
| | | | 2.56 (m, 2H), 2.48-2.42 (m, 7H), 2.36- |
| | | | 2.17 (m, 2H), 2.10 (s, 5H), 2.03-1.81 |
| | | | (d, J = 12.6 Hz, 5H), 1.51 (m, J = 41.7, |
| | | | 9.7 Hz, 2H), 1.38 (d, J = 7.0 Hz, 3H), |
| | | | 0.97 (t, J = 6.1 Hz, 3H), 0.81 (d, J = 6.6 |
| | | | Hz, 3H). |
| 108 | (2S,4R)-4-hydroxy-1-((R)-2-(3-(4- | 875.3 | 1H NMR (400 MHz, Methanol-d4) δ |
| | ((3-(3-(2-hydroxyphenyl)-5- | | 8.95-8.79 (m, 1H),8.52 (d, J = 2.5 Hz, |
| | methylthieno[2,3-c]pyridazin-6- | | 1H), 8.05 (d, J = 7.9 Hz, 1H), 7.52-7.28 |
| | yl)azetidin-1-yl)methyl)piperidin-1- | | (m, 5H), 7.02 (s, 2H), 6.15-6.00 (m, |
| | yl)isoxazol-5-yl)-3- | | 1H), 5.03 (d, J = 7.2 Hz, 2H), 4.65-4.38 |
| | methylbutanoyl)-N-((S)-1-(4-(4- | | (m, 3H), 4.24 (s, 1H), 3.85 (d, J = 8.6 Hz, |
| | methylthiazol-5- | | 3H), 3.70 (d, J = 12.7 Hz, 2H), 3.62 (d, J = |
| | yl)phenyl)ethyl)pyrrolidine-2- | | 8.8 Hz, 2H), 2.92-2.78 (m, 2H), 2.48 |
| | carboxamide | | (p, J = 4.8 Hz, 4H), 2.44-2.31 (m, 3H), |
| | | | 2.18 (s, 1H), 1.98 (d, J = 11.3 Hz, 1H), |
| | | | 1.85 (d, J = 12.8 Hz, 2H), 1.59 (d, J = 9.5 |
| | | | Hz, 2H), 1.56-1.46 (m, 2H), 1.31 (d, J = |
| | | | 15.3 Hz, 3H), 1.13-0.98 (m, 3H), 0.96- |
| | | | 0.80 (m, 3H). |
| 110 | (2S,4R)-4-hydroxy-1-((R)-2-(3-(2- | 836.15 | 1H NMR (300 MHz, DMSO-d6) δ 12.69 |
| | (3-((3-(2-hydroxyphenyl)-6- | | (s, 1H), 8.99 (s, 1H), 8.68 (s, 1H), 8.42 |
| | methylthieno[2,3-c]pyridazin-5- | | (d, J = 7.7 Hz, 1H), 8.17 (d, J = 8.2, 1.7 |
| | yl)methyl)azetidin-1- | | Hz, 1H), 7.49-7.40 (m, 2H), 7.40-7.33 |
| | yl)ethoxy)isoxazol-5-yl)-3- | | (m, 3H), 7.09-6.98 (m, 2H), 6.05 (s, |
| | methylbutanoyl)-N-((S)-1-(4-(4- | | 1H), 5.10 (d, J = 3.6 Hz, 1H), 4.98-4.85 |
| | methylthiazol-5- | | (m, 1H), 4.37 (t, J = 7.9 Hz, 1H), 4.28 (s, |
| | yl)phenyl)ethyl)pyrrolidine-2- | | 1H), 4.09 (t, J = 5.4 Hz, 2H), 3.74-3.60 |
| | carboxamide | | (m, 2H), 3.44 (d, J = 11.2 Hz, 2H), 3.29- |
| | | | 3.27 (m, 1H), 3.14 (d, J = 7.5 Hz, 2H), |
| | | | 2.94 (s, 2H), 2.83-2.70 (m, 3H), 2.67 |
| | | | (s, 3H), 2.46 (s, 3H), 2.32-2.13 (m, 1H), |
| | | | 2.11-1.95 (m, 1H), 1.87-1.69 (m, 1H), |
| | | | 1.38 (d, J = 7.0 Hz, 3H), 0.95 (d, J = 6.4 |
| | | | Hz, 3H), 0.78 (d, J = 6.7 Hz, 3H). |
| 113 | (2S,4R)-4-hydroxy-1-((R)-2-(3-(4- | 861.1 | 1H NMR (400 MHz, DMSO-d6) δ 12.84 |
| | (3-(3-(2-hydroxyphenyl)-5- | | (s, 1H), 8.99 (s, 1H), 8.68 (s, 1H), 8.41 |
| | methylthieno[2,3-c]pyridazin-6- | | (d, J = 7.6 Hz, 1H), 8.17 (d, J = 7.9 Hz, |
| | yl)azetidin-1-yl)piperidin-1- | | 1H), 7.56-7.22 (m, 5H), 7.03 (d, J = 7.9 |
| | yl)isoxazol-5-yl)-3- | | Hz, 2H), 6.09 (d, J = 53.0 Hz, 1H), 5.19- |
| | methylbutanoyl)-N-((S)-1-(4-(4- | | 4.85 (m, 2H), 4.38 (t, J = 7.8 Hz, 1H), |
| | methylthiazol-5- | | 4.29 (s, 1H), 4.15 (d, J = 8.3 Hz, 1H), |
| | yl)phenyl)ethyl)pyrrolidine-2- | | 3.72 (t, J = 7.3 Hz, 3H), 3.62-3.48 (m, |
| | carboxamide | | 3H), 3.43 (d, J = 10.9 Hz, 1H), 3.22 (d, J = |
| | | | 6.5 Hz, 2H), 2.94 (s, 2H), 2.46 (s, 2H), |
| | | | 2.39 (s, 3H), 2.23 (s, 2H), 2.05 (d, J = |
| | | | 19.3 Hz, 1H), 1.76 (d, J = 28.1 Hz, 3H), |
| | | | 1.42 (d, J = 7.0 Hz, 3H), 1.23 (s, 1H), |
| | | | 0.96 (t, J = 6.7 Hz, 3H), 0.82 (dd, J = |
| | | | 15.9, 6.7 Hz, 4H). |
| 69 | (2S,4R)-4-hydroxy-1-((R)-2-(3-(3- | 822.1 | 1H NMR (300 MHz, Methanol-d4) δ 8.89 |
| | (3-(2-hydroxyphenyl)-7,8- | | (s, 1H), 8.58 (s, 1H), 8.06 (dd, J = 8.3, |
| | dihydropyrido[3′,4′:4,5]thieno[2,3- | | 1.6 Hz, 1H), 7.49-7.32 (m, 4H), 7.29- |
| | c]pyridazin-6(5H)- | | 7.19 (m, 1H), 7.07-6.96 (m, 2H), 5.88 |
| | yl)propoxy)isoxazol-5-yl)-3- | | (s, 1H), 5.04-4.94 (m, 1H), 4.56 (t, J = |
| | methylbutanoyl)-N-((S)-1-(4-(4- | | 8.3 Hz, 1H), 4.51-4.35 (m, 1H), 4.10 (t, |
| | methylthiazol-5- | | J = 6.5 Hz, 2H), 3.92-3.77 (m, 3H), 3.67 |
| | yl)phenyl)ethyl)pyrrolidine-2- | | (d, J = 9.8 Hz, 2H), 3.20-3.07 (m, 3H), |
| | carboxamide | | 3.05-2.95 (m, 1H), 2.74 (t, J = 7.2 Hz, |
| | | | 2H), 2.49 (s, 2H), 2.47-2.30 (m, 2H), |
| | | | 2.28-2.16 (m, 1H), 2.15-2.03 (m, 2H), |
| | | | 2.03-1.89 (m, 1H), 1.47 (d, J = 7.0 Hz, |
| | | | 3H), 1.08 (d, J = 6.5 Hz, 3H), 0.98 (d, J = |
| | | | 6.7 Hz, 3H). |
| 70 | (2S,4R)-4-hydroxy-1-((R)-2-(3-(4- | 847.2 | 1H NMR (300 MHz, DMSO-d6) δ 13.03 |
| | (3-(2-hydroxyphenyl)-7,8- | | (br s, 1H), 8.99 (s, 1H), 8.75 (s, 1H), 8.41 |
| | dihydropyrido[3′,4′:4,5]thieno[2,3- | | (d, J = 7.7 Hz, 1H), 8.14 (d, J = 7.9 Hz, |
| | c]pyridazin-6(5H)-yl)piperidin-1- | | 1H), 7.54-7.30 (m, 5H), 7.06-6.97 (m, |
| | yl)isoxazol-5-yl)-3- | | 2H), 6.18 (s, 1H), 5.13 (d, J = 3.5 Hz, |
| | methylbutanoyl)-N-((S)-1-(4-(4- | | 1H), 4.94- 4.88 (m, 1H), 4.40- 4.26 (m, |
| | methylthiazol-5- | | 2H), 4.00-3.84 (m, 2H), 3.81-3.66 (m, |
| | yl)phenyl)ethyl)pyrrolidine-2- | | 3H), 3.58 (d, J = 9.8 Hz, 1H), 3.53-3.40 |
| | carboxamide | | (m, 2H), 3.02-2.92 (m, 3H), 2.85 (t, J = |
| | | | 12.1 Hz, 2H), 2.73 (s, 1H), 2.47-2.41 |
| | | | (m, 3H), 2.27-2.17 (m, 1H), 2.10-1.84 |
| | | | (m, 3H), 1.84-1.55 (m, 3H), 1.38 (d, J = |
| | | | 7.0 Hz, 3H), 0.96 (d, J = 6.4 Hz, 3H), |
| | | | 0.81 (d, J = 6.8 Hz, 3H). |
| 92 | (2S,4R)-4-hydroxy-1-((R)-2-(3-(4- | 861.3 | 1H NMR (400 MHz, Methanol-d4) δ 8.87 |
| | ((3-(2-hydroxyphenyl)-7,8- | | (d, J = 2.5 Hz, 1H), 8.53 (d, J = 2.9 Hz, |
| | dihydropyrido[3′,4′:4,5]thieno[2,3- | | 1H), 8.07-8.01 (m, 1H), 7.47-7.36 (m, |
| | c]pyridazin-6(5H)- | | 4H), 7.38-7.32 (m, 1H), 7.00 (dd, J = |
| | yl)methyl)piperidin-1-yl)isoxazol-5- | | 8.2, 6.4 Hz, 2H), 6.10 (s, 1H), 5.03 (d, J = |
| | yl)-3-methylbutanoyl)-N-((S)-1-(4- | | 6.9 Hz, 1H), 4.54-4.41 (m, 2H), 3.87- |
| | (4-methylthiazol-5- | | 3.80 (m, 3H), 3.71 (d, J = 13.2 Hz, 2H), |
| | yl)phenyl)ethyl)pyrrolidine-2- | | 3.61 (dd, J = 10.3, 6.2 Hz, 2H), 3.12 (s, |
| | carboxamide | | 2H), 3.00-2.86 (m, 4H), 2.56 (d, J = 6.9 |
| | | | Hz, 2H), 2.47 (d, J = 4.4 Hz, 3H), 2.42- |
| | | | 2.35 (m, 1H), 2.22-2.15 (m, 1H), 2.00- |
| | | | 1.86 (m, 4H), 1.52 (d, J = 7.0 Hz, 3H), |
| | | | 1.31-1.39 (m, 1H), 1.05 (d, J = 6.5 Hz, |
| | | | 3H), 0.89 (d, J = 6.7 Hz, 3H). |
| 109 | (2S,4R)-4-hydroxy-1-((R)-2-(3-(2- | 808.2 | 1H NMR (400 MHz, Methanol-d4) δ 8.86 |
| | (3-(2-hydroxyphenyl)-7,8- | | (d, J = 5.5 Hz, 1H), 8.57-8.48 (m, 1H), |
| | dihydropyrido[3′,4′:4,5]thieno[2,3- | | 8.07-7.96 (m, 1H), 7.38 (d, J = 32.7, |
| | c]pyridazin-6(5H)- | | 8.5 Hz, 5H), 7.01 (tt, J = 7.9, 3.8 Hz, 2H), |
| | yl)ethoxy)isoxazol-5-yl)-3- | | 6.05 (s, 1H), 5.01(d, 1H), 4.49 (q, J = 4.9, |
| | methylbutanoyl)-N-((S)-1-(4-(4- | | 3.4 Hz, 3H), 4.42 (s, 1H), 3.97 (s, 2H), |
| | methylthiazol-5- | | 3.82 (dd, J = 11.0, 4.2 Hz, 1H), 3.65 (dd, |
| | yl)phenyl)ethyl)pyrrolidine-2- | | J = 20.4, 11.3 Hz, 2H), 3.13 (s, 6H), 2.46 |
| | carboxamide | | (d, J = 11.7 Hz, 3H), 2.21-2.11 (m, 1H), |
| | | | 1.93 (ddd, J = 19.6, 9.7, 5.4 Hz, 1H), |
| | | | 1.53 (dd, J = 21.1, 7.1 Hz, 3H), 1.04 (d, |
| | | | J = 6.5 Hz, 3H), 0.88 (t, J = 7.5 Hz, 3H). |

Preparation of (2S,4R)-4-hydroxy-1-[(2R)-2-[3-(3-{4-[3-(2-hydroxyphenyl)-5-methylthieno[2,3-c]pyridazin-6-yl]piperidin-1-yl}cyclobutyl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 34) and (2S,4R)-4-hydroxy-1-[(2S)-2-[3-(3-{4-[3-(2-hydroxyphenyl)-5-methylthieno[2,3-c]pyridazin-6-yl]piperidin-1-yl}cyclobutyl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 114)

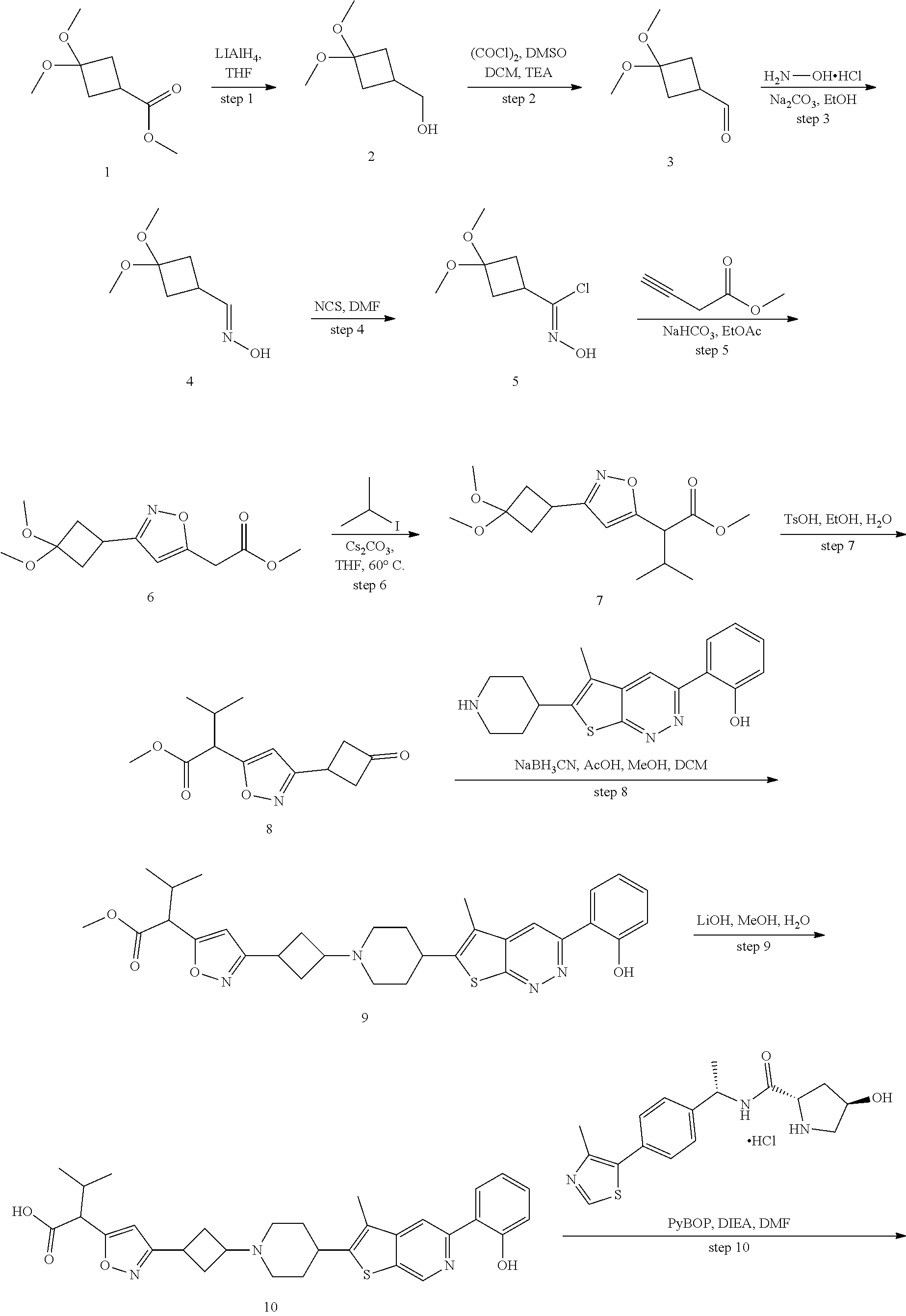

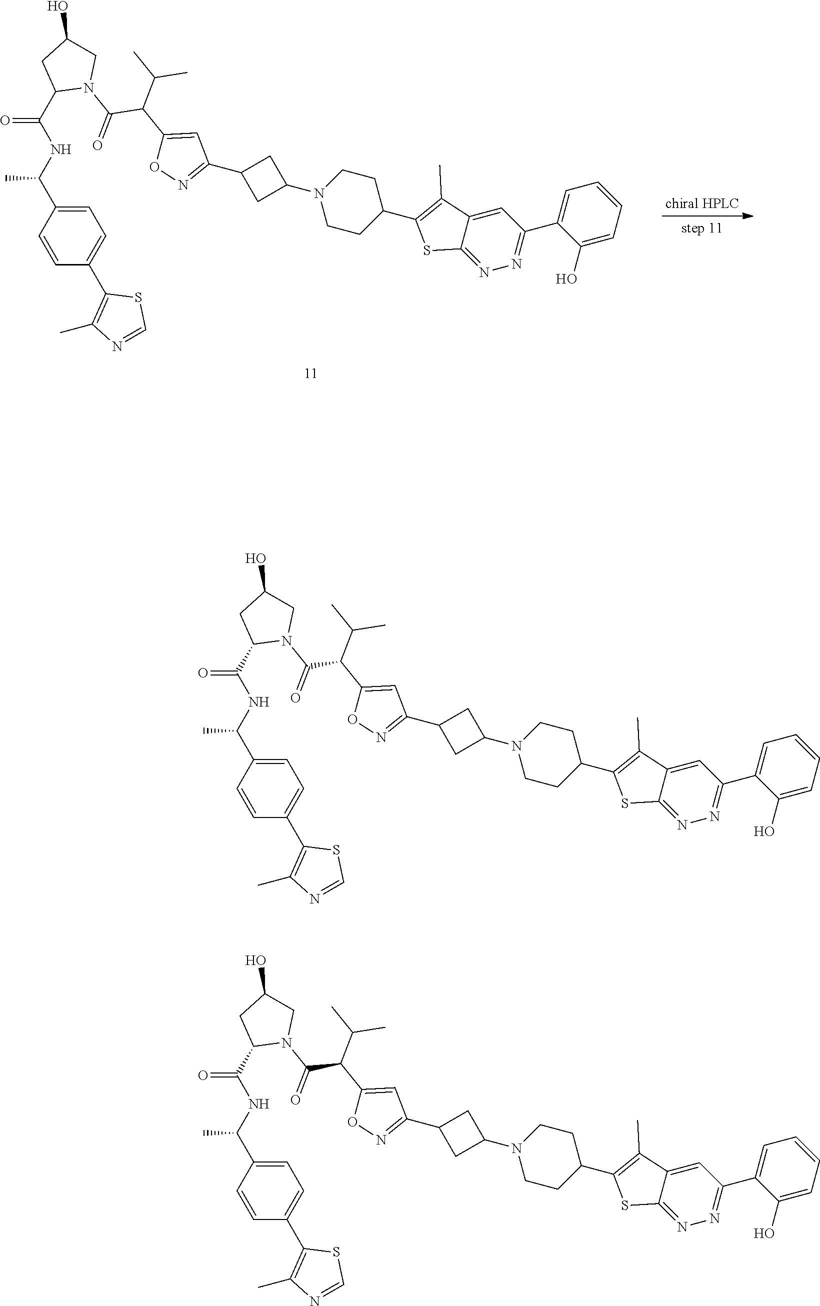 [SMILES: Cc1ncsc1-c1ccc([C@H](C)NC(=O)C2C[C@@H](O)CN2C(=O)C(c2cc(C3CC(N4CCC(c5sc6nnc(-c7ccccc7O)cc6c5C)CC4)C3)no2)C(C)C)cc1 ; Cc1ncsc1-c1ccc([C@H](C)NC(=O)[C@@H]2C[C@@H](O)CN2C(=O)[C@@H](c2cc(C3CC(N4CCC(c5sc6nnc(-c7ccccc7O)cc6c5C)CC4)C3)no2)C(C)C)cc1 ; Cc1ncsc1-c1ccc([C@H](C)NC(=O)[C@@H]2C[C@@H](O)CN2C(=O)[C@H](c2cc(C3CC(N4CCC(c5sc6nnc(-c7ccccc7O)cc6c5C)CC4)C3)no2)C(C)C)cc1]

Step 1: Preparation of (3,3-dimethoxycyclobutyl)methanol (intermediate 2)

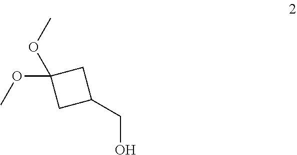

To a solution of methyl 3,3-dimethoxycyclobutane-1-carboxylate (10 g, 57.407 mmol, 1 equiv) in THF (100 mL) was added LiAlH₄ (3.27 g, 86.111 mmol, 1.5 equiv) at 0° C. The resulting solution was stirred at room temperature for 2 hours. The mixture was quenched by 10% w/v sodium hydroxide (aq). The mixture was filtered and the filtrate was concentrated under reduced pressure to give intermediate 2 (8.3 g, crude) as a yellow oil. LCMS (ESI) m/z: [M+H]⁺=147.

Step 2: Preparation of 3,3-dimethoxycyclobutane-1-carbaldehyde (intermediate 3)

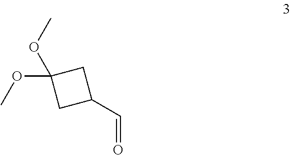

To a solution of (COCl)₂ (13.9 g, 109.448 mmol, 2 equiv) in DCM (120 mL) was added DMSO (8.55 g, 109.448 mmol, 2 equiv) in DCM (10 mL) at −78° C. The resulting solution was stirred at −78° C. for 5 min. The intermediate 2 (8 g, 54.724 mmol, 1 equiv) in DCM (20 mL) was added to the mixture at −78° C. and the resulting solution was stirred at −78° C. for 30 min. To above solution was added TEA (27.68 g, 273.61 mmol, 5 equiv) and the resulting solution was stirred at room temperature for 1 h. The mixture was diluted with DCM (400 mL) and washed with water (3×400 mL). The organic layer was dried over anhydrous sodium sulfate, filtered and concentrated under reduced pressure to give intermediate 3 (7.66 g, crude) as a yellow oil. LCMS (ESI) m/z: [M+H]⁺=145.

Step 3: Preparation of (E)-N-[(3,3-dimethoxycyclobutyl)methylidene]hydroxylamine (intermediate 4)

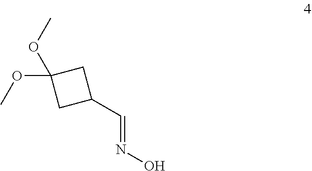

To a solution of intermediate 3 (7.66 g, 53.132 mmol, 1 equiv) and hydroxylamine hydrochloride (7.38 g, 106.264 mmol, 2 equiv) in EtOH (60 mL) was added Na₂CO₃ (16.89 g, 159.396 mmol, 3 equiv). The resulting mixture was stirred at 25° C. for 6 hours. The mixture was diluted with EtOAc (400 mL) and washed with water (3×400 mL). The organic layer was dried over anhydrous sodium sulfate, filtered and concentrated under reduced pressure to give intermediate 4 (4.72 g, crude) as a yellow oil. LCMS (ESI) m/z: [M+H]⁺=160.

Step 4: Preparation of (Z)—N-hydroxy-3,3-dimethoxycyclobutane-1-carbonimidoyl chloride (intermediate 5)

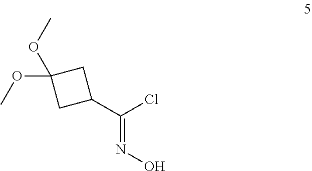

To a solution of intermediate 4 (4.72 g, 29.651 mmol, 1 equiv) in DMF (50 mL) was added NCS (5.94 g, 44.477 mmol, 1.5 equiv). The resulting solution was stirred at 25° C. for 6 hours. The mixture was diluted with EtOAc (300 mL) and washed with water (3×300 mL). The organic layer was dried over anhydrous sodium sulfate, filtered and concentrated under reduced pressure to give intermediate 5 (6.57 g, crude) as a yellow oil. LCMS (ESI) m/z: [M+H]⁺=194.

Step 5: Preparation of methyl 2-[3-(3,3-dimethoxycyclobutyl)-1,2-oxazol-5-yl]acetate (intermediate 6)

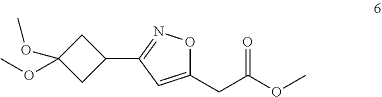

To a solution of intermediate 5 (6.57 g, 33.931 mmol, 1 equiv) and methyl but-3-ynoate (3.99 g, 40.717 mmol, 1.2 equiv) in EtOAc (50 mL) was added NaHCO₃ (5.70 g, 67.862 mmol, 2 equiv). The resulting mixture was stirred at 25° C. for 16 hours. The mixture was diluted with EtOAc (300 mL) and washed with water (3×300 mL). The organic layer was dried over anhydrous sodium sulfate, filtered and concentrated under reduced pressure to give a crude product. The crude product was purified by reverse phase flash C18 chromatography, elution gradient 0% to 53% ACN in H₂O, to give intermediate 6 (2.4 g, 27.71%) as a yellow oil. LCMS (ESI) m/z: [M+H]⁺=256.

Step 6: Preparation of methyl 2-[3-(3,3-dimethoxycyclobutyl)-1,2-oxazol-5-yl]-3-methylbutanoate (intermediate 7)

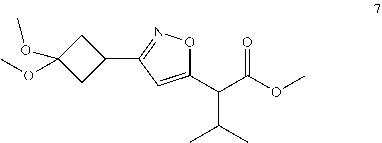

To a solution of intermediate 6 (2.4 g, 9.402 mmol, 1 equiv) and 2-iodopropane (3.20 g, 18.804 mmol, 2 equiv) in THF (20 mL) was added Cs₂CO₃ (6.13 g, 18.804 mmol, 2 equiv). The resulting mixture was stirred at 60° C. for 16 hours. The mixture was diluted with EtOAc (300 mL) and washed with water (3×300 mL). The organic layer was dried over anhydrous sodium sulfate, filtered and concentrated under reduced pressure to give a crude product. The crude product was purified by reverse phase flash C18 chromatography, elution gradient 0% to 48% ACN in H₂O, to give intermediate 7 (538 mg, 19.24%) as a yellow oil. LCMS (ESI) m/z: [M+H]⁺=298.

Step 7: Preparation of methyl 3-methyl-2-[3-(3-oxocyclobutyl)-1,2-oxazol-5-yl]butanoate (intermediate 8)

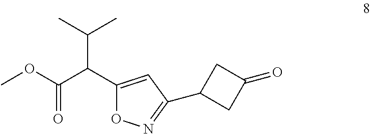 [SMILES: COC(=O)C(c1cc(C2CC(=O)C2)no1)C(C)C]

To a solution of intermediate 7 (538 mg, 1.809 mmol, 1 equiv) in EtOH (5 mL) and H₂O (0.5 mL) was added TsOH (311.56 mg, 1.809 mmol, 1 equiv). The resulting solution was stirred at 25° C. for 6 hours. The mixture was diluted with EtOAc (200 mL) and washed with water (3×200 mL). The organic layer was dried over anhydrous sodium sulfate, filtered and concentrated under reduced pressure to give a crude product. The crude product was purified by reverse phase flash C18 chromatography, elution gradient 0% to 45% ACN in H₂O, to give intermediate 8 (368 mg, 80.94%) as a yellow oil. LCMS (ESI) m/z: [M+H]⁺=252.

Step 8: Preparation of methyl 2-[3-(3-{4-[3-(2-hydroxyphenyl)-5-methylthieno[2,3-c]pyridazin-6-yl]piperidin-1-yl}cyclobutyl)-1,2-oxazol-5-yl]-3-methylbutanoate (intermediate 9)

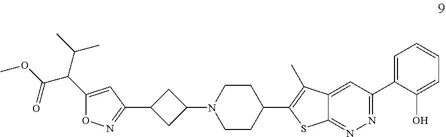 [SMILES: COC(=O)C(c1cc(C2CC(N3CCC(c4sc5nnc(-c6ccccc6O)cc5c4C)CC3)C2)no1)C(C)C]

To a solution of intermediate 8 (180 mg, 0.716 mmol, 1 equiv) and 2-[5-methyl-6-(piperidin-4-yl)thieno[2,3-c]pyridazin-3-yl]phenol (349.67 mg, 1.074 mmol, 1.5 equiv) in MeOH (2 mL) and DCM (2 mL) were added AcOH (0.1 mL, 1.745 mmol, 2.44 equiv) and NaBH3CN (90.03 mg, 1.432 mmol, 2 equiv). The resulting solution was stirred at 25° C. for 3 hours. The mixture was diluted with EtOAc (200 mL) and washed with water (3×200 mL). The organic layer was dried over anhydrous sodium sulfate, filtered and concentrated under reduced pressure to give a crude product. The crude product was purified by reverse phase flash C18 chromatography, elution gradient 0% to 53% ACN in H₂O, to give intermediate 9 (165 mg, 41.08%) as a brown solid. LCMS (ESI) m/z: [M+H]⁺=561.

Step 9: Preparation of 2-[3-(3-{4-[3-(2-hydroxyphenyl)-5-methylthieno[2,3-c]pyridazin-6-yl]piperidin-1-yl}cyclobutyl)-1,2-oxazol-5-yl]-3-methylbutanoic acid (intermediate 10)

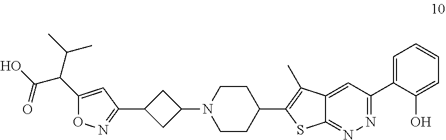 [SMILES: Cc1c(C2CCN(C3CC(c4cc(C(C(=O)O)C(C)C)on4)C3)CC2)sc2nnc(-c3ccccc3O)cc12]

To a solution of intermediate 9 (165 mg, 0.294 mmol, 1 equiv) in MeOH (2 mL) and H₂O (0.4 mL) was added LiOH (35.24 mg, 1.470 mmol, 5 equiv). The resulting solution was stirred at 25° C. for 6 hours. The mixture was acidified to pH 5-6 with HCl (1 M in H₂O). The mixture was diluted with EtOAc (200 mL) and washed with water (3×200 mL). The organic layer was dried over anhydrous sodium sulfate, filtered and concentrated under reduced pressure to give a crude product. The crude product was purified by reverse phase flash C18 chromatography, elution gradient 0% to 53% ACN in H₂O, to give intermediate 10 (182 mg, crude) as a brown solid. LCMS (ESI) m/z: [M+H]⁺=547.

Step 10: Preparation of (2S,4R)-4-hydroxy-1-{2-[3-(3-{4-[3-(2-hydroxyphenyl)-5-methylthieno[2,3-c]pyridazin-6-yl]piperidin-1-yl}cyclobutyl)-1,2-oxazol-5-yl]-3-methylbutanoyl}-N-[(1S)-1-[4-(4-methyl-1,3-thiazol-5-yl)phenyl]ethyl]pyrrolidine-2-carboxamide (intermediate 11)

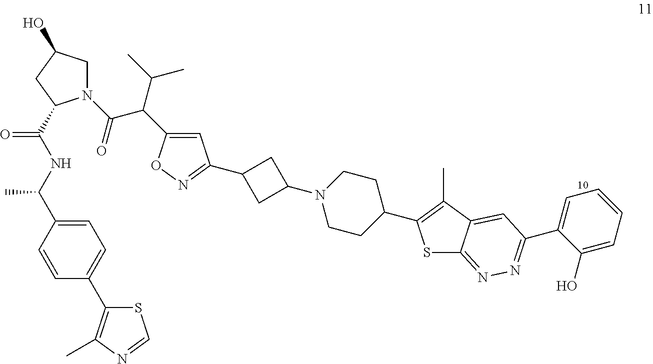 [SMILES: Cc1ncsc1-c1ccc([C@H](C)NC(=O)[C@@H]2C[C@@H](O)CN2C(=O)C(c2cc(C3CC(N4CCC(c5sc6nnc(-c7ccccc7O)cc6c5C)CC4)C3)no2)C(C)C)cc1]

To a stirred solution of intermediate 10 (180 mg, 0.329 mmol, 1 equiv) and (2S,4R)-4-hydroxy-N-[(1S)-1-[4-(4-methyl-1,3-thiazol-5-yl)phenyl]ethyl]pyrrolidine-2-carboxamide hydrochloride (109.12 mg, 0.329 mmol, 1 equiv) in DMF (3 mL) was added PyBOP (342 mg, 0.658 mmol, 2 equiv). The resulting solution was stirred at 25° C. for 10 minutes, then DIEA (212.78 mg, 1.645 mmol, 5 equiv) was added to the mixture. The resulting solution was stirred at 25° C. for 6 hours. Without additional work-up, the crude reaction mixture was purified by reverse phase flash C18 chromatography, elution gradient 0% to 46% ACN in H₂O, to give intermediate 11 (14 mg, 4.94%) as a brown solid. LCMS (ESI) m/z: [M+H]⁺=860.

Step 11: Preparation of (2S,4R)-4-hydroxy-1-[(2R)-2-[3-(3-{4-[3-(2-hydroxyphenyl)-5-methylthieno[2,3-c]pyridazin-6-yl]piperidin-1-yl}cyclobutyl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 34) and (2S,4R)-4-hydroxy-1-[(2S)-2-[3-(3-{4-[3-(2-hydroxyphenyl)-5-methylthieno[2,3-c]pyridazin-6-yl]piperidin-1-yl}cyclobutyl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 114)

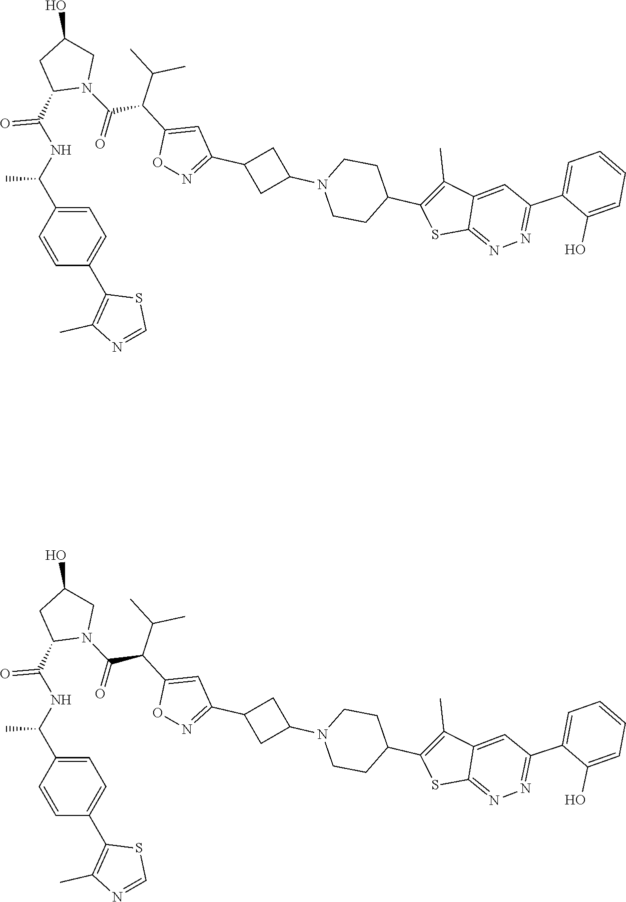 [SMILES: Cc1ncsc1-c1ccc([C@H](C)NC(=O)[C@@H]2C[C@@H](O)CN2C(=O)[C@@H](c2cc(C3CC(N4CCC(c5sc6nnc(-c7ccccc7O)cc6c5C)CC4)C3)no2)C(C)C)cc1 ; Cc1ncsc1-c1ccc([C@H](C)NC(=O)[C@@H]2C[C@@H](O)CN2C(=O)[C@H](c2cc(C3CC(N4CCC(c5sc6nnc(-c7ccccc7O)cc6c5C)CC4)C3)no2)C(C)C)cc1]

Intermediate 11 (14 mg) was purified by chiral Prep HPLC: Column, CHIRALPAK ID, 2*25 cm, 5 μm; Mobile Phase A: MtBE (10 mM NH3-MeOH), Mobile Phase B: MeOH; Flow rate: 20 mL/min; Gradient: 5% B to 15% B in 20 min; Detector, UV 254/220 nm; RT1 (min): 10.3; RT2 (min): 15.4. This resulted in:

Compound 34 (2.1 mg, 15.00%) (second peak) as an off-white solid. ¹H NMR (300 MHz, Methanol-d4) δ 8.89 (d, J=2.4 Hz, 1H), 8.56 (s, 1H), 8.08 (d, J=8.2 Hz, 1H), 7.52-7.38 (m, 5H), 7.09-6.99 (m, 2H), 6.42 (d, J=11.8 Hz, 1H), 5.07 (d, J=7.0 Hz, 1H), 4.55 (t, J=8.2 Hz, 1H), 4.47 (s, 1H), 3.94-3.77 (m, 2H), 3.66 (d, J=2.1 Hz, 3H), 3.63-3.51 (m, 1H), 3.13 (d, J=12.8 Hz, 2H), 3.03-2.92 (m, 1H), 2.61 (d, J=9.0 Hz, 2H), 2.50 (s, 6H), 2.47-2.29 (m, 1H), 2.26-2.03 (m, 8H), 2.01-1.78 (m, 4H), 1.67-1.47 (m, 5H), 1.10 (d, J=6.6 Hz, 4H), 0.90 (d, J=6.7 Hz, 8H). LCMS (ESI) m/z: [M+H]⁺=860.30

Compound 114 (2.1 mg, 15.00%) (first peak) as a yellow solid. ¹H NMR (300 MHz, Methanol-d4) δ 8.88 (d, J=12.2 Hz, 1H), 8.55 (d, J=1.9 Hz, 11H), 8.07 (d, J=7.5 Hz, 1H), 7.53-7.32 (m, 5H), 7.09-6.98 (m, 2H), 6.43 (d, J=9.7 Hz, 1H), 5.09-4.95 (m, 1H), 4.61 (t, J=8.1 Hz, 1H), 4.46 (s, 1H), 3.92 (d, J=9.1 Hz, 1H), 3.80-3.63 (m, 4H), 3.23 (s, 1H), 3.12 (d, J=12.3 Hz, 2H), 3.04-2.92 (m, 1H), 2.67-2.49 (m, 3H), 2.48-2.40 (m, 6H), 2.30-2.04 (m, 8H), 2.006-1.82 (m, 3H), 1.66-1.45 (m, 5H), 1.10 (d, J=6.6 Hz, 3H), 1.04-0.82 (m, 6H). LCMS (ESI) m/z: [M+H]⁺=860.30.

The compounds in Table 8 were prepared using procedures similar to those used above for the preparation of compound 34 using the appropriate amine and ketone.

TABLE 8

| | | LCMS | |
|---|---|---|---|
| Compound | | (ESI) | |
| No. | Name | m/z | ¹H NMR |
| 37 | (2S,4R)-4-hydroxy-1-((R)-2-(3- | 888.4 | 1H NMR (400 MHz, DMSO-d6) δ 12.74 |
| | ((1s,4S)-4-(4-(3-(2- | | (s, 1H), 8.92 (d, J = 5.4 Hz, 1H), 8.61 (s, |
| | hydroxyphenyl)-5- | | 1H), 8.21 (dd, J = 93.5, 7.8 Hz, 1H), 7.43- |
| | methylthieno[2,3-c]pyridazin-6- | | 7.26 (m, 5H), 6.95 (t, J = 7.3 Hz, 2H), |
| | yl)piperidin-1- | | 6.22 (s, 1H), 5.11 (d, J = 3.7 Hz, 2H), |
| | yl)cyclohexyl)isoxazol-5-yl)-3- | | 4.98-4.85 (m, 1H), 4.43 (t, J = 7.7 Hz, |
| | methylbutanoyl)-N-((S)-1-(4-(4- | | 1H), 4.28 (s, 2H), 3.85 (d, J = 8.5 Hz, |
| | methylthiazol-5- | | 1H), 3.61-3.53 (m, 1H), 3.17-3.11 (m, |
| | yl)phenyl)ethyl)pyrrolidine-2- | | 1H), 2.99 (s, 2H), 2.60-2.52 (m, 2H), |
| | carboxamide | | 2.49-2.39 (m, 7H), 2.10 (d, J = 37.6 Hz, |
| | | | 2H), 1.99-1.75 (m, 8H), 1.69 (d, J = |
| | | | 13.0 Hz, 1H), 1.47 (d, J = 7.1 Hz, 3H), |
| | | | 1.39 (s, 3H), 1.36 (d, J = 7.0 Hz, 2H), |
| | | | 0.97 (d, J = 6.6 Hz, 3H), 0.83-0.71 (m, |
| | | | 3H). |
| 38 | (2S,4R)-4-hydroxy-1-((R)-2-(3- | 888.35 | 1H NMR (400 MHz, Methanol-d4) δ 8.74 |
| | ((1r,4R)-4-(4-(3-(2-hydroxyphenyl)- | | (s, 1H), 8.48 (s, 1H), 8.03 (d, J = 7.6 Hz, |
| | 5-methylthieno[2,3-c]pyridazin-6- | | 1H), 7.47 (s, 1H), 7.34 (s, 4H), 7.06- |
| | yl)piperidin-1- | | 6.98 (m, 2H), 6.36 (s, 1H), 4.99 (d, J = |
| | yl)cyclohexyl)isoxazol-5-yl)-3- | | 7.0 Hz, 1H), 4.59 (t, J = 8.1 Hz, 1H), 4.44 |
| | methylbutanoyl)-N-((S)-1-(4-(4- | | (s, 1H), 3.89 (d, J = 9.2 Hz, 1H), 3.73 (d, |
| | methylthiazol-5- | | J = 11.1 Hz, 1H), 3.64 (dd, J = 11.1, 4.2 |
| | yl)phenyl)ethyl)pyrrolidine-2- | | Hz, 1H), 3.16 (s, 2H), 3.04 (s, 1H), 2.67- |
| | carboxamide | | 2.45 (m, 2H), 2.45-2.35 (m, 9H), 2.23 |
| | | | (s, 3H), 1.97 (td, J = 14.1, 13.0, 7.7 Hz, |
| | | | 3H), 1.88-1.77 (m, 6H), 1.74-1.55 (m, |
| | | | 2H), 1.50 (d, J = 7.0 Hz, 3H), 1.08 (d, J = |
| | | | 6.6 Hz, 3H), 0.91 (d, J = 6.7 Hz, 3H). |

Preparation of (2S,4R)-4-hydroxy-1-[(2R)-2-{3-[(1S,2S)-2-{4-[3-(2-hydroxyphenyl)-5-methylthieno[2,3-c]pyridazin-6-yl]piperidin-1-yl}cyclobutoxy]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 36), (2S,4R)-4-hydroxy-1-[(2S)-2-{3-[(1S,2S)-2-{4-[3-(2-hydroxyphenyl)-5-methylthieno[2,3-c]pyridazin-6-yl]piperidin-1-yl}cyclobutoxy]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 115) and (2S,4R)-4-hydroxy-1-[(2R)-2-{3-[(1R,2S)-2-{4-[3-(2-hydroxyphenyl)-5-methylthieno[2,3-c]pyridazin-6-yl]piperidin-1-yl}cyclobutoxy]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 35)

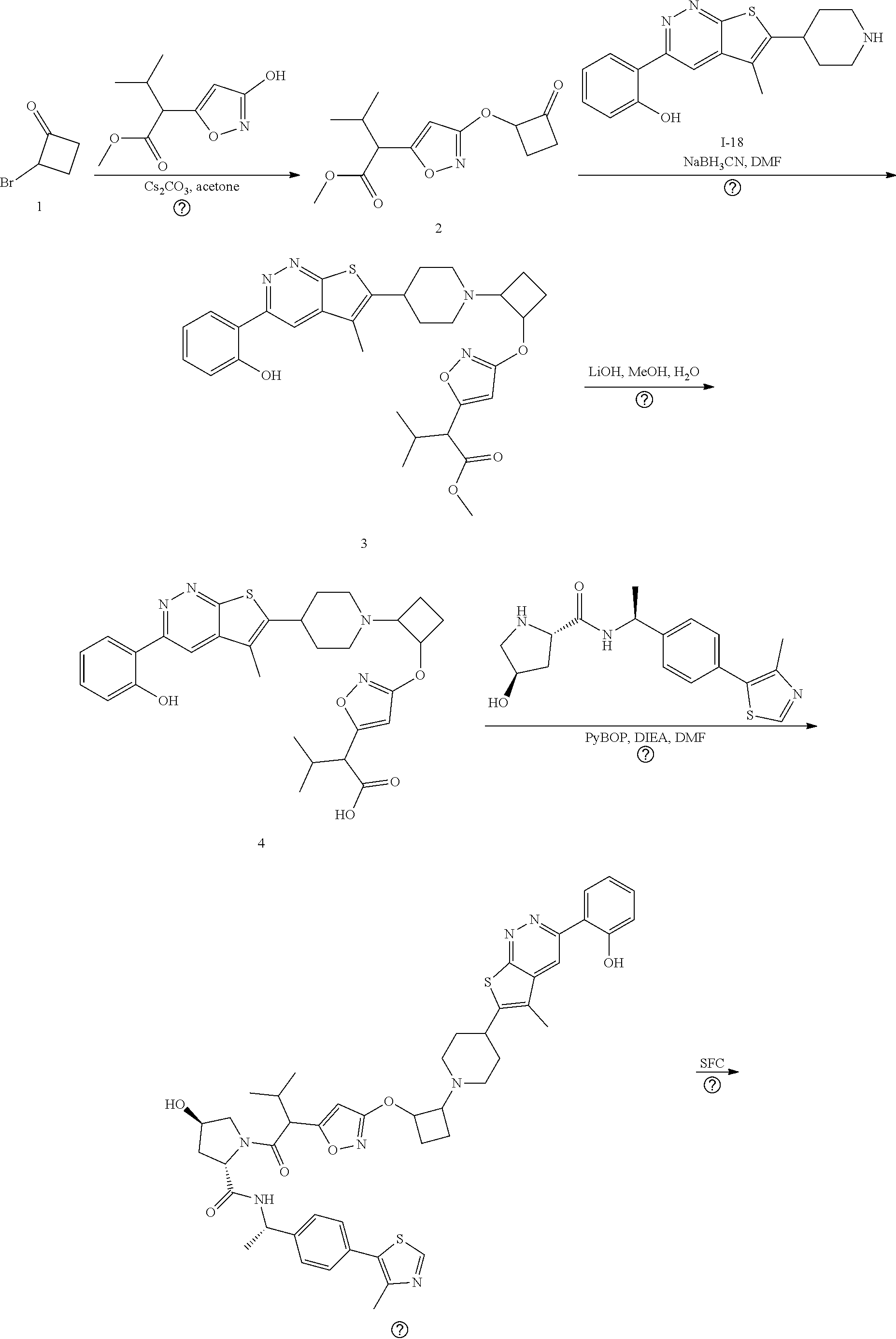

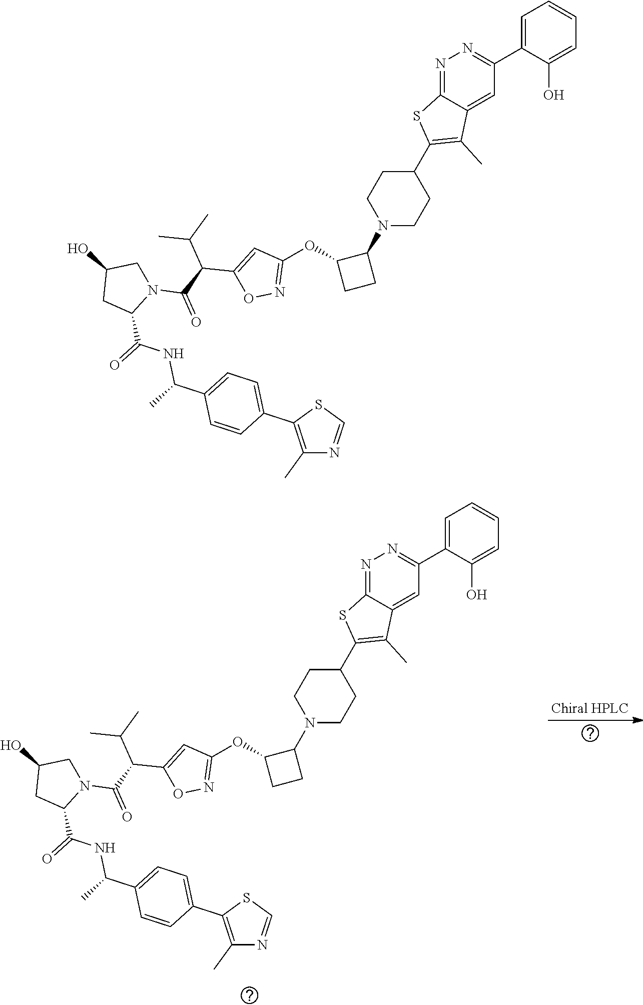 [SMILES: Cc1ncsc1-c1ccc([C@H](C)NC(=O)[C@@H]2C[C@@H](O)CN2C(=O)[C@H](c2cc(O[C@H]3CC[C@@H]3N3CCC(c4sc5nnc(-c6ccccc6O)cc5c4C)CC3)no2)C(C)C)cc1 ; Cc1ncsc1-c1ccc([C@H](C)NC(=O)[C@@H]2C[C@@H](O)CN2C(=O)[C@@H](c2cc(O[C@H]3CCC3N3CCC(c4sc5nnc(-c6ccccc6O)cc5c4C)CC3)no2)C(C)C)cc1]

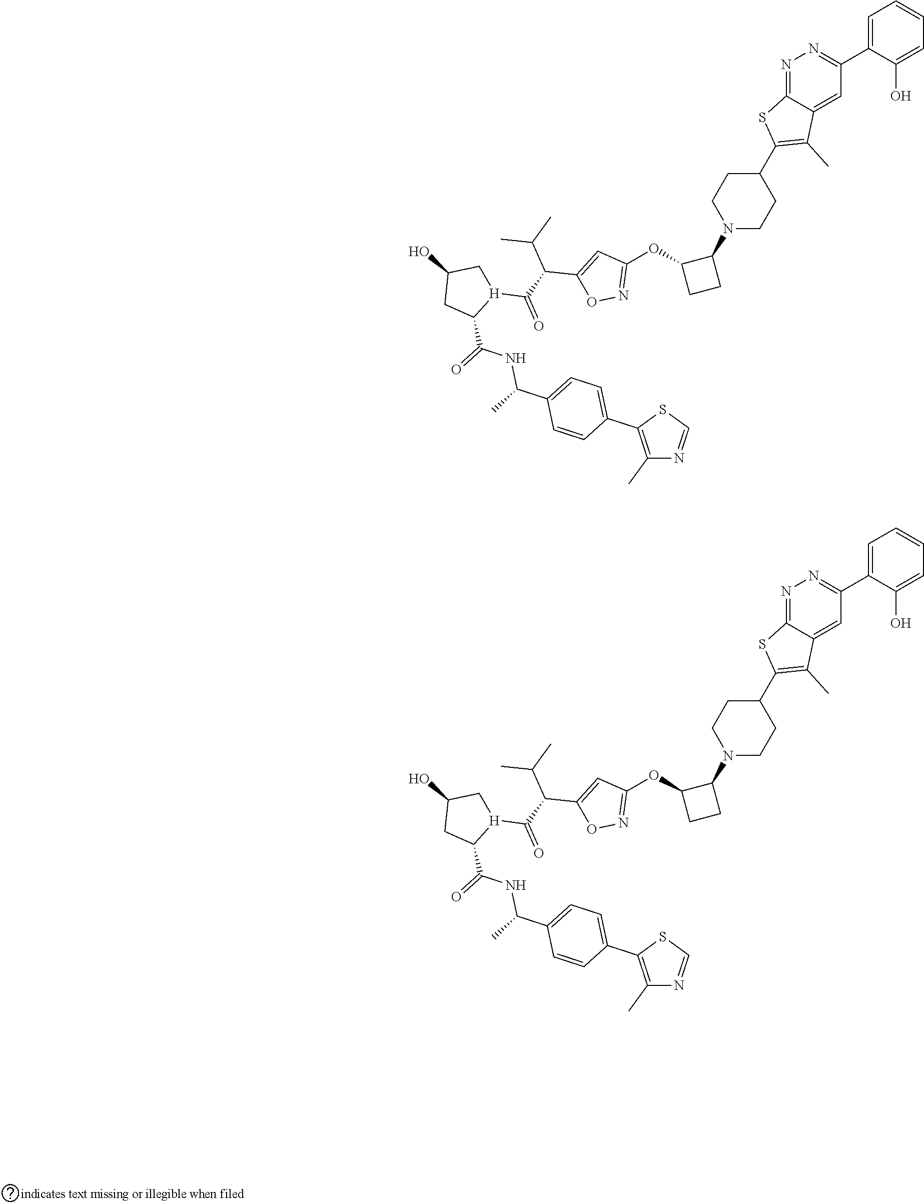

Step 1: Preparation of methyl 3-methyl-2-[3-(2-oxocyclobutoxy)-1,2-oxazol-5-yl]butanoate (Intermediate 2)

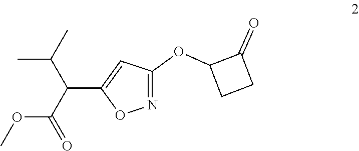 [SMILES: COC(=O)C(c1cc(OC2CCC2=O)no1)C(C)C]

A mixture of methyl 2-(3-hydroxy-1,2-oxazol-5-yl)-3-methylbutanoate (400 mg, 2.008 mmol, 1 equiv), 2-bromocyclobutan-1-one (448.74 mg, 3.012 mmol, 1.5 equiv) and Cs2CO3 (13.1 g, 4.016 mmol, 2 equiv) in acetone (3 mL) was stirred for 2 h at room temperature. The resulting mixture was filtered and the filter cake was washed with acetonitrile (3×2 mL). The filtrate was concentrated under reduced pressure to afford Intermediate 2 (860 mg, crude) as a reddish brown solid. LCMS (ESI) m/z: [M+H]⁺=268.

Step 2: Preparation of methyl 2-[3-(2-{4-[3-(2-hydroxyphenyl)-5-methylthieno[2,3-c]pyridazin-6-yl]piperidin-1-yl}cyclobutoxy)-1,2-oxazol-5-yl]-3-methylbutanoate (Intermediate 3)

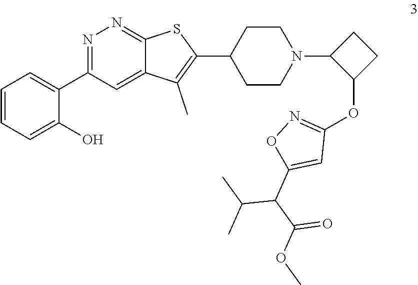 [SMILES: COC(=O)C(c1cc(OC2CCC2N2CCC(c3sc4nnc(-c5ccccc5O)cc4c3C)CC2)no1)C(C)C]

A mixture of Intermediate 2 (400 mg, 1.497 mmol, 1 equiv) and compound I-10 (200 mg, 0.615 mmol, 0.41 equiv) in DMF (5 mL) was stirred for 30 min at room temperature. To the above mixture was added NaBH3CN (282.13 mg, 4.491 mmol, 3 equiv) at room temperature. The resulting mixture was stirred for 3 h at room temperature. The mixture was purified by reverse phase flash chromatography with the following conditions: column, C18 silica gel; mobile phase, MeCN in water (10 mmol/L NH₄HCO₃), 0% to 100% gradient in 30 min; detector, UV 254 nm. This resulted in Intermediate 3 (91 mg, 10.54%) as a yellow solid. LCMS (ESI) m/z [M+H]⁺=577.

Step 3: Preparation of 2-[3-(2-{4-[3-(2-hydroxyphenyl)-5-methylthieno[2,3-c]pyridazin-6-yl]piperidin-1-yl}cyclobutoxy)-1,2-oxazol-5-yl]-3-methylbutanoic acid (Intermediate 4)

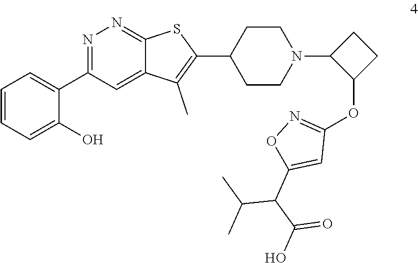 [SMILES: Cc1c(C2CCN(C3CCC3Oc3cc(C(C(=O)O)C(C)C)on3)CC2)sc2nnc(-c3ccccc3O)cc12]

A mixture of Intermediate 3 (91 mg, 0.158 mmol, 1 equiv) and LiOH·H₂O (33.10 mg, 0.790 mmol, 5 equiv) in MeOH (2 mL) and H₂O (2 mL) was stirred for 2 h at room temperature. The mixture was acidified to pH 6 with conc. HCl. The resulting mixture was concentrated under reduced pressure to afford Intermediate 4 (96 mg, crude) as a yellow green solid. LCMS (ESI) m/z: [M+H]⁺=563.

Step 4: Preparation of (2S,4R)-4-hydroxy-1-{2-[3-(2-{4-[3-(2-hydroxyphenyl)-5-methylthieno[2,3-c]pyridazin-6-yl]piperidin-1-yl}cyclobutoxy)-1,2-oxazol-5-yl]-3-methylbutanoyl}-N-[(1S)-1-[4-(4-methyl-1,3-thiazol-5-yl)phenyl]ethyl]pyrrolidine-2-carboxamide (Intermediate 5)

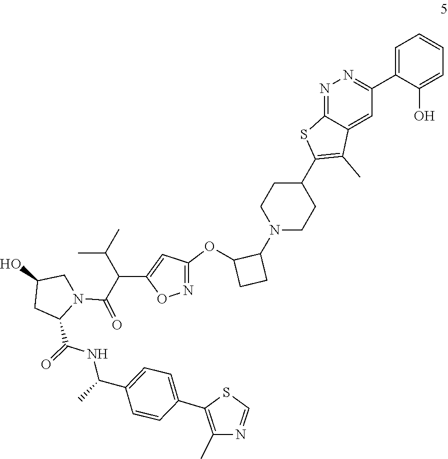 [SMILES: Cc1ncsc1-c1ccc([C@H](C)NC(=O)[C@@H]2C[C@@H](O)CN2C(=O)C(c2cc(OC3CCC3N3CCC(c4sc5nnc(-c6ccccc6O)cc5c4C)CC3)no2)C(C)C)cc1]

A mixture of Intermediate 4 (90 mg, 0.160 mmol, 1 equiv), (2S,4R)-4-hydroxy-N-[(1S)-1-[4-(4-methyl-1,3-thiazol-5-yl)phenyl]ethyl]pyrrolidine-2-carboxamide hydrochloride (53.01 mg, 0.160 mmol, 1 equiv), PyBOP (124.85 mg, 0.240 mmol, 1.5 equiv) and DIEA (103.36 mg, 0.800 mmol, 5 equiv) in DMF (3 mL) was stirred for 3 h at room temperature. The mixture was purified by reverse phase flash chromatography with the following conditions: column, C18 silica gel; mobile phase, MeCN in water (10 mmol/L NH₄HCO₃), 0% to 100% gradient in 30 min; detector, UV 254 nm. This resulted in Intermediate 5 (80 mg, 57.09%) as a light yellow solid. LCMS (ESI) m/z: [M+H]⁺=876.

Step 5: Preparation of (2S,4R)-4-hydroxy-1-[(2S)-2-{3-[(1S,2S)-2-{4-[3-(2-hydroxyphenyl)-5-methylthieno[2,3-c]pyridazin-6-yl]piperidin-1-yl}cyclobutoxy]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 115) and (2S,4R)-4-hydroxy-1-[(2R)-2-[3-(2-{4-[3-(2-hydroxyphenyl)-5-methylthieno[2,3-c]pyridazin-6-yl]piperidin-1-yl}cyclobutoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Intermediate 6)

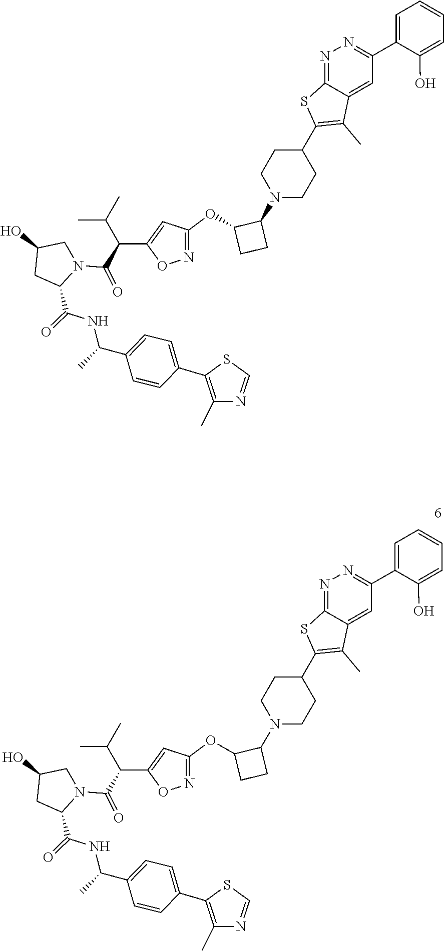 [SMILES: Cc1ncsc1-c1ccc([C@H](C)NC(=O)[C@@H]2C[C@@H](O)CN2C(=O)[C@H](c2cc(O[C@H]3CC[C@@H]3N3CCC(c4sc5nnc(-c6ccccc6O)cc5c4C)CC3)no2)C(C)C)cc1 ; Cc1ncsc1-c1ccc([C@H](C)NC(=O)[C@@H]2C[C@@H](O)CN2C(=O)[C@@H](c2cc(OC3CCC3N3CCC(c4sc5nnc(-c6ccccc6O)cc5c4C)CC3)no2)C(C)C)cc1]

Intermediate 5 was purified by SFC with the following conditions: Column, CHIRAL ART Amylose-SA, 3*25 cm, 5 μm; Mobile Phase A: CO₂, Mobile Phase B: MeOH/DCM 1:1; Flow rate: 60 mL/min; Gradient: isocratic 50% B; Column Temperature (° C.): 35; Back Pressure (bar): 100; Detector: 206 nm; RT1 (min): 5.77; RT2 (min): 10.18. This resulted in:

Compound 115 (35.6 mg, 38.53%) as a white solid. ¹H NMR (400 MHz, DMSO-d6) δ 12.80 (s, 1H), 9.01-8.95 (m, 1H), 8.70-8.65 (m, 1H), 8.28-8.24 (m, 1H), 8.16 (d, J=7.8 Hz, 1H), 7.51-7.27 (m, 5H), 7.06-6.97 (m, 2H), 6.18-6.08 (m, 1H), 5.12-4.84 (m, 2H), 4.69-4.64 (m, 1H), 4.42 (t, J=7.8 Hz, 1H), 4.27 (s, 1H), 3.77 (d, J=8.3 Hz, 1H), 3.57-3.44 (m, 2H), 3.10-2.86 (m, 3H), 2.49-2.40 (m, 7H), 2.28-2.23 (m, 2H), 2.20-2.00 (m, 3H), 2.00-1.54 (m, 7H), 1.49-1.39 (m, 1H), 1.38-1.31 (m, 3H), 1.00-0.74 (m, 6H). LCMS (ESI) m/z: [M+H]⁺=876.30.

Intermediate 6 (20.0 mg, 21.64%) as a white solid. LCMS (ESI) m/z: [M+H]⁺=876.30

Step 6: Preparation of (2S,4R)-4-hydroxy-1-[(2R)-2-{3-[(1S,2S)-2-{4-[3-(2-hydroxyphenyl)-5-methylthieno[2,3-c]pyridazin-6-yl]piperidin-1-yl}cyclobutoxy]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 36) and (2S,4R)-4-hydroxy-1-[(2R)-2-{3-[(1R,2S)-2-{4-[3-(2-hydroxyphenyl)-5-methylthieno[2,3-c]pyridazin-6-yl]piperidin-1-yl}cyclobutoxy]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 35)

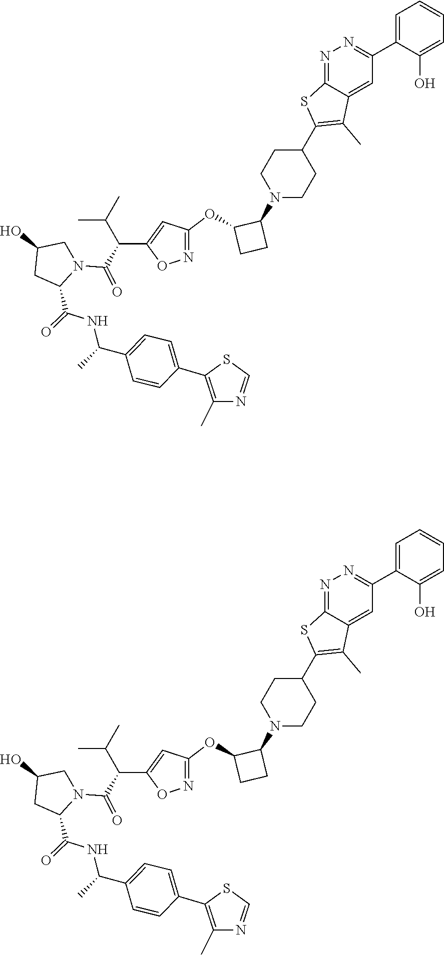 [SMILES: Cc1ncsc1-c1ccc([C@H](C)NC(=O)[C@@H]2C[C@@H](O)CN2C(=O)[C@@H](c2cc(O[C@H]3CC[C@@H]3N3CCC(c4sc5nnc(-c6ccccc6O)cc5c4C)CC3)no2)C(C)C)cc1 ; Cc1ncsc1-c1ccc([C@H](C)NC(=O)[C@@H]2C[C@@H](O)CN2C(=O)[C@@H](c2cc(O[C@@H]3CC[C@@H]3N3CCC(c4sc5nnc(-c6ccccc6O)cc5c4C)CC3)no2)C(C)C)cc1]

Intermediate 6 was purified by chiral HPLC with the following conditions: Column, CHIRALPAK ID, 2*25 cm, 5 μm; Mobile Phase A: MtBE (10 mM NH₃-MeOH), Mobile Phase B: MeOH; Flow rate: 20 mL/min; Gradient: 20% B to 50% B in 11.5 min; Detector: 208/268 nm; RT1 (min): 4.555; RT2 (min): 7.69. This resulted in:

Compound 36 (10.7 mg, 53.07%) as a white solid. ¹H NMR (400 MHz, DMSO-d₆) δ 12.80 (s, 1H), 8.98 (s, 1H), 8.68 (d, J=1.9 Hz, 1H), 8.40 (d, J=7.7 Hz, 1H), 8.19-8.13 (m, 1H), 7.49-7.41 (m, 2H), 7.41-7.33 (m, 3H), 7.06-6.97 (m, 2H), 6.10 (s, 1H), 5.12-5.07 (m, 1H), 4.92 (q, J=7.3 Hz, 1H), 4.74-4.64 (m, 1H), 4.37 (t, J=7.9 Hz, 1H), 4.29 (s, 1H), 3.74-3.62 (m, 2H), 3.60-3.43 (m, 1H), 3.26-3.18 (m, 1H), 3.07-2.92 (m, 3H), 2.48-2.41 (m, 6H), 2.36-2.23 (m, 2H), 2.21-2.00 (m, 3H), 1.99-1.92 (m, 3H), 1.84-1.74 (m, 1H), 1.74-1.55 (m, 3H), 1.52-1.35 (m, 4H), 1.01-0.93 (m, 3H), 0.87-0.77 (m, 3H). LCMS (ESI) m/z: [M+H]⁺=876.40.

Compound 35 (3.2 mg, 15.68%) as a white solid. ¹H NMR (400 MHz, Methanol-d4) δ 8.86 (s, 1H), 8.52 (s, 1H), 8.08-7.98 (m, 1H), 7.48-7.24 (m, 5H), 7.05-6.97 (m, 2H), 6.00 (s, 1H), 5.08-4.93 (m, 1H), 4.79 (s, 1H), 4.55-4.35 (m, 2H), 3.88-3.80 (m, 1H), 3.76-3.58 (m, 3H), 3.56-3.44 (m, 1H), 3.17 (d, J=11.7 Hz, 1H), 3.13-3.03 (m, 2H), 2.49-2.45 (m, 4H), 2.43-2.10 (m, 7H), 2.09-2.00 (m, 3H), 1.99-1.89 (m, 1H), 1.89-1.78 (m, 2H), 1.77-1.67 (m, 1H), 1.62-1.54 (m, 1H), 1.51 (d, J=7.0 Hz, 3H), 1.08-1.02 (m, 3H), 0.94-0.86 (m, 3H). LCMS (ESI) m/z: [M+H]⁺=876.40.

The compounds in Table 9 were prepared using procedures similar to those used above for the preparation of compound Compound 115 using the appropriate amine and ketone.

TABLE 9

| | | LCMS | |
|---|---|---|---|
| Compound | | (ESI) | |
| No. | Name | m/z | ¹H NMR |
| 59 | (2S,4R)-4-hydroxy-1-((R)-2-(3- | 876.5 | 1H NMR (300 MHz, Methanol-d4) δ 8.90 |
| | ((1r,3R)-3-(4-(3-(2- | | (s, 1H), 8.58 (s, 1H), 8.12-8.04 (m, 1H), |
| | hydroxyphenyl)-5- | | 7.51-7.34 (m, 5H), 7.09-6.99 (m, 2H), |
| | methylthieno[2,3-c]pyridazin-6- | | 5.90 (s, 1H), 5.12-4.96 (m, 2H), 4.65- |
| | yl)piperidin-1- | | 4.46 (m, 2H), 3.91-3.66 (m, 4H), 3.61- |
| | yl)cyclobutoxy)isoxazol-5-yl)-3- | | 3.40 (m, 3H), 2.85-2.65 (m, 6H), 2.57- |
| | methylbutanoyl)-N-((S)-1-(4-(4- | | 2.42 (m, 7H), 2.35-2.18 (m, 3H), 2.12- |
| | methylthiazol-5- | | 1.93 (m, 3H), 1.56 (d, J = 7.0 Hz, 3H), |
| | yl)phenyl)ethyl)pyrrolidine-2- | | 1.13 (d, J = 6.6 Hz, 3H), 1.04 (d, J = 6.7 |
| | carboxamide | | Hz, 3H). |
| 61 | (2S,4R)-4-hydroxy-1-((R)-2-(3- | 876.35 | 1H NMR (300 MHz, DMSO-d6) δ 12.82 |
| | ((1s,3S)-3-(4-(3-(2- | | (s, 1H), 8.99 (s, 1H), 8.71 (s, 1H), 8.46 |
| | hydroxyphenyl)-5- | | (d, J = 7.6 Hz, 1H), 8.17 (d, J = 8.0 Hz, |
| | methylthieno[2,3-c]pyridazin-6- | | 1H), 7.50-7.33 (m, 5H), 7.10-6.97 (m, |
| | yl)piperidin-1- | | 2H), 5.88-5.60 (m, 1H), 5.22-5.05 (m, |
| | yl)cyclobutoxy)isoxazol-5-yl)-3- | | 1H), 4.99-4.86 (m, 1H), 4.71-4.21 (m, |
| | methylbutanoyl)-N-((S)-1-(4-(4- | | 3H), 3.78-3.46 (m, 4H), 3.29-2.81 (m, |
| | methylthiazol-5- | | 3H), 2.82-2.63 (m, 2H), 2.49-2.35 (m, |
| | yl)phenyl)ethyl)pyrrolidine-2- | | 7H), 2.33-1.90 (m, 7H), 1.87-1.54 (m, |
| | carboxamide | | 3H), 1.39 (d, J = 7.0 Hz, 3H), 1.00 (d, J = |
| | | | 6.3 Hz, 3H), 0.92 (d, J = 6.7 Hz, 3H). |

Preparation of 3-methyl-2-[3-(morpholin-4-yl)-1,2-oxazol-5-yl]butanoic acid (intermediate A)

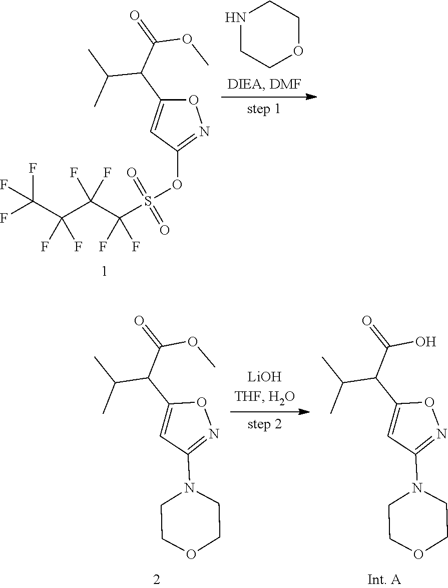

Step 1: Preparation of methyl 3-methyl-2-[3-(morpholin-4-yl)-1,2-oxazol-5-yl]butanoate (intermediate 2)

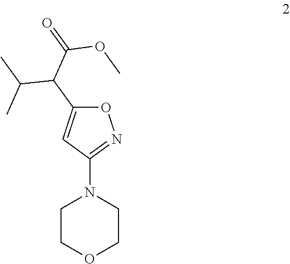 [SMILES: COC(=O)C(c1cc(N2CCOCC2)no1)C(C)C]

To a solution of intermediate 1 (3.5 g, 7.276 mmol, 1 equiv) and DIEA (1184.27 mg, 21.829 mmol, 3 equiv) in DMF (40 mL) was added morpholine (1.33 g, 36.382 mmol, 5 equiv). The solution was stirred at 120° C. for 2 h. The mixture was allowed to cool down to room temperature and was purified by reverse phase flash chromatography with the following conditions: column, C18 silica gel; mobile phase, MeCN in water, 0% to 100% gradient in 20 min; detector, UV 254 nm. This resulted in intermediate 2 (1.06 g, 54.10%) as a colorless oil. LCMS (ESI) m/z: [M+H]⁺=269.

Step 2: Preparation of 3-methyl-2-[3-(morpholin-4-yl)-1,2-oxazol-5-yl]butanoic acid (intermediate A)

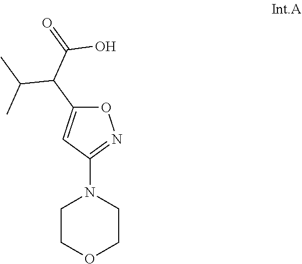 [SMILES: CC(C)C(C(=O)O)c1cc(N2CCOCC2)no1]

To a solution of intermediate 2 (1 g, 3.727 mmol, 1 equiv) in THF (5 mL) and H₂O (5 mL) was added lithium hydroxide (0.13 g, 5.590 mmol, 1.5 equiv). The mixture was stirred at 25° C. for 2 h. The mixture was acidified to pH 6 with conc. HCl. The aqueous layer was extracted with EtOAc (3×30 mL). The combined organic layers were dried over anhydrous sodium sulfate, filtered and concentrated under reduced pressure. This resulted in intermediate A (852 mg, 89.90%) as a white solid. LCMS (ESI) m/z: [M+H]⁺=255.

Preparation of (2S,4R)-4-hydroxy-N-(2-hydroxy-4-(4-methylthiazol-5-yl)benzyl)-1-((R)-3-methyl-2-(3-morpholinoisoxazol-5-yl)butanoyl)pyrrolidine-2-carboxamide (intermediate B)

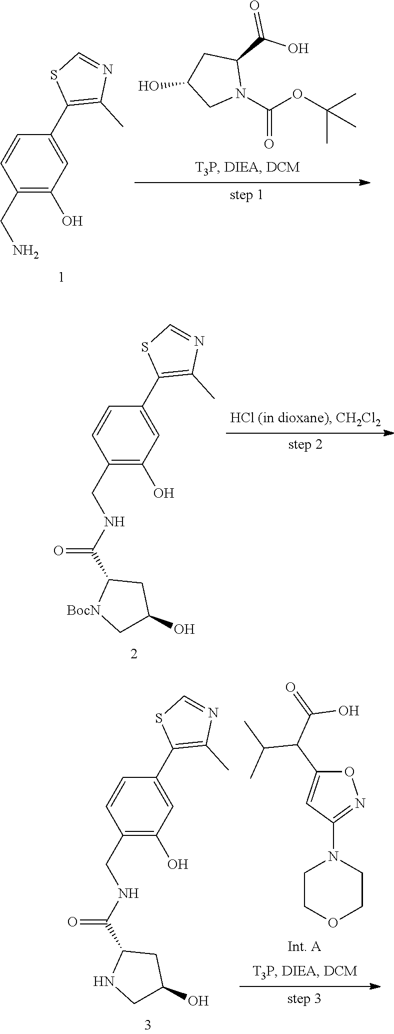

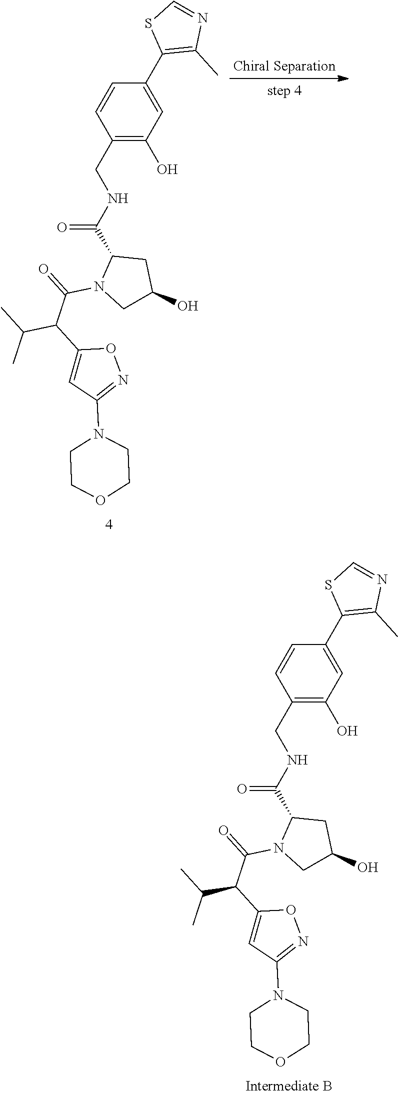 [SMILES: Cc1ncsc1-c1ccc(CNC(=O)[C@@H]2C[C@@H](O)CN2C(=O)C(c2cc(N3CCOCC3)no2)C(C)C)c(O)c1 ; Cc1ncsc1-c1ccc(CNC(=O)[C@@H]2C[C@@H](O)CN2C(=O)[C@@H](c2cc(N3CCOCC3)no2)C(C)C)c(O)c1]

Step 1: Preparation of tert-butyl (2S,4R)-4-hydroxy-2-({[2-hydroxy-4-(4-methyl-1,3-thiazol-5-yl)phenyl]methyl}carbamoyl)pyrrolidine-1-carboxylate (intermediate 2)

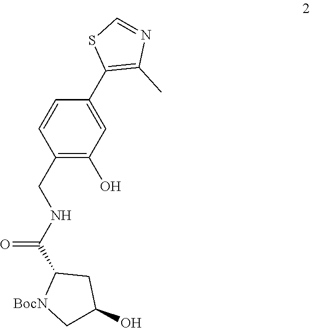 [SMILES: Cc1ncsc1-c1ccc(CNC(=O)[C@@H]2C[C@@H](O)CN2C(=O)OC(C)(C)C)c(O)c1]

To a solution of (2S,4R)-1-(tert-butoxycarbonyl)-4-hydroxypyrrolidine-2-carboxylic acid (2.97 g, 12.853 mmol, 1 equiv) and DIEA (8.31 g, 64.265 mmol, 5 equiv) in DCM (100 mL) at 0° C. was added T₃P (6.13 g, 19.279 mmol, 1.5 equiv). After 30 min, 2-(aminomethyl)-5-(4-methyl-1,3-thiazol-5-yl)phenol hydrochloride (3.3 g, 12.853 mmol, 1 equiv) was added. The solution was stirred at room temperature for 16 h. The resulting mixture was concentrated under reduced pressure. The residue was purified by silica gel column chromatography, eluted with PE/EA (1:1), to afford intermediate 2 (2.85 g, 51.15%) as a yellow oil. LCMS (ESI) m/z: [M+H]⁺=434.

Step 2: Preparation of (2S,4R)-4-hydroxy-N-{[2-hydroxy-4-(4-methyl-1,3-thiazol-5-yl)phenyl]methyl}pyrrolidine-2-carboxamide (intermediate 3)

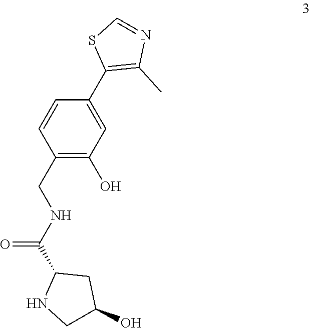 [SMILES: Cc1ncsc1-c1ccc(CNC(=O)[C@@H]2C[C@@H](O)CN2)c(O)c1]

To a solution of intermediate 2 (2.75 g, 6.343 mmol, 1 equiv) in DCM (3 mL) was added HCl in 1,4-dioxane (15 mL) in portion. The solution was stirred at room temperature for 2 h. The resulting mixture was concentrated under reduced pressure. This resulted in intermediate 3 (2.4 g, crude) as a white solid. LCMS (ESI) m/z: [M+H]⁺=334.

Step 3: Preparation of (2S,4R)-4-hydroxy-N-(2-hydroxy-4-(4-methylthiazol-5-yl)benzyl)-1-(3-methyl-2-(3-morpholinoisoxazol-5-yl)butanoyl)pyrrolidine-2-carboxamide (intermediate 4)

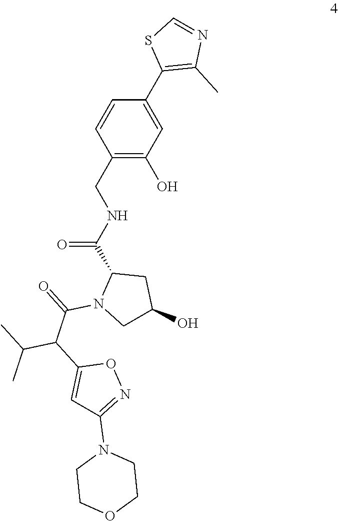 [SMILES: Cc1ncsc1-c1ccc(CNC(=O)[C@@H]2C[C@@H](O)CN2C(=O)C(c2cc(N3CCOCC3)no2)C(C)C)c(O)c1]

To a solution of intermediate A (0.92 g, 3.599 mmol, 1 equiv) and DIEA (2.33 g, 17.995 mmol, 5 equiv) in DCM (20 mL) was added T₃P (1.72 g, 5.399 mmol, 1.5 equiv) dropwise at 0° C. After 30 min, the intermediate 3 (1.2 g, 3.599 mmol, 1.00 equiv) was added. The mixture was stirred at room temperature for 3 h. The resulting mixture was concentrated under reduced pressure. The residue was purified by reverse phase flash chromatography with the following conditions: column, C18 silica gel; mobile phase, MeCN in water (0.1% FA), 10% to 100% gradient in 30 min; detector, UV 254 nm. This resulted in intermediate 4 (800 mg, 39.02%) as a yellow solid. LCMS (ESI) m/z: [M+H]⁺=570.

Step 4: Preparation of (2S,4R)-4-hydroxy-N-(2-hydroxy-4-(4-methylthiazol-5-yl)benzyl)-1-((R)-3-methyl-2-(3-morpholinoisoxazol-5-yl)butanoyl)pyrrolidine-2-carboxamide (intermediate B)

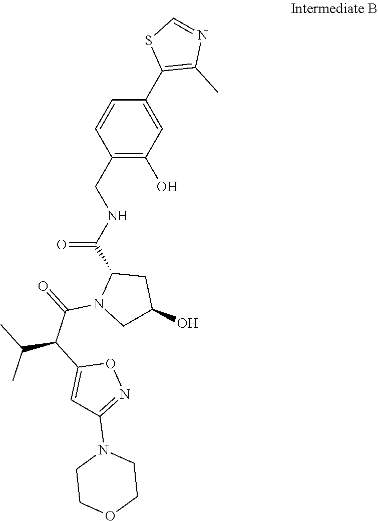 [SMILES: Cc1ncsc1-c1ccc(CNC(=O)[C@@H]2C[C@@H](O)CN2C(=O)[C@@H](c2cc(N3CCOCC3)no2)C(C)C)c(O)c1]

Intermediate 4 (800 mg) was separated by chiral separation to afford intermediate B (second peak) (302.9 mg, 37.37%) as a white solid. LCMS (ESI) m/z: [M+H]⁺=570.10.

Preparation of (2S,4R)-4-hydroxy-N-{[2-(3-{4-[3-(2-hydroxyphenyl)-5-methylthieno[2,3-c]pyridazin-6-yl]piperidin-1-yl}propoxy)-4-(4-methyl-1,3-thiazol-5-yl)phenyl]methyl}-1-[(2S)-3-methyl-2-[3-(morpholin-4-yl)-1,2-oxazol-5-yl]butanoyl]pyrrolidine-2-carboxamide (Compound 55) and (2S,4R)-4-hydroxy-N-{[2-(3-{4-[3-(2-hydroxyphenyl)-5-methylthieno[2,3-c]pyridazin-6-yl]piperidin-1-yl}propoxy)-4-(4-methyl-1,3-thiazol-5-yl)phenyl]methyl)-1-[(2R)-3-methyl-2-[3-(morpholin-4-yl)-1,2-oxazol-5-yl]butanoyl]pyrrolidine-2-carboxamide (Compound 56)

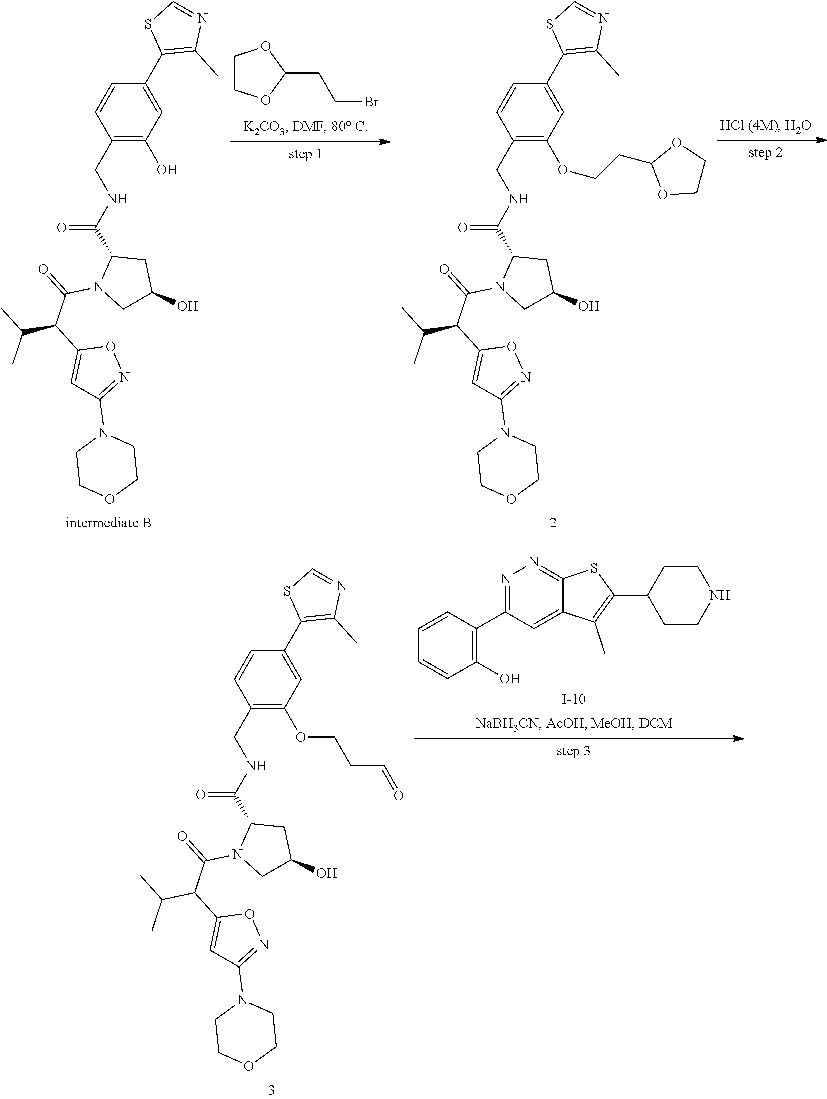

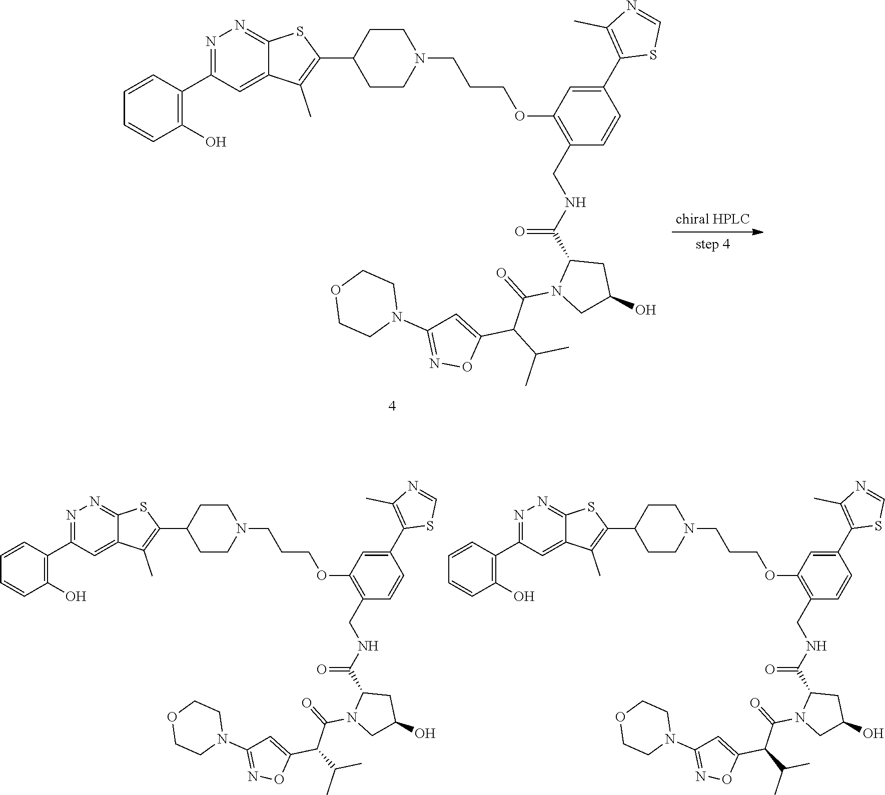

Step 1: Preparation of (2S,4R)—N-({2-[2-(1,3-dioxolan-2-yl)ethoxy]-4-(4-methyl-1,3-thiazol-5-yl)phenyl}methyl)-4-hydroxy-1-{3-methyl-2-[3-(morpholin-4-yl)-1,2-oxazol-5-yl]butanoyl}pyrrolidine-2-carboxamide (Intermediate 2)

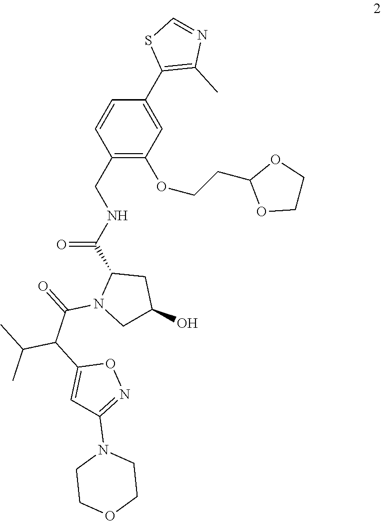 [SMILES: Cc1ncsc1-c1ccc(CNC(=O)[C@@H]2C[C@@H](O)CN2C(=O)C(c2cc(N3CCOCC3)no2)C(C)C)c(OCCC2OCCO2)c1]

To a solution of intermediate B (150.0 mg, 0.263 mmol, 1.00 equiv) and 2-(2-bromoethyl)-1,3-dioxolane (47.7 mg, 0.263 mmol, 1.00 equiv) in DMF (3.0 mL) was added K2CO3 (109.2 mg, 0.789 mmol, 3.00 equiv). After stirring for 2 h at 80° C., the mixture was cooled down to room temperature and was extracted with EA (3×3 mL). The combined organic layers were washed with water (5 mL), then dried over anhydrous Na₂SO₄. After filtration, the filtrate was concentrated under reduced pressure to afford Intermediate 2 (200.0 mg, crude) as a white solid. LCMS (ESI) m/z: [M+H]⁺=670.

Step 2: Preparation of (2S,4R)-4-hydroxy-N-{[4-(4-methyl-1,3-thiazol-5-yl)-2-(3-oxopropoxy)phenyl]methyl}-1-{3-methyl-2-[3-(morpholin-4-yl)-1,2-oxazol-5-yl]butanoyl}pyrrolidine-2-carboxamide (Intermediate 3)

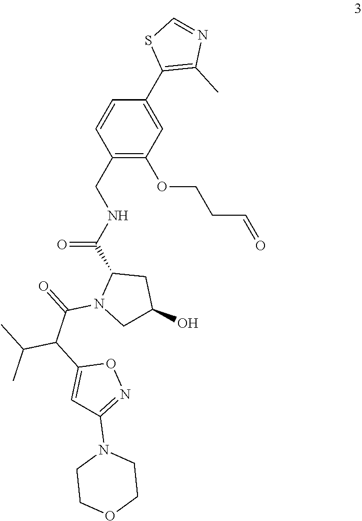 [SMILES: Cc1ncsc1-c1ccc(CNC(=O)[C@@H]2C[C@@H](O)CN2C(=O)C(c2cc(N3CCOCC3)no2)C(C)C)c(OCCC=O)c1]

To a solution of Intermediate 2 (200 mg, crude) in H₂O (3 mL) was added HCl (4 M) (3 mL). After stirring for 1 h at room temperature, the mixture was extracted with EA (3×5 mL). The combined organic layers were washed with brine (5 mL), dried over anhydrous sodium sulfate, filtered and the filtrate was concentrated under reduced pressure. This resulted in Intermediate 3 (147.0 mg, crude) as a white solid. LCMS (ESI) m/z: [M+H]⁺=626.

Step 3: Preparation of (2S,4R)-4-hydroxy-N-{[2-(3-{4-[3-(2-hydroxyphenyl)-5-methylthieno[2,3-c]pyridazin-6-yl]piperidin-1-yl}propoxy)-4-(4-methyl-1,3-thiazol-5-yl)phenyl]methyl}-1-{3-methyl-2-[3-(morpholin-4-yl)-1,2-oxazol-5-yl]butanoyl}pyrrolidine-2-carboxamide (Intermediate 4)

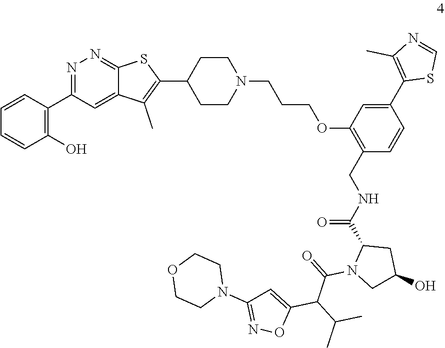 [SMILES: Cc1ncsc1-c1ccc(CNC(=O)[C@@H]2C[C@@H](O)CN2C(=O)C(c2cc(N3CCOCC3)no2)C(C)C)c(OCCCN2CCC(c3sc4nnc(-c5ccccc5O)cc4c3C)CC2)c1]

To a solution of Intermediate 3 (147.0 mg, 0.235 mmol, 1.00 equiv) and compound I-10 (91.8 mg, 0.282 mmol, 1.20 equiv) in MeOH (2.0 mL) and DCM (2.0 mL) was added AcOH (0.05 mL, 0.873 mmol, 3.71 equiv). After stirring for 20 min at room temperature, NaBH₃CN (44.3 mg, 0.705 mmol, 3.00 equiv) was added. After stirring for 2 h at room temperature, the reaction mixture was concentrated under reduced pressure. The residue was purified by reverse phase flash chromatography with the following conditions: column, C18 silica gel; mobile phase, MeCN in water (0.05% FA), 0% to 100% gradient in 30 min; detector, UV 254 nm). This resulted in Intermediate 4 (75.5 mg, 34.37%) as a white solid. LCMS (ESI) m/z: [M+H]⁺=935.

Step 4: Preparation of (2S,4R)-4-hydroxy-N-{[2-(3-{4-[3-(2-hydroxyphenyl)-5-methylthieno[2,3-c]pyridazin-6-yl]piperidin-1-yl}propoxy)-4-(4-methyl-1,3-thiazol-5-yl)phenyl]methyl}-1-[(2S)-3-methyl-2-[3-(morpholin-4-yl)-1,2-oxazol-5-yl]butanoyl]pyrrolidine-2-carboxamide (Compound 55) and (2S,4R)-4-hydroxy-N-{[2-(3-{4-[3-(2-hydroxyphenyl)-5-methylthieno[2,3-c]pyridazin-6-yl]piperidin-1-yl}propoxy)-4-(4-methyl-1,3-thiazol-5-yl)phenyl]methyl}-1-[(2R)-3-methyl-2-[3-(morpholin-4-yl)-1,2-oxazol-5-yl]butanoyl]pyrrolidine-2-carboxamide (Compound 56)

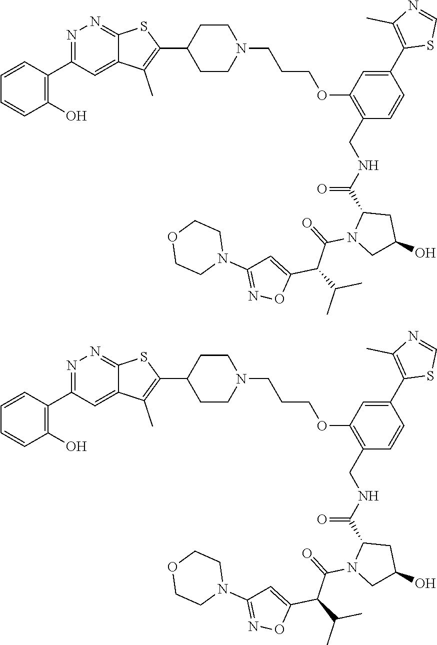 [SMILES: Cc1ncsc1-c1ccc(CNC(=O)[C@@H]2C[C@@H](O)CN2C(=O)[C@@H](c2cc(N3CCOCC3)no2)C(C)C)c(OCCCN2CCC(c3sc4nnc(-c5ccccc5O)cc4c3C)CC2)c1 ; Cc1ncsc1-c1ccc(CNC(=O)[C@@H]2C[C@@H](O)CN2C(=O)[C@H](c2cc(N3CCOCC3)no2)C(C)C)c(OCCCN2CCC(c3sc4nnc(-c5ccccc5O)cc4c3C)CC2)c1]

Intermediate 4 (75.5 mg) was purified by chiral HPLC with the following conditions: Column, CHIRALPAK ID, 2*25 cm, 5 μm; Mobile phase A: MtBE (10 mM NH3-MeOH), Mobile Phase B: EtOH; Flow rate: 18 mL/min; Gradient: 20% B to 50% B in 23 min; Detector: 210/268 nm; RT1 (min): 8.815; RT2 (min): 16.57. This resulted in:

Compound 55 (second peak) (18.7 mg, 24.7%) as a white solid. ¹H NMR (400 MHz, DMSO-d6) δ 12.81 (s, 1H), 9.00 (s, 1H), 8.69 (s, 1H), 8.31 (t, J=6.0 Hz, 1H), 8.20-8.12 (m, 1H), 7.43-7.34 (m, 1H), 7.34-7.26 (m, 1H), 7.10-6.94 (m, 4H), 6.15 (s, 1H), 5.15 (d, J=3.7 Hz, 1H), 4.61-4.45 (m, 1H), 4.40-4.32 (m, 1H), 4.33-4.19 (m, 2H), 4.20-4.05 (m, 2H), 3.72 (d, J=8.9 Hz, 1H), 3.66 (t, J=4.8 Hz, 1H), 3.63-3.49 (m, 5H), 3.22-3.10 (m, 3H), 3.10-2.96 (m, 5H), 2.47-2.42 (m, 6H), 2.36-1.82 (m, 10H), 1.82-1.58 (m, 2H), 0.97 (d, J=6.6 Hz, 3H), 0.83 (d, J=6.7 Hz, 3H). LCMS (ESI) m/z: [M+H]⁺=935.15.

Compound 56 (first peak) (5.5 mg, 7.3%) as a white solid. ¹H NMR (400 MHz, DMSO-d6) δ 12.80 (s, 1H), 9.00 (s, 1H), 8.69 (s, 1H), 8.39 (t, J=5.9 Hz, 1H), 8.20-8.12 (m, 1H), 7.43-7.29 (m, 2H), 7.14-6.95 (m, 4H), 6.18 (s, 1H), 5.14 (d, J=3.7 Hz, 1H), 4.47-4.20 (m, 4H), 4.14 (t, J=6.0 Hz, 2H), 3.83-3.73 (m, 1H), 3.73-3.56 (m, 5H), 3.51 (s, 1H), 3.49-3.39 (m, 2H), 3.17-3.11 (m, 4H), 3.12-3.04 (m, 2H), 2.72-2.63 (m, 1H), 2.47-2.42 (m, 6H), 2.38-2.12 (m, 3H), 2.10-1.86 (m, 6H), 1.82-1.62 (m, 2H), 0.94 (d, J=9.3, 6.6 Hz, 3H), 0.80 (d, J=7.8 Hz, 3H). LCMS (ESI) m/z: [M+H]⁺=935.15.

The compounds in Table 10 were prepared using procedures similar to those used above for the preparation of compound 55 using the appropriate amine and aldehyde.

TABLE 10

| | | LCMS | |
|---|---|---|---|
| Compound | | (ESI) | |
| No. | Name | m/z | ¹H NMR |
| 57 | (2S,4R)-4-hydroxy-N-(2-(3-(4-(3- | 864.15 | 1H NMR (400 MHz, DMSO-d6) δ 12.84 |
| | (2-hydroxyphenyl)-5- | | (br s, 1H), 9.00 (s, 1H), 8.69 (s, 1H), 8.39 |
| | methylthieno[2,3-c]pyridazin-6- | | (t, J = 5.9 Hz, 1H), 8.21-8.11 (m, 1H), |
| | yl)piperidin-1-yl)propoxy)-4-(4- | | 7.43-7.29 (m, 2H), 7.11-6.93 (m, 4H), |
| | methylthiazol-5-yl)benzyl)-1-((R)- | | 6.23 (s, 1H), 5.21-4.98 (m, 1H), 4.51- |
| | 3-methyl-2-(3-methylisoxazol-5- | | 4.17 (m, 4H), 4.13 (t, J = 5.8 Hz, 2H), |
| | yl)butanoyl)pyrrolidine-2- | | 3.84-3.69 (m, 2H), 3.61-3.49 (m, 1H), |
| | carboxamide | | 3.28-3.18 (m, 1H), 3.05 (d, J = 10.9 Hz, |
| | | | 2H), 2.57-2.52 (m, 2H), 2.49 (s, 3H), |
| | | | 2.44 (s, 3H), 2.33-2.23 (m, 1H), 2.20 (s, |
| | | | 2H), 2.16-2.12 (m, 1H), 2.11-2.09 (m, |
| | | | 1H), 2.08 (s, 1H), 2.06-2.01 (m, 1H), |
| | | | 2.00-1.88 (m, 5H), 1.76-1.62 (m, 2H), |
| | | | 0.95 (d, J = 6.5 Hz, 3H), 0.77 (d, J = 6.5 |
| | | | Hz, 3H). |
| 76 | (2S,4R)-1-((S)-2-(1- | 884.2 | 1H NMR (300 MHz, DMSO-d6) δ 12.99 |
| | fluorocyclopropane-1- | | (s, 1H), 9.14 (s, 1H), 8.98 (s, 1H), 8.51 (t, |
| | carboxamido)-3,3- | | J = 5.8 Hz, 1H), 8.06-7.98 (m, 1H), 7.69 |
| | dimethylbutanoyl)-4-hydroxy-N-(2- | | (s, 1H), 7.46-7.33 (m, 2H), 7.32-7.25 |
| | (3-(4-(3-(2- | | (m, 1H), 7.07-7.00 (m, 3H), 7.00-6.92 |
| | hydroxyphenyl)thieno[3,2- | | (m, 1H), 5.18 (d, J = 3.6 Hz, 1H), 4.60 (d, |
| | c]pyridazin-6-yl)piperidin-1- | | J = 9.2 Hz, 1H), 4.52 (t, J = 8.3 Hz, 1H), |
| | yl)propoxy)-4-(4-methylthiazol-5- | | 4.38-4.21 (m, 3H), 4.15-4.07 (m, 2H), |
| | yl)benzyl)pyrrolidine-2- | | 3.67-3.59 (m, 2H), 3.08-2.98 (m, 3H), |
| | carboxamide | | 2.58-2.54 (m, 2H), 2.47 (s, 3H), 2.17- |
| | | | 2.03 (m, 5H), 1.99-1.90 (m, 3H), 1.84- |
| | | | 1.71 (m, 2H), 1.44-1.29 (m, 2H), 1.27- |
| | | | 1.18 (m, 2H), 0.96 (s, 9H). |
| 77 | (2S,4R)-1-((S)-2-(1- | 870.25 | 1H NMR (400 MHz, DMSO-d6) δ 12.98 |
| | fluorocyclopropane-1- | | (s, 1H), 9.14 (s, 1H), 8.99 (s, 1H), 8.51 (t, |
| | carboxamido)-3,3- | | J = 6.0 Hz, 1H), 8.08-7.96 (m, 1H), 7.69 |
| | dimethylbutanoyl)-4-hydroxy-N-(2- | | (s, 1H), 7.45-7.33 (m, 2H), 7.29 (d, J = |
| | (2-(4-(3-(2- | | 9.1 Hz, 1H), 7.10-6.95 (m, 4H), 5.18 (d, |
| | hydroxyphenyl)thieno[3,2- | | J = 3.6 Hz, 1H), 4.59 (d, J = 9.3 Hz, 1H), |
| | c]pyridazin-6-yl)piperidin-1- | | 4.52 (t, J = 8.3 Hz, 1H), 4.39-4.28 (m, |
| | yl)ethoxy)-4-(4-methylthiazol-5- | | 2H), 4.23 (d, J = 12.5 Hz, 3H), 3.63 (q, J = |
| | yl)benzyl)pyrrolidine-2- | | 11.0 Hz, 2H), 3.11 (d, J = 11.4 Hz, 3H), |
| | carboxamide | | 2.84 (d, J = 6.0 Hz, 2H), 2.47 (s, 3H), |
| | | | 2.29 (t, J = 12.1 Hz, 2H), 2.08 (d, J = 12.0 |
| | | | Hz, 3H), 1.94 (d, J = 8.9 Hz, 1H), 1.78 (q, |
| | | | J = 12.1, 11.7 Hz, 2H), 1.35 (dt, J = 17.4, |
| | | | 10.7 Hz, 2H), 1.21 (d, J = 9.6 Hz, 2H), |
| | | | 0.95 (s, 9H). |
| 83 | (2S,4R)-1-((S)-2-(1- | 884.5 | 1H NMR (400 MHz, DMSO-d6) δ 12.82 |
| | fluorocyclopropane-1- | | (s, 1H), 9.00 (s, 1H), 8.69 (s, 1H), 8.52 (t, |
| | carboxamido)-3,3- | | J = 5.9 Hz, 1H), 8.21-8.11 (m, 1H), 7.44- |
| | dimethylbutanoyl)-4-hydroxy-N-(2- | | 7.34 (m, 2H), 7.29 (dd, J = 9.1, 2.8 Hz, |
| | (2-(4-(3-(2-hydroxyphenyl)-5- | | 1H), 7.08 (d, J = 1.7 Hz, 1H), 7.05-6.95 |
| | methylthieno[2,3-c]pyridazin-6- | | (m, 3H), 5.18 (d, J = 3.6 Hz, 1H), 4.62- |
| | yl)piperidin-1-yl)ethoxy)-4-(4- | | 4.48 (m, 2H), 4.28 (ddd, J = 35.1, 17.7, |
| | methylthiazol-5- | | 6.7 Hz, 5H), 3.69-3.56 (m, 2H), 3.25 (s, |
| | yl)benzyl)pyrrolidine-2- | | 1H), 3.13 (d, J = 10.9 Hz, 2H), 2.85 (s, |
| | carboxamide | | 2H), 2.48 (s, 3H), 2.44 (s, 3H), 2.33 (dd, |
| | | | J = 4.0, 2.0 Hz, 2H), 2.16-1.88 (m, 4H), |
| | | | 1.72 (q, J = 12.1 Hz, 2H), 1.45-1.29 (m, |
| | | | 2H), 1.27-1.13 (m, 2H), 0.94 (s, 9H). |
| 81 | (2S,4R)-1-((S)-2-(1- | 898.3 | 1H NMR (300 MHz, DMSO-d6) δ 12.90 |
| | fluorocyclopropane-1- | | (s, 1H), 9.07 (s, 1H), 8.77 (s, 1H), 8.62- |
| | carboxamido)-3,3- | | 8.56 (m, 1H), 8.29-8.20 (m, 1H), 7.53- |
| | dimethylbutanoyl)-4-hydroxy-N-(2- | | 7.33 (m, 3H), 7.15-7.00 (m, 4H), 5.25 |
| | (3-(4-(3-(2-hydroxyphenyl)-5- | | (d, J = 3.5 Hz, 1H), 4.72-4.54 (m, 2H), |
| | methylthieno[2,3-c]pyridazin-6- | | 4.46-4.15 (m, 5H), 3.73-3.67 (m, 2H), |
| | yl)piperidin-1-yl)propoxy)-4-(4- | | 3.36-3.29 (m, 1H), 3.17-3.07 (m, 2H), |
| | methylthiazol-5- | | 2.69-2.64 (m, 2H), 2.55 (s, 3H), 2.52 (s, |
| | yl)benzyl)pyrrolidine-2- | | 3H), 2.27-2.13 (m, 3H), 2.07-1.97 (m, |
| | carboxamide | | 5H), 1.82-1.73 (m, 2H), 1.49-1.36 (m, |
| | | | 2H), 1.34-1.25 (m, 2H), 1.03 (s, 9H). |

Preparation of (2S,4R)-4-hydroxy-1-[(2R)-2-{3-[3-({4-[3-(2-hydroxyphenyl)-5-methylthieno[2,3-c]pyridazin-6-yl]piperidin-1-yl}methyl)bicyclo[1.1.1]pentan-1-yl]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 72)

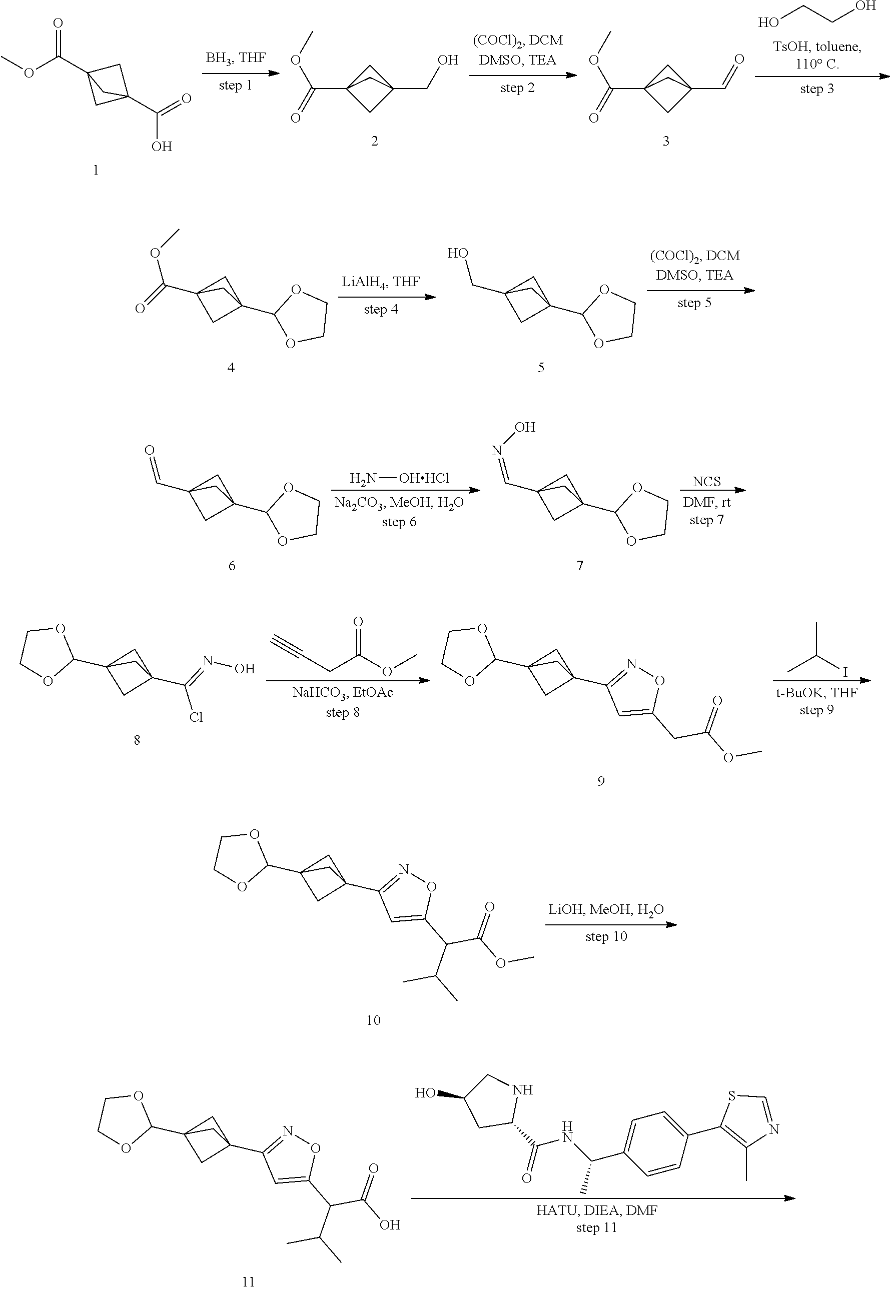

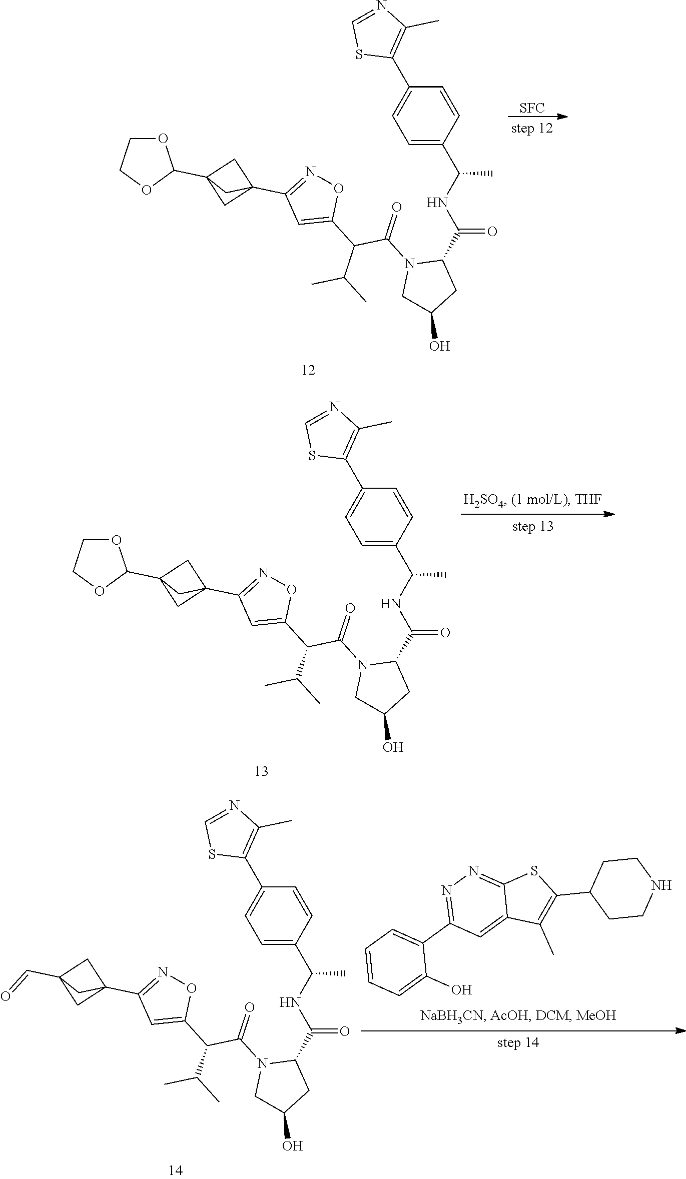

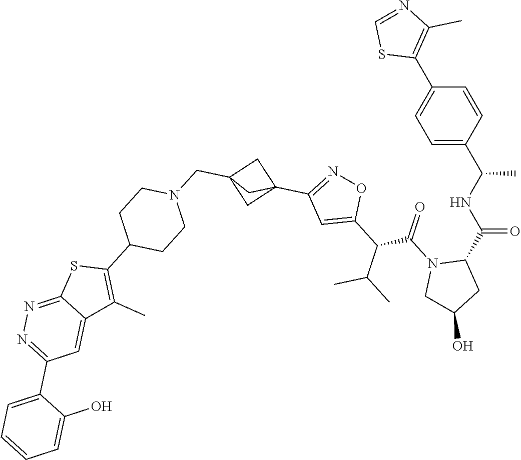 [SMILES: Cc1ncsc1-c1ccc([C@H](C)NC(=O)[C@@H]2C[C@@H](O)CN2C(=O)[C@@H](c2cc(C34CC(CN5CCC(c6sc7nnc(-c8ccccc8O)cc7c6C)CC5)(C3)C4)no2)C(C)C)cc1]

Step 1: Preparation of methyl 3-(hydroxymethyl)bicyclo[1.1.1]pentane-1-carboxylate (Intermediate 2)

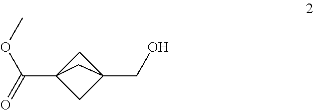

To a stirred solution of 3-(methoxycarbonyl)bicyclo[1.1.1]pentane-1-carboxylic acid (10 g, 58.767 mmol, 1.00 equiv) in THF (50 mL) was added BH₃-THF (147 mL, 293.835 mmol, 5.0 equiv) dropwise at 0° C. under nitrogen atmosphere. The resulting mixture was stirred for 16 h at room temperature under nitrogen atmosphere. The reaction was quenched with water at 0° C. The resulting mixture was extracted with EA (2×100 mL). The combined organic layers were washed with brine (100 mL), then dried over anhydrous Na₂SO₄. After filtration, the filtrate was concentrated under reduced pressure. This resulted in Intermediate 2 (8.4 g, 91.52%) as a colorless oil. LCMS (ESI) m/z: [M+H]⁺=157.

Step 2: Preparation of methyl 3-formylbicyclo[1.1.1]pentane-1-carboxylate (Intermediate 3)

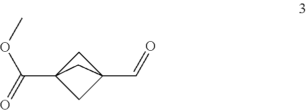

To a stirred solution of (COCl)₂ (11.39 g, 89.74 mmol, 2.00 equiv) in CH₂Cl₂ (100 mL) was added DMSO (10.5 g, 134.62 mmol, 3 equiv) at −78° C. under argon atmosphere. The resulting mixture was stirred for 30 min at −78° C. under nitrogen atmosphere. To the above mixture was added Intermediate 2 (7.0 g, 44.820 mmol, 1.00 equiv) at −78° C. under nitrogen atmosphere. The resulting mixture was stirred for additional 30 min at −78° C. under nitrogen atmosphere. To the above mixture was added TEA (31 mL, 223.026 mmol, 4.98 equiv) dropwise at −78° C. The resulting mixture was stirred for additional 1 h from −78° C. to room temperature under nitrogen atmosphere. The reaction was quenched with water at room temperature and extracted with DCM (2×100 mL). The combined organic layers were washed with brine (100 mL), then dried over anhydrous Na₂SO₄. After filtration, the filtrate was concentrated under reduced pressure. This resulted in Intermediate 3 (7 g, crude) as a colorless oil. LCMS (ESI) m/z: [M+H]⁺=155.

Step 3: Preparation of methyl 3-(1,3-dioxolan-2-yl)bicyclo[1.1.1]pentane-1-carboxylate (Intermediate 4)

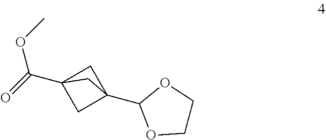

To a stirred mixture of Intermediate 3 (3.90 g, 25.298 mmol, 1 equiv) and ethylene glycol (3.14 g, 50.596 mmol, 2.0 equiv) in toluene (50 mL) was added TsOH (435.63 mg, 2.530 mmol, 0.1 equiv) at room temperature. The resulting mixture was stirred for 5 h at 110° C. The resulting mixture was concentrated under reduced pressure. The residue was purified by silica gel column chromatography, eluted with PE/EA (5:1), to afford Intermediate 4 (1.8 g, 34.46%) as a light yellow oil. LCMS (ESI) m/z: [M+H]⁺=199.

Step 4: Preparation of (3-(1,3-dioxolan-2-yl)bicyclo[1.1.1]pentan-1-yl)methanol (Intermediate 5)

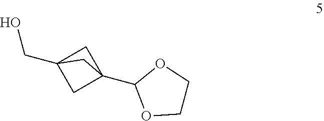 [SMILES: OCC12CC(C3OCCO3)(C1)C2]

To a stirred solution of Intermediate 4 (1.6 g, 8.072 mmol, 1 equiv) in THF (20 mL) was added LiAlH₄ (612.72 mg, 16.144 mmol, 2.0 equiv) dropwise at 0° C. The resulting mixture was stirred overnight at room temperature. The resulting mixture was diluted with EtOAc (20 mL). The resulting mixture was filtered and the filter cake was washed with EtOAc (3×30 mL). The filtrate was concentrated under reduced pressure. The residue was purified by silica gel column chromatography, eluted with PE/EA (3:1), to afford intermediate 5 (1.1 g, 76.86%) as a light yellow oil. LCMS (ESI) m/z: [M+H]⁺=171.

Step 5: Preparation of 3-(1,3-dioxolan-2-yl)bicyclo[1.1.1]pentane-1-carbaldehyde (Intermediate 6)

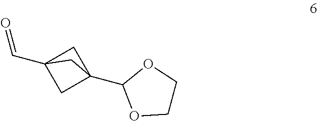 [SMILES: O=CC12CC(C3OCCO3)(C1)C2]

To a stirred solution of (COCl)₂ (1.36 g, 10.693 mmol, 1.3 equiv) in DCM (50 mL) was added DMSO (1.67 g, 21.385 mmol, 2.6 equiv) dropwise at −60° C. under nitrogen atmosphere. The resulting mixture was stirred for 40 min at −60° C. under nitrogen atmosphere. Then intermediate 5 (1.4 g, 8.225 mmol, 1 equiv) was added at −60° C. under nitrogen atmosphere and the mixture was stirred for 40 min at −60° C. under nitrogen atmosphere. To the above mixture was added TEA (4.99 g, 49.350 mmol, 6.0 equiv) dropwise over 4 min at −60° C. The resulting mixture was stirred for additional 30 min at −50° C. The resulting mixture was diluted with water (100 mL). The resulting mixture was extracted with CH₂Cl₂ (2×300 mL). The combined organic layers were washed with brine (100 mL), then dried over anhydrous Na₂SO₄. After filtration, the filtrate was concentrated under reduced pressure to afford Intermediate 6 (1.2 g, 74.60%) as a light yellow oil. LCMS (ESI) m/z: [M+H]⁺=169.

Step 6: Preparation of (Z)-3-(1,3-dioxolan-2-yl)bicyclo[1.1.1]pentane-1-carbaldehyde oxime (Intermediate 7)

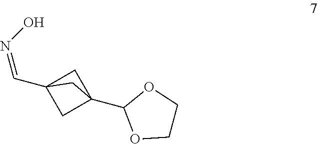 [SMILES: O/N=C\C12CC(C3OCCO3)(C1)C2]

To a stirred solution of Intermediate 6 (970 mg, 5.767 mmol, 1 equiv) and hydroxylamine (228.59 mg, 6.920 mmol, 1.2 equiv) in MeOH (5 mL) and H₂O (5 mL) was added Na2CO3 (305.63 mg, 2.884 mmol, 0.5 equiv) at room temperature. The resulting mixture was stirred for 4 h at room temperature. The resulting mixture was diluted with water (100 mL). The resulting mixture was extracted with EtOAc (2×300 mL). The combined organic layers were washed with brine (100 mL), then dried over anhydrous Na₂SO₄. After filtration, the filtrate was concentrated under reduced pressure to afford Intermediate 7 (960 mg, 82.68%) as a light yellow oil. The crude product was used in the next step directly without further purification. LCMS (ESI) m/z: [M+H]⁺=184.

Step 7: Preparation of (Z)-3-(1,3-dioxolan-2-yl)-N-hydroxybicyclo[1.1.1]pentane-1-carbonimidoyl chloride (Intermediate 8)

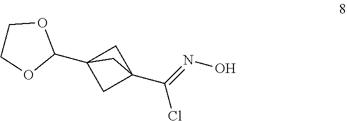 [SMILES: O/N=C(\Cl)C12CC(C3OCCO3)(C1)C2]

To a stirred solution of Intermediate 7 (950.00 mg, 5.185 mmol, 1 equiv) in DMF (10 mL) was added NCS (761.66 mg, 5.704 mmol, 1.1 equiv) at room temperature. The resulting mixture was stirred for 4 h at room temperature. The resulting mixture was diluted with water (100 mL). The resulting mixture was extracted with EtOAc (2×300 mL). The combined organic layers were washed with brine (100 mL), then dried over anhydrous Na₂SO₄. After filtration, the filtrate was concentrated under reduced pressure to afford Intermediate 8 (1.1 g, 88.69%) as a light yellow oil. The crude product was used in the next step directly without further purification. LCMS (ESI) m/z: [M+H]⁺=218.

Step 8: Preparation of methyl 2-{3-[3-(1,3-dioxolan-2-yl)bicyclo[1.1.1]pentan-1-yl]-1,2-oxazol-5-yl}acetate (Intermediate 9)

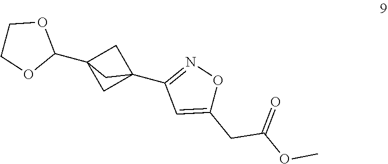 [SMILES: COC(=O)Cc1cc(C23CC(C4OCCO4)(C2)C3)no1]

A solution of Intermediate 8 (980.00 mg, 4.503 mmol, 1 equiv) and NaHCO₃ (567.38 mg, 6.755 mmol, 1.5 equiv) in EtOAc (10 mL) was stirred for 1 h at room temperature. To the above mixture was added methyl but-3-ynoate (441.71 mg, 4.503 mmol, 1.0 equiv) at 0° C. The resulting mixture was stirred overnight at room temperature. The resulting mixture was concentrated under reduced pressure. The residue was purified by reverse phase flash chromatography with the following conditions: column, C18 silica gel; mobile phase, ACN in water, 0% to 100% gradient in 30 min; detector, UV 220 nm. This resulted in Intermediate 9 (270 mg, 20.61%) as a light yellow oil. LCMS (ESI) m/z: [M+H]⁺=280.

Step 9: Preparation of methyl 2-(3-(3-(1,3-dioxolan-2-yl)bicyclo[1.1.1]pentan-1-yl)isoxazol-5-yl)-3-methylbutanoate (Intermediate 10)

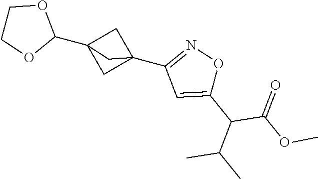 [SMILES: COC(=O)C(c1cc(C23CC(C4OCCO4)(C2)C3)no1)C(C)C]

To a stirred mixture of Intermediate 9 (200.00 mg, 0.716 mmol, 1 equiv) and 2-iodopropane (182.60 mg, 1.074 mmol, 1.5 equiv) in THF (5 mL) were added t-BuOK (241.07 mg, 2.148 mmol, 3.0 equiv) and Na₂SO₄ (200.00 mg, 1.408 mmol, 1.97 equiv) at 0° C. The resulting mixture was stirred overnight at room temperature. The resulting mixture was concentrated under reduced pressure. The residue was dissolved in DMF (3 mL) and was purified by reverse phase flash chromatography with the following conditions: column, C18 silica gel; mobile phase, MeCN in water, 0% to 100% gradient in 30 min; detector, UV 254 nm. This resulted in Intermediate 10 (40 mg, 17.39%) as a light yellow oil. LCMS (ESI) m/z: [M+H]⁺=322.

Step 10: Preparation of 2-(3-(3-(1,3-dioxolan-2-yl)bicyclo[1.1.1]pentan-1-yl)isoxazol-5-yl)-3-methylbutanoic acid (Intermediate 11)

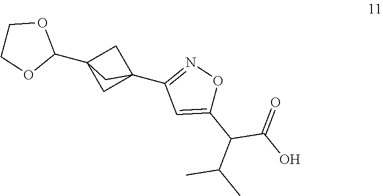 [SMILES: CC(C)C(C(=O)O)c1cc(C23CC(C4OCCO4)(C2)C3)no1]

A mixture of Intermediate 10 (600 mg, 1.87 mmol, 1.00 equiv) and LiOH (224 mg, 9.34 mmol, 5 equiv) in MeOH (2 mL) and H₂O (6 mL) was stirred for 2 h at room temperature. The mixture was acidified to pH 5 with conc. HCl, then extracted with EtOAc (3×20 mL). The combined organic layers were washed with brine (50 mL), then dried over anhydrous Na₂SO₄ and filtered. The filtrate was concentrated under reduced pressure. The crude Intermediate 11 (516 mg) was used in the next step directly without further purification. LCMS (ESI) m/z: [M+H]+=308.

Step 11: Preparation of (2S,4R)-1-(2-{3-[3-(1,3-dioxolan-2-yl)bicyclo[1.1.1]pentan-1-yl]-1,2-oxazol-5-yl}-3-methylbutanoyl)-4-hydroxy-N-[(1S)-1-[4-(4-methyl-1,3-thiazol-5-yl)phenyl]ethyl]pyrrolidine-2-carboxamide (Intermediate 12)

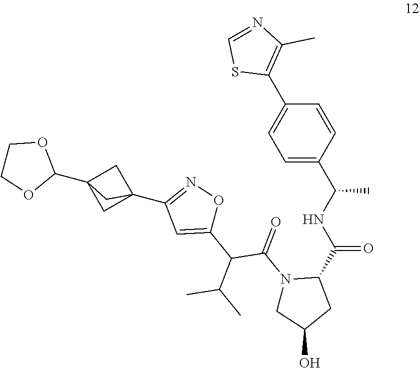 [SMILES: Cc1ncsc1-c1ccc([C@H](C)NC(=O)[C@@H]2C[C@@H](O)CN2C(=O)C(c2cc(C34CC(C5OCCO5)(C3)C4)no2)C(C)C)cc1]

A mixture of Intermediate 11 (400 mg, 1.301 mmol, 1 equiv) and HATU (742.29 mg, 1.951 mmol, 1.5 equiv) in DMF (5 mL) was stirred for 30 min at room temperature. To the above mixture was added (2S,4R)-4-hydroxy-N-[(1S)-1-[4-(4-methyl-1,3-thiazol-5-yl)phenyl]ethyl]pyrrolidine-2-carboxamide (517.61 mg, 1.561 mmol, 1.2 equiv) and DIEA (672.84 mg, 5.204 mmol, 4 equiv) at room temperature. The resulting mixture was stirred for additional 2 h at room temperature. The mixture was purified by reverse phase flash chromatography with the following conditions: column, C18 silica gel; mobile phase, MeCN in water, 0% to 100% gradient in 30 min; detector, UV 254 nm. This resulted in Intermediate 12 (560 mg, 69.31%) as a light brown solid. LCMS (ESI) m/z: [M+H]⁺=621.

Step 12: Preparation of (2S,4R)-1-((R)-2-(3-(3-(1,3-dioxolan-2-yl)bicyclo[1.1.1]pentan-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (Intermediate 13)

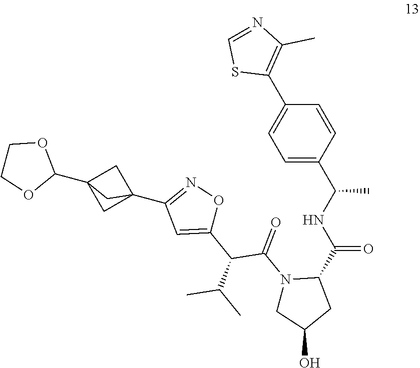 [SMILES: Cc1ncsc1-c1ccc([C@H](C)NC(=O)[C@@H]2C[C@@H](O)CN2C(=O)[C@@H](c2cc(C34CC(C5OCCO5)(C3)C4)no2)C(C)C)cc1]

Intermediate 12 was purified by Prep-SFC with the following conditions: Column, CHIRAL ART Amylose-SA, 3*25 cm, 5 μm; Mobile Phase A: CO₂, Mobile Phase B: IPA; Flow rate: 50 mL/min; Gradient: isocratic 30% B; Column Temperature (° C.): 35; Back Pressure (bar): 100; Detector: 211 nm; RT1 (min): 6.2; RT2 (min): 7.24. This resulted in Intermediate 13 (first peak) (300 mg, 53.57%) as a light yellow oil and (2S,4R)-1-[(2S)-2-{3-[3-(1,3-dioxolan-2-yl)bicyclo[1.1.1]pentan-1-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (second peak) (260 mg, 46.43%) as a white solid. LCMS (ESI) m/z: [M+H]⁺=621.

Step 13: Preparation of (2S,4R)-1-[(2R)-2-(3-{3-formylbicyclo[1.1.1]pentan-1-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (Intermediate 14)

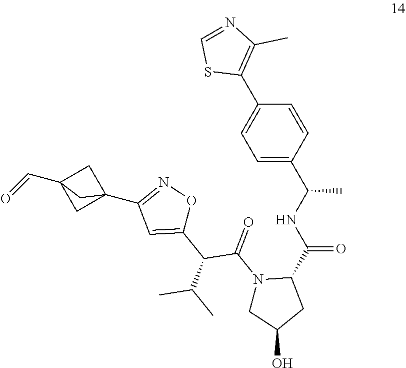 [SMILES: Cc1ncsc1-c1ccc([C@H](C)NC(=O)[C@@H]2C[C@@H](O)CN2C(=O)[C@@H](c2cc(C34CC(C=O)(C3)C4)no2)C(C)C)cc1]

A mixture of Intermediate 13 (13 mg, 0.021 mmol, 1 equiv) in H₂SO₄ (1 mol/L) (1 mL) and THF (1 mL) was stirred for 1 h at 60° C. The mixture was basified to pH 8 with saturated NaHCO₃ (aq.). The resulting mixture was extracted with EtOAc (3×10 mL). The combined organic layers were washed with brine (3×5 mL), then dried over anhydrous Na₂SO₄. After filtration, the filtrate was concentrated under reduced pressure to afford Intermediate 14 (10 mg, 82.80%) as a white solid. LCMS (ESI) m/z: [M+H]⁺=577.

Step 14: Preparation of (2S,4R)-4-hydroxy-1-[(2R)-2-{3-[3-({4-[3-(2-hydroxyphenyl)-5-methylthieno[2,3-c]pyridazin-6-yl]piperidin-1-yl}methyl)bicyclo[1.1.1]pentan-1-yl]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 72)

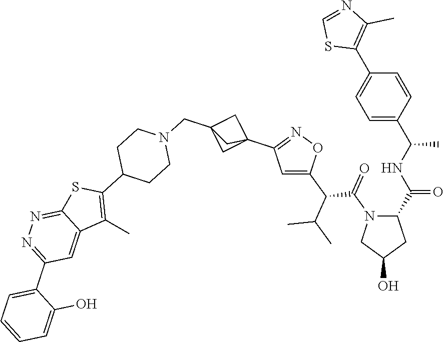 [SMILES: Cc1ncsc1-c1ccc([C@H](C)NC(=O)[C@@H]2C[C@@H](O)CN2C(=O)[C@@H](c2cc(C34CC(CN5CCC(c6sc7nnc(-c8ccccc8O)cc7c6C)CC5)(C3)C4)no2)C(C)C)cc1]

A mixture of Intermediate 14 (10 mg, 0.017 mmol, 1 equiv), compound I-10 (5.64 mg, 0.017 mmol, 1 equiv), AcOH (3.12 mg, 0.051 mmol, 3 equiv) and NaOAc (4.27 mg, 0.051 mmol, 3 equiv) in DCM (1 mL) and MeOH (1 mL) was stirred for 30 min at room temperature. To the above mixture was added NaBH3CN (3.27 mg, 0.051 mmol, 3 equiv) at room temperature. The resulting mixture was stirred for additional 2 h at room temperature. The mixture was purified by Prep-HPLC with the following conditions: Column, XBridge Shield RP18 OBD, 19*150 mm, 5 μm; Mobile Phase A: water (10 mmol/L NH₄HCO₃), Mobile Phase B: ACN; Flow rate: 25 mL/min; Gradient: 47% B to 73% B in 7 min, then 73% B; Detector: 254/220 nm; RT (min): 6.47. This resulted in Compound 72 (1.6 mg, 10.12%) as a white solid. ¹H NMR (400 MHz, Methanol-d₄) δ 8.87 (s, 1H), 8.53 (s, 1H), 8.09-8.02 (m, 1H), 7.48-7.31 (m, 5H), 7.06-6.97 (m, 2H), 6.29-6.21 (m, 1H), 5.08-4.98 (m, 1H), 4.55-4.37 (m, 2H), 3.89-3.75 (m, 2H), 3.65-3.57 (m, 1H), 3.28-3.26 (m, 1H), 3.21-3.13 (m, 2H), 2.63 (s, 2H), 2.50-2.46 (m, 6H), 2.45-2.24 (m, 3H), 2.21-2.12 (m, 7H), 2.07-1.87 (m, 5H), 1.56 (dd, J=30.6, 7.0 Hz, 3H), 1.06 (d, J=6.6 Hz, 3H), 0.91-0.83 (m, 3H). LCMS (ESI) m/z [M+H]⁺=886.2.

Preparation of (2S,4R)-4-hydroxy-1-((R)-2-(3-((1R,4R)-5-((S)-3-(2-hydroxyphenyl)-5,6,7,8-tetrahydrobenzo[4,5]thieno[2,3-c]pyridazine-6-carbonyl)-2,5-diazabicyclo[2.2.1]heptan-2-yl)isoxazol-5-yl)-3-methylbutanoyl)-N—((S)-1-(4-(4-methylthiazol-5-yl)phenyl)ethyl)pyrrolidine-2-carboxamide (Compound 41)

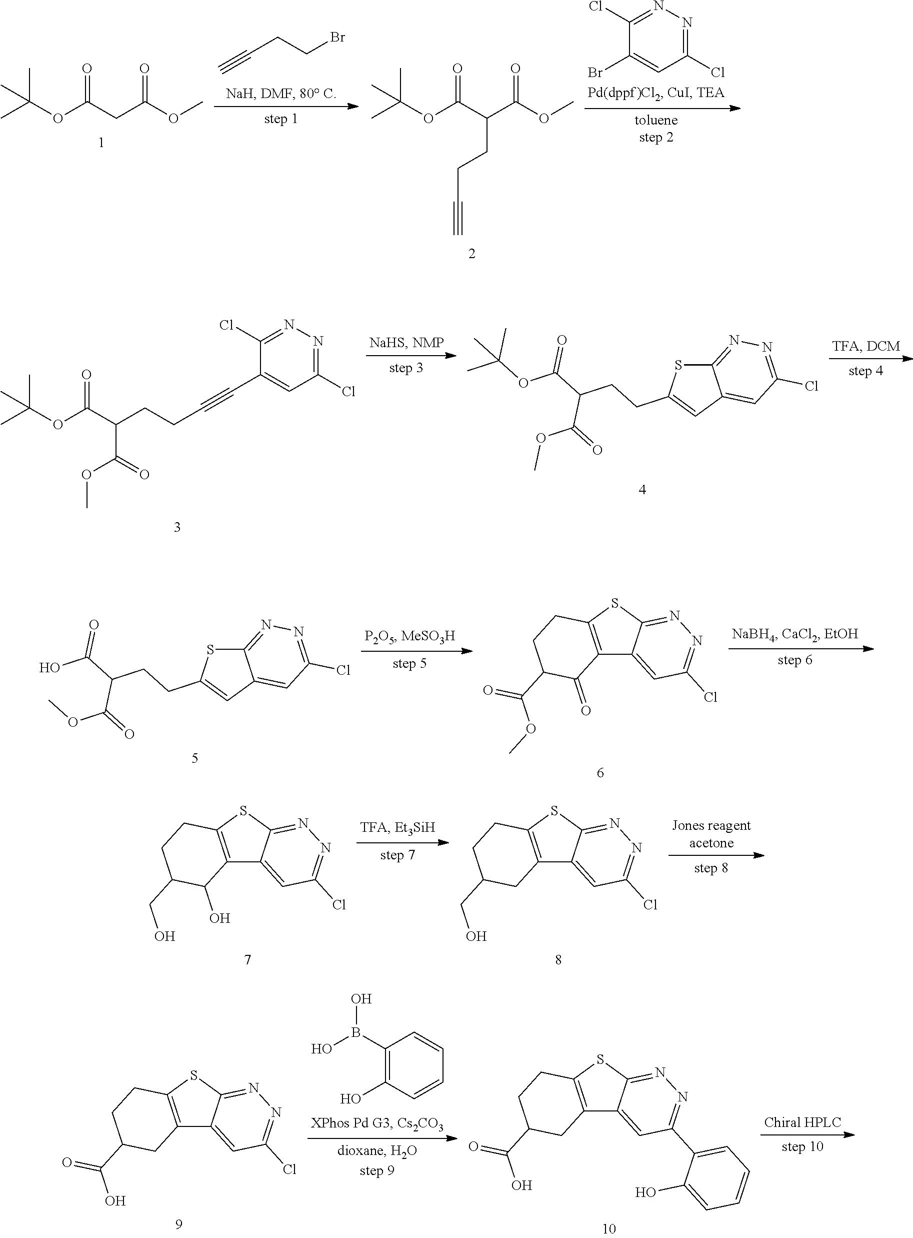

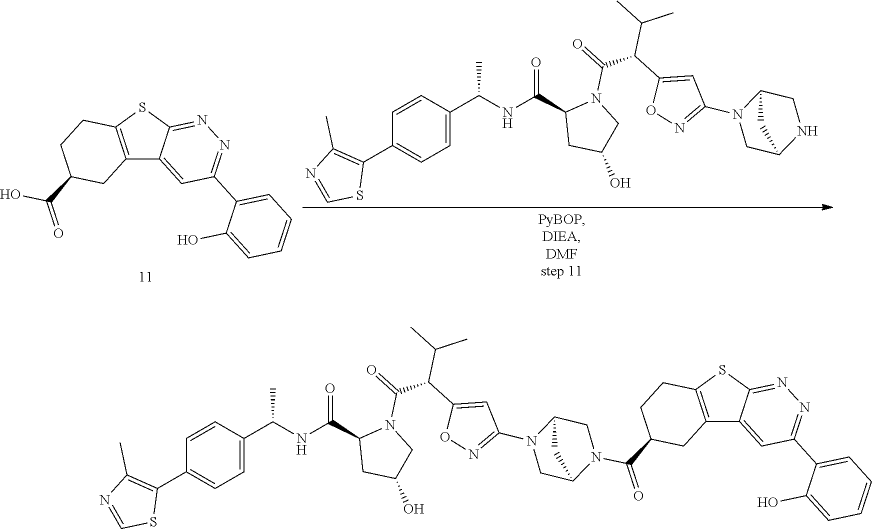

Step 1: Preparation of 1-(tert-butyl) 3-methyl 2-(but-3-yn-1-yl) malonate (Intermediate 2)

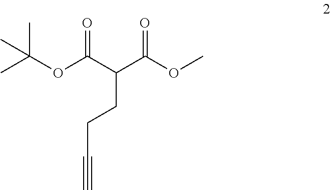 [SMILES: C#CCCC(C(=O)OC)C(=O)OC(C)(C)C]

To a solution of 1-tert-butyl 3-methyl propanedioate (80.00 g, 459.253 mmol, 1.00 equiv) in DMF (300 mL) was added NaH (22.0 g, 918.506 mmol, 2.00 equiv) at 0° C. After stirring for an hour at this temperature, 4-bromobut-1-yne (61.08 g, 459.253 mmol, 1.00 equiv) was added. The resulting mixture was stirred overnight at 80° C. The resulting mixture was diluted with water (800 mL). The resulting mixture was extracted with EtOAc (3×500 mL). The combined organic layers were washed with brine (3×300 mL), then dried over anhydrous sodium sulfate. After filtration, the filtrate was concentrated under reduced pressure. The residue was purified by silica gel column chromatography, eluted with EtOAc in PE from 0% to 30%, to afford intermediate 2 (75.00 g, 64.9%) as a yellow oil. No mass signal observed in LCMS, product was detected by GCMS.

Step 2: Preparation of 1-(tert-butyl) 3-methyl 2-(4-(3,6-dichloropyridazin-4-yl)but-3-yn-1-yl)malonate (Intermediate 3)

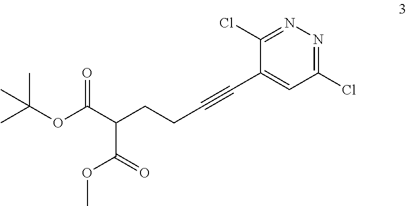 [SMILES: COC(=O)C(CCC#Cc1cc(Cl)nnc1Cl)C(=O)OC(C)(C)C]

To a solution of 4-bromo-3,6-dichloropyridazine (75.53 g, 331.459 mmol, 1.00 equiv), Pd(dppf)Cl₂—CH₂Cl₂ (27.00 g, 33.146 mmol, 0.10 equiv) and CuI (12.63 g, 66.292 mmol, 0.2 equiv) in toluene (400 mL) were added TEA (100.62 g, 994.377 mmol, 3.00 equiv) and intermediate 2 (75.00 g, 331.459 mmol, 1.00 equiv). After stirring overnight at room temperature under a nitrogen atmosphere, the resulting mixture was concentrated under reduced pressure. The residue was purified by silica gel column chromatography, eluted with EtOAc in PE from 0% to 30%, to afford intermediate 3 (40.00 g, 29.1%) as yellow oil. LCMS (ESI) m/z: [M+H]⁺=373.

Step 3: Preparation of 1-(tert-butyl) 3-methyl 2-(2-(3-chlorothieno[2,3-c]pyridazin-6-yl)ethyl)malonate (Intermediate 4)

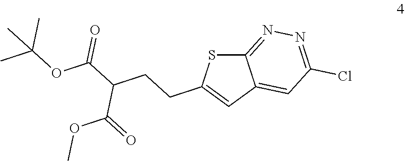 [SMILES: COC(=O)C(CCc1cc2cc(Cl)nnc2s1)C(=O)OC(C)(C)C]

To a solution of intermediate 3 (40.00 g, 107.173 mmol, 1.00 equiv) in NMP (600 mL) was added NaHS (7.81 g, 139.325 mmol, 1.30 equiv). After stirring for 25 min at 100° C., the resulting mixture was diluted with water (800 mL). The resulting mixture was extracted with EtOAc (3×500 mL). The combined organic layers were washed with brine (3×300 mL), then dried over anhydrous sodium sulfate. After filtration, the filtrate was concentrated under reduced pressure. The residue was purified by silica gel column chromatography, eluted with EtOAc in PE from 0% to 50%, to afford intermediate 4 (27.00 g, 61.1%) as a yellow solid. LCMS (ESI) m/z: [M+H]⁺=371.

Step 4: Preparation of 4-(3-chlorothieno[2,3-c]pyridazin-6-yl)-2-(methoxycarbonyl)butanoic acid (intermediate 5)

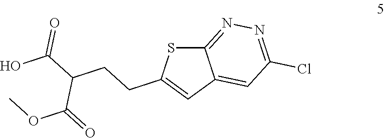 [SMILES: COC(=O)C(CCc1cc2cc(Cl)nnc2s1)C(=O)O]

To a solution of intermediate 4 (27.00 g, 72.806 mmol, 1.00 equiv) in DCM (150 mL) was added TFA (50 mL, 673.154 mmol, 9.25 equiv). After stirring for one hour at room temperature, the resulting mixture was concentrated under reduced pressure. The residue was purified by reverse phase flash chromatography with the following conditions: column, C18 silica gel; mobile phase, ACN in water, 10% to 50% gradient in 10 min; detector, UV 254 nm. This resulted in intermediate 5 (19 g, 74.62%) as a yellow solid. LCMS (ESI) m/z: [M+H]⁺=315.

Step 5: Preparation of methyl 3-chloro-5-oxo-5,6,7,8-tetrahydrobenzo[4,5]thieno[2,3-c]pyridazine-6-carboxylate (intermediate 6)

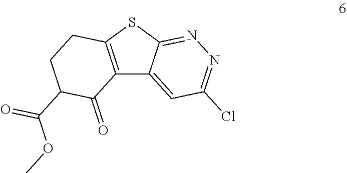 [SMILES: COC(=O)C1CCc2sc3nnc(Cl)cc3c2C1=O]

To a solution of intermediate 5 (19.00 g, 60.367 mmol, 1.00 equiv) in methanesulfonic acid (60 mL) was added P₂O₅ (17.14 g, 120.753 mmol, 2.00 equiv). After stirring for 1.5 hours at 80° C., the reaction was quenched by the addition of water (200 mL) at 0° C. The mixture was neutralized to pH 7 with saturated sodium carbonate solution. The resulting mixture was extracted with EtOAc (3×500 mL). The combined organic layers were washed with brine (3×200 mL), then dried over anhydrous sodium sulfate. After filtration, the filtrate was concentrated under reduced pressure to afford intermediate 6 (7.00 g, 35.1%) as a yellow solid. LCMS (ESI) m/z: [M+H]⁺=297.

Step 6: Preparation of 3-chloro-6-(hydroxymethyl)-5,6,7,8-tetrahydrobenzo[4,5]thieno[2,3-c]pyridazin-5-ol (intermediate 7)

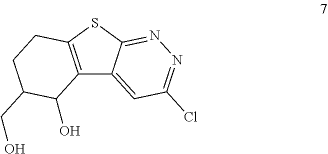 [SMILES: OCC1CCc2sc3nnc(Cl)cc3c2C1O]

To a mixture of intermediate 6 (7.00 g, 23.590 mmol, 1.00 equiv) and CaCl₂ (5.24 g, 47.180 mmol, 2.00 equiv) in EtOH (150 mL) was added NaBH4 (2.68 g, 70.770 mmol, 3.00 equiv) at 0° C. After stirring for 6 h at room temperature, the reaction was quenched with 1N HCl at 0° C. The resulting mixture was extracted with DCM/MeOH (10:1) (3×500 mL). The combined organic layers were dried over anhydrous Na₂SO₄, filtered and concentrated under reduced pressure. The residue was purified by reverse phase flash chromatography with the following conditions: column, Cl₈ silica gel; mobile phase, ACN in water, 10% to 50% gradient in 10 min; detector, UV 254 nm. This resulted in intermediate 7 (4.20 g, 59.1%) as a white solid. LCMS (ESI) m/z: [M+H]⁺=271.

Step 7: Preparation of (3-chloro-5,6,7,8-tetrahydrobenzo[4,5]thieno[2,3-c]pyridazin-6-yl)methanol (intermediate 8)

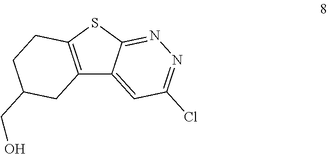 [SMILES: OCC1CCc2sc3nnc(Cl)cc3c2C1]

To a solution of intermediate 7 (4.20 g, 15.514 mmol, 1.00 equiv) in TFA (50 mL) was added Et₃SiH (25 mL, 154.800 mmol, 9.98 equiv). After stirring for 2.5 h at 90° C., the resulting mixture was concentrated under reduced pressure. The residue was dissolved in EtOAc (500 mL), washed with saturated sodium bicarbonate, dried over anhydrous Na₂SO₄, filtered and concentrated under reduced pressure to afford intermediate 8 (2.50 g, 56.9%) as a yellow solid. LCMS (ESI) m/z: [M+H]⁺=255.

Step 8: Preparation of 3-chloro-5,6,7,8-tetrahydrobenzo[4,5]thieno[2,3-c]pyridazine-6-carboxylic acid (intermediate 9)

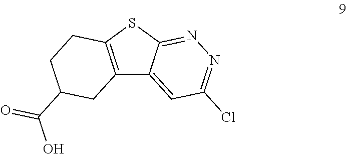 [SMILES: O=C(O)C1CCc2sc3nnc(Cl)cc3c2C1]

To a solution of intermediate 8 (2.50 g, 9.814 mmol, 1.00 equiv) in acetone (50 mL) was added Jones reagent (3.89 g, 19.628 mmol, 2.00 equiv) at 0° C. After stirring for one hour at room temperature, the reaction was quenched by the addition of NaHSO₃ solution and diluted with water (50 mL). The resulting mixture was extracted with EtOAc (3×100 mL). The combined organic layers were washed with brine (100 mL), then dried over anhydrous sodium sulfate. After filtration, the filtrate was concentrated under reduced pressure to afford intermediate 9 (1.5 g, 51.19%) as a yellow solid. LCMS (ESI) m/z: [M+H]⁺=269.

Step 9: Preparation of 3-(2-hydroxyphenyl)-5,6,7,8-tetrahydrobenzo [4,5] thieno [2,3-c]pyridazine-6-carboxylic acid (intermediate 10)

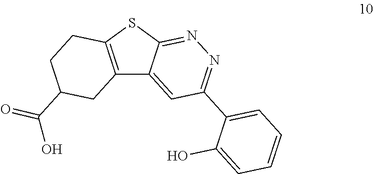 [SMILES: O=C(O)C1CCc2sc3nnc(-c4ccccc4O)cc3c2C1]

To a solution of intermediate 9 (1.50 g, 5.582 mmol, 1.00 equiv) and 2-hydroxyphenylboronic acid (1.15 g, 8.373 mmol, 1.50 equiv) in dioxane (35 mL) and water (7 mL) were added Cs₂CO₃ (5.46 g, 16.746 mmol, 3.00 equiv) and XPhos Pd G3 (0.47 g, 0.558 mmol, 0.10 equiv). After stirring for one hour at 100° C. under a nitrogen atmosphere, the resulting mixture was concentrated under reduced pressure. The residue was purified by reverse phase flash chromatography with the following conditions: column, C₁₈ silica gel; mobile phase, ACN in water, 10% to 50% gradient in 10 min; detector, UV 254 nm. This resulted in intermediate 10 (910.0 mg, 44.9%) as a yellow solid. LCMS (ESI) m/z: [M+H]⁺=327.

Step 10: Preparation of (S)-3-(2-hydroxyphenyl)-5,6,7,8-tetrahydrobenzo[4,5] thieno[2,3-c]pyridazine-6-carboxylic acid (intermediate 11)

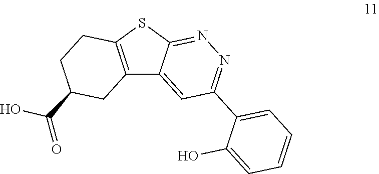 [SMILES: O=C(O)[C@H]1CCc2sc3nnc(-c4ccccc4O)cc3c2C1]

Intermediate 10 (500 mg) was separated by chiral HPLC with the following conditions: Column: CHIRALPAK IG-3, 4.6*50 mm, 3 μm; Mobile Phase A: Hexane (0.1% TFA), Mobile Phase B: EtOH; Flow rate: 1 mL/min; Gradient: 0% B to 70% B. This resulted in intermediate 11 (169.0 mg, 33.8%) as a yellow solid. LCMS (ESI) m/z: [M+H]⁺=327.

Step 11: Preparation of (2S,4R)-4-hydroxy-1-((R)-2-(3-((1R,4R)-5-((S)-3-(2-hydroxyphenyl)-5,6,7,8-tetrahydrobenzo[4,5]thieno[2,3-c]pyridazine-6-carbonyl)-2,5-diazabicyclo[2.2.1]heptan-2-yl)isoxazol-5-yl)-3-methylbutanoyl)-N—((S)-1-(4-(4-methylthiazol-5-yl)phenyl)ethyl)pyrrolidine-2-carboxamide (Compound 41)

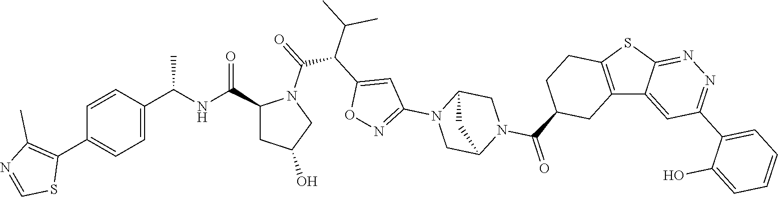 [SMILES: Cc1ncsc1-c1ccc([C@H](C)NC(=O)[C@@H]2C[C@@H](O)CN2C(=O)[C@@H](c2cc(N3C[C@H]4C[C@@H]3CN4C(=O)[C@H]3CCc4sc5nnc(-c6ccccc6O)cc5c4C3)no2)C(C)C)cc1]

A solution of intermediate 11 (15 mg, 0.046 mmol, 1 equiv), DIEA (17.82 mg, 0.138 mmol, 3 equiv), PyBOP (96 mg, 0.184 mmol, 4 equiv) and (2S,4R)-1-[(2R)-2-{3-[(1R,4R)-2,5-diazabicyclo[2.2.1]heptan-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (26.60 mg, 0.046 mmol, 1 equiv) in DMF (1 mL) was stirred at 25° C. for 2 h. The mixture was purified by Prep HPLC with the following conditions: Column: X Bridge Prep OBD C18, 30*150 mm, 5 μm; Mobile Phase A: Water (10 mmol/L NH₄HCO₃+0.1% NH₃·H₂O), Mobile Phase B: ACN; Flow rate: 25 mL/min; Gradient: 40% B to 60% B in 7 min, then 60% B; Detector: 254/220 nm; RT (min): 5.8. This resulted in compound 41 (12.4 mg, 30.41%) as a white solid. 1H NMR (300 MHz, Methanol-d4) δ 8.89 (s, 1H), 8.46 (dd, J=13.6, 11.7 Hz, 1H), 8.10-7.98 (m, 1H), 7.51-7.45 (m, 4H), 7.41-7.23 (m, 2H), 7.08-6.96 (m, 2H), 6.10 (d, J=7.9 Hz, 1H), 5.10-4.99 (m, 2H), 4.57-4.49 (m, 3H), 3.94-3.86 (m, 2H), 3.76-3.56 (m, 2H), 3.50-3.36 (m, 1H), 3.28 (d, J=10.2 Hz, 2H), 3.18 (td, J=6.6, 3.7 Hz, 2H), 3.01-2.89 (m, 1H), 2.64-2.46 (m, 4H), 2.32-2.22 (m, 1H), 2.20-2.15 (m, 1H), 2.01-1.96 (m, 3H), 1.89-1.82 (m, 2H), 1.65-1.49 (m, 3H), 1.09 (dd, J=6.6, 1.7 Hz, 3H), 1.02-0.89 (m, 3H). LCMS (ESI) m/z: [M+H]⁺=887.30.

Preparation of (2S,4R)-4-hydroxy-1-((R)-2-(3-((1R,4R)-5-((R)-3-(2-hydroxyphenyl)-5,6,7,8-tetrahydrobenzo[4,5]thieno[2,3-c]pyridazine-6-carbonyl)-2,5-diazabicyclo[2.2.1]heptan-2-yl)isoxazol-5-yl)-3-methylbutanoyl)-N—((S)-1-(4-(4-methylthiazol-5-yl)phenyl)ethyl)pyrrolidine-2-carboxamide (Compound 42)

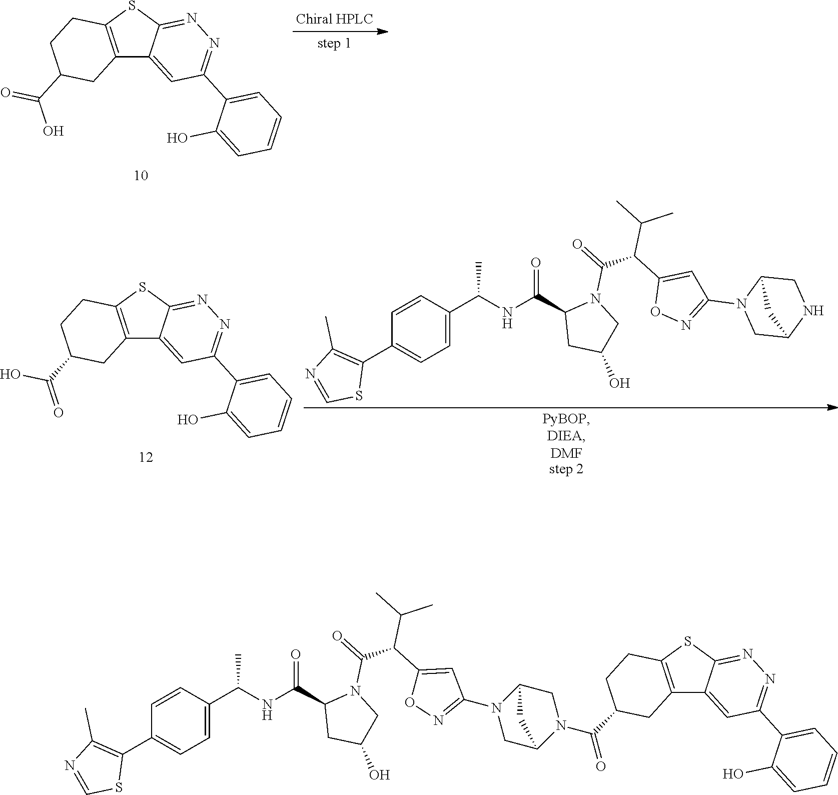

Step 1: Preparation of (R)-3-(2-hydroxyphenyl)-5,6,7,8-tetrahydrobenzo[4,5] thieno [2,3-c]pyridazine-6-carboxylic acid (intermediate 12)

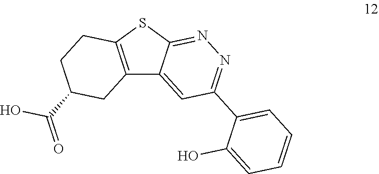 [SMILES: O=C(O)[C@@H]1CCc2sc3nnc(-c4ccccc4O)cc3c2C1]

Intermediate 10 (500 mg) was separated by chiral HPLC with the following conditions: Column: CHIRALPAK IG-3, 4.6*50 mm, 3 μm; Mobile Phase A: Hexane (0.1% TFA), Mobile Phase B: EtOH; Flow rate: 1 mL/min; Gradient: 0% B to 70% B. This resulted in intermediate 12 (180.1 mg, 36.0%) as a yellow solid. LCMS (ESI) m/z: [M+H]⁺=327.

Step 2: Preparation of (2S,4R)-4-hydroxy-1-((R)-2-(3-((1R,4R)-5-((R)-3-(2-hydroxyphenyl)-5,6,7,8-tetrahydrobenzo[4,5]thieno[2,3-c]pyridazine-6-carbonyl)-2,5-diazabicyclo[2.2.1]heptan-2-yl)isoxazol-5-yl)-3-methylbutanoyl)-N—((S)-1-(4-(4-methylthiazol-5-yl)phenyl)ethyl)pyrrolidine-2-carboxamide (Compound 42)

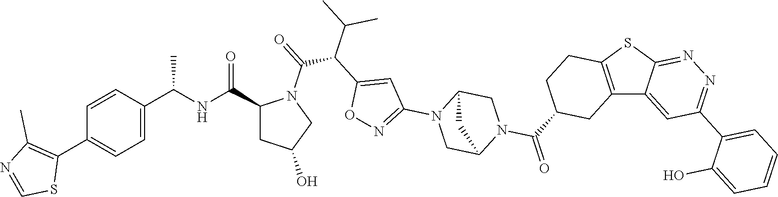 [SMILES: Cc1ncsc1-c1ccc([C@H](C)NC(=O)[C@@H]2C[C@@H](O)CN2C(=O)[C@@H](c2cc(N3C[C@H]4C[C@@H]3CN4C(=O)[C@@H]3CCc4sc5nnc(-c6ccccc6O)cc5c4C3)no2)C(C)C)cc1]

A solution of intermediate 12 (15 mg, 0.046 mmol, 1 equiv), DIEA (17.82 mg, 0.138 mmol, 3 equiv), PyBOP (96 mg, 0.184 mmol, 4 equiv) and (2S,4R)-1-[(2R)-2-{3-[(1R,4R)-2,5-diazabicyclo[2.2.1] heptan-2-yl]-1,2-oxazol-5-yl}-3-methylbutanoyl]-4-hydroxy-N-[(1S)-1-[4-(4-methyl-1,3-thiazol-5-yl)phenyl]ethyl]pyrrolidine-2-carboxamide (26.60 mg, 0.046 mmol, 1 equiv) in DMF (1 mL) was stirred at 25° C. for 2 h. The mixture was purified by Prep HPLC with the following conditions: Column: X Bridge Prep OBD C18, 30*150 mm, 5 μm; Mobile Phase A: Water (10 mmol/L NH₄HCO₃+0.1% NH₃·H₂O), Mobile Phase B: ACN; Flow rate: 25 mL/min; Gradient: 40% B to 60% B in 7 min, then 60% B; Detector: 254/220 nm; RT (min): 6.4. This resulted in compound 42 (10.8 mg, 26.47%) as a white solid. 1H NMR (400 MHz, DMSO-d₆) δ 12.95 (s, 1H), 8.98 (d, J=1.6 Hz, 1H), 8.63 (d, J=12.7 Hz, 1H), 8.39 (dd, J=16.5, 7.7 Hz, 1H), 8.16 (ddd, J=8.1, 3.2, 1.6 Hz, 1H), 7.44-7.32 (m, 5H), 7.06-6.99 (m, 2H), 6.09 (d, J=7.7 Hz, 1H), 5.20-4.84 (m, 3H), 4.48 (s, 1H), 4.43-4.23 (m, 2H), 3.87-3.73 (m, 2H), 3.70-3.65 (m, 1H), 3.58-3.41 (m, 2H), 3.40-3.38 (m, 1H), 3.36-3.25 (m, 1H), 3.18-3.08 (m, 4H), 2.84 (d, J=14.4 Hz, 2H), 2.45 (d, J=3.7 Hz, 3H), 2.42-2.15 (m, 1H), 2.08 (d, J=7.5 Hz, 1H), 2.04-1.98 (m, 1H), 1.95-1.90 (m, 2H), 1.87-1.77 (m, 1H), 1.49-1.33 (m, 3H), 0.97 (t, J=6.1 Hz, 3H), 0.93-0.84 (m, 3H). LCMS (ESI) m/z: [M+H]⁺=887.30.

The compounds in Table 11 were prepared using procedures similar to those used above for the preparation of compound 41 using the appropriate amine and carboxylic acid.

TABLE 11

| | | LCMS | |
|---|---|---|---|
| Compound | | (ESI) | |
| No. | Name | m/z | ¹H NMR |
| 43 | (2S,4R)-4-hydroxy-1-((R)-2-(3- | 887.25 | 1H NMR (400 MHz, DMSO-d6) δ 12.94 |
| | ((1S,4S)-5-((S)-3-(2- | | (d, J = 25.6 Hz, 1H), 8.99 (d, J = 1.9 Hz, |
| | hydroxyphenyl)-5,6,7,8- | | 1H), 8.84-8.59 (m, 1H), 8.39 (dd, J = |
| | tetrahydrobenzo[4,5]thieno[2,3- | | 34.3, 7.7 Hz, 1H), 8.22-8.04 (m, 1H), |
| | c]pyridazine-6-carbonyl)-2,5- | | 7.60-7.23 (m, 5H), 7.10-6.87 (m, |
| | diazabicyclo[2.2.1]heptan-2- | | 2H), 6.19-5.92 (m, 1H), 5.08 (dd, J = |
| | yl)isoxazol-5-yl)-3-methylbutanoyl)- | | 25.2, 3.6 Hz, 1H), 4.99-4.80 (m, 2H), |
| | N-((S)-1-(4-(4-methylthiazol-5- | | 4.56-4.16 (m, 3H), 3.75-3.40 (m, |
| | yl)phenyl)ethyl)pyrrolidine-2- | | 5H), 3.29-2.93 (m, 5H), 2.83 (d, J = |
| | carboxamide | | 13.5 Hz, 1H), 2.45 (d, J = 4.5 Hz, 3H), |
| | | | 2.30-1.60 (m, 8H), 1.38 (td, J = 14.2, |
| | | | 13.0, 6.5 Hz, 3H), 1.03-0.89 (m, 3H), |
| | | | 0.81 (ddd, J = 22.1, 15.3, 6.4 Hz, 3H). |
| 44 | (2S,4R)-4-hydroxy-1-((R)-2-(3- | 887.25 | 1H NMR (400 MHz, DMSO-d6) δ 12.96 |
| | ((1S,4S)-5-((R)-3-(2- | | (s, 1H), 8.99 (s, 1H), 8.86-8.63 (m, |
| | hydroxyphenyl)-5,6,7,8- | | 1H), 8.42 (dd, J = 12.3, 7.7 Hz, 1H), |
| | tetrahydrobenzo[4,5]thieno[2,3- | | 8.21-8.10 (m, 1H), 7.44 (d, J = 8.1 Hz, |
| | c]pyridazine-6-carbonyl)-2,5- | | 2H), 7.41-7.32 (m, 3H), 7.01 (dd, J = |
| | diazabicyclo[2.2.1]heptan-2- | | 11.4, 7.7 Hz, 2H), 6.16-5.97 (m, 1H), |
| | yl)isoxazol-5-yl)-3-methylbutanoyl)- | | 5.12 (s, 1H), 4.99-4.65 (m, 2H), 4.47 |
| | N-((S)-1-(4-(4-methylthiazol-5- | | (d, J = 23.6 Hz, 1H), 4.38 (q, J = 7.7 Hz, |
| | yl)phenyl)ethyl)pyrrolidine-2- | | 1H), 4.30 (s, 1H), 3.77-3.43 (m, 4H), |
| | carboxamide | | 3.29-2.95 (m, 6H), 2.86 (dd, J = 27.3, |
| | | | 13.3 Hz, 1H), 2.46 (s, 3H), 2.36-2.11 |
| | | | (m, 2H), 2.08-1.70 (m, 6H), 1.51- |
| | | | 1.34 (m, 3H), 0.97 (dd, J = 6.7, 2.3 Hz, |
| | | | 3H), 0.83 (q, J = 7.0 Hz, 3H). |
| 45 | (S)-N-(2-((5-((R)-1-((2S,4R)-4- | 850.3 | 1H NMR (300 MHz, DMSO-d6) δ 13.0 |
| | hydroxy-2-(((S)-1-(4-(4- | | (s, 1H), 9.06 (s, 1H), 8.80 (s, 1H), 8.49 |
| | methylthiazol-5- | | (d, J = 7.6 Hz, 1H), 8.41 (s, 1H), 8.30- |
| | yl)phenyl)ethyl)carbamoyl)pyrrolidin- | | 8.21 (m, 1H), 7.51 (d, J = 8.2 Hz, 5H), |
| | 1-yl)-3-methyl-1-oxobutan-2- | | 7.08 (t, J = 8.1 Hz, 2H), 6.19 (s, 1H), |
| | yl)isoxazol-3-yl)oxy)ethyl)-3-(2- | | 5.19 (s, 1H), 4.98 (t, J = 7.2 Hz, 1H), |
| | hydroxyphenyl)-5,6,7,8- | | 4.44-4.35 (m, 4H), 3.75 (t, J = 9.2 Hz, |
| | tetrahydrobenzo[4,5]thieno[2,3- | | 2H), 3.65-3.56 (m, 3H), 3.22-3.10 |
| | c]pyridazine-6-carboxamide | | (m, 3H), 2.95-2.90 (m, 2H), 2.53 (d, J = |
| | | | 1.5 Hz, 3H), 2.22 (d, J = 12.0 Hz, 2H), |
| | | | 1.95-1.93 (m, 2H), 1.53 (d, J = 7.1 Hz, |
| | | | 1H), 1.45 (d, J = 7.0 Hz, 3H), 1.03 (d, J = |
| | | | 6.5 Hz, 3H), 0.90 (dd, J = 13.1, 6.6 |
| | | | Hz, 3H). |
| 46 | (R)-N-(2-((5-((R)-1-((2S,4R)-4- | 850.25 | 1H NMR (400 MHz, DMSO-d6) δ 12.93 |
| | hydroxy-2-(((S)-1-(4-(4- | | (s, 1H), 8.99 (s, 1H), 8.72 (s, 1H), 8.41 |
| | methylthiazol-5- | | (d, J = 7.7 Hz, 2H), 8.18 (dd, J = 8.0, |
| | yl)phenyl)ethyl)carbamoyl)pyrrolidin- | | 1.6 Hz, 1H), 7.49-7.41 (m, 2H), 7.40- |
| | 1-yl)-3-methyl-1-oxobutan-2- | | 7.32 (m, 3H), 7.06-6.95 (m, 2H), |
| | yl)isoxazol-3-yl)oxy)ethyl)-3-(2- | | 6.11 (s, 1H), 5.11 (s, 1H), 4.91 (t, J = |
| | hydroxyphenyl)-5,6,7,8- | | 7.5, 7.0 Hz, 1H), 4.37 (t, J = 7.9 Hz, |
| | tetrahydrobenzo[4,5]thieno[2,3- | | 1H), 4.29 (s, 3H), 3.74-3.63 (m, 2H), |
| | c]pyridazine-6-carboxamide | | 3.62-3.58 (m, 3H), 3.55-3.35 (m, |
| | | | 2H), 3.22-3.01 (m, 2H), 2.90-2.85 |
| | | | (m, 1H), 2.65-2.44 (m, 3H), 2.32- |
| | | | 2.21 (m, 1H), 2.20-2.12 (m, 1H), 2.03 |
| | | | (t, J = 10.0 Hz, 1H), 2.01-1.99 (m, 1H), |
| | | | 1.98-1.94 (m, 1H), 1.92 (dd, J = 10.9, |
| | | | 5.5 Hz, 3H), 0.96 (d, J = 6.4 Hz, 3H), |
| | | | 0.82 (dd, J = 14.6, 6.7 Hz, 3H). |
| 47 | (2S,4R)-4-hydroxy-1-((R)-2-(3-(1- | 874.25 | 1H NMR (400 MHz, DMSO-d6) δ 12.99 |
| | ((S)-3-(2-hydroxyphenyl)-5,6,7,8- | | (s, 1H), 8.99 (s, 1H), 8.73 (d, J = 9.1 |
| | tetrahydrobenzo[4,5]thieno[2,3- | | Hz, 1H), 8.42 (d, J = 7.8 Hz, 1H), 8.18 |
| | c]pyridazine-6-carbonyl)piperidin-4- | | (d, J = 7.9 Hz, 1H), 7.44 (d, J = 8.0 Hz, |
| | yl)isoxazol-5-yl)-3-methylbutanoyl)- | | 2H), 7.37 (d, J = 8.0 Hz, 3H), 7.01 (t, J = |
| | N-((S)-1-(4-(4-methylthiazol-5- | | 8.7 Hz, 2H), 6.39 (d, J = 1.8 Hz, 1H), |
| | yl)phenyl)ethyl)pyrrolidine-2- | | 5.29-4.64 (m, 2H), 4.48 (d, J = 12.8 |
| | carboxamide | | Hz, 1H), 4.42-4.20 (m, 2H), 4.13 (d, J = |
| | | | 13.5 Hz, 1H), 3.80-3.68 (m, 2H), |
| | | | 3.48 (d, J = 10.6 Hz, 1H), 3.26 (s, 2H), |
| | | | 3.11-2.97 (m, 4H), 2.92-2.76 (m, |
| | | | 2H), 2.46 (s, 3H), 2.32-2.19 (m, 1H), |
| | | | 2.14-1.85 (m, 5H), 1.84-1.73 (m, |
| | | | 1H), 1.66 (t, J = 12.8 Hz, 1H), 1.56- |
| | | | 1.43 (m, 1H), 1.38 (dd, J = 7.0, 2.8 Hz, |
| | | | 3H), 0.98 (dd, J = 6.7, 3.5 Hz, 3H), 0.80 |
| | | | (dd, J = 11.7, 6.5 Hz, 3H). |
| 48 | (2S,4R)-4-hydroxy-1-((R)-2-(3-(1- | 874.25 | 1H NMR (400 MHz, DMSO-d6) δ 12.95 |
| | ((R)-3-(2-hydroxyphenyl)-5,6,7,8- | | (s, 1H), 8.99 (s, 1H), 8.73 (d, J = 7.5 |
| | tetrahydrobenzo[4,5]thieno[2,3- | | Hz, 1H), 8.42 (dd, J = 8.0, 3.4 Hz, 1H), |
| | c]pyridazine-6-carbonyl)piperidin-4- | | 8.18 (dd, J = 8.0, 1.6 Hz, 1H), 7.44 (d, |
| | yl)isoxazol-5-yl)-3-methylbutanoyl)- | | J = 8.1 Hz, 2H), 7.37 (d, J = 8.0 Hz, |
| | N-((S)-1-(4-(4-methylthiazol-5- | | 3H), 7.01 (t, J = 8.7 Hz, 2H), 6.38 (d, J = |
| | yl)phenyl)ethyl)pyrrolidine-2- | | 7.6 Hz, 1H), 5.25-4.64 (m, 2H), 4.49 |
| | carboxamide | | (d, J = 12.9 Hz, 1H), 4.40-4.23 (m, |
| | | | 2H), 4.13 (d, J = 13.5 Hz, 1H), 3.80- |
| | | | 3.68 (m, 2H), 3.47 (d, J = 10.6 Hz, 1H), |
| | | | 3.27 (d, J = 11.0 Hz, 2H), 3.12-2.97 |
| | | | (m, 4H), 2.94-2.71 (m, 2H), 2.46 (s, |
| | | | 3H), 2.26 (q, J = 7.0 Hz, 1H), 2.14- |
| | | | 1.83 (m, 5H), 1.78 (t, J = 7.9 Hz, 1H), |
| | | | 1.72-1.65 (m, 1H), 1.54-1.30 (m, |
| | | | 4H), 0.98 (dd, J = 6.7, 3.5 Hz, 3H), 0.80 |
| | | | (dd, J = 11.7, 6.6 Hz, 3H). |
| 52 | (2S,4R)-4-hydroxy-1-((R)-2-(3-(4- | 875.25 | 1H NMR (400 MHz, DMSO-d6) δ 12.94 |
| | ((S)-3-(2-hydroxyphenyl)-5,6,7,8- | | (d, J = 5.2 Hz, 1H), 8.99 (d, J = 2.3 Hz, |
| | tetrahydrobenzo[4,5]thieno[2,3- | | 1H), 8.84-8.67 (m, 1H), 8.40 (d, J = |
| | c]pyridazine-6-carbonyl)piperazin-1- | | 7.7 Hz, 1H), 8.17 (dd, J = 8.0, 1.6 Hz, |
| | yl)isoxazol-5-yl)-3-methylbutanoyl)- | | 1H), 7.55-7.29 (m, 4H), 7.01 (td, J = |
| | N-((S)-1-(4-(4-methylthiazol-5- | | 8.0, 1.5 Hz, 2H), 6.24 (s, 1H), 5.12 (d, |
| | yl)phenyl)ethyl)pyrrolidine-2- | | J = 3.6 Hz, 1H), 5.02-4.85 (m, 1H), |
| | carboxamide | | 4.42-4.23 (m, 2H), 3.86-3.55 (m, |
| | | | 5H), 3.44 (d, J = 11.0 Hz, 3H), 3.31- |
| | | | 3.13 (m, 5H), 3.09 (s, 3H), 2.92-2.76 |
| | | | (m, 1H), 2.46 (s, 3H), 2.36-2.07 (m, |
| | | | 2H), 2.07-1.86 (m, 2H), 1.79 (ddd, J = |
| | | | 12.7, 7.9, 4.7 Hz, 1H), 1.47 (d, J = 6.9 |
| | | | Hz, 1H), 1.38 (d, J = 7.0 Hz, 2H), 0.97 |
| | | | (t, J = 6.6 Hz, 3H), 0.82 (dd, J = 14.3, |
| | | | 6.7 Hz, 3H). |
| 53 | (2S,4R)-4-hydroxy-1-((R)-2-(3-(4- | 875.25 | 1H NMR (400 MHz, DMSO-d6) δ 12.94 |
| | ((R)-3-(2-hydroxyphenyl)-5,6,7,8- | | (d, J = 6.9 Hz, 1H), 8.99 (s, 1H), 8.84- |
| | tetrahydrobenzo[4,5]thieno[2,3- | | 8.69 (m, 1H), 8.40 (d, J = 7.8 Hz, 1H), |
| | c]pyridazine-6-carbonyl)piperazin-1- | | 8.17 (dd, J = 8.0, 1.6 Hz, 1H), 7.55- |
| | yl)isoxazol-5-yl)-3-methylbutanoyl)- | | 7.27 (m, 4H), 7.12-6.92 (m, 2H), 6.23 |
| | N-((S)-1-(4-(4-methylthiazol-5- | | (s, 1H), 5.11 (d, J = 3.6 Hz, 1H), 5.03- |
| | yl)phenyl)ethyl)pyrrolidine-2- | | 4.87 (m, 1H), 4.43-4.22 (m, 2H), 3.83- |
| | carboxamide | | 3.53 (m, 5H), 3.42 (s, 3H), 3.28 (s, |
| | | | 5H), 3.09 (s, 3H), 2.91-2.80 (m, 1H), |
| | | | 2.46 (d, J = 1.1 Hz, 3H), 2.35-2.08 (m, |
| | | | 2H), 2.07-1.86 (m, 2H), 1.85-1.74 |
| | | | (m, 1H), 1.42 (dd, J = 33.7, 6.9 Hz, 3H), |
| | | | 0.96 (d, J = 6.5 Hz, 3H), 0.82 (dd, J = |
| | | | 14.4, 6.5 Hz, 3H). |

Preparation of (R)-2-(6-amino-5,6,7,8-tetrahydrobenzo[4,5]thieno[2,3-c]pyridazin-3-yl)phenol (intermediate A) and (S)-2-(6-amino-5,6,7,8-tetrahydrobenzo[4,5]thieno[2,3-c]pyridazin-3-yl)phenol (Intermediate B)

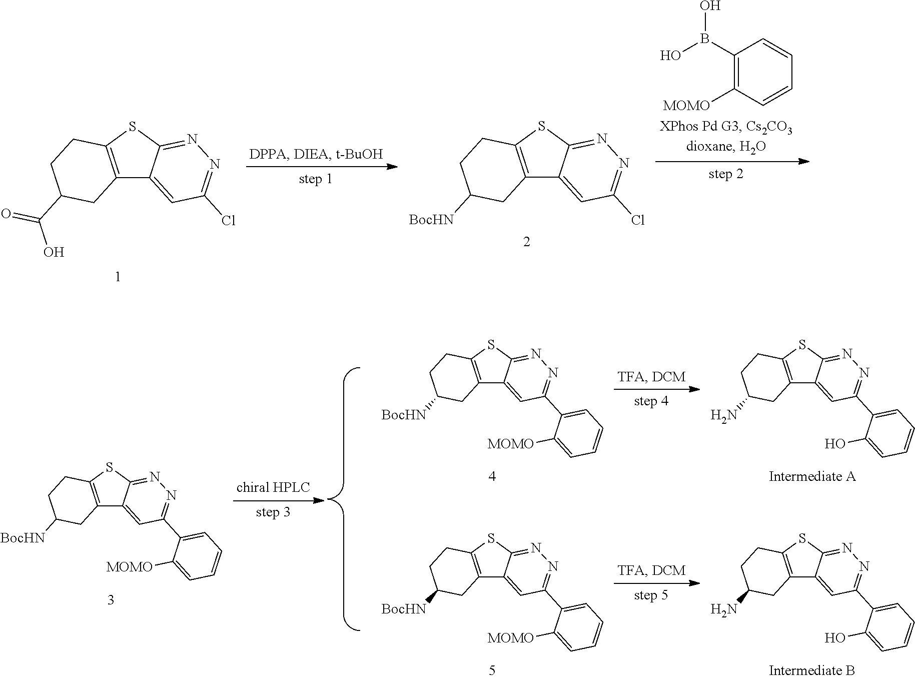

Step 1: Preparation of tert-butyl (3-chloro-5,6,7,8-tetrahydrobenzo[4,5]thieno[2,3-c]pyridazin-6-yl)carbamate (intermediate 2)

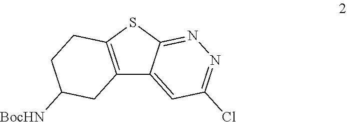 [SMILES: CC(C)(C)OC(=O)NC1CCc2sc3nnc(Cl)cc3c2C1]

To a solution of 3-chloro-5,6,7,8-tetrahydrobenzo[4,5]thieno[2,3-c]pyridazine-6-carboxylic acid (1.30 g, 4.838 mmol, 1.00 equiv) and DIEA (1.88 g, 14.514 mmol, 3.00 equiv) in t-BuOH (15 mL, 157.846 mmol, 32.63 equiv) was added DPPA (2.00 g, 7.257 mmol, 1.5 equiv). After stirring overnight at 80° C., the resulting mixture was concentrated under reduced pressure. The residue was purified by silica gel column chromatography, eluted with EtOAc in PE from 0% to 100%, to afford intermediate 2 (1.20 g, 65.6%) as a yellow solid. LCMS (ESI) m/z: [M+H]⁺=340.

Step 2: Preparation of tert-butyl (3-(2-(methoxymethoxy)phenyl)-5,6,7,8-tetrahydrobenzo[4,5]thieno[2,3-c]pyridazin-6-yl)carbamate (intermediate 3)

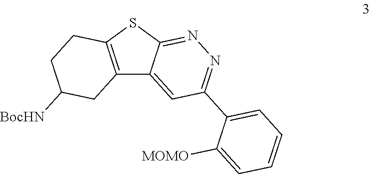

To a solution of intermediate 2 (1.20 g, 3.531 mmol, 1.00 equiv) and 2-(methoxymethoxy)phenylboronic acid (0.96 g, 5.296 mmol, 1.50 equiv) in dioxane (30 mL) and water (6 mL) were added Cs2CO3 (3.45 g, 10.593 mmol, 3.00 equiv) and XPhos Pd G3 (0.30 g, 0.353 mmol, 0.10 equiv). After stirring for one hour at 100° C. under a nitrogen atmosphere, the resulting mixture was concentrated under reduced pressure. The residue was purified by silica gel column chromatography, eluted with EtOAc in PE from 0% to 50%, to afford intermediate 3 (1.20 g, 69.2%) as a yellow solid. LCMS (ESI) m/z: [M+H]⁺=442.

Step 3: Preparation of tert-butyl (R)-(3-(2-(methoxymethoxy)phenyl)-5,6,7,8-tetrahydrobenzo[4,5]thieno[2,3-c]pyridazin-6-yl)carbamate (intermediate 4) and tert-butyl (S)-(3-(2-(methoxymethoxy)phenyl)-5,6,7,8-tetrahydrobenzo[4,5]thieno[2,3-c]pyridazin-6-yl)carbamate (intermediate 5)

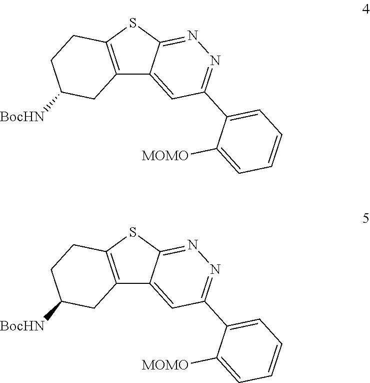

Intermediate 3 (1.20 g) was separated by chiral HPLC with the following conditions: Column: CHIRALPAK IC-3, 4.6*50 mm, 3 μm; Mobile Phase A: Hexane (0.1% DEA), Mobile Phase B: EtOH; Flow rate: 1 mL/min; Gradient: 0% B to 70% B. This resulted in intermediate 4 (475.0 mg, 39.5%) and intermediate 5 (490.0 mg, 40.8%) as yellow solids. LCMS (ESI) m/z: [M+H]⁺=442.

Steps 4 and 5: Preparation of (R)-2-(6-amino-5,6,7,8-tetrahydrobenzo[4,5]thieno[2,3-c]pyridazin-3-yl)phenol (intermediate A) and (S)-2-(6-amino-5,6,7,8-tetrahydrobenzo[4,5]thieno[2,3-c]pyridazin-3-yl)phenol (intermediate B)

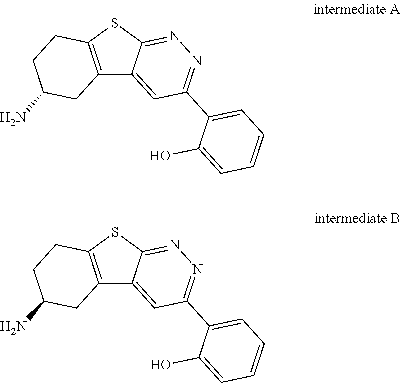 [SMILES: N[C@@H]1CCc2sc3nnc(-c4ccccc4O)cc3c2C1 ; N[C@H]1CCc2sc3nnc(-c4ccccc4O)cc3c2C1]

Intermediate 4 (10.0 mg, 0.023 mmol, 1.00 equiv) was dissolved in DCM (1 mL) and TFA (0.5 mL, 6.732 mmol, 297.23 equiv) was added. The mixture was stirred for one hour at room temperature. The resulting mixture was concentrated under reduced pressure. The residue was purified by Prep HPLC with the following conditions: Column: XBridge Shield RP18 OBD, 19*150 mm, 5 μm; Mobile Phase A: Water (0.1% FA), Mobile Phase B: ACN; Flow rate: 25 mL/min; Gradient: 11% B to 26% B in 7 min, then 26% B; Detector: 254/220 nm. This resulted in intermediate A (2.2 mg, 32.4%) as a light yellow solid. LCMS (ESI) m/z: [M+H]⁺=298.05.

Intermediate 5 (10.0 mg, 0.023 mmol, 1.00 equiv) was dissolved in DCM (1 mL) and TFA (0.5 mL, 6.732 mmol, 297.23 equiv) was added. The mixture was stirred for one hour at room temperature. The resulting mixture was concentrated under reduced pressure. The residue was purified by Prep HPLC with the following conditions: Column: XBridge Shield RP₁₈ OBD, 19*150 mm, 5 μm; Mobile Phase A: Water (0.1% FA), Mobile Phase B: ACN; Flow rate: 25 mL/min; Gradient: 11% B to 26% B in 7 min, then 26% B; Detector: 254/220 nm. This resulted in intermediate B (2.1 mg, 30.8%) as a light yellow solid. LCMS (ESI) m/z: [M+H]⁺=298.10.

Preparation of (2S,4R)-4-hydroxy-1-((R)-2-(3-(2-(((R)-3-(2-hydroxyphenyl)-5,6,7,8-tetrahydrobenzo[4,5]thieno[2,3-c]pyridazin-6-yl)amino)ethoxy)isoxazol-5-yl)-3-methylbutanoyl)-N—((S)-1-(4-(4-methylthiazol-5-yl)phenyl)ethyl)pyrrolidine-2-carboxamide (Compound 40)

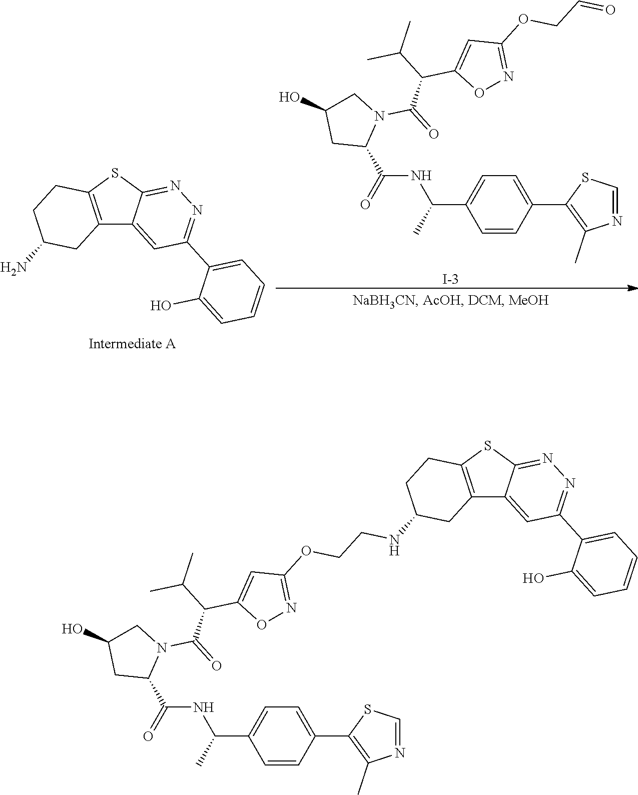

To a stirred mixture of Intermediate A (25 mg, 0.046 mmol, 1 equiv) and Intermediate I-3 (27.50 mg, 0.092 mmol, 2 equiv) in DCM (1.5 mL) were added AcOH (0.1 mL) and MeOH (0.5 mL) dropwise at room temperature. To the above mixture was added NaBH₃CN (8.72 mg, 0.138 mmol, 3 equiv) in portions at room temperature. The resulting mixture was stirred for 1 h at room temperature. The reaction mixture was purified by Prep HPLC with the following conditions: Column, XBridge Shield RP18 OBD, 19*150 mm, 5 μm; Mobile phase, water (0.1% FA) and ACN, 21% ACN up to 38% in 7 min; Detector, UV 254 nm. This resulted in Compound 40 (12.6 mg, 18.23%) as a light yellow solid. ¹H NMR (400 MHz, Methanol-d₄) δ 8.87 (d, J=4.9 Hz, 1H), 8.55-8.37 (m, 2H), 8.03 (d, J=7.9 Hz, 1H), 7.48-7.31 (m, 5H), 7.06-6.98 (m, 2H), 6.08 (s, 1H), 5.03 (d, J=7.0 Hz, 1H), 4.54-4.33 (m, 4H), 3.83 (dd, J=11.1, 4.2 Hz, 1H), 3.73-3.59 (m, 2H), 3.46-3.36 (m, 4H), 3.18-3.09 (m, 2H), 2.75 (s, 1H), 2.46 (d, J=13.4 Hz, 3H), 2.39 (s, 2H), 2.23-2.13 (m, 1H), 2.05-1.90 (m, 2H), 1.51 (d, J=7.0 Hz, 3H), 1.05 (d, J=6.5 Hz, 3H), 0.94-0.85 (m, 3H). LCMS (ESI) m/z: [M+H]⁺=822.25.

Preparation of (2S,4R)-4-hydroxy-1-((R)-2-(3-(2-(((S)-3-(2-hydroxyphenyl)-5,6,7,8-tetrahydrobenzo[4,5]thieno[2,3-c]pyridazin-6-yl)amino)ethoxy)isoxazol-5-yl)-3-methylbutanoyl)-N—((S)-1-(4-(4-methylthiazol-5-yl)phenyl)ethyl)pyrrolidine-2-carboxamide (Compound 39)

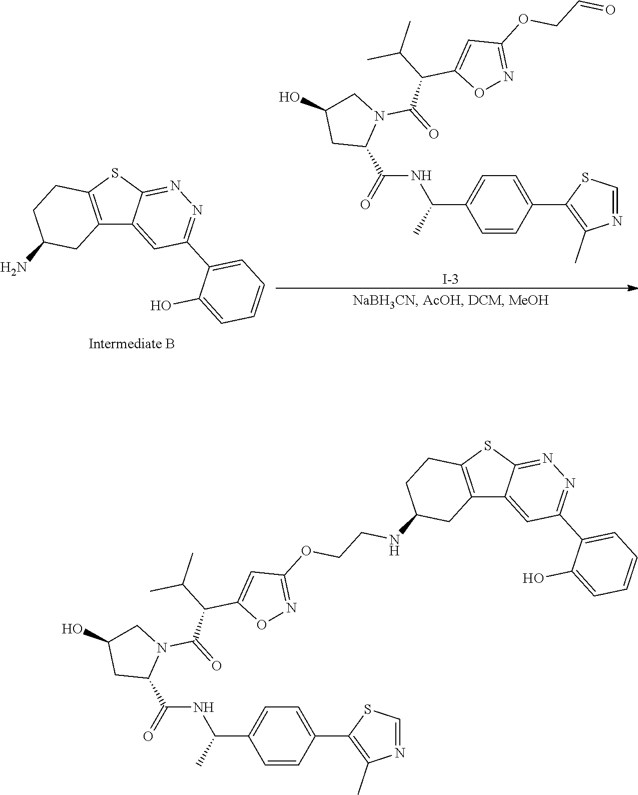

To a stirred solution of Intermediate B (25 mg, 0.046 mmol, 1 equiv) and Intermediate I-3 (27.50 mg, 0.092 mmol, 2 equiv) in DCM (1.5 mL) were added AcOH (0.1 mL) and MeOH (0.5 mL) dropwise at room temperature. To the above mixture was added NaBH₃CN (8.72 mg, 0.138 mmol, 3 equiv) in portions at room temperature. The resulting mixture was stirred for 1 h at room temperature. The reaction mixture was purified by Prep HPLC with the following conditions: Column, XBridge Shield RP18 OBD, 19*150 mm, 5 μm; Mobile phase, water (0.1% FA) and ACN, 21% ACN up to 38% in 7 min; Detector, UV 254 nm. This resulted in Compound 39 (11.8 mg, 17.08%) as an off-white solid. ¹H NMR (400 MHz, Methanol-d₄) δ 8.87 (d, J=4.6 Hz, 1H), 8.52-8.43 (m, 2H), 8.00 (dd, J=17.6, 7.9 Hz, 1H), 7.48-7.30 (m, 5H), 7.01 (dd, J=8.1, 4.9 Hz, 2H), 6.07 (s, 1H), 5.03 (t, J=7.0 Hz, 1H), 4.54-4.38 (m, 4H), 3.83 (dd, J=10.8, 4.1 Hz, 1H), 3.77-3.58 (m, 2H), 3.36 (s, 4H), 3.14 (d, J=10.1 Hz, 2H), 2.72 (s, 1H), 2.46 (d, J=13.1 Hz, 3H), 2.37 (s, 2H), 2.23-2.13 (m, 1H), 2.02-1.89 (m, 2H), 1.61-1.48 (m, 3H), 1.05 (d, J=6.5 Hz, 3H), 0.90 (dd, J=9.8, 6.7 Hz, 3H). LCMS (ESI) m/z: [M+H]⁺=822.25.

Preparation of 2-(5,6,7,8-tetrahydropyrido[3′,4′:4,5]thieno[2,3-c]pyridazin-3-yl)phenol (intermediate C)

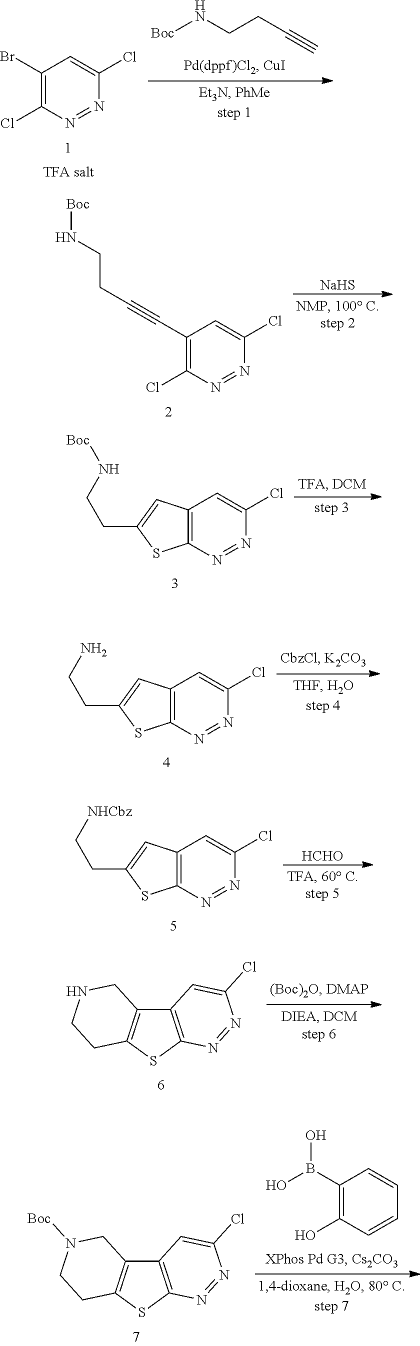

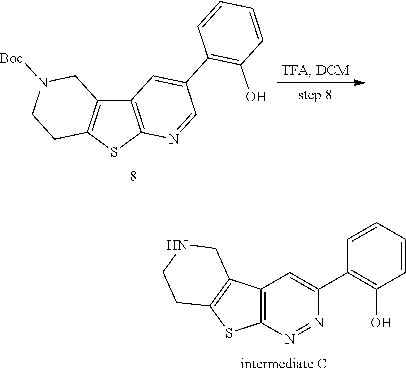 [SMILES: CC(C)(C)OC(=O)N1CCc2sc3ncc(-c4ccccc4O)cc3c2C1 ; Oc1ccccc1-c1cc2c3c(sc2nn1)CCNC3]

Step 1: Preparation of tert-butyl (4-(3,6-dichloropyridazin-4-yl)but-3-yn-1-yl)carbamate (Intermediate 2)

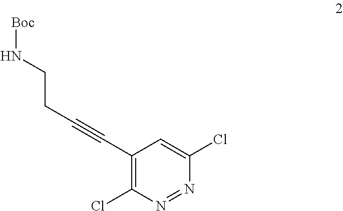 [SMILES: CC(C)(C)OC(=O)NCCC#Cc1cc(Cl)nnc1Cl]

A mixture of 4-bromo-3,6-dichloropyridazine (10.00 g, 43.885 mmol, 1.00 equiv), tert-butyl N-(but-3-yn-1-yl)carbamate (11.14 g, 65.828 mmol, 1.50 equiv), Pd(dppf)Cl₂ (6.42 g, 8.777 mmol, 0.20 equiv), CuI (2.51 g, 13.165 mmol, 0.30 equiv) and Et₃N (13.32 g, 131.655 mmol, 3.00 equiv) in toluene (100 mL) was stirred for 4 h at 60° C. under nitrogen atmosphere. After cooling down to room temperature, the resulting mixture was filtered and the filter cake was washed with EtOAc (3×100 mL). The filtrate was concentrated under reduced pressure. The residue was purified by silica gel column chromatography, eluted with PE/EA (1:1), to afford intermediate 2 (11 g, crude) as a brown solid. LCMS (ESI) m/z: [M+H]⁺=316.

Step 2: Preparation of tert-butyl (2-(3-chlorothieno[2,3-c]pyridazin-6-yl)ethyl)carbamate (Intermediate 3)

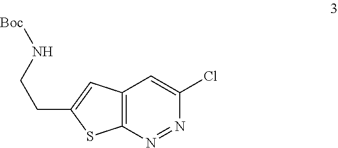 [SMILES: CC(C)(C)OC(=O)NCCc1cc2cc(Cl)nnc2s1]

A mixture of intermediate 2 (11.00 g, 34.790 mmol, 1.00 equiv) and sodium hydrosulfide (2.26 g, 27.832 mmol, 0.8 equiv) in NMP (50 mL) was stirred overnight at 100° C. After concentration under reduced pressure, the residue was purified by reverse phase flash chromatography with the following conditions: column, C18 silica gel; mobile phase, MeCN in water (0.1% FA), 20% to 80% gradient in 20 min; detector, UV 254 and 220 nm. This resulted in intermediate 3 (9.3 g, 85.19%) as a light yellow solid. LCMS (ESI) m/z: [M+H]⁺=314.

Step 3: Preparation of 2-(3-chlorothieno[2,3-c]pyridazin-6-yl)ethan-1-amine TFA salt (Intermediate 4)

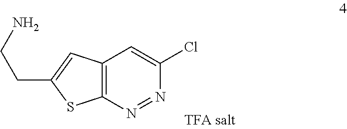 [SMILES: NCCc1cc2cc(Cl)nnc2s1]

A mixture of intermediate 3 (9.20 g, 29.318 mmol, 1.00 equiv) and TFA (3 mL) in DCM (9 mL) was stirred for 2 h at room temperature. The resulting mixture was concentrated under reduced pressure. The residue was diluted with water (5 mL). The resulting solid was filtered off and dried by lyophilization to afford intermediate 4 (3.2 g, crude) as a brown solid. LCMS (ESI) m/z: [M+H]⁺=214.

Step 4: Preparation of benzyl (2-(3-chlorothieno[2,3-c]pyridazin-6-yl)ethyl)carbamate (Intermediate 5)

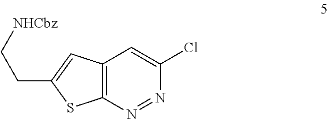 [SMILES: O=C(NCCc1cc2cc(Cl)nnc2s1)OCc1ccccc1]

A mixture of intermediate 4 (3.00 g, 14.040 mmol, 1.00 equiv), CbzCl (4.79 g, 28.080 mmol, 2.00 equiv) and K2CO3 (5.82 g, 42.120 mmol, 3.00 equiv) in THF (15 mL) and H₂O (15 mL) was stirred for 2 h at room temperature under nitrogen atmosphere. The resulting mixture was diluted with water (300 mL). The resulting mixture was extracted with EtOAc (3×200 mL). The combined organic layers were washed with water (300 mL), then dried over anhydrous Na₂SO₄. After filtration, the filtrate was concentrated under reduced pressure. The residue was dissolved in DMF (10 mL) and purified by reverse phase flash chromatography with the following conditions: column, C18 silica gel; mobile phase, MeCN in water (0.1% FA), 30% to 80% gradient in 20 min; detector, UV 254 and 220 nm. This resulted in intermediate 5 (497 mg, 10.18%) as a brown solid. LCMS (ESI) m/z: [M+H]⁺=348.

Step 5: Preparation of 3-chloro-5,6,7,8-tetrahydropyrido[3′,4′:4,5]thieno[2,3-c]pyridazine (Intermediate 6)

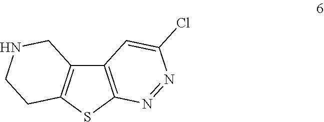 [SMILES: Clc1cc2c3c(sc2nn1)CCNC3]

A mixture of intermediate 5 (497.0 mg, 1.429 mmol, 1.00 equiv) and HCHO (128.1 mg, 4.287 mmol, 3.00 equiv) in TFA (4 mL) was stirred for 2 h at 60° C. The resulting mixture was concentrated under reduced pressure. The residue was dissolved in DMF (2 mL) and purified by reverse phase flash chromatography with the following conditions: column, C18 silica gel; mobile phase, MeCN in water (0.1% NH₃·H₂O), 0% to 50% gradient in 20 min; detector, UV 254 and 220 nm. This resulted in intermediate 6 (196 mg, 60.78%) as a light brown solid. LCMS (ESI) m/z: [M+H]⁺=226.

Step 6: Preparation of tert-butyl 3-chloro-7,8-dihydropyrido[3′,4′:4,5]thieno[2,3-c]pyridazine-6(5H)-carboxylate (Intermediate 7)

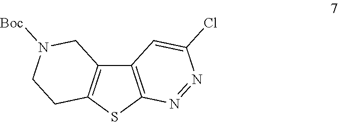 [SMILES: CC(C)(C)OC(=O)N1CCc2sc3nnc(Cl)cc3c2C1]

A mixture of intermediate 6 (186.0 mg, 0.824 mmol, 1.00 equiv), (Boc)₂O (359.7 mg, 1.648 mmol, 2.00 equiv), DMAP (20.1 mg, 0.165 mmol, 0.20 equiv) and DIEA (319.6 mg, 2.472 mmol, 3.00 equiv) in DCM (3 mL) was stirred for 2 h at room temperature. The resulting mixture was concentrated under reduced pressure. The residue was diluted with water (50 mL). The resulting mixture was extracted with EtOAc (3×25 mL). The combined organic layers were washed with water (50 mL), then dried over anhydrous Na₂SO₄. After filtration, the filtrate was concentrated under reduced pressure to afford intermediate 7 (247 mg, crude) as a brown solid. LCMS (ESI) m/z: [M+H]⁺=326.

Step 7: Preparation of tert-butyl 3-(2-hydroxyphenyl)-7,8-dihydrothieno[2,3-b:4,5-c′]dipyridine-6(5H)-carboxylate (Intermediate 8)

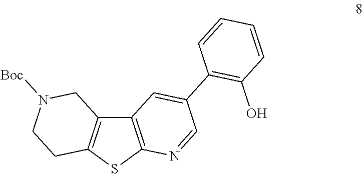 [SMILES: CC(C)(C)OC(=O)N1CCc2sc3ncc(-c4ccccc4O)cc3c2C1]

A mixture of intermediate 7 (237.0 mg, 0.727 mmol, 1.00 equiv), 2-hydroxyphenylboronic acid (150.5 mg, 1.091 mmol, 1.50 equiv), XPhos Pd G3 (123.2 mg, 0.145 mmol, 0.20 equiv) and Cs2CO3 (711.0 mg, 2.181 mmol, 3.00 equiv) in 1,4-dioxane (3 mL) and H₂O (0.6 mL) was stirred overnight at 80° C. under nitrogen atmosphere. The resulting mixture was concentrated under reduced pressure. The residue was dissolved in DMF (5 mL) and purified by reverse phase flash chromatography with the following conditions: column, C18 silica gel; mobile phase, MeCN in water (0.1% FA), 0% to 80% gradient in 20 min; detector, UV 254 and 220 nm. This resulted in intermediate 8 (61 mg, 21.87%) as a brown solid. LCMS (ESI) m/z: [M+H]⁺=383.

Step 8: Preparation of 2-(5,6,7,8-tetrahydropyrido[3′,4′:4,5]thieno[2,3-c]pyridazin-3-yl)phenol (Intermediate C)

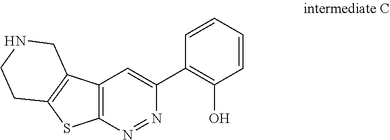 [SMILES: Oc1ccccc1-c1cc2c3c(sc2nn1)CCNC3]

A mixture of intermediate 8 (56.0 mg, 0.146 mmol, 1.00 equiv) and TFA (0.5 mL) in DCM (1.5 mL) was stirred for 2 h at room temperature. The resulting mixture was concentrated under reduced pressure. The residue was diluted with water (5 mL). The resulting solid was filtered off and dried by lyophilization to afford intermediate C (76 mg, crude) as a yellow solid. LCMS (ESI) m/z: [M+H]⁺=284.

Preparation of (2S,4R)-4-hydroxy-1-((R)-2-(3-(4-(3-(2-hydroxyphenyl)-5,6,7,8-tetrahydropyrido[3′,4′:4,5]thieno[2,3-c]pyridazine-6-carbonyl)piperidin-1-yl)isoxazol-5-yl)-3-methylbutanoyl)-N—((S)-1-(4-(4-methylthiazol-5-yl)phenyl)ethyl)pyrrolidine-2-carboxamide (Compound 11)

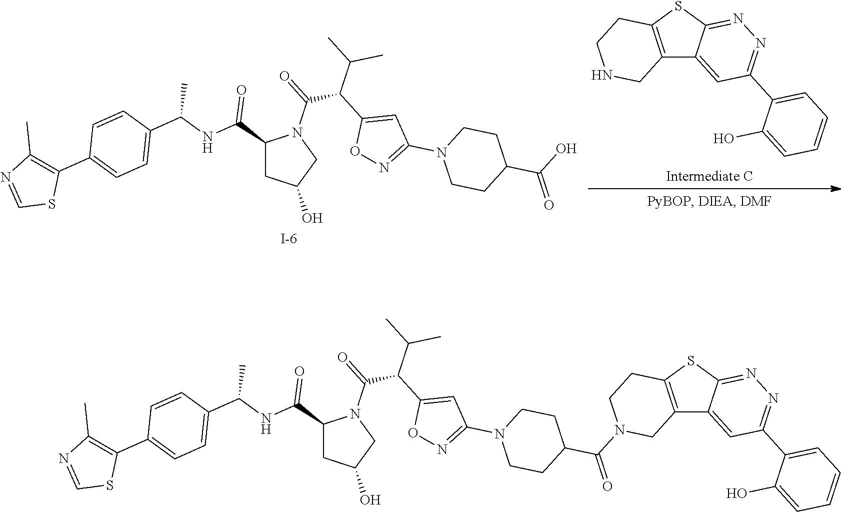

To a stirred solution of I-6 (15 mg, 0.025 mmol, 11.00 equiv) and intermediate C (8.36 mg, 0.030 mmol, 1.2 equiv) in DMF (2 mL) were added PyBOP (19.20 mg, 0.038 mmol, 1.5 equiv) and DIEA (9.54 mg, 0.075 mmol, 3 equiv) at room temperature. The resulting mixture was stirred for 2 h at room temperature. The mixture was purified by Prep HPLC with the following conditions: Column: XBridge Prep Phenyl OB, 19*150 mm, 5 μm; Mobile Phase A: water (0.05% NH₃—H₂O), Mobile Phase B: ACN; Flow rate: 25 mL/min; Gradient: 50% B to 63% B in 7 min; Detector: 254/220 nm. This resulted in Compound 11 (4.0 mg, 18.26%) as a white solid. ¹H NMR (400 MHz, DMSO-d6) 12.80 (s, 1H), 8.99 (s, 1H), 8.84 (d, J=11.0 Hz, 1H), 8.41 (d, J=7.5 Hz, 1H), 8.21 (d, J=8.0 Hz, 1H), 7.44 (d, J=8.2 Hz, 2H), 7.37 (d, J=8.2 Hz, 3H), 7.03 (q, J=7.9, 7.3 Hz, 2H), 6.16 (s, 1H), 5.11 (d, 1H), 5.00-4.77 (m, 3H), 4.37 (s, 1H), 4.29 (s, 1H), 4.00 (s, 1H), 3.93 (s, 1H), 3.69 (d, J=13.7 Hz, 3H), 3.58 (d, J=10.1 Hz, 1H), 3.48 (s, 1H), 3.18 (s, 1H), 3.04 (s, 2H), 2.93 (d, J=12.6 Hz, 2H), 2.46 (s, 3H), 2.23 (s, 1H), 2.02 (s, 1H), 1.70 (d, J=27.2 Hz, 5H), 1.38 (d, J=7.0 Hz, 3H), 0.95 (d, J=6.6 Hz, 3H), 0.81 (d, J=6.3 Hz, 3H). LCMS (ESI) m/z: [M+H]⁺=875.60.

The compounds in Table 12 was prepared using a procedure similar to the one used above for the preparation of compound 11 using the appropriate amine and carboxylic acid.

TABLE 12

| | | LCMS | |
|---|---|---|---|
| | | (ESI) | |
| No. | Name | m/z | ¹H NMR |
| 91 | (2S,4R)-4-hydroxy-1-((R)-2-(3-(2- | 822.1 | 1H NMR (400 MHz, Methanol-d4) δ 8.87 (s, |
| | (3-(2-hydroxyphenyl)-7,8- | | 1H), 8.61 (t, J = 16.1 Hz, 1H), 8.06 (dd, J = |
| | dihydropyrido[3′,4′:4, 5]thieno[2,3- | | 8.2, 1.6 Hz, 1H), 7.48-7.29 (m, 5H), 7.01 |
| | c]pyridazin-6(5H)-yl)-2- | | (q, J = 11.9, 9.3 Hz, 2H), 6.16-6.04 (m, |
| | oxoethoxy)isoxazol-5-yl)-3- | | 1H), 5.17 (s, 2H), 5.02 (d, J = 7.0 Hz, 1H), |
| | methylbutanoyl)-N-((S)-1-(4-(4- | | 4.94 (s, 1H), 4.50 (t, J = 8.3 Hz, 1H), 4.43 |
| | methylthiazol-5- | | (s, 1H), 4.07 (t, J = 5.5 Hz, 1H), 3.95 (t, J = |
| | yl)phenyl)ethyl)pyrrolidine-2- | | 5.7 Hz, 1H), 3.82 (dd, J = 10.9, 4.2 Hz, 1H), |
| | carboxamide | | 3.68 (d, J = 10.0 Hz, 1H), 3.64-3.57 (m, |
| | | | 1H), 3.23 (s, 2H), 2.48 (s, 3H), 2.41-2.31 |
| | | | (m, 1H), 2.23-2.12 (m, 1H), 2.00-1.90 |
| | | | (m, 1H), 1.51 (d, J = 7.0 Hz, 3H), 1.04 (d, J = |
| | | | 6.5 Hz, 3H), 0.88 (d, J = 6.9 Hz, 3H). |

Preparation of (2S,4R)-4-hydroxy-1-((R)-2-(3-(2-(3-(3-(2-hydroxyphenyl)thieno[3,2-c]pyridazin-6-yl)azetidin-1-yl)ethoxy)isoxazol-5-yl)-3-methylbutanoyl)-N—((S)-1-(4-(4-methylthiazol-5-yl)phenyl)ethyl)pyrrolidine-2-carboxamide

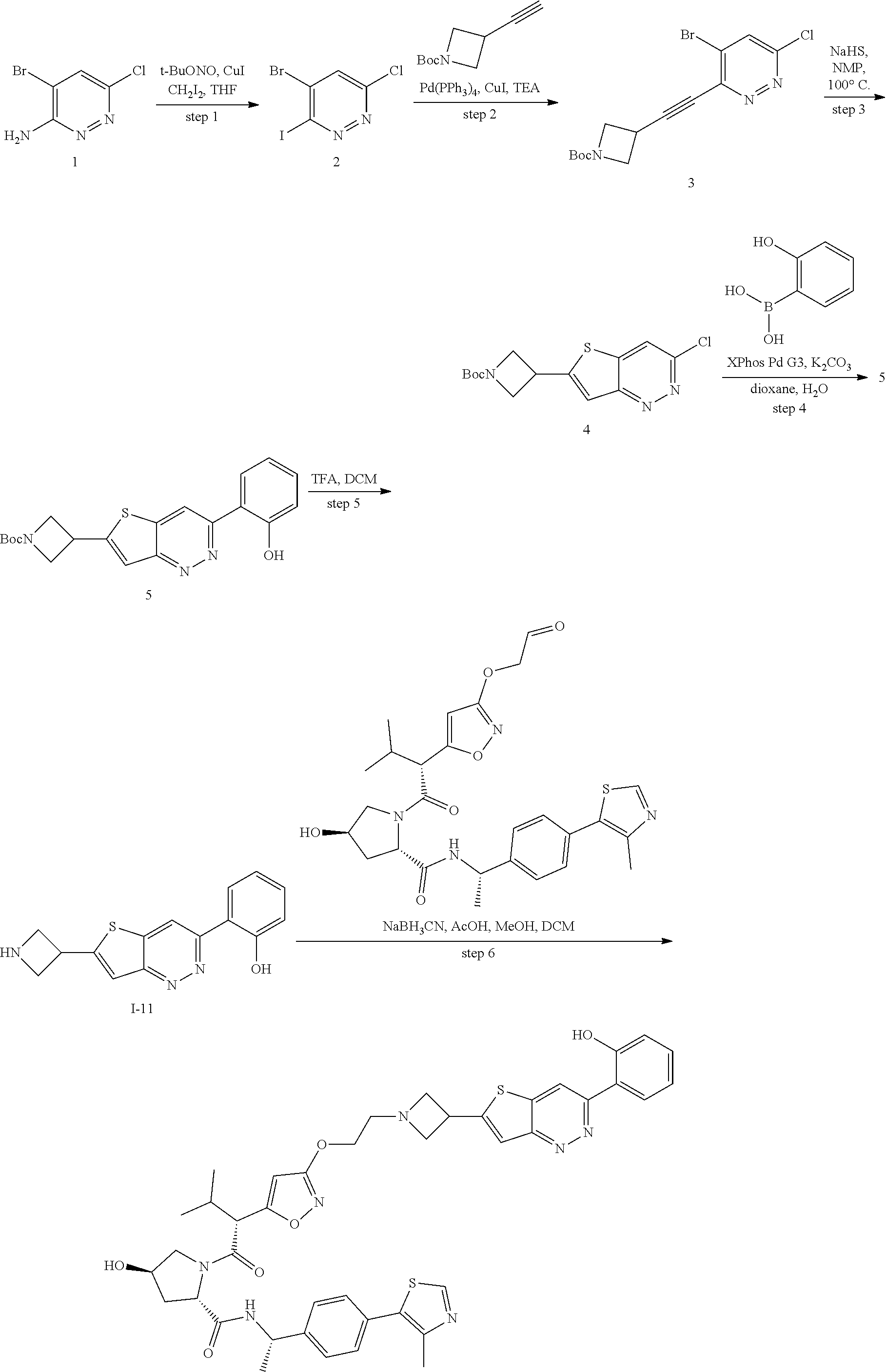

Step 1: Preparation of 4-bromo-6-chloro-3-iodopyridazine (intermediate 2)

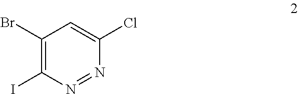

To a stirred solution of 4-bromo-6-chloropyridazin-3-amine (2.00 g, 9.595 mmol, 1.00 equiv) and t-BuONO (1.09 g, 10.555 mmol, 1.10 equiv) in THF (20 mL) was added CuI (1.83 g, 9.595 mmol, 1.00 equiv) and diiodomethane (2.57 g, 9.595 mmol, 1.00 equiv). The resulting mixture was stirred for overnight at 60 degrees C. The resulting mixture was diluted with EtOAc (300 mL), washed with sat. NH₄Cl (aq.) (100 mL×3), and the organic layer was dried over anhydrous Na₂SO₄. After filtration, the filtrate was concentrated under reduced pressure. The residue was purified by silica gel column chromatography, eluted with PE/EtOAc (4:1) to afford intermediate 2 (1.1 g, 35.90%) as a brown solid. LCMS (ESI) m/z: [M+H]⁺=319.

Step 2: Preparation of tert-butyl 3-[2-(4-bromo-6-chloropyridazin-3-yl)ethynyl]azetidine-1-carboxylate (intermediate 3)

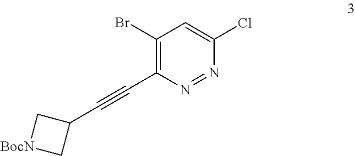 [SMILES: CC(C)(C)OC(=O)N1CC(C#Cc2nnc(Cl)cc2Br)C1]

To a stirred solution of intermediate 2 (1.10 g, 3.445 mmol, 1.00 equiv) and tert-butyl 3-ethynylazetidine-1-carboxylate (624.3 mg, 3.445 mmol, 1.00 equiv) in THF (15 mL) was added Pd(PPh₃)₄ (796.1 mg, 0.689 mmol, 0.20 equiv), CuI (196.8 mg, 1.033 mmol, 0.30 equiv) and TEA (1.05 g, 10.335 mmol, 3.00 equiv). The resulting mixture was stirred for 2 h at 60 degrees C. under nitrogen atmosphere. The reaction mixture was filtered through a short pad of Celite and concentrated in vacuo. The residue was purified by silica gel column chromatography, eluted with PE/EtOAc (1:1) to afford intermediate 3 (702 mg, 54.69%) as a brown solid. LCMS (ESI) m/z: [M+H]⁺=372.

Step 3: Preparation of tert-butyl 3-{3-chlorothieno[3,2-c]pyridazin-6-yl}azetidine-1-carboxylate (intermediate 4)

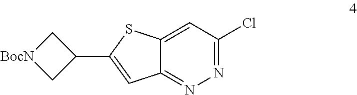 [SMILES: CC(C)(C)OC(=O)N1CC(c2cc3nnc(Cl)cc3s2)C1]

To a stirred solution of intermediate 3 (702.0 mg, 1.884 mmol, 1.00 equiv) in NMP (10 mL) was added NaSH (116.0 mg, 2.072 mmol, 1.10 equiv). The resulting mixture was stirred for 1 h at 100 degrees C. The mixture was purified by directly by reverse phase flash chromatography, elution gradient 0 to 70% MeCN in water (containing 0.1% formic acid) to afford intermediate 4 (450 mg, 73.32%) as a white solid. LCMS (ESI) m/z: [M+H]⁺=326.

Step 4: Preparation of tert-butyl 3-[3-(2-hydroxyphenyl)thieno[3,2-c]pyridazin-6-yl]azetidine-1-carboxylate (intermediate 5)

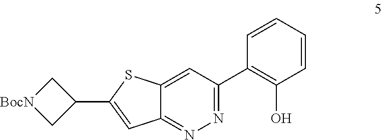 [SMILES: CC(C)(C)OC(=O)N1CC(c2cc3nnc(-c4ccccc4O)cc3s2)C1]

To a stirred solution of intermediate 4 (450.0 mg, 1.381 mmol, 1.00 equiv) and 2-hydroxyphenylboronic acid (571.5 mg, 4.143 mmol, 3.00 equiv) in dioxane (10 mL) and H₂O (2 mL) was added XPhos Pd G3 (233.8 mg, 0.276 mmol, 0.20 equiv) and K2CO3 (572.6 mg, 4.143 mmol, 3.00 equiv). The resulting mixture was stirred for 2 h at 100 degrees C. under nitrogen atmosphere. The resulting mixture was filtered, and the filter cake was washed with EtOAc (30 mL×3). The filtrate was concentrated under reduced pressure. The residue was purified by silica gel column chromatography, eluted with PE/EtOAc (1:1) to afford intermediate 5 (435 mg, 82.13%) as a white solid. LCMS (ESI) m/z: [M+H]⁺=384.

Step 5: Preparation of 2-[6-(azetidin-3-yl)thieno[3,2-c]pyridazin-3-yl]phenol (I-11)

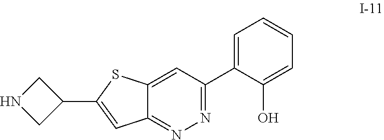 [SMILES: Oc1ccccc1-c1cc2sc(C3CNC3)cc2nn1]

To a stirred solution of intermediate 5 (435.0 mg, 1.134 mmol, 1.00 equiv) in DCM (5 mL) was added TFA (5 mL). The resulting mixture was stirred for 1 h at room temperature. The resulting mixture was concentrated under vacuum. The crude product was purified by Prep-HPLC with the following conditions (Column: XBridge Prep C18 OBD Column, 19*150 mm, 5 μm; Mobile Phase A: Water(10 mmol/L NH₄HCO₃), Mobile Phase B: ACN; Flow rate: 25 mL/min; Gradient: 18% B to 31% B in 8 min, 31% B; Wave Length: 254/220 nm; RT1(min): 6.80) to afford I-11 (233.1 mg, 72.49%) as a white solid. ¹H NMR (400 MHz, DMSO-d₆) δ 12.94 (s, 1H), 9.16 (s, 1H), 8.03 (dd, J=8.0, 1.6 Hz, 1H), 7.78 (s, 1H), 7.51-7.21 (m, 1H), 7.12-6.76 (m, 2H), 4.36 (d, J=8.3 Hz, 1H), 4.30-4.26 (p, J=6.9 Hz, 1H), 3.95 (t, J=7.7 Hz, 2H), 3.68 (t, J=7.0 Hz, 2H). LCMS (ESI) m/z: [M+H]⁺=284.10.

Step 6: Preparation (2S,4R)-4-hydroxy-1-((R)-2-(3-(2-(3-(3-(2-hydroxyphenyl)thieno[3,2-c]pyridazin-6-yl)azetidin-1-yl)ethoxy)isoxazol-5-yl)-3-methylbutanoyl)-N—((S)-1-(4-(4-methylthiazol-5-yl)phenyl)ethyl)pyrrolidine-2-carboxamide (Compound 9)

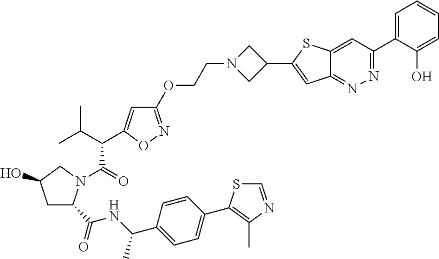 [SMILES: Cc1ncsc1-c1ccc([C@H](C)NC(=O)[C@@H]2C[C@@H](O)CN2C(=O)[C@@H](c2cc(OCCN3CC(c4cc5nnc(-c6ccccc6O)cc5s4)C3)no2)C(C)C)cc1]

A mixture of compound I-11 (42.0 mg, 0.148 mmol, 1.00 equiv), (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (80.1 mg, 0.148 mmol, 1.00 equiv) and AcOH (8.9 mg, 0.148 mmol, 1.00 equiv) in MeOH (3 mL) and DCM (3 mL) was stirred for 30 min at room temperature. To the above mixture was added NaBH3CN (46.5 mg, 0.740 mmol, 5.00 equiv) at room temperature. The resulting mixture was stirred for additional 3 h at room temperature, then concentrated under vacuum. The crude product was purified by Prep-HPLC with the following conditions (Column: XBridge Prep C18 OBD Column, 19*150 mm, 5 μm; Mobile Phase A: Water(10 mmol/L NH₄HCO₃), Mobile Phase B: ACN; Flow rate: 25 mL/min; Gradient: 40% B to 57% B in 8 min, 57% B to afford Compound 9 (67.4 mg, 55.71%) as an off-white solid. ¹H NMR (300 MHz, DMSO-d6) δ 12.96 (s, 1H), 9.14 (s, 1H), 8.99 (s, 1H), 8.42 (d, J=7.6 Hz, 1H), 8.07-7.97 (m, 1H), 7.77 (s, 1H), 7.44 (d, J=8.3 Hz, 2H), 7.39-7.33 (m, 3H), 7.07-6.97 (m, 2H), 6.11 (s, 1H), 5.06 (dd, J=26.0, 3.2 Hz, 1H), 4.98-4.84 (m, 1H), 4.38 (t, J=7.9 Hz, 1H), 4.29 (s, 1H), 4.18 (t, J=5.3 Hz, 2H), 4.09 (t, J=6.6 Hz, 1H), 3.82-3.71 (m, 2H), 3.70-3.63 (m, 2H), 3.50-3.42 (m, 1H), 3.41-3.39 (m, 2H), 2.85 (t, J=5.4 Hz, 2H), 2.45 (d, J=3.5 Hz, 3H), 2.33-2.13 (m, 1H), 2.12-1.96 (m, 1H), 1.85-1.73 (m, 1H), 1.38 (d, J=7.0 Hz, 3H), 0.97-0.94 (m, 3H), 0.80 (d, J=6.6 Hz, 3H). LCMS (ESI) m/z [M+H]⁺=808.30.

The compounds in Table 13 were prepared using procedures similar to those used above for the preparation of compound 9 using the appropriate amine and aldehyde (or ketone).

TABLE 13

| | | LCMS | |
|---|---|---|---|
| | | (ESI) | |
| No. | Name | m/z | ¹H NMR |
| 49 | (2S,4R)-4-hydroxy-1-((R)-2-(3-(2- | 837.15 | 1H NMR (300 MHz, Methanol-d4) δ 8.87 |
| | (4-(3-(2-hydroxyphenyl)thieno[3,2- | | (d, J = 16.0 Hz, 1H), 8.64 (d, J = 6.5 Hz, |
| | c]pyridazin-6-yl)piperazin-1- | | 1H), 8.12-7.75 (m, 1H), 7.58-7.25 (m, |
| | yl)ethoxy)isoxazol-5-yl)-3- | | 5H), 6.99 (td, J = 7.3, 6.9, 1.4 Hz, 2H), 6.47 |
| | methylbutanoyl)-N-((S)-1-(4-(4- | | (d, J = 4.5 Hz, 1H), 6.04 (d, J = 17.2 Hz, |
| | methylthiazol-5- | | 1H), 5.16-4.98 (m, 1H), 4.63-4.32 (m, |
| | yl)phenyl)ethyl)pyrrolidine-2- | | 4H), 3.86 (dd, J = 10.9, 4.1 Hz, 1H), 3.78- |
| | carboxamide | | 3.65 (m, 2H), 3.59 (p, J = 5.3 Hz, 4H), 2.99 |
| | | | (t, J = 5.2 Hz, 2H), 2.89 (dt, J = 10.0, 5.2 |
| | | | Hz, 4H), 2.48 (d, J = 8.8 Hz, 3H), 2.45- |
| | | | 2.13 (m, 2H), 1.98 (ddd, J = 13.3, 8.7, 4.6 |
| | | | Hz, 1H), 1.58 (dd, J = 22.5, 7.0 Hz, 3H), |
| | | | 1.08 (d, J = 6.6 Hz, 3H), 0.93 (t, J = 6.9 Hz, |
| | | | 3H). |
| 73 | (2S,4R)-4-hydroxy-1-((R)-2-(3-(2- | 852.1 | 1H NMR (400 MHz, DMSO-d6) δ 13.05 (d, |
| | (4-hydroxy-4-(3-(2- | | J = 8.5 Hz, 1H), 9.15 (s, 1H), 8.99 (s, 1H), |
| | hydroxyphenyl)thieno[3,2- | | 8.44 (d, J = 7.7 Hz, 1H), 8.03-7.95 (m, |
| | c]pyridazin-6-yl)piperidin-1- | | 1H), 7.77 (s, 1H), 7.61-7.20 (m, 5H), 7.82- |
| | yl)ethoxy)isoxazol-5-yl)-3- | | 7.02 (m, 2H), 6.13 (s, 1H), 5.98 (d, J = |
| | methylbutanoyl)-N-((S)-1-(4-(4- | | 17.5 Hz, 1H), 5.12 (d, J = 3.6 Hz, 1H), 4.98- |
| | methylthiazol-5- | | 4.78 (m, 1H), 4.48-4.12 (m, 4H), 3.78- |
| | yl)phenyl)ethyl)pyrrolidine-2- | | 3.62 (m, 2H), 3.49-3.42 (m, 1H), 2.98- |
| | carboxamide | | 2.69 (m, 4H), 2.48-2.40 (m, 4H), 2.29- |
| | | | 2.00 (m, 4H), 2.08-1.99 (m, 3H), 1.46 (d, |
| | | | J = 6.9 Hz, 1H), 1.37 (d, J = 7.0 Hz, 3H), |
| | | | 0.96 (d, J = 6.4 Hz, 3H), 0.90-0.83 (m, |
| | | | 3H). |
| 87 | (2S,4R)-4-hydroxy-1-((R)-2-(3-(4- | 875.2 | 1H NMR (400 MHz, Methanol-d4) δ 8.99- |
| | (3-(2-hydroxyphenyl)thieno[3,2- | | 8.84 (m, 2H), 7.97 (d, J = 7.6 Hz, 1H), 7.57 |
| | c]pyridazin-6-yl)-[1,4′-bipiperidin]- | | (d, J = 7.0 Hz, 1H), 7.49-7.33 (m, 5H), |
| | 1′-yl)isoxazol-5-yl)-3- | | 7.01 (t, J = 8.0 Hz, 2H), 6.12 (s, 1H), 5.04 |
| | methylbutanoyl)-N-((S)-1-(4-(4- | | (d, J = 6.8 Hz, 1H), 4.60-4.48 (m, 2H), |
| | methylthiazol-5- | | 3.87-3.71 (m, 3H), 3.67-3.57 (m, 2H), |
| | yl)phenyl)ethyl)pyrrolidine-2- | | 3.18-3.12 (m, 2H), 2.89 (t, J = 12.3 Hz, |
| | carboxamide | | 2H), 2.65 (s, 3H), 2.52-2.46 (m, J = 7.0 |
| | | | Hz, 4H), 2.42-2.31 (m, 1H), 2.23-2.20 (m, |
| | | | 3H), 1.99-1.90 (m, 5H), 1.65 (d, J = 13.5 |
| | | | Hz, 2H), 1.52 (d, J = 7.0 Hz, 3H), 1.05 (d, J = |
| | | | 6.5 Hz, 3H), 0.93-0.88 (m, 3H). |
| 97 | (2S,4R)-4-hydroxy-1-((R)-2-(3-(2- | 836.1 | 1H NMR (300 MHz, Methanol-d4) δ 9.08- |
| | (4-(3-(2-hydroxyphenyl)thieno[3,2- | | 8.90 (m, 2H), 8.06 (d, J = 7.7 Hz, 1H), 7.69- |
| | c]pyridazin-6-yl)piperidin-1- | | 7.62 (m, 1H), 7.58-7.38 (m, 5H), 7.15- |
| | yl)ethoxy)isoxazol-5-yl)-3- | | 7.04 (m, 2H), 6.13 (s, 1H), 5.12 (d, J = 7.0 |
| | methylbutanoyl)-N-((S)-1-(4-(4- | | Hz, 1H), 4.65-4.45 (m, 4H), 3.93 (dd, J = |
| | methylthiazol-5- | | 10.8, 4.1 Hz, 1H), 3.79-3.76 (m, 2H), 3.26 |
| | yl)phenyl)ethyl)pyrrolidine-2- | | (d, J = 11.8 Hz, 3H), 2.97 (t, J = 5.3 Hz, |
| | carboxamide | | 2H), 2.56 (d, J = 6.1 Hz, 3H), 2.44 (d, J = |
| | | | 11.8 Hz, 3H), 2.26 (d, J = 12.1 Hz, 3H), |
| | | | 2.12-1.94 (m, 3H), 1.65 (dd, J = 22.3, 7.0 |
| | | | Hz, 3H), 1.15 (d, J = 6.6 Hz, 3H), 1.00 (t, J = |
| | | | 6.9 Hz, 3H). |
| 103 | (2S,4R)-4-hydroxy-1-((R)-2-(3-(4- | 861.3 | 1H NMR (400 MHz, Methanol-d4) δ 8.95 |
| | ((3-(3-(2-hydroxyphenyl)thieno[3,2- | | (d, J = 3.1 Hz, 1H), 8.87 (d, J = 4.3 Hz, 1H), |
| | c]pyridazin-6-yl)azetidin-1- | | 7.97 (d, J = 8.0 Hz, 1H), 7.66 (s, 1H), 7.53- |
| | yl)methyl)piperidin-1-yl)isoxazol-5- | | 7.29 (m, 5H), 7.02 (d, J = 7.9 Hz, 2H), |
| | yl)-3-methylbutanoyl)-N-((S)-1-(4- | | 6.17-6.00 (m, 1H), 5.03 (d, J = 7.0 Hz, |
| | (4-methylthiazol-5- | | 1H), 4.51 (t, J = 8.2 Hz, 1H), 4.44 (s, 1H), |
| | yl)phenyl)ethyl)pyrrolidine-2- | | 4.15 (d, J = 7.2 Hz, 1H), 3.96-3.81 (m, |
| | carboxamide | | 3H), 3.70 (d, J = 12.7 Hz, 2H), 3.61 (dd, J = |
| | | | 10.4, 6.6 Hz, 2H), 3.54 (d, J = 7.9 Hz, |
| | | | 2H), 2.85 (t, J = 12.1 Hz, 2H), 2.58 (d, J = |
| | | | 7.1 Hz, 2H), 2.47 (d, J = 4.9 Hz, 3H), 2.43- |
| | | | 2.29 (m, 1H), 2.18 (t, J = 10.6 Hz, 1H), |
| | | | 1.96 (ddd, J = 13.2, 8.6, 4.8 Hz, 1H), 1.83 |
| | | | (d, J = 12.9 Hz, 2H), 1.65 (s, 1H), 1.56 (d, |
| | | | J = 7.0 Hz, 3H), 1.40-1.29 (m, 2H), 1.05 |
| | | | (d, J = 6.5 Hz, 3H), 0.90 (dd, J = 12.0, 6.6 |
| | | | Hz, 3H). |
| 104 | (2S,4R)-4-hydroxy-1-((R)-2-(3-(3- | 822.2 | 1H NMR (400 MHz, Methanol-d4) δ 8.96 |
| | (3-(3-(2-hydroxyphenyl)thieno[3,2- | | (s, 1H), 8.92-8.81 (m, 1H), 7.98 (d, J = |
| | c]pyridazin-6-yl)azetidin-1- | | 8.0 Hz, 1H), 7.64 (d, J = 9.5 Hz, 1H), 7.47- |
| | yl)propoxy)isoxazol-5-yl)-3- | | 7.30 (m, 5H), 7.05-6.93 (m, 2H), 6.02 |
| | methylbutanoyl)-N-((S)-1-(4-(4- | | (s, 1H), 5.02 (d, J = 7.0 Hz, 1H), 4.51 (t, J = |
| | methylthiazol-5- | | 8.2 Hz, 1H), 4.41 (d, J = 17.3 Hz, 1H), |
| | yl)phenyl)ethyl)pyrrolidine-2- | | 4.28 (t, J = 6.3 Hz, 2H), 4.11 (q, J = 7.0 Hz, |
| | carboxamide | | 1H), 3.82 (p, J = 8.1, 7.4 Hz, 3H), 3.65 (d, |
| | | | J = 9.9 Hz, 2H), 3.42 (t, J = 6.9 Hz, 2H), |
| | | | 2.71 (t, J = 7.2 Hz, 2H), 2.45-2.31 (m, |
| | | | 4H), 2.22-2.13 (m, 1H), 2.01-1.83 (m, |
| | | | 3H), 1.58-1.50 (d, J = 7.0 Hz, 3H), 1.05 (d, |
| | | | J = 6.5 Hz, 3H), 0.94-0.86 (m, 3H). |
| 105 | (2S,4R)-4-hydroxy-1-((R)-2-(3-(4- | 847.25 | 1H NMR (400 MHz, DMSO-d6, with a drop |
| | (3-(3-(2-hydroxyphenyl)thieno[3,2- | | of D2O) δ 9.20-9.14 (m, 1H), 8.99 (s, 1H), |
| | c]pyridazin-6-yl)azetidin-1- | | 8.44-8.36 (m, 1H), 8.20-7.71 (m, 2H), |
| | yl)piperidin-1-yl)isoxazol-5-yl)-3- | | 7.49-7.42 (m, 2H), 7.42-7.34 (m, 3H), |
| | methylbutanoyl)-N-((S)-1-(4-(4- | | 7.09-6.98 (m, 2H), 6.25-6.13 (m, 1H), |
| | methylthiazol-5- | | 4.97-4.86 (m, 1H), 4.82-4.21 (m, 5H), |
| | yl)phenyl)ethyl)pyrrolidine-2- | | 4.15-3.67 (m, 3H), 3.67-3.49 (m, 3H), |
| | carboxamide | | 3.49-3.41 (m, 1H), 3.06-2.73 (m, 2H), |
| | | | 2.46 (s, 3H), 2.29-2.13 (m, 1H), 2.10- |
| | | | 1.88 (m, 2H), 1.86-1.60 (m, 2H), 1.44- |
| | | | 1.26 (m, 5H), 0.97 (t, J = 6.7 Hz, 3H), 0.82 |
| | | | (d, J = 6.7 Hz, 3H). |
| 112 | (2S,4R)-4-hydroxy-1-((R)-2-(3-((2- | 821.4 | 1H NMR (400 MHz, DMSO-d6) δ 12.97 (s, |
| | (3-(3-(2-hydroxyphenyl)thieno[3,2- | | 1H), 9.13 (d, J = 6.8 Hz, 1H), 8.98 (d, J = |
| | c]pyridazin-6-yl)azetidin-1- | | 4.4 Hz, 1H), 8.41 (d, J = 7.7 Hz, 1H), 8.02 |
| | yl)ethyl)(methyl)amino)isoxazol-5- | | (d, J = 9.2, 4.6 Hz, 1H), 7.74 (d, J = 5.8 Hz, |
| | yl)-3-methylbutanoyl)-N-((S)-1-(4- | | 1H), 7.47-7.41 (m, 2H), 7.36 (dd, J = 8.9, |
| | (4-methylthiazol-5- | | 3.2 Hz, 3H), 7.01 (dd, J = 11.8, 7.8 Hz, 2H), |
| | yl)phenyl)ethyl)pyrrolidine-2- | | 6.05 (s, 1H), 5.11 (s, 1H), 4.91 (t, J = 7.2 |
| | carboxamide | | Hz, 1H), 4.38 (t, J = 7.8 Hz, 1H), 4.29 (s, |
| | | | 1H), 4.07-4.01 (m, 1H), 3.75-3.61 (m, |
| | | | 3H), 3.54 (dd, 1H), 3.43 (d, J = 10.2 Hz, |
| | | | 1H), 3.32-3.28 (m, 2H), 3.21 (t, J = 6.4 Hz, |
| | | | 2H), 2.90 (s, 3H), 2.45 (d, J = 3.5 Hz, 3H), |
| | | | 2.33-2.13 (m, 1H), 2.12-1.96 (m, 1H), |
| | | | 1.85-1.73 (m, 1H), 1.38 (d, J = 7.0 Hz, |
| | | | 3H), 0.97-0.94 (m, 3H), 0.80 (d, J = 6.6 |
| | | | Hz, 3H). |

### Example 2. Degradation of BRM and BRG1 by Compounds of the Invention

This example demonstrates the ability of the compounds of the disclosure to degrade a HiBit-BRM or HiBit-BRG1 fusion protein in a cell-based degradation assay.

Procedure: A stable HeLa cell line expressing HiBiT-BRM was generated. On day 0, 5000 cells were seeded in 40 μL of media into each well of 384-well cell culture plates. On day 1, cells were treated with 120 nL DMSO or 120 nL of 3-fold serially DMSO-diluted compounds (10 points in duplicate with 30 μM as final top dose). Subsequently plates were incubated for 24 h in a standard tissue culture incubator and equilibrated at room temperature for 15 minutes. Nano-Glo HiBiT Lytic Detection System (Promega N3050) reagent was freshly prepared and 20 ul was added to each well. Upon addition of this LgBit-containing reagent, the HiBiT and LgBiT proteins associate to form the luminescent NanoBiT luciferase. The plates were shaken for 10 minutes at room temperature and the bioluminescence read using an EnVision plate reader (PerkinElmer).

For measurement of BRG1 degradation, a stable HeLa cell line expressing HiBit-BRG1 and LgBit was generated. The same protocol as above was then followed.

The degradation % was calculated using the following formula: % degradation=100%−100%×(Lum_{Sample}−Lum_{LC})/(Lum_{HC}−Lum_{LC}). DMSO treated cells are employed as High Control (HC) and 2 μM of a known BRM/BRG1 degrader standard treated cells are employed as Low Control (LC). The data was fit to a four parameter, non-linear curve fit to calculate IC₅₀ (μM) values as shown in Table 14.

Results: As shown in Table 14 below, the compounds of the invention degraded both BRM and BRG1.

TABLE 14

| | BRM HiBit | BRM HiBit | BRMG1 HiBit | BRG1 HiBit |
|---|---|---|---|---|
| Compound | Degradation | Degradation | Degradation | Degradation |
| No. | IC₅₀ (nM) | Maximum (%) | IC₅₀ (nM) | Maximum (%) |
| 1 | +++ | B | + | C |
| 2 | +++ | A | + | C |
| 3 | ++ | A | + | C |
| 4 | + | C | + | C |
| 5 | +++ | A | + | C |
| 6 | ++ | B | + | C |
| 7 | +++ | A | + | C |
| 8 | ++++ | A | + | C |
| 9 | +++ | A | + | C |
| 10 | +++ | A | + | C |
| 11 | ++ | A | + | C |
| 12 | + | C | + | C |
| 13 | ++ | B | + | C |
| 14 | + | C | + | C |
| 15 | +++ | A | + | C |
| 16 | +++ | A | + | C |
| 17 | + | C | + | C |
| 18 | + | C | + | C |
| 19 | + | A | + | A |
| 20 | + | C | + | C |
| 21 | + | C | + | C |
| 22 | +++ | A | +++ | B |
| 23 | ++ | B | + | C |
| 24 | + | C | + | C |
| 25 | + | C | + | C |
| 26 | + | C | + | C |
| 27 | + | B | + | C |
| 28 | + | B | + | C |
| 29 | + | C | + | C |
| 30 | + | C | + | C |
| 31 | ++ | C | + | C |
| 32 | +++ | A | + | C |
| 33 | + | C | + | C |
| 34 | +++ | A | + | C |
| 35 | +++ | A | + | C |
| 36 | +++ | B | + | C |
| 37 | ++++ | A | + | C |
| 38 | ++ | A | + | C |
| 39 | + | C | + | C |
| 40 | + | C | + | C |
| 41 | +++ | A | ++ | B |
| 42 | ++ | A | + | C |
| 43 | + | C | + | C |
| 44 | ++ | B | + | C |
| 45 | ++ | B | + | C |
| 46 | + | C | + | C |
| 47 | ++ | A | + | C |
| 48 | + | C | + | C |
| 49 | ++++ | A | +++ | A |
| 50 | + | C | + | C |
| 51 | + | C | + | C |
| 52 | + | C | + | C |
| 53 | + | C | + | C |
| 54 | +++ | A | + | C |
| 55 | + | A | + | C |
| 56 | +++ | B | + | C |
| 57 | +++ | B | + | C |
| 58 | +++ | A | + | C |
| 59 | + | A | + | C |
| 60 | +++ | A | ++ | B |
| 61 | + | C | + | C |
| 62 | + | C | + | C |
| 63 | +++ | A | + | C |
| 64 | ++++ | A | ++++ | A |
| 65 | +++ | A | + | C |
| 66 | ++++ | A | +++ | A |
| 67 | ++ | A | + | C |
| 68 | ++ | B | + | C |
| 69 | + | C | + | C |
| 70 | + | C | + | C |
| 71 | +++ | A | + | C |
| 72 | ++++ | A | +++ | A |
| 73 | +++ | A | + | C |
| 74 | + | C | + | C |
| 75 | + | C | + | C |
| 76 | +++ | B | ++ | C |
| 77 | + | C | + | C |
| 78 | ++ | B | + | A |
| 79 | ++ | C | + | C |
| 80 | ++ | B | + | C |
| 81 | +++ | B | +++ | C |
| 82 | + | C | + | C |
| 83 | + | C | + | C |
| 84 | ++ | C | + | C |
| 85 | +++ | A | ++ | A |
| 86 | + | C | + | C |
| 87 | ++++ | A | + | C |
| 88 | +++ | A | + | C |
| 89 | + | C | + | C |
| 90 | +++ | A | + | C |
| 91 | + | C | + | C |
| 92 | + | C | + | B |
| 93 | +++ | A | + | C |
| 94 | ++++ | A | + | C |
| 95 | ++++ | A | + | C |
| 96 | ++++ | A | + | C |
| 97 | ++++ | A | + | C |
| 98 | ++++ | A | + | B |
| 99 | +++ | A | + | C |
| 100 | +++ | A | + | C |
| 101 | + | C | + | C |
| 102 | + | C | + | C |
| 103 | +++ | A | + | C |
| 104 | +++ | B | + | C |
| 105 | ++++ | A | + | C |
| 106 | + | C | + | B |
| 107 | +++ | A | + | C |
| 108 | +++ | A | + | C |
| 109 | + | C | + | C |
| 110 | + | B | + | C |
| 111 | + | C | + | C |
| 112 | ++ | B | + | C |
| 113 | ++++ | A | + | C |

“+” indicates inhibitory effect of ≥1000 nM;
“++” indicates inhibitory effect of ≥100 nM;
“+++” indicates inhibitory effect of ≥10 nM;
“++++” indicates inhibitory effect of <10 nM;
“NT” indicates not tested;
“A” indicates maximum degradation ≥ 75%;
“B” indicates maximum degradation ≥ 50%; and
“C” indicates maximum degradation < 50%.

### Other Embodiments

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A compound, or a pharmaceutically acceptable salt thereof, having the structure of Formula I or II: 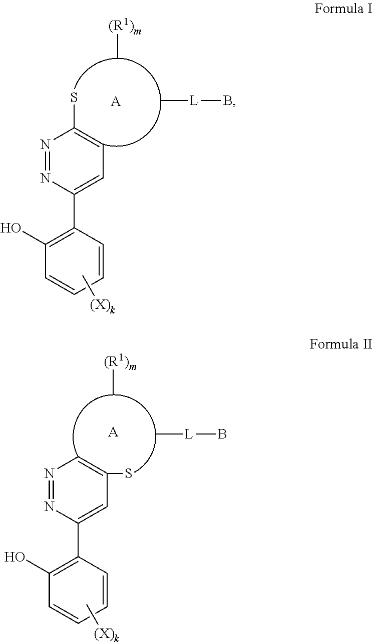
wherein
ring system A is a 5 to 9-membered heterocyclyl or heteroaryl;
m is 0, 1, 2, or 3;
k is 0, 1, or 2;
each R¹ is, independently, halo, optionally substituted C₁-C₆ alkyl, optionally substituted C₁-C₆ heteroalkyl, optionally substituted C₃-C₈ cycloalkyl, or optionally substituted C₂-C₉ heterocyclyl;
each X is, independently, halo;
L is a linker; and
B is a degradation moiety.

2. The compound of claim 1, or a pharmaceutically acceptable salt thereof, wherein the compound has the structure of Formula I-A or II-A: 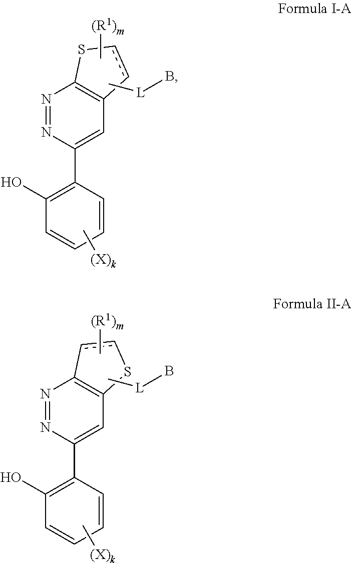
wherein the dashed bond represents a single or double bond.

3. The compound of claim 1, or a pharmaceutically acceptable salt thereof, wherein the compound has the structure of Formula I-B:

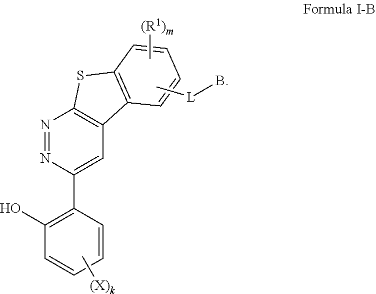

4. The compound of claim 1, or a pharmaceutically acceptable salt thereof, wherein the compound has the structure of Formula I-C: 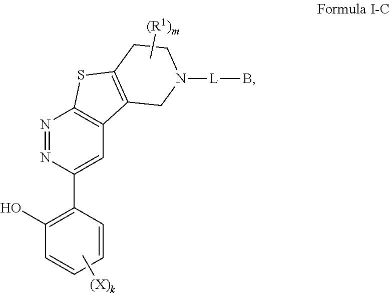
wherein each R¹ is, independently, optionally substituted C₁-C₆ alkyl.

5. The compound of claim 1, or a pharmaceutically acceptable salt thereof, wherein the compound has the structure of Formula I-D: 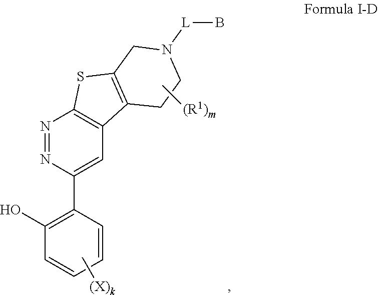
wherein each R¹ is, independently, optionally substituted C₁-C₆ alkyl.

6. The compound of claim 1, or a pharmaceutically acceptable salt thereof, wherein the compound has the structure of Formula I-E:

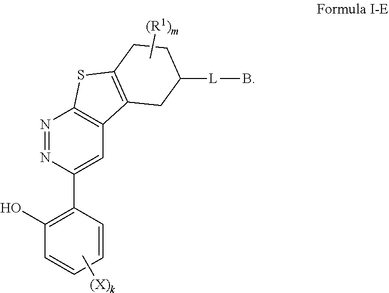

7. The compound of claim 1, or a pharmaceutically acceptable salt thereof, wherein the compound has the structure of Formula I-F:

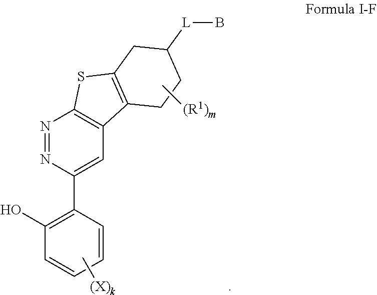

8. The compound of claim 1, or a pharmaceutically acceptable salt thereof, wherein m is 0.

9. The compound of claim 1, or a pharmaceutically acceptable salt thereof, wherein the compound has the structure of Formula I-G or II-G:

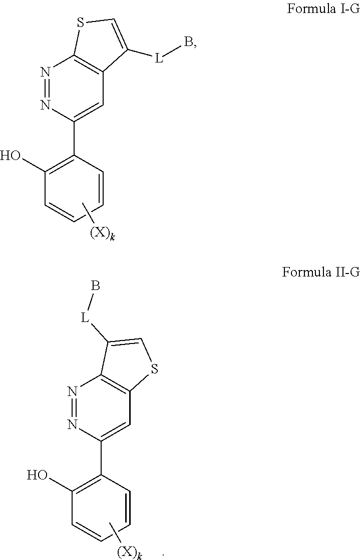

10. The compound of claim 1, or a pharmaceutically acceptable salt thereof, wherein the compound has the structure of Formula I-H or II-H:

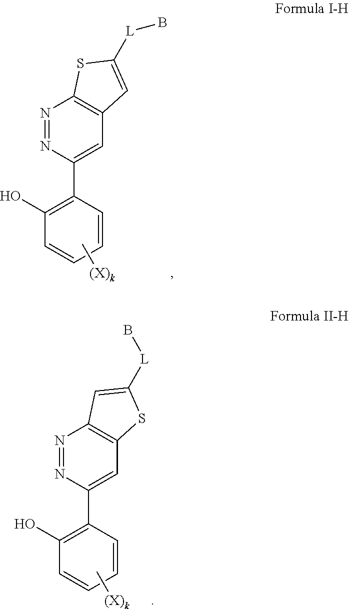

11. The compound of claim 1, or a pharmaceutically acceptable salt thereof, wherein the degradation moiety, B, has the structure of Formula A-1: 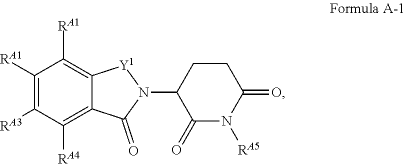
wherein
Y¹ is 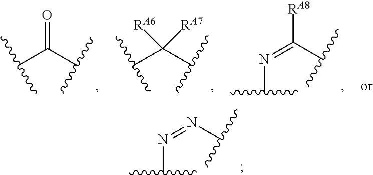
R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆ alkyl; or R^{A6} and R^{A7}, together with the carbon atom to which each is bound, combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form 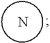
and 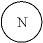
is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,
where one of R^{A1}, R^{A2}, R^{A3}, and R^{A4} is A², or 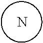
is substituted with A²; and
A² is a bond between the degradation moiety and the linker.

12. The compound of claim 11, or a pharmaceutically acceptable salt thereof, wherein R^{A5} is H or methyl.

13. (canceled)

14. The compound of claim 11, or a pharmaceutically acceptable salt thereof, wherein each of R^{A1}, R^{A2}, R^{A3}, and R^{A4} is, independently, H or A².

15.-18. (canceled)

19. The compound of claim 11, or a pharmaceutically acceptable salt thereof, wherein Y¹ is

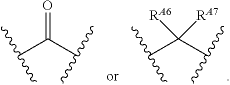

20. The compound of claim 19, or a pharmaceutically acceptable salt thereof, wherein R^{A6} is H.

21. The compound of claim 19, or a pharmaceutically acceptable salt thereof, wherein R^{A7} is H.

22. The compound of claim 11, or a pharmaceutically acceptable salt thereof, wherein Y¹ is

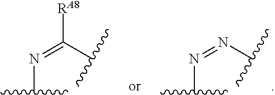

23. The compound of claim 22, or a pharmaceutically acceptable salt thereof, wherein R^{A8} is H or optionally substituted C₁-C₆ alkyl.

24. The compound of claim 23, or a pharmaceutically acceptable salt thereof, wherein R^{A8} is H or methyl.

25. (canceled)

26. The compound of claim 11, or a pharmaceutically acceptable salt thereof, wherein the degradation moiety comprises the structure of Formula A2:

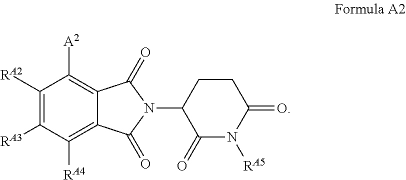

27. (canceled)

28. The compound of claim 11, or a pharmaceutically acceptable salt thereof, wherein the degradation moiety comprises the structure of Formula A4:

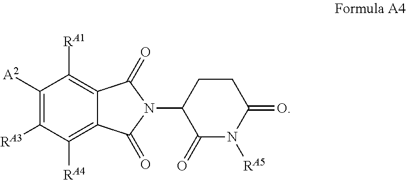

29. (canceled)

30. The compound of claim 11, or a pharmaceutically acceptable salt thereof, wherein the degradation moiety comprises the structure of Formula A5:

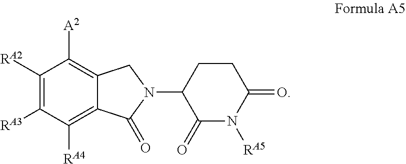

31. The compound of claim 11, or a pharmaceutically acceptable salt thereof, wherein the degradation moiety comprises the structure of Formula A6:

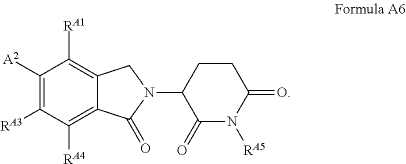

32. The compound of claim 11, or a pharmaceutically acceptable salt thereof, wherein the degradation moiety comprises the structure of Formula A8:

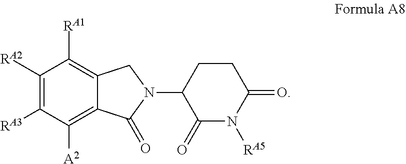

33. The compound of claim 11, or a pharmaceutically acceptable salt thereof, wherein the degradation moiety comprises the structure of Formula A10:

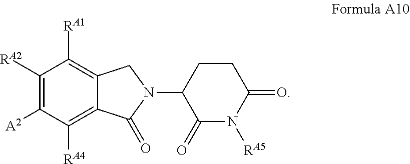

34. (canceled)

35. (canceled)

36. The compound of claim 1, or a pharmaceutically acceptable salt thereof, wherein the degradation moiety has the structure of Formula C′: 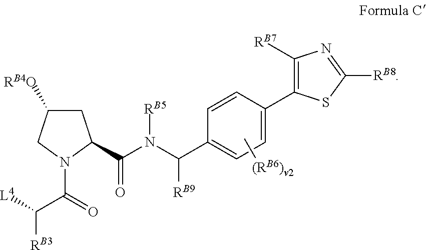
wherein
L⁴ is —N(R^{B1})(R^{B2}), 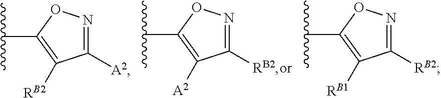
R^{B1} is H, A², optionally substituted C₁-C₆ alkyl, or optionally substituted C₁-C₆ heteroalkyl; R^{B2} is H, optionally substituted C₁-C₆ alkyl, optionally substituted C₂-C₉ heterocyclyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
wherein one and only one of R^{B1}, R^{B3}, and R^{B6} is A²,
or a pharmaceutically acceptable salt thereof.

37. The compound of claim 1, or a pharmaceutically acceptable salt thereof, wherein the degradation moiety has the structure of Formula C: 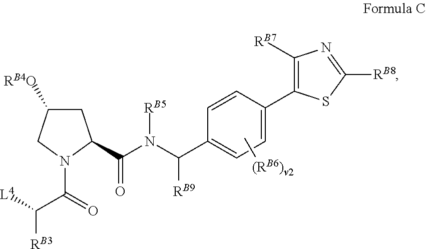
wherein
L⁴ is —N(R^{B1})(R^{B2}), 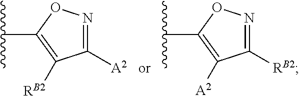
R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
wherein one and only one of R^{B1}, R^{B3}, and R^{B6} is A²,
or a pharmaceutically acceptable salt thereof.

38. The compound of claim 36, or a pharmaceutically acceptable salt thereof, wherein the degradation moiety has the structure of Formula C1:

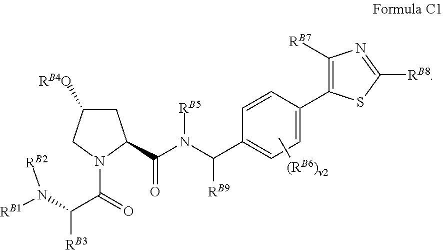

39.-41. (canceled)

42. The compound of claim 36, or a pharmaceutically acceptable salt thereof, wherein the degradation moiety has the structure of Formula C2:

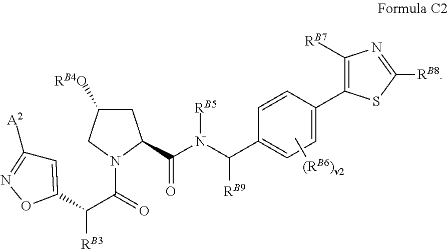

43. The compound of claim 36, or a pharmaceutically acceptable salt thereof, wherein R^{B9} is optionally substituted C₁-C₆ alkyl.

44. The compound of claim 43, or a pharmaceutically acceptable salt thereof, wherein R^{B9} is methyl.

45. The compound of claim 36, or a pharmaceutically acceptable salt thereof, wherein R^{B9} is bonded to (S)-stereogenic center.

46. (canceled)

47. The compound of claim 36, or a pharmaceutically acceptable salt thereof, wherein the linker has the structure of Formula III:
A¹-(B¹)_{f}—(C¹)_{g}—(B²)ₕ-(D)-(B³)ᵢ—(C²)ⱼ—(B⁴)ₖ-A², Formula III
or a pharmaceutically acceptable salt thereof,
wherein
A¹ is a bond between the linker and ring system A;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₂-C₆ heterocyclyl, optionally substituted C₂-C₉ heteroaryl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

48. The compound of claim 26, or a pharmaceutically acceptable salt thereof, wherein the linker has the structure of Formula III:
A¹-(B¹)_{f}—(C¹)_{g}—(B²)ₕ-(D)-(B³)ᵢ—(C²)ⱼ—(B⁴)ₖ-A², Formula III
or a pharmaceutically acceptable salt thereof,
wherein
A¹ is a bond between the linker and ring system A;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₂-C₆ heterocyclyl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

49. The compound of claim 47, or a pharmaceutically acceptable salt thereof, wherein each of B¹, B², B³, and B⁴ is, independently, optionally substituted C₁-C₂ alkyl, optionally substituted C₁-C₃ heteroalkyl, optionally substituted C₂-C₆ heterocyclyl, or NR^{N}.

50. The compound of claim 47, or a pharmaceutically acceptable salt thereof, wherein each R^{N} is, independently, H or optionally substituted C₁-C₄ alkyl.

51. (canceled)

52. The compound of claim 47, or a pharmaceutically acceptable salt thereof, wherein each of B¹ and B⁴ is, independently,

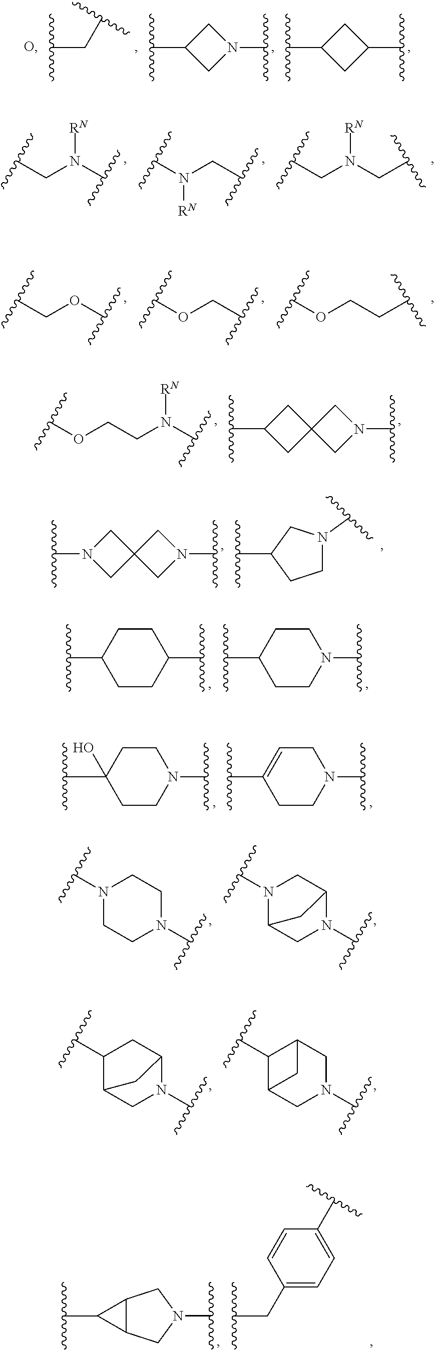

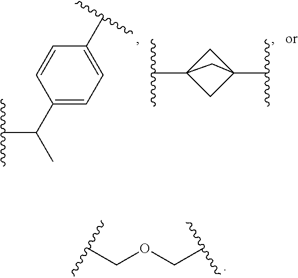

53. (canceled)

54. The compound of claim 52, or a pharmaceutically acceptable salt thereof, wherein B¹ is

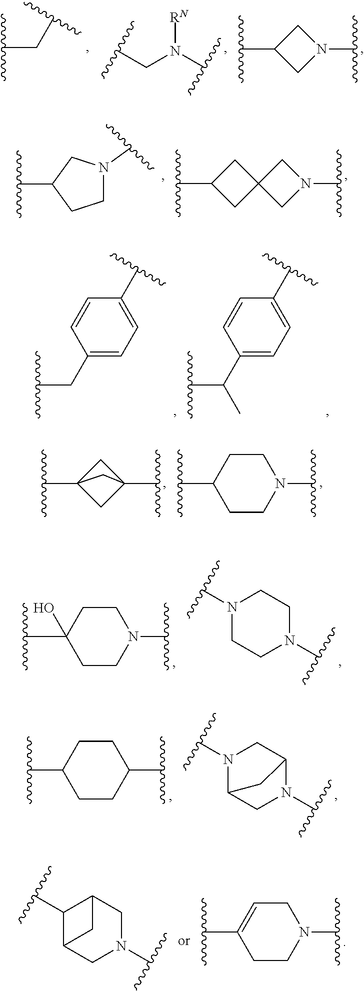

55. (canceled)

56. The compound of any one of claim 52, or a pharmaceutically acceptable salt thereof, wherein B⁴ is O,

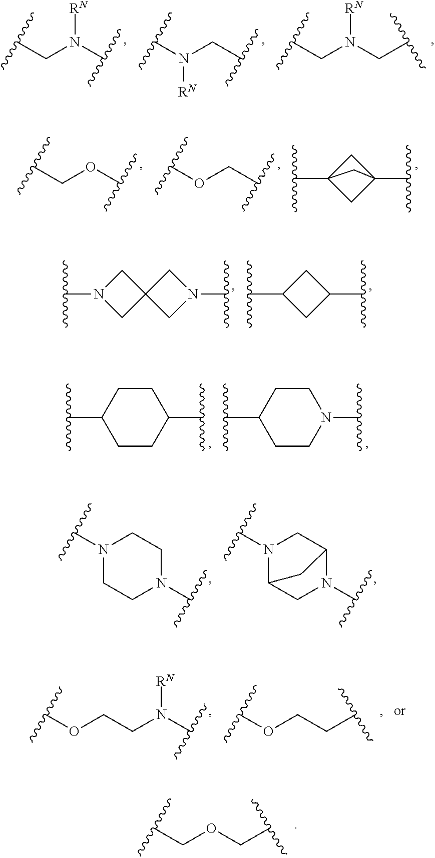

57. (canceled)

58. The compound of claim 47, or a pharmaceutically acceptable salt thereof, wherein each of C¹ and C² is

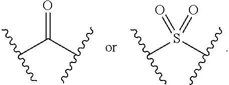

59. (canceled)

60. (canceled)

61. The compound of claim 47, or a pharmaceutically acceptable salt thereof, wherein B² is optionally substituted C₁-C₄ alkyl optionally substituted C₂-C₆ heterocyclyl or optionally substituted C₂-C₉ heteroaryl.

62. (canceled)

63. The compound of claim 47, or a pharmaceutically acceptable salt thereof, wherein B² is

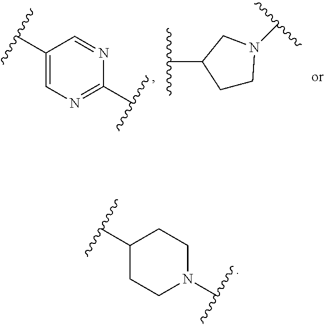 [SMILES: *c1cnc(*)nc1 ; *C1CCN(*)C1 ; *C1CCN(*)CC1]

64. (canceled)

65. (canceled)

66. The compound of claim 47, or a pharmaceutically acceptable salt thereof, wherein B³ is optionally substituted C₃-C₁₀ cycloalkyl.

67. The compound of claim 66, or a pharmaceutically acceptable salt thereof, wherein B³ is

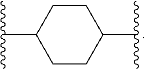 [SMILES: *C1CCC(*)CC1]

68. The compound of claim 47, or a pharmaceutically acceptable salt thereof, wherein D is optionally substituted C₁-C₁₀ alkyl.

69. The compound of claim 47, or a pharmaceutically acceptable salt thereof, wherein f is 1.

70. The compound of claim 47, or a pharmaceutically acceptable salt thereof, wherein g is 0.

71. The compound of claim 47, or a pharmaceutically acceptable salt thereof, wherein g is 1.

72. The compound of claim 47, or a pharmaceutically acceptable salt thereof, wherein h is 0.

73. The compound of claim 47, or a pharmaceutically acceptable salt thereof, wherein h is 1.

74. The compound of claim 47, or a pharmaceutically acceptable salt thereof, wherein i is 0.

75. The compound of claim 47, or a pharmaceutically acceptable salt thereof, wherein i is 1.

76. The compound of claim 47, or a pharmaceutically acceptable salt thereof, wherein j is 0.

77. The compound of claim 47, or a pharmaceutically acceptable salt thereof, wherein j is 1.

78. The compound of claim 47, or a pharmaceutically acceptable salt thereof, wherein k is 0.

79. The compound of claim 47, or a pharmaceutically acceptable salt thereof, wherein k is 1.

80. The compound of claim 47, or a pharmaceutically acceptable salt thereof, wherein the linker has the structure of

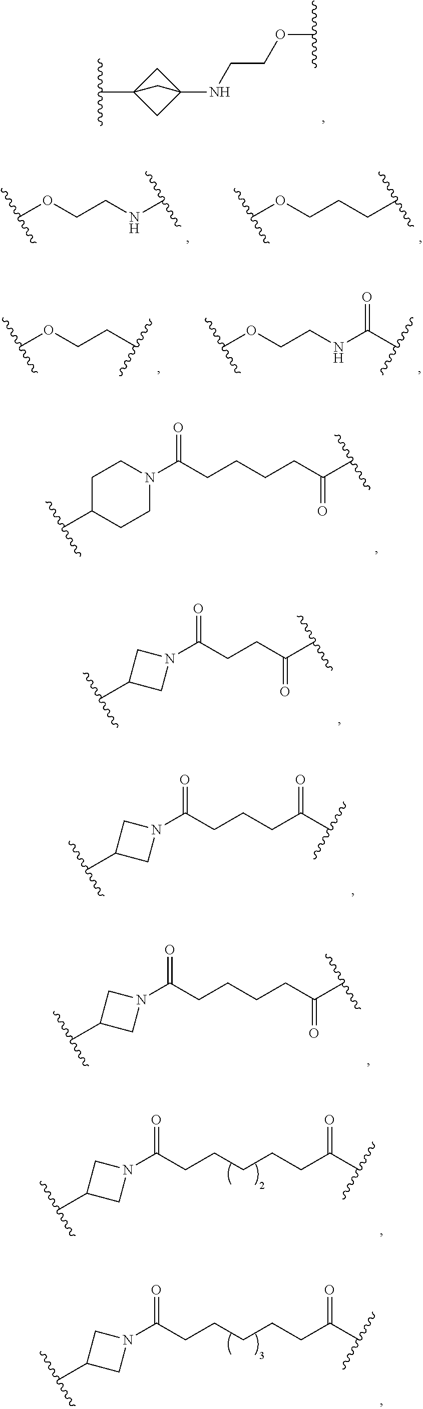

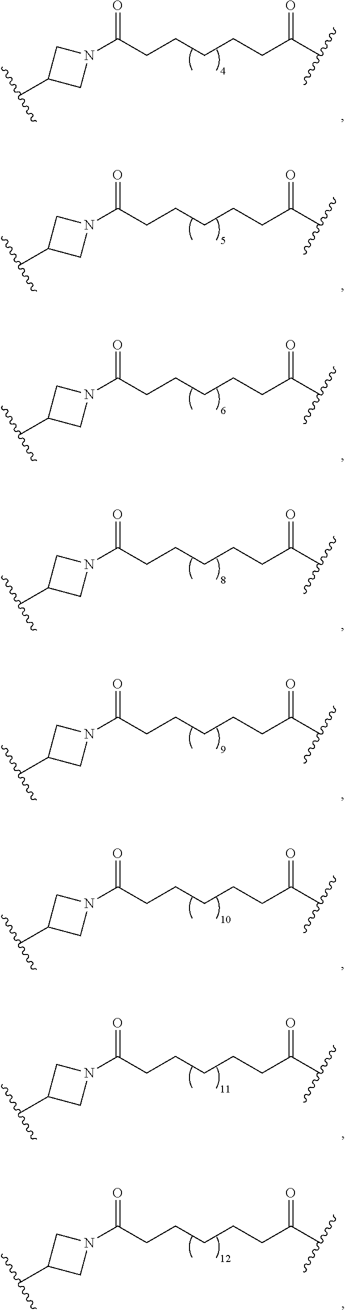

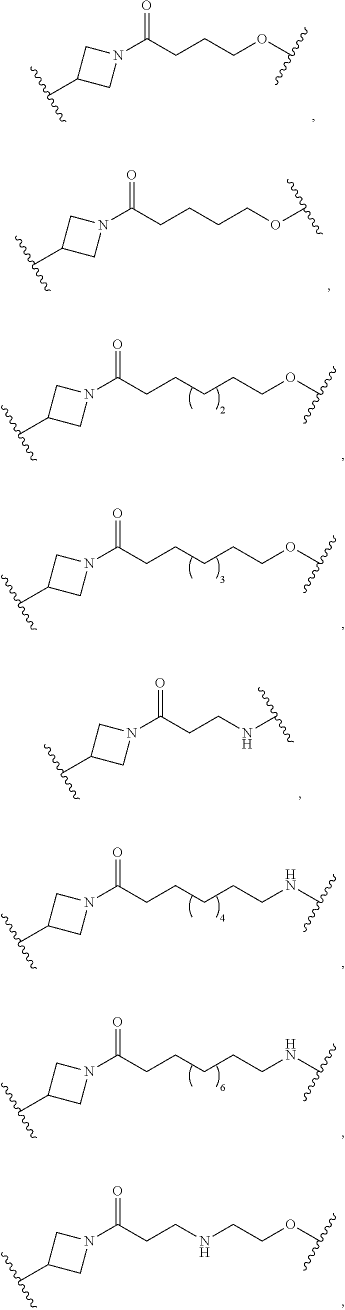

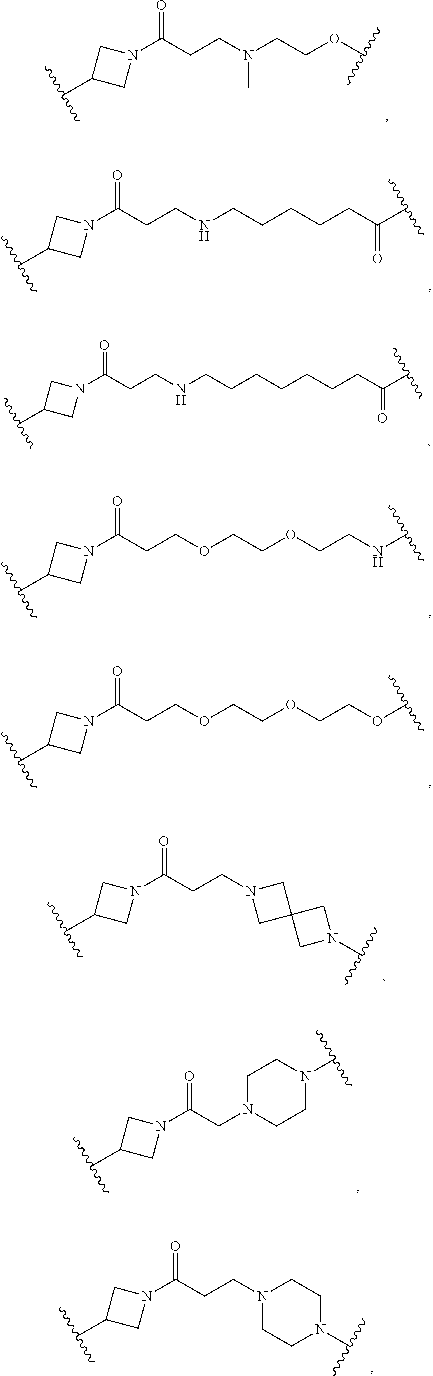

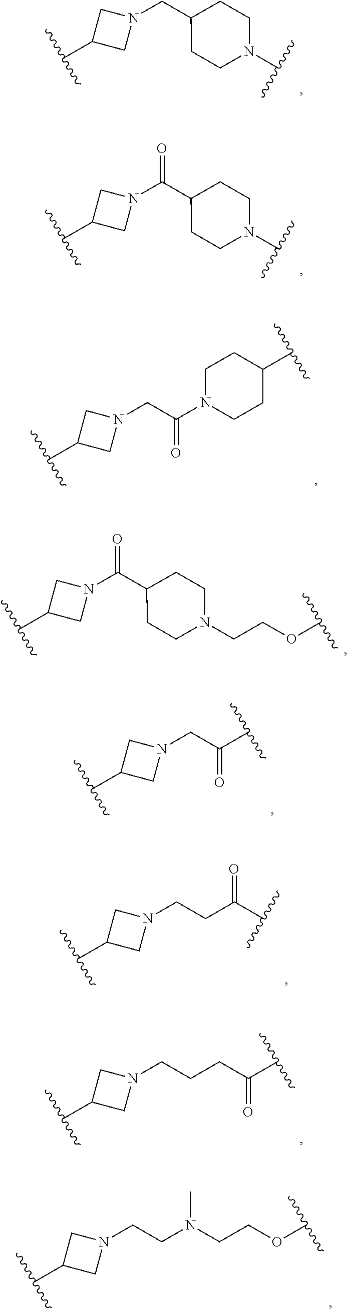

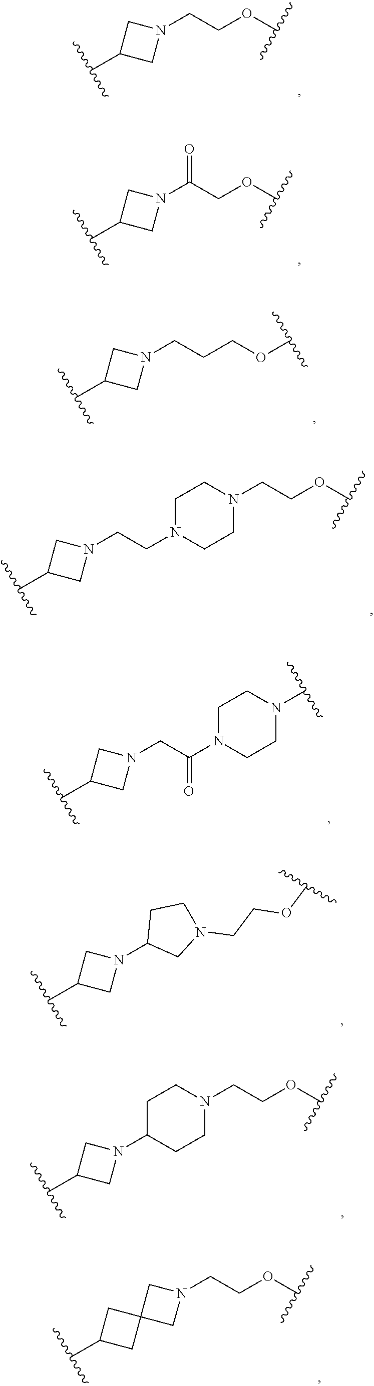

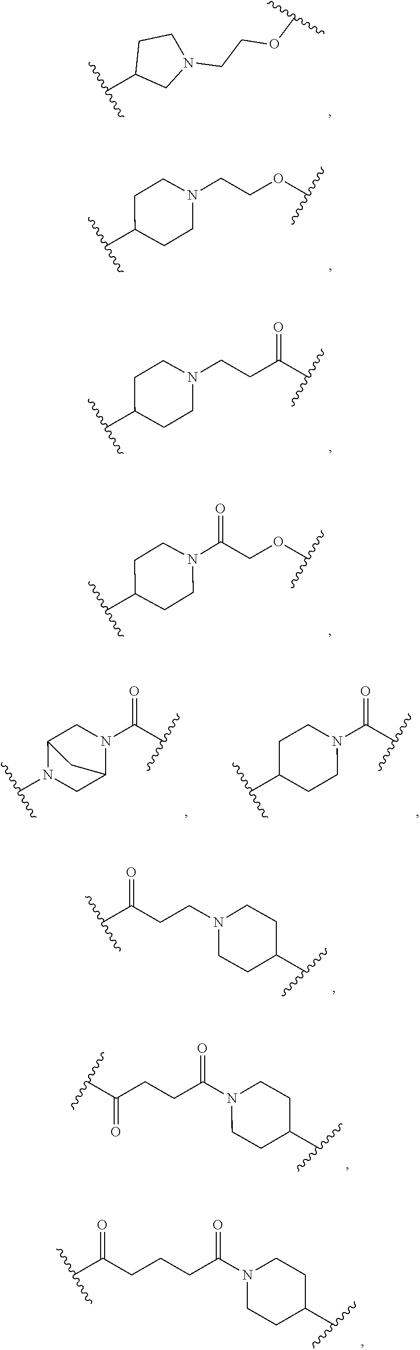

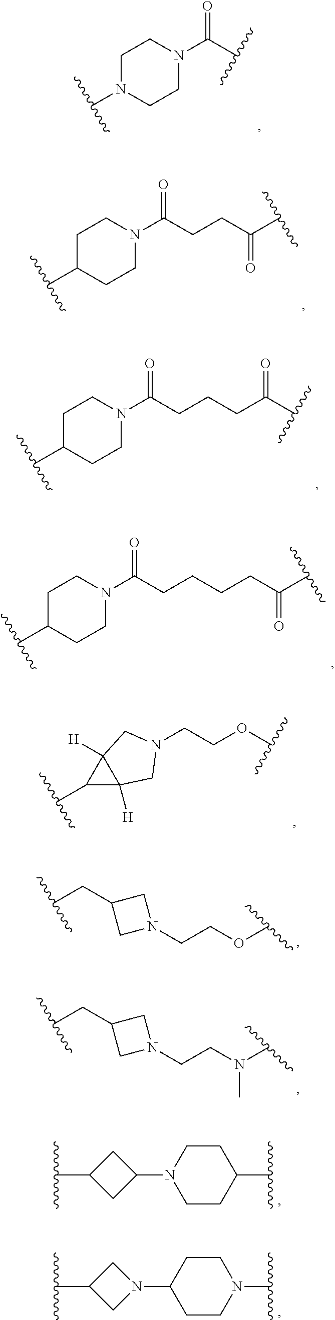

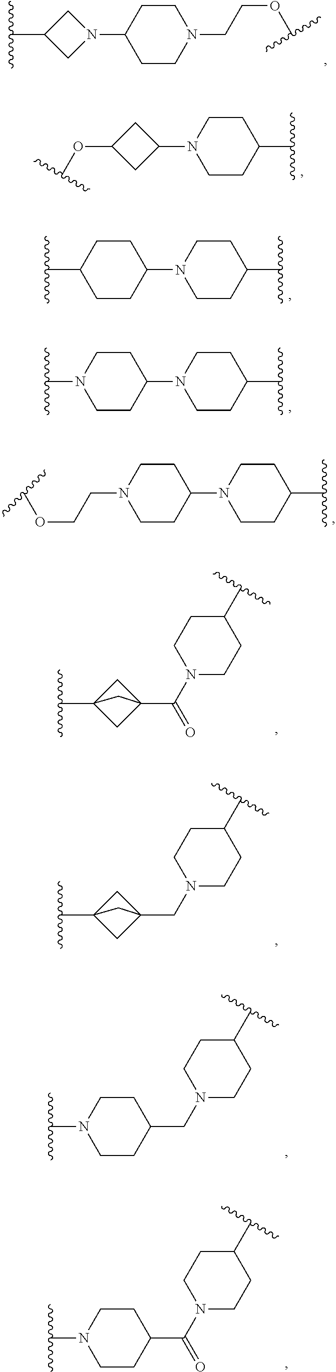

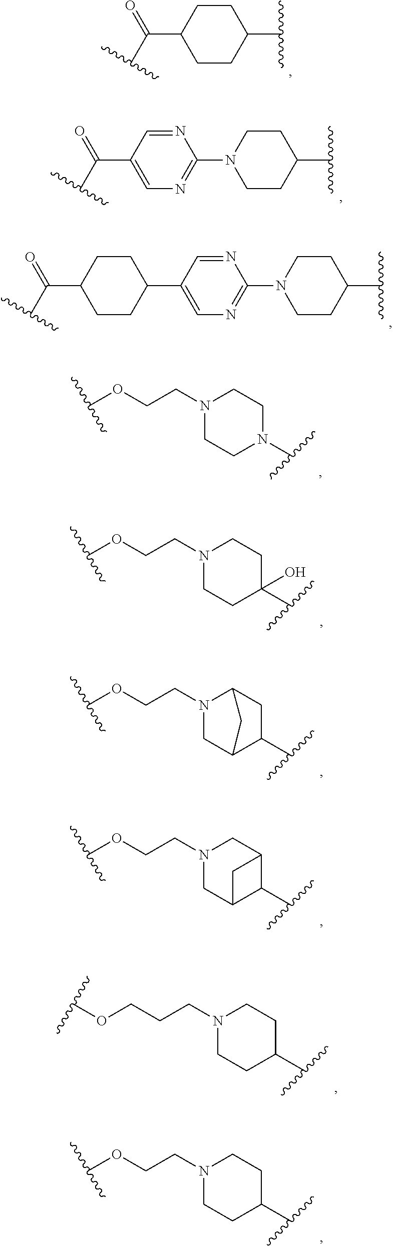

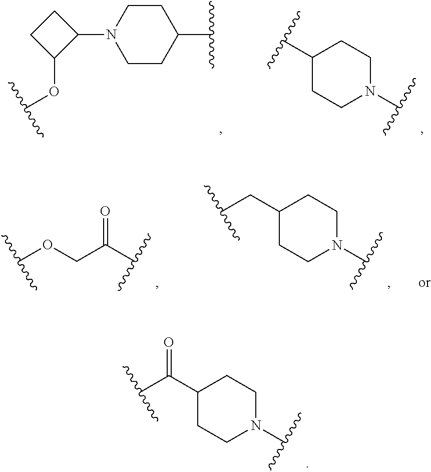 [SMILES: *OC1CCC1N1CCC(*)CC1 ; *C1CCN(*)CC1 ; *OCC(*)=O ; *CC1CCN(*)CC1 ; *C(=O)C1CCN(*)CC1]

81.-83. (canceled)

84. The compound of claim 47, or a pharmaceutically acceptable salt thereof, wherein the linker has the structure of

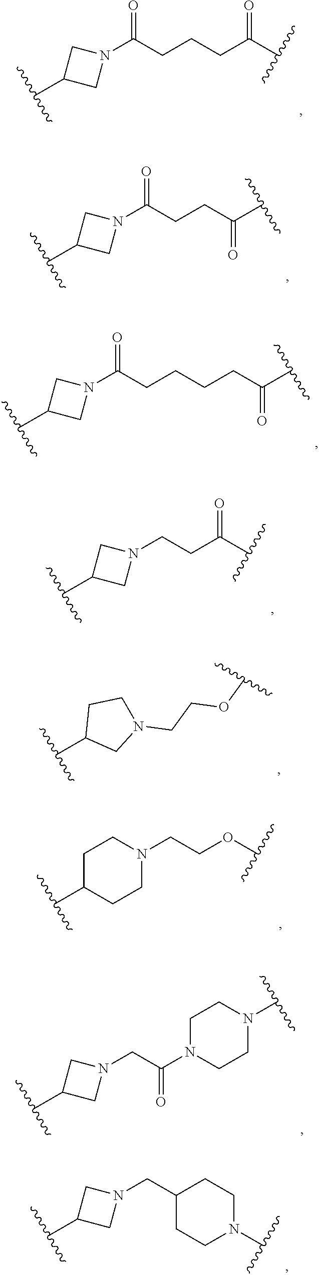

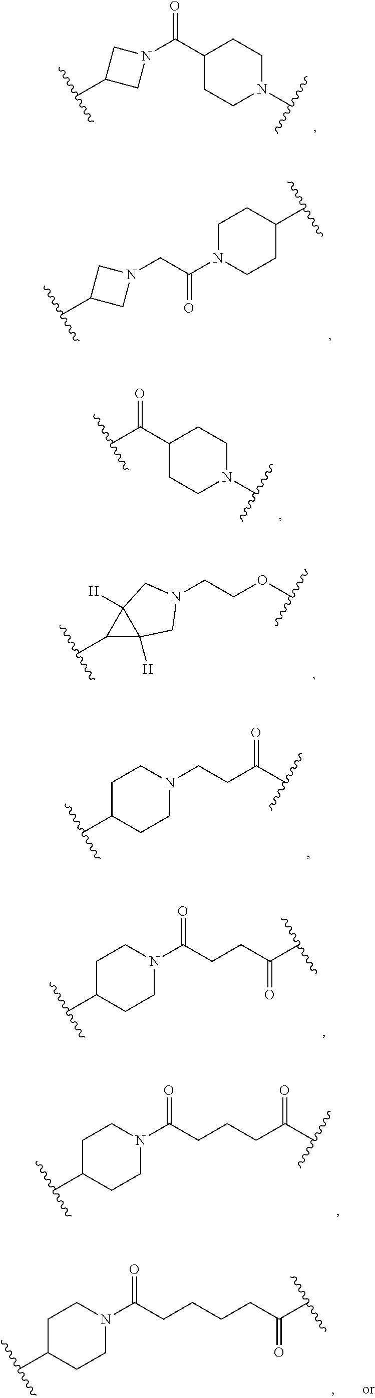

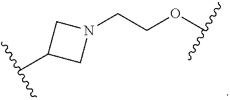 [SMILES: *OCCN1CC(*)C1]

85.-93. (canceled)

94. A pharmaceutical composition comprising a compound of claim 1, and a pharmaceutically acceptable excipient.

95.-104. (canceled)

105. A method of treating cancer in a subject in need thereof, the method comprising administering to the subject an effective amount of a compound of claim 1.

106. The method of claim 105, wherein the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

107. The method of claim 105, wherein the cancer is non-small cell lung cancer, colorectal cancer, bladder cancer, cancer of unknown primary, glioma, breast cancer, melanoma, non-melanoma skin cancer, endometrial cancer, or penile cancer.

108. The method of claim 105, wherein the cancer is non-small cell lung cancer.

109. The method of claim 105, wherein the cancer is soft tissue sarcoma.

110. A method of treating a cancer selected from the group consisting of melanoma, prostate cancer, breast cancer, bone cancer, renal cell carcinoma, and a hematologic cancer in a subject in need thereof, the method comprising administering to the subject an effective amount of a compound of claim 1.

111.-116. (canceled)

117. The method of claim 110, wherein the method further comprises administering to the subject or contacting the cell with an anticancer therapy.

118.-142. (canceled)
